(19) United States
(12) Patent Application Publication
(10) Pub. No.: US 2025/0275959 A1
(43) Pub. Date: Sep. 4, 2025

(54) COMPOUNDS AND METHODS OF THEIR USE

(71) Applicant: LEXICON PHARMACEUTICALS, INC., The Woodlands, TX (US)

(72) Inventors: Thomas Bernard Levy, Harlow (GB); Alan Joseph Main, The Woodlands, TX (US); Darren Orton, The Woodlands, TX (US); Thomas David Pallin, Harlow (GB); Adele Elisa Pasqua, Harlow (GB); Pascal Pierre Alexandre Savy, Harlow (GB); James Robin Smith, Harlow (GB)

(21) Appl. No.: 19/052,650

(22) Filed: Feb. 13, 2025

Related U.S. Application Data
(60) Provisional application No. 63/554,467, filed on Feb. 16, 2024.

(51) Int. Cl. A61K 31/496; A61K 31/4439; A61K 31/444; A61K 31/4545; A61P 3/04; C07D 213/63; C07D 401/12; C07D 401/14; C07D 405/12; C07D 471/08

(52) U.S. Cl. CPC A61K 31/496; A61K 31/4439; A61K 31/444; A61K 31/4545; A61P 3/04; C07D 213/63; C07D 401/12; C07D 401/14; C07D 405/12; C07D 471/08

(57) ABSTRACT

Compounds useful as ACSL5 inhibitors, methods of their preparation and use, and pharmaceutical compositions comprising them are disclosed. Particular compounds are of the formula:

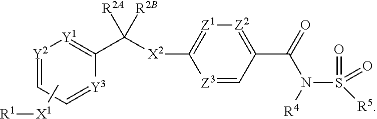

This application claims priority to U.S. provisional patent application No. 63/554,467, filed Feb. 16, 2024, the entirety of which is incorporated herein by reference.

### 1. BACKGROUND

Aberrations in fatty acid trafficking and metabolism have been associated with obesity, non-alcoholic fatty liver disease (NAFLD), and type 2 diabetes. Bowman, T. A., et al., “Acyl CoA synthetase 5 (ACSL5) ablation in mice increases energy expenditure and insulin sensitivity and delays fat absorption”, Mol. Metab., 2016 Jan 11; 5(3):210-220. Critical to that metabolism are acyl-CoA synthetases.

Acyl-CoA synthases have been categorized as very long-chain acyl-CoA synthases, long-chain acyl-CoA synthases (ACSL5), and medium and short-chain acyl-CoA synthases, according to the carbon chain length of fatty acids upon which they act. Luo, Q., et al., “Role of ACSL5 in fatty acid metabolism”, Heliyon, 2023 9:e13316. Five isoforms of ACSL5, which mainly catalyze the reaction of C₁₂₋₂₀ fatty acids, have been identified and accorded the numbers 1, 3, 4, 5, and 6. Each has a unique tissue expression, subcellular localization, and substrate preference. Id.

Acyl-CoA synthetase long-chain family member 5 (ACSL5) is widely expressed in mammalian liver, small intestine, fat, spleen, uterus, lung, and skeletal muscle. Id. The enzyme catalyzes the formation of acyl-CoAs using long-chain fatty acids in the cytoplasm. These are then used to synthesize complex lipids and other products in the endoplasmic reticulum or enter mitochondria for P-oxidation. Fatty acids catalyzed by ACSL5 include palmitic acid, palmitoleic acid, oleic acid, and linoleic acid. Id. at e13317.

Studies suggest that the enzyme is involved in glucose and lipid metabolism, and may play an essential role in metabolic disorders. For example, ACSL5−/− mice have increased hepatic and serum fibroblast growth factor 21 levels, reduced adiposity, improved insulin sensitivity, increased energy expenditure, and delayed triglyceride absorption. Bowman, supra, at 210.

It has also been suggested that ACSL dysregulation plays a role in some cancers. See, e.g., Sebastiano, M. R., and Konstantinidou, G., Int. J. Mol. Sci., 2019, 20:3624. For example, the expression of ACSL5 is reportedly higher in bone marrow cells obtained from acute myeloid leukemia (AML) patients than in those from healthy donors. Ye, et al., “ACSL5, a prognostic factor in acute myeloid leukemia, modulates the activity of Wnt/0-catenin signaling by palmitoylation modification”, Front Med., 2023 Aug; 17(4):685-698. Consequently, inhibition of ACSL5 may be effective in treating a variety of metabolic and oncologic diseases and disorders.

### 2. SUMMARY

This invention is directed to compounds useful as ACSL5 inhibitors, methods of their preparation and use, and pharmaceutical compositions comprising them.

One embodiment of the invention is directed to compounds of formula I:

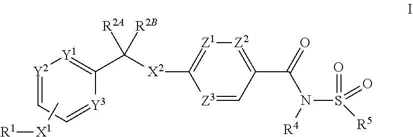

and pharmaceutically acceptable salts thereof, wherein:
X¹ and X² is independently O or CHR⁶;
each of Y¹, Y², and Y³ is independently N, CR³, or C—X¹—R¹, provided that only one of Y¹, Y², and Y³ may be attached to X¹;
each of Z¹, Z², and Z³ is independently N or CR³;
each R¹ is independently optionally substituted C₁₋₆ hydrocarbyl or 2-9 membered heterocarbyl, which optional substitution is with one or more of halo, R^{1A}, or OR^{1A};
each R^{1A} is independently optionally substituted C₁₋₆ hydrocarbyl, which optional substitution is with one or more of halo;
each of R^{2A} and R^{2B} is independently hydrogen or C₁₋₆ alkyl optionally substituted with one or more halo, or R^{2A} and R^{2B} are taken together to form a C₃₋₅ cycloalkyl or 3-5 membered cycloheteroalkyl; each R³ is independently hydrogen, halo, or C₁₋₆ alkyl optionally substituted with one or more halo;
R⁴ is hydrogen or C₁₋₆ alkyl;
R⁵ is N(R^{5A})₂, NHR^{5A}, or optionally substituted C₁₋₆ hydrocarbyl or 2-9 membered heterocarbyl, which optional substitution is with one or more of halo, hydroxyl, nitrile, RA, OR^{5A}, N(R^{5A})₂, NHR^{5A}, or NH₂;
each R^{5A} is independently optionally substituted C₁₋₆ hydrocarbyl or 2-9 membered heterocarbyl, which optional substitution is with one or more of halo, hydroxyl, nitrile, R^{5B}, OR^{5B}, N(R^{5B})₂, NHR^{5B}, or NH₂;
each R^{5B} is independently optionally substituted C₁₋₈ hydrocarbyl or 2-9 membered heterocarbyl, 25 which optional substitution is with one or more of halo, hydroxyl, nitrile, R^{C}, OR^{5C}, N(R^{5C})₂, NHR^{5C}, or NH₂;
each R^{5C} is independently optionally substituted C₁₋₈ hydrocarbyl or 2-9 membered heterocarbyl, which optional substitution is with one or more of halo, hydroxyl, nitrile, or NH₂; and each R⁶ is independently H or C₁₋₆ alkyl.

Another embodiment is directed to pharmaceutical compositions comprising a compound disclosed herein (e.g., a compound of Formula I).

Another embodiment is directed to methods of preparing compounds disclosed herein.

Another embodiment is directed to methods of treating or managing a disease or disorder mediated by ACSL5 function, which comprise administering to a patient in need thereof a compound of the invention. Certain embodiments are directed to methods of treating metabolic diseases or disorders, such as metabolic dysfunction-associated steatohepatitis (MASH), metabolic syndrome, non-alcoholic fatty liver disease (NAFLD), obesity, and type 2 diabetes. Others are directed to methods of increasing insulin sensitivity. Others are directed to methods of treating a cancer characterized by the overexpression of ACSL5, such as acute myeloid leukemia (AML), colorectal cancer, and breast cancer.

### 3. BRIEF DESCRIPTION OF THE FIGURES

Aspects of some embodiments of the invention may be understood from the attached figures. As used herein, the terms “Figure” and “FIG.” are used interchangeably and their case (e.g., lower, upper) is irrelevant. Portions of a figure X that are identified with a letter (e.g., A, B) may be referred to as FIG. XA and FIG. XB even if not explicitly identified as “FIG. XA” or “FIG. XB” on the figure. Thus, if FIG. 1 has parts identified as A and B, those parts may be referred to as “FIG. 1A” and “FIG. 1B” or “FIG. 1A” and “FIG. 1B”.

FIG. 1A shows the percent reduction in the mean body weights of diet-induced obesity (DIO) mice treated with Compound A versus those in the control group as a function of time.

FIG. 1B shows the percent reduction in the mean body weight of DIO mice in the control group and those in the two group treated with Compound A versus baseline as a function of time.

FIG. 2A shows the percent reduction in the mean body weights of DIO mice treated with Compounds B-D versus those of the control group as a function of time.

FIG. 2B shows the percent reduction in the mean body weight of DIO mice in the control group and those in the group treated with Compounds B-D versus baseline as a function of time.

### 4. DETAILED DESCRIPTION

This invention is directed to compounds useful as ACSL5 inhibitors, methods of their preparation and use, and pharmaceutical compositions comprising them. Aspects of the invention are described using terms well known in the art.

### 4.1. DEFINITIONS

Unless otherwise indicated, the term “about” means±10% of the indicated range.

When used herein, the term “alkenyl” is accorded its conventional meaning. Examples of alkenyl moieties include straight-chain and branched C₂₋₂₀, C₂₋₁₂ and C₂₋₆ alkenyl such as vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, 3-nonenyl, 1-decenyl, 2-decenyl and 3-decenyl.

The term “alkyl” is accorded its conventional meaning. Examples of alkyl moieties include straight-chain and branched C₁₋₂₀ alkyl, C₁₋₁₂ alkyl, C₁₋₆ alkyl, C₁₋₄ alkyl, and C₁₋₃ alkyl, such as methyl, ethyl, propyl, isopropyl, n-butyl, t-butyl, isobutyl, pentyl, hexyl, isohexyl, heptyl, 4,4-dimethylpentyl, octyl, 2,2,4-trimethylpentyl, nonyl, decyl, undecyl, and dodecyl.

The term “alkynyl” is accorded its conventional meaning. Examples of alkynyl moieties include straight-chain and branched C₂₋₂₀, C₂₋₁₂ and C₂₋₆ alkynyl, such as ethynyl and 2-propynyl (propargyl).

The term “aryl” refers to a single all-carbon-backbone aromatic ring or a multiple condensed all-carbon-backbone ring system wherein at least one of the rings is aromatic. Examples include C₆₋₂₀, C₆₋₁₄, C₆₋₁₂, and C₂₋₁₀ rings and multiple condensed carbon ring systems (e.g., ring systems comprising 2, 3 or 4 rings) having 9 to 20 carbon atoms in which at least one ring is aromatic and wherein the other rings may be aromatic or not aromatic. The rings of multiple condensed ring systems may be connected to each other via fused, spiro, or bridged bonds when valency allows. Examples of aryl moieties include anthracenyl, azulenyl, fluorenyl, indanyl, indenyl, naphthyl, phenyl, phenanthrenyl, and 1, 2, 3, 4-tetrahydronaphthyl.

The term “cycloalkyl” refers to a saturated or partially unsaturated all carbon-backbone ring having 3 to 20 carbon atoms (e.g., C₃₋₂₀, C₃₋₁₅, C₃₋₇, C₄₋₆ and C₆ cycloalkyl). Examples include multicyclic carbocyles such as bicyclo[3.1.0]hexane and bicyclo[2.1.1]hexane, and polycyclic carbocycles, such as tricyclic and tetracyclic carbocycles. The rings of multiple condensed ring systems may be connected to each other via fused, spiro, and bridged bonds when valency allows. For example, multicyclic carbocycles may be connected to each other via a single carbon atom to form a spiro connection (e.g., spiropentane, spiro[4,5]decane), via two adjacent carbon atoms to form a fused connection (e.g., decahydronaphthalene, norsabinane, norcarane), or via two non-adjacent carbon atoms to form a bridged connection (e.g., norbornane, bicyclo[2.2.2]octane). Examples of cycloalkyls include cyclopropyl, cyclobutyl, cyclopentyl, cyclohexyl, bicyclo[2.2.1]heptane, pinane, adamantane.

The term “cycloheteroalkyl” refers to a cyclic heteroalkyl moiety, defined below.

The term “halo” encompass fluoro, chloro, bromo, and iodo.

The term “heterocarbyl” refers to a moiety having a backbone made up of one or more carbon atoms and one or more heteroatoms. Particular heteroatoms are nitrogen, oxygen and sulfur. A heterocarbyl moiety may be thought of as a hydrocarbyl moiety wherein at least one carbon atom, CH, CH₂, or CH₃ group is replaced with one or more heteroatoms and the requisite number of hydrogen atoms to satisfy valences. Heterocarbyl moieties may be nonaromatic, aromatic, partially aromatic, linear, branched, cyclic, or a combination thereof. Examples of heterocarbyl include 2-20, 2-12,2-8, 2-6 and 2-4 membered heterocarbyl moieties, wherein the number range refers to the sum total of carbon, nitrogen, oxygen, and/or sulfur atoms in the moiety's backbone. The term “2-12 membered heterocarbyl” thus refers to a heterocarbyl moiety having a total of 2-12 carbon, nitrogen, oxygen, and/or sulfur atoms. Examples of heterocarbyl moieties include straight chain and branched heteroalkyl, heteroalkenyl, and heteroalkynyl, as well as heterocycle and heteroaryl.

The term “heteroalkyl” refers to a saturated or partially unsaturated moiety having 3- to 20-atom backbone of carbon and at least one heteroatom (e.g., O, N, S). Examples of heteroalkyl moieties include 2-8-membered, 2-6-membered, and 2-4-membered heteroalkyl moieties. Particular examples include alkoxyl, acyl (e.g., formyl, acetyl, benzoyl), alkylamino (e.g., di-(C₁₋₃-alkyl)amino), arylamino, aryloxime, carbamates, carbamides, alkylcarbonyl, arylcarbonyl, aminocarbonyl, alkylaminocarbonyl, alkylsulfanyl, arylsulfanyl, alkylsulfinyl, arylsulfinyl, alkylsulfonyl, arylsulfonyl, alkylsulfonylamino, and arylsulfonylamino.

The term “heteroaryl” encompasses a single aromatic ring that has at least one atom other than carbon in the ring, wherein the atom is selected from the group consisting of oxygen, nitrogen and sulfur (e.g., single aromatic rings of from 1 to 6 carbon atoms and 1-4 heteroatoms). The term also encompasses multiple condensed ring systems that have at least one such aromatic ring. Examples include acridinyl, benzimidazolyl, benzofuranyl, benzoisothiazolyl, benzoisoxazolyl, benzoquinazolinyl, benzothiazolyl, benzoxazolyl, furyl, imidazolyl, indazolyl, indolyl, isoquinolyl, isothiazolyl, isoxazolyl, oxadiazolyl, oxazolyl, phthalazinyl, pyrazinyl, pyrazolyl, pyridazinyl, pyridyl, pyrimidinyl, pyrimidyl, pyrrolyl, quinolyl, quinoxalyl, quinazolinyl, quinolinyl, thienyl, tetrazolyl, thiadiazolyl, thiazolyl, triazinyl, and triazolyl.

The term “hydrocarbyl” refers to a moiety having an all-carbon backbone and consisting of carbon and hydrogen atoms. Examples of hydrocarbyl groups include those having 1-20, 1-12, 1-6, and 1-4 carbon atoms (referred to as C₁₋₂₀ hydrocarbyl, C₁₋₁₂ hydrocarbyl, C₁₋₆ hydrocarbyl, and C₁₋₄ hydrocarbyl, respectively). Hydrocarbyl moieties may be aliphatic, aromatic, partially aromatic, linear, branched, cyclic, or a combination thereof. Examples include alkyl, alkenyl, alkynyl, aryl, benzyl, cycloalkyl, cycloalkenyl, cycloalkylalkyl, cycloalkenylalkyl, naphthyl, phenyl, and phenylethyl.

The term “heterocycle” refers to a cyclic (monocyclic or polycyclic) heterocarbyl moiety which may be aromatic, partially aromatic, or non-aromatic. Heterocycles include heteroaryls. Examples include 4-10-membered, 4-7-membered, 6-membered, and 5-membered heterocycles such as aziridinyl, azetidinyl, benzo[1,3]dioxolyl, benzoxazinyl, 1,3-benzodioxolyl, 1,4-benzodioxanyl, cinnolinyl, chromanyl, 2,3-dihydro-benzo[1,4]dioxinyl, dihydrooxazolyl, 1,2-dihydropyridinyl, 2,3-dihydrobenzofuranyl, 1,4-dioxane, dioxolane, butyrolactam, furanyl, homopiperidinyl, hydantoinyl, isoindolinyl-1-one, 2-oxa-6-azaspiro[3,3]heptanyl, hydantoin, imidazolidin-2-one, imidazolidine, imidazolidinone, morpholinyl, oxetanyl, oxiranyl, phthalimidyl, piperazinyl, piperidinyl, pyrrolidinonyl, piperidinyl, pyrrolidinyl, pyrazolidine, spiro[cyclopropane-1,1′-isoindolinyl]-3′-one, tetrahydrofuranyl, tetrahydropyranyl, tetrahydropyridinyl, tetrahydropyrimidinyl, tetrahydrothiophenyl, 1,2,3,4-tetrahydroquinolyl, tetrahydrothiopyranyl, thiomorpholinyl, and valerolactamyl.

The term “include” has the same meaning as “include, but are not limited to,” and the term “includes” has the same meaning as “includes, but is not limited to.” Similarly, the term “such as” has the same meaning as the term “such as, but not limited to.”

The term “lower alkyl” refers to alkyl having from 1 to 6 carbon atoms, e.g., C₁₋₄ alkyl. Examples include methyl, ethyl, propyl, isopropyl, and cyclobutyl.

The terms “manage,” “managing” and “management” mean preventing the recurrence of the specified disease or disorder in a patient who has already suffered from the disease or disorder, and/or lengthening the time that a patient who has suffered from the disease or disorder remains in remission.

The terms encompass modulating the threshold, development and/or duration of the disease or disorder or changing the way that a patient responds to the disease or disorder.

The term “pharmaceutically acceptable salt” refers to a salt that is generally recognized as safe to administer to a subject. Examples of pharmaceutically acceptable salts include acetate, chloride, diphosphate, hydrochloride, maleate, phosphate, potassium, sodium, and sulfate.

The terms “prevent,” “preventing” and “prevention” contemplate an action that occurs before a patient begins to suffer from the specified disease or disorder, which inhibits or reduces the severity of the disease or disorder. The terms encompass prophylaxis.

A “prophylactically effective amount” of a compound is an amount sufficient to prevent a disease or condition, or one or more symptoms associated with the disease or condition, or prevent its recurrence.

A “prophylactically effective amount” of a compound means an amount, alone or in combination with other agents, that provides a prophylactic benefit in the prevention of the disease. The term “prophylactically effective amount” can encompass an amount that improves overall prophylaxis or enhances the prophylactic efficacy of another prophylactic agent.

The terms “subject” and “patient” are used interchangeably. The terms “subject” and “subjects” refer to an animal, such as a non-primate mammal (e.g., cow, pig, horse, cat, dog, rat, and mouse) and a primate (e.g., monkey, a chimpanzee, human). Preferred subjects are human (e.g., adult humans).

A “therapeutically effective amount” of a compound is an amount sufficient to provide a therapeutic benefit in the treatment or management of a disease or condition, or to delay or minimize one or more symptoms associated with the disease or condition. A “therapeutically effective amount” of a compound means an amount, alone or in combination with other therapies, that provides a therapeutic benefit in the treatment or management of the disease or condition. The term “therapeutically effective amount” can encompass an amount that improves overall therapy, reduces, or avoids symptoms or causes of a disease or condition, or enhances the therapeutic efficacy of another therapeutic agent.

The terms “treat,” “treating” and “treatment” contemplate an action that occurs while a patient is suffering from a specified disease or disorder, which reduces the severity of the disease or disorder or retards or slows the progression of the disease or disorder.

Unless otherwise indicated, an adjective before a string of nouns should be construed to apply to each. For example, the phrase “optionally substituted pyridyl, pyrazyl, or furanyl” means the same as “optionally substituted pyridyl, optionally substituted pyrazyl, or optionally substituted furanyl”.

A wavy line [symbol] that intersects a bond in a chemical structure indicates the point of attachment of the bond that the wavy bond intersects in the chemical structure to the remainder of a molecule.

Compounds disclosed herein may exist as tautomeric isomers. Although only one delocalized resonance structure may be depicted, all such forms are contemplated within the scope of the invention.

Unless explicitly noted, chemical structures named or shown herein encompass derivatives wherein one or more of their constituent atoms is/are replaced or enriched (i.e., present in a quantity measurably greater than its natural abundance) with an isotope thereof. Thus, unless noted otherwise, a chemical structure that contains hydrogen atoms encompasses deuterated forms of the represented molecule or moiety.

Compounds disclosed herein may exist as zwitterions (e.g., at pharmacological pH). Unless otherwise indicated, it should be understood that a chemical drawing depicting the structure of such a compound encompasses all of its zwitterionic forms.

Some compounds may exist as stereoisomers. When a stereoisomer of compound is defined by its name (e.g., with the use of R or S) or is depicted in a drawn structure (e.g., using a bold, bold-wedge, dashed, or dashed-wedge to depict the relevant chemical bond), the enantiomeric excess (ee) of that compound, unless otherwise indicated, is to be understood to be at least 60, 70, 80, 90, 95, or 99%. A compound or composition enriched with one stereoisomer of the compound has that one stereoisomer in an amount measurably greater than the compound′ other stereoisomer(s). For example, a compound enriched with an R enantiomer will have an enantiomeric excess of that enantiomer versus the S enantiomer.

### 4.2. COMPOUNDS

This invention encompasses compounds of formula I:

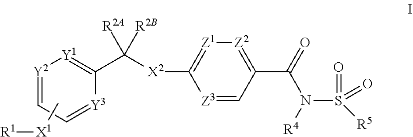

and pharmaceutically acceptable salts thereof, wherein:
X¹ and X² is independently O or CHR⁶;
each of Y¹, Y², and Y³ is independently N, CR³, or C—X¹—R¹, provided that only one of Y¹, Y², and Y³ may be attached to X¹;
each of Z¹, Z², and Z³ is independently N or CR³;
each R¹ is independently optionally substituted C₁₋₆ hydrocarbyl or 2-9 membered heterocarbyl, which optional substitution is with one or more of halo, R^{1A}, or OR^{1A};
each R^{1A} is independently optionally substituted C₁₋₆ hydrocarbyl, which optional substitution is with one or more of halo;
each of R^{2A} and R^{2B} is independently hydrogen or C₁₋₆ alkyl optionally substituted with one or more halo, or R^{2A} and R^{2B} are taken together to form a C₃₋₅ cycloalkyl or 3-5 membered cycloheteroalkyl;
each R³ is independently hydrogen, halo, C₁₋₆ alkyl optionally substituted with one or more halo;
R⁴ is hydrogen or C₁₋₆ alkyl;
R⁵ is N(R^{5A})₂, NHR^{5A}, or optionally substituted C₁₋₈ hydrocarbyl or 2-9 membered heterocarbyl, which optional substitution is with one or more of halo, hydroxyl, nitrile, RA, OR^{5A}, N(R^{5A})₂, NHR^{5A}, or NH₂;
each R⁶ is independently optionally substituted C₁₋₈ hydrocarbyl or 2-9 membered heterocarbyl, which optional substitution is with one or more of halo, hydroxyl, nitrile, R^{5B}, OR^{5B}, N(R^{5B})₂, NHR^{5B}, or NH₂;
each R^{5B} is independently optionally substituted C₁₋₈ hydrocarbyl or 2-9 membered heterocarbyl, which optional substitution is with one or more of halo, hydroxyl, nitrile, R^{C}, OR^{5C}, N(R^{5C})₂, NHR^{C}, or NH₂;
each R^{5C} is independently optionally substituted C₁₋₈ hydrocarbyl or 2-9 membered heterocarbyl, which optional substitution is with one or more of halo, hydroxyl, nitrile, or NH₂; and
each R⁶ is independently H or C₁₋₆ alkyl.

In some embodiments of the invention, R^{2A} and R^{2B} are taken together to form a C₃₋₅ cycloalkyl or 3-5 membered cycloheteroalkyl (e.g., cyclopropyl or cyclobutyl).

In some embodiments of the invention, X¹ is O. In some embodiments, X² is O. In some embodiments, Y is N. In some embodiments, n is 1 or 2.

Some embodiments of the invention are directed to compounds of formula II:

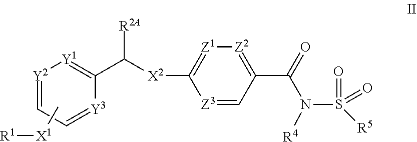

and pharmaceutically acceptable salts thereof.

In some embodiments of the invention (e.g., as represented in any of the applicable formulae herein), at least one of Y¹, Y², and Y³ is N. In some embodiments, each of Y¹ and Y² is independently CR³. In some embodiments, at least one of Z¹, Z², and Z³ is N. In some embodiments, each of Z² and Z³ is N. In some embodiments, each of Z¹ and Z³ is N.

Some embodiments are directed to compounds of formula III:

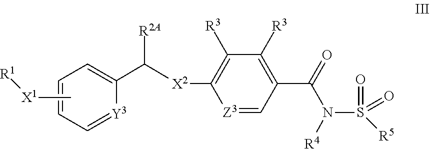

and pharmaceutically acceptable salts thereof.

Some embodiments are directed to compounds of formula IV:

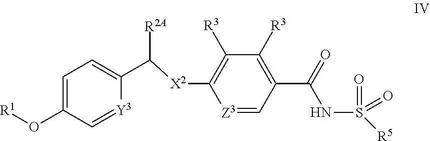

and pharmaceutically acceptable salts thereof.

In some embodiments of the invention, X² is O.

Preferred compounds of the invention are enantiomerically pure or enriched with (R) stereochemistry at the carbon to which R^{2A} is attached. For example, some embodiments of the invention are directed to compounds of formula V(a):

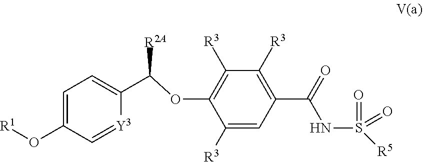

and pharmaceutically acceptable salts thereof, while others are directed to compounds of formula V(b):

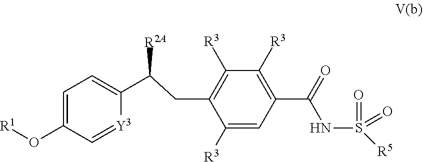

and pharmaceutically acceptable salts thereof.

In some embodiments of the invention, Y³ is N.

In some embodiments of the invention, R is one of:

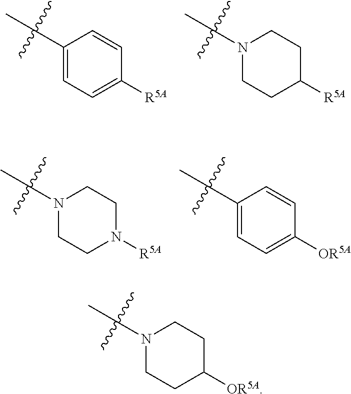

Some embodiments are directed to compounds of formula VI(a):

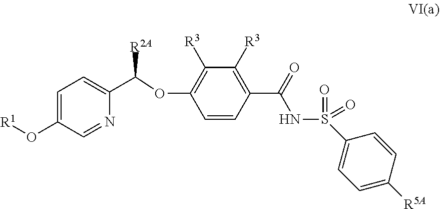

and pharmaceutically acceptable salts thereof. Others are directed to compounds of formula VI(b):

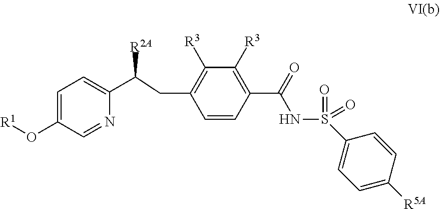

Some embodiments are directed to compounds of formula VII(a):

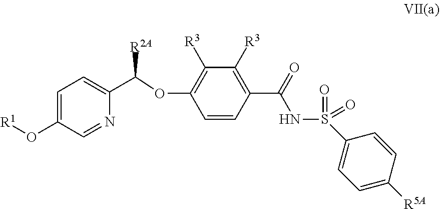

and pharmaceutically acceptable salts thereof. Others are directed to compounds of formula VII(b):

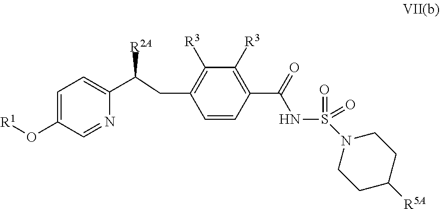

and pharmaceutically acceptable salts thereof.

Some embodiments are directed to compounds of formula VIII(a):

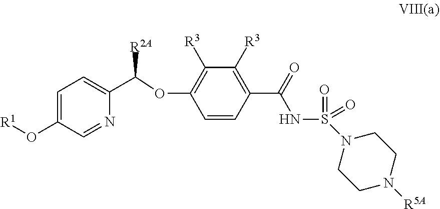

and pharmaceutically acceptable salts thereof. Others are directed to compounds of formula VIII(b):

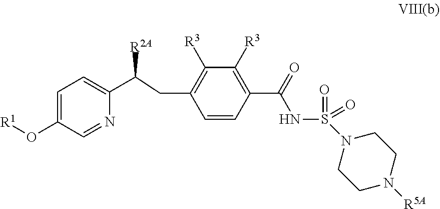

and pharmaceutically acceptable salts thereof.

Certain embodiments of the invention are directed to compounds encompassed by the generic structures disclosed herein (e.g., structures I-VIII) wherein R¹ is optionally substituted C₁₋₆ hydrocarbyl.

Others are directed to compounds wherein R¹ is cyclopropylmethyl, phenyl, or cyclopropyl. Others are directed to compounds wherein R¹ is optionally substituted 2-9 membered heterocarbyl. Others are directed to compounds wherein R¹ is pyridyl, pyrazyl, thiophenyl, or furanyl. Others are directed to compounds wherein R¹ is substituted with OR^{1A}. Certain embodiments are directed to compounds wherein R^{IA} is C₁₋₆ alkyl. In some embodiments, R² is methyl or ethyl. In some embodiments, R³ is fluoro. In some embodiments, R³ is hydrogen.

In some embodiments, R^{5A} is optionally substituted alkyl or 4- or 5-membered heterocycle. In some embodiments, R^{S} is optionally substituted with R^{5B}. In some embodiments, R^{5B} is optionally substituted C₁₋₆ alkyl.

Specific compounds of the invention include:
(R)-4-(1-(5-(cyclopropylmethoxy)pyridin-2-yl)propoxy)-2,3-difluoro-N-((4-(2-(pyrrolidin-1-yl)ethoxy)piperidin-1-yl)sulfonyl)benzamide;
4-((R)-1-(5-(cyclopropylmethoxy)pyridin-2-yl)propoxy)-2,3-difluoro-N-((5-(2-(pyrrolidin-1-yl)ethoxy)-2-azabicyclo[2.2.1]heptan-2-yl)sulfonyl)benzamide;
(R)-4-(1-(5-(cyclopropylmethoxy)pyridin-2-yl)propoxy)-2,3-difluoro-N-((6-((1-methylpiperidin-4-yl)oxy)-2-azaspiro[3,3]heptan-2-yl)sulfonyl)benzamide;
(R)-2,3-difluoro-N-((4-((1-methylpiperidin-4-yl)oxy)piperidin-1-yl)sulfonyl)-4-(1-(5-(pyridin-2-yloxy)pyridin-2-yl)propoxy)benzamide;
(R)-4-(1-(5-(cyclopropylmethoxy)pyridin-2-yl)propoxy)-2,3-difluoro-N-((4-((1-methylpiperidin-4-yl)oxy)piperidin-1-yl)sulfonyl)benzamide;
(R)-4-(1-(5-(cyclopropylmethoxy)pyridin-2-yl)propoxy)—N-((6-(2-(dimethylamino)ethoxy)-2-azaspiro[3,3]heptan-2-yl)sulfonyl)-2,3-difluorobenzamide;
(R)-4-(1-(5-(cyclopropylmethoxy)pyridin-2-yl)propoxy)-2,3-difluoro-N-((4-((1-(2-hydroxy-2-methylpropyl)piperidin-4-yl)oxy)piperidin-1-yl)sulfonyl)benzamide;
(R)-4-(1-(5-(cyclopropylmethoxy)pyridin-2-yl)propoxy)-2,3-difluoro-N-((6-(2-(pyrrolidin-1-yl)ethoxy)-2-azaspiro[3,3]heptan-2-yl)sulfonyl)benzamide;
(R)-4-(1-(5-(cyclopropylmethoxy)pyridin-2-yl)propoxy)-2,3-difluoro-N-((4-((1-(2-methoxyethyl)piperidin-4-yl)oxy)piperidin-1-yl)sulfonyl)benzamide;
(R)-4-(1-(5-(cyclopropylmethoxy)pyridin-2-yl)propoxy)—N-((4-((1-(cyclopropylmethyl)piperidin-4-yl)oxy)piperidin-1-yl)sulfonyl)-2,3-difluorobenzamide;
(R)-4-(1-(5-(cyclopropylmethoxy)pyridin-2-yl)propoxy)—N-((4-((4-ethylpiperazin-1-yl)methyl)piperidin-1-yl)sulfonyl)-2,3-difluorobenzamide;
(R)—N-((4-(2-(dimethylamino)ethoxy)piperidin-1-yl)sulfonyl)-2,3-difluoro-4-(1-(5-(2-methoxyethoxy)pyridin-2-yl)propoxy)benzamide;
(R)-2,3-difluoro-4-(1-(5-(2-methoxyethoxy)pyridin-2-yl)propoxy)—N-((4-((1-methylpiperidin-4-yl)oxy)piperidin-1-yl)sulfonyl)benzamide;
(R)—N-((4-(2-(dimethylamino)ethoxy)piperidin-1-yl)sulfonyl)-2,3-difluoro-4-(1-(5-(pyridin-2-yloxy)pyridin-2-yl)propoxy)benzamide;
(R)-4-(1-(5-(cyclopropylmethoxy)pyridin-2-yl)propoxy)-N-((4-(2-(dimethylamino)ethoxy)piperidin-1-yl)sulfonyl)-2,3-difluorobenzamide;
(R)-2,3-difluoro-N-((4-methylpiperazin-1-yl)sulfonyl)-4-(1-(5-(pyridin-2-yloxy)pyridin-2-yl)propoxy)benzamide;
(R)—N-((4-(2-(1H-imidazol-1-yl)ethoxy)phenyl)sulfonyl)-2,3-difluoro-4-(1-(5-(2-methoxyethoxy)pyridin-2-yl)propoxy)benzamide; or
(R)—N-((4-(2-(dimethylamino)ethoxy)phenyl)sulfonyl)-2,3-difluoro-4-(1-(5-(2-methoxyethoxy)pyridin-2-yl)propoxy)benzamide.

Other compounds of the invention include:
(R)-4-(1-(5-(cyclopropylmethoxy)pyridin-2-yl)propoxy)-2,3-difluoro-N-((4-(2-(pyrrolidin-1-yl)ethoxy)phenyl)sulfonyl)benzamide;
(R)-4-(1-(5-(cyclopropylmethoxy)pyridin-2-yl)propoxy)-2,3-difluoro-N-((4-(2-morpholinoethoxy)phenyl)sulfonyl)benzamide;
(R)-4-(1-(5-(cyclopropylmethoxy)pyridin-2-yl)propoxy)-2,3-difluoro-N-((4-((1-methylpiperidin-4-yl)oxy)phenyl)sulfonyl)benzamide;
(R)-4-(1-(5-(cyclopropylmethoxy)pyridin-2-yl)propoxy)-2,3-difluoro-N-((4-((1-methylpiperidin-4-yl)oxy)phenyl)sulfonyl)benzamide;
(R)-4-(1-(5-(cyclopropylmethoxy)pyridin-2-yl)propoxy)-2,3-difluoro-N-((4-((1-methylpiperidin-4-yl)oxy)phenyl)sulfonyl)benzamide;
(R)-4-(1-(5-(cyclopropylmethoxy)pyridin-2-yl)propoxy)-N-((4-(2-(dimethylamino)ethoxy)phenyl)sulfonyl)-2,3-difluorobenzamide;
(R)-4-(1-(5-(cyclopropylmethoxy)pyridin-2-yl)propoxy)-2,3-difluoro-N-((4-(2-(4-methylpiperazin-1-yl)ethoxy)phenyl)sulfonyl)benzamide;
(R)-4-(1-(5-(cyclopropylmethoxy)pyridin-2-yl)propoxy)-N-((4-(2-(dimethylamino)ethoxy)phenyl)sulfonyl)-2,3-difluoro-N-methylbenzamide;
4-((R)-1-(5-(cyclopropylmethoxy)pyridin-2-yl)propoxy)-2,3-difluoro-N-((4-(2-((S)-3-fluoropyrrolidin-1-yl)ethoxy)phenyl)sulfonyl)benzamide;
(R)-4-(1-(5-(cyclopropylmethoxy)pyridin-2-yl)propoxy)-2,3-difluoro-N-((4-(2-(oxetan-3-ylamino)ethoxy)phenyl)sulfonyl)benzamide;
(R)-4-(1-(5-(cyclopropylmethoxy)pyridin-2-yl)propoxy)-2,3-difluoro-N-((6-((1-methylpiperidin-4-yl)oxy)pyridazin-3-yl)sulfonyl)benzamide;
(R)-4-(1-(5-(cyclopropylmethoxy)pyridin-2-yl)propoxy)-2,3-difluoro-N-((6-((1-methylpiperidin-4-yl)oxy)pyridin-3-yl)sulfonyl)benzamide;
(R)—N-((4-((1H-imidazol-4-yl)methoxy)phenyl)sulfonyl)-2,3-difluoro-4-(1-(5-(2-methoxyethoxy)pyridin-2-yl)propoxy)benzamide;
(R)-4-(1-(5-(cyclopropylmethoxy)pyridin-2-yl)propoxy)-2,3-difluoro-N-((4-((1-(methyl-d₃)piperidin-4-yl)oxy)phenyl)sulfonyl)benzamide;
(R)—N-((4-(2-(cyclopropylamino)ethoxy)phenyl)sulfonyl)-4-(1-(5-(cyclopropylmethoxy)pyridin-2-yl)propoxy)-2,3-difluorobenzamide;
4-((R)-1-(5-(cyclopropylmethoxy)pyridin-2-yl)propoxy)-2,3-difluoro-N-((4-(((S)-1-methylpyrrolidin-3-yl)oxy)phenyl)sulfonyl)benzamide;
(R)-4-(1-(5-(cyclopropylmethoxy)pyridin-2-yl)propoxy)-N-((4-((1-(cyclopropylmethyl)piperidin-4-yl)oxy)phenyl)sulfonyl)-2,3-difluorobenzamide;
(R)-4-(1-(5-(cyclopropylmethoxy)pyridin-2-yl)propoxy)-2,3-difluoro-N-((4-((1-(2-hydroxyethyl)piperidin-4-yl)oxy)phenyl)sulfonyl)benzamide;
4-((R)-1-(5-(cyclopropylmethoxy)pyridin-2-yl)propoxy)-2,3-difluoro-N-((4-(((R)-1-methylpyrrolidin-3-yl)oxy)phenyl)sulfonyl)benzamide; or
(R)-4-(1-(5-(cyclopropylmethoxy)pyridin-2-yl)propoxy)-N-((4-((1-ethylpiperidin-4-yl)oxy)phenyl)sulfonyl)-2,3-difluorobenzamide.

Other compounds of the invention include:
(R)-4-(1-(5-(cyclopropylmethoxy)pyridin-2-yl)propoxy)-2,3-difluoro-N-((4-((1-isopropylpiperidin-4-yl)oxy)phenyl)sulfonyl)benzamide;
(R)-4-(1-(5-(cyclopropylmethoxy)pyridin-2-yl)propoxy)-2,3-difluoro-N-((4-((1-methylazetidin-3-yl)oxy)phenyl)sulfonyl)benzamide;
(R)-4-(1-(5-(cyclopropylmethoxy)pyridin-2-yl)propoxy)-2,3-difluoro-N-((6-((1-methylpiperidin-4-yl)amino)pyridin-3-yl)sulfonyl)benzamide;
(R)-4-(1-(5-(cyclopropylmethoxy)pyridin-2-yl)propoxy)-2,3-difluoro-N-((6-(2-(pyrrolidin-1-yl)ethoxy)pyridazin-3-yl)sulfonyl)benzamide;
4-((R)-1-(5-(cyclopropylmethoxy)pyridin-2-yl)propoxy)-2,3-difluoro-N-((4-((1,2,2-trimethylpiperidin-4-yl)oxy)phenyl)sulfonyl)benzamide;
(R)-4-(1-(5-(cyclopropylmethoxy)pyridin-2-yl)propoxy)-2,3-difluoro-N-((6-((2-(pyrrolidin-1-yl)ethyl)amino)pyridin-3-yl)sulfonyl)benzamide;
(R)-4-(1-(5-(cyclopropylmethoxy)pyridin-2-yl)propoxy)-2,3-difluoro-N-((4-(piperidin-4-yloxy)phenyl)sulfonyl)benzamide;
(R)-4-(1-(5-(cyclopropylmethoxy)pyridin-2-yl)propoxy)-2,3-difluoro-N-((5-((1-methylpiperidin-4-yl)oxy)pyridin-2-yl)sulfonyl)benzamide;
(R)-4-(1-(5-(cyclopropylmethoxy)pyridin-2-yl)propoxy)-2,3-difluoro-N-((2-((1-methylpiperidin-4-yl)oxy)pyrimidin-5-yl)sulfonyl)benzamide;
4-((R)-1-(5-(cyclopropylmethoxy)pyridin-2-yl)propoxy)-2,3-difluoro-N-((4-(((1R,4R,5R)-2-methyl-2-azabicyclo[2.2.1]heptan-5-yl)oxy)phenyl)sulfonyl)benzamide;
(R)-4-(1-(5-(cyclopropylmethoxy)pyridin-2-yl)propoxy)-2,3-difluoro-N-((5-((1-methylpiperidin-4-yl)amino)pyridin-2-yl)sulfonyl)benzamide;
(R)-4-(1-(5-(cyclopropylmethoxy)pyridin-2-yl)propoxy)-N-((4-((1-(2,2-difluoroethyl)piperidin-4-yl)oxy)phenyl)sulfonyl)-2,3-difluorobenzamide;
(R)-4-(1-(5-(cyclopropylmethoxy)pyridin-2-yl)propoxy)-2,3-difluoro-N-((6-((1-isopropylpiperidin-4-yl)amino)pyridin-3-yl)sulfonyl)benzamide;
N-((4-(((1R,4R,5R)-2-azabicyclo[2.2.1]heptan-5-yl)oxy)phenyl)sulfonyl)-4-((R)-1-(5-(cyclopropylmethoxy)pyridin-2-yl)propoxy)-2,3-difluorobenzamide;
(R)-4-(1-(5-(cyclopropylmethoxy)pyridin-2-yl)propoxy)-2,3-difluoro-N-((6-(2-(pyrrolidin-1-yl)ethoxy)pyridin-3-yl)sulfonyl)benzamide;
(R)-4-(1-(5-(cyclopropylmethoxy)pyridin-2-yl)propoxy)-2,3-difluoro-N-((4-((1-(2-methoxyethyl)piperidin-4-yl)oxy)phenyl)sulfonyl)benzamide; or
(R)-4-(1-(5-(cyclopropylmethoxy)pyridin-2-yl)propoxy)-2,3-difluoro-N-((4-((1-(2-hydroxy-2-methylpropyl)piperidin-4-yl)oxy)phenyl)sulfonyl)benzamide.

Other compounds of the invention include:
(R)-4-(1-(5-(cyclopropylmethoxy)pyridin-2-yl)propoxy)-2,3-difluoro-N-((6-((1-isopropylpiperidin-4-yl)oxy)pyridin-3-yl)sulfonyl)benzamide;
(R)-2,3-difluoro-4-(1-(5-(2-methoxyethoxy)pyridin-2-yl)propoxy)-N-((4-((1-methylpiperidin-4-yl)oxy)phenyl)sulfonyl)benzamide;
(R)—N-((4-((1-azabicyclo[2.2.1]heptan-4-yl)oxy)phenyl)sulfonyl)-4-(1-(5-(cyclopropylmethoxy)pyridin-2-yl)propoxy)-2,3-difluorobenzamide;
4-((R)-1-(5-(cyclopropylmethoxy)pyridin-2-yl)propoxy)-2,3-difluoro-N-((4-(2-((R)-1-methylpiperidin-2-yl)ethoxy)phenyl)sulfonyl)benzamide;
(R)-4-(1-(5-(cyclopropylmethoxy)pyridin-2-yl)propoxy)-2,3-difluoro-N-((1-methylpiperidin-4-yl)sulfonyl)benzamide;
(R)-4-(1-(5-(cyclopropylmethoxy)pyridin-2-yl)propoxy)-2,3-difluoro-N-((4-(2-(piperidin-1-yl)ethoxy)phenyl)sulfonyl)benzamide;
(R)-2,3-difluoro-4-(1-(5-(pyridin-2-yloxy)pyridin-2-yl)propoxy)-N-((2,2,4-trimethylpiperazin-1-yl)sulfonyl)benzamide;
(R)-4-(1-(5-(cyclopropylmethoxy)pyridin-2-yl)propoxy)-N-((6-((4-ethylpiperazin-1-yl)methyl)pyridin-3-yl)sulfonyl)-2,3-difluorobenzamide;
(R)-2,3-difluoro-N-((1-methylpiperidin-4-yl)sulfonyl)-4-(1-(5-(pyridin-2-yloxy)pyridin-2-yl)propoxy)benzamide;
(R)-4-(1-(5-(cyclopropylmethoxy)pyridin-2-yl)propoxy)-2,3-difluoro-N-((2,2,4-trimethylpiperazin-1-yl)sulfonyl)benzamide;
(R)-4-(1-(5-(cyclopropylmethoxy)pyridin-2-yl)propoxy)-N-((1-(dimethylamino)-2-methylpropan-2-yl)sulfonyl)-2,3-difluorobenzamide;
(R)—N-(tert-butylsulfonyl)-4-(1-(3-phenoxyphenyl)ethoxy)-3-(trifluoromethyl)benzamide;
(R)-4-(1-(5-(cyclopropylmethoxy)pyridin-2-yl)propoxy)-2,3-difluoro-N-((4-methylpiperazin-1-yl)sulfonyl)benzamide; or
(R)—N-((1-(dimethylamino)-2-methylpropan-2-yl)sulfonyl)-2,3-difluoro-4-(1-(5-(pyridin-2-yloxy)pyridin-2-yl)propoxy)benzamide.

Other compounds of the invention include:
4-(1-(5-ethoxypyridin-2-yl)ethoxy)-2,3-difluoro-N-((4-methylpiperazin-1-yl)sulfonyl)benzamide; rac-(R)-2,3-dichloro-N-((4-methylpiperazin-1-yl)sulfonyl)-4-(1-(3-phenoxyphenyl)ethoxy)benzamide;
N-((4-(aminomethyl)phenyl)sulfonyl)-2,3-difluoro-4-(1-(3-(pyridin-3-yloxy)phenyl)ethoxy)benzamide;
4-(1-(5-ethoxypyridin-2-yl)ethoxy)-2,3-difluoro-N-(piperidin-4-ylsulfonyl)benzamide; 2-amino-4-(1-(5-ethoxypyridin-2-yl)ethoxy)-3-fluoro-N-(piperidin-4-ylsulfonyl)benzamide;
N-((3-morpholinopropyl)sulfonyl)-4-(1-(5-(pyridazin-3-yloxy)pyridin-2-yl)ethoxy)-3-(trifluoromethyl)benzamide;
N-((2-aminoethyl)sulfonyl)-2,3-dichloro-4-(1-(3-phenoxyphenyl)ethoxy)benzamide;
4-(1-(5-ethoxypyridin-2-yl)ethoxy)-2,3-difluoro-N-((1-methylpiperidin-4-yl)sulfonyl)benzamide;
rac-(R)—N-((4-(aminomethyl)phenyl)sulfonyl)-2,3-dichloro-4-(1-(3-phenoxyphenyl)ethoxy)benzamide;
N-((4-(2-aminoethyl)phenyl)sulfonyl)-2,3-difluoro-4-((3-phenoxybenzyl)oxy)benzamide;
rac-(R)—N-((2-aminoethyl)sulfonyl)-2,3-dichloro-4-(1-(3-phenoxyphenyl)ethoxy)benzamide;
rac-(R)—N-((4-(aminomethyl)phenyl)sulfonyl)-3-methyl-4-(1-(3-phenoxyphenyl)ethoxy)benzamide;
2,3-difluoro-N-((4-((methylamino)methyl)phenyl)sulfonyl)-4-((3-phenoxybenzyl)oxy)benzamide;
rac-(R)—N-((2-aminoethyl)sulfonyl)-4-(1-(3-phenoxyphenyl)ethoxy)-3-(trifluoromethyl)benzamide;
4-(1-(5-ethoxypyridin-2-yl)propoxy)-N-((3-morpholinopropyl)sulfonyl)-3-(trifluoromethyl)benzamide;
4-(1-(5-ethoxypyridin-2-yl)propoxy)-N-((4-(2-(methylamino)ethoxy)phenyl)sulfonyl)-3-(trifluoromethyl)benzamide;
N-((4-(aminomethyl)phenyl)sulfonyl)-4-(1-(3-(cyclopentyloxy)phenyl)ethoxy)-3-(trifluoromethyl)benzamide;
N-((4-(aminomethyl)phenyl)sulfonyl)-4-(1-(3-(pentyloxy)phenyl)ethoxy)-3-(trifluoromethyl)benzamide;
N-((4-((dimethylamino)methyl)phenyl)sulfonyl)-2,3-difluoro-4-((3-phenoxybenzyl)oxy)benzamide;
N-((4-(aminomethyl)phenyl)sulfonyl)-2,3-difluoro-4-(1-(3-phenoxyphenyl)ethoxy)benzamide;
N-((4-(aminomethyl)phenyl)sulfonyl)-4-(1-(3-(pyrimidin-5-yloxy)phenyl)ethoxy)-3-(trifluoromethyl)benzamide;
N-((4-(aminomethyl)phenyl)sulfonyl)-4-(1-(3-(cyclopentylmethoxy)phenyl)ethoxy)-3-(trifluoromethyl)benzamide;
N-((4-(aminomethyl)phenyl)sulfonyl)-4-(1-(3-(thiophen-3-yloxy)phenyl)ethoxy)-3-(trifluoromethyl)benzamide; or
N-((4-(aminomethyl)phenyl)sulfonyl)-4-(1-(3-(pyridin-2-yloxy)phenyl)ethoxy)-3-(trifluoromethyl)benzamide.

Other compounds of the invention include:
N-((4-(aminomethyl)phenyl)sulfonyl)-2,3-difluoro-4-((4-phenoxybenzyl)oxy)benzamide;
N-((4-(aminomethyl)phenyl)sulfonyl)-4-(1-(3-(pyrazin-2-yloxy)phenyl)ethoxy)-3-(trifluoromethyl)benzamide;
N-((4-(aminomethyl)phenyl)sulfonyl)-4-(1-(3-(pyridin-3-yloxy)phenyl)ethoxy)-3-(trifluoromethyl)benzamide;
N-((4-(aminomethyl)phenyl)sulfonyl)-4-(cyclopentylmethoxy)-3-(trifluoromethyl)benzamide;
N-((4-(aminomethyl)phenyl)sulfonyl)-4-(2-(3-phenoxyphenyl)propoxy)-3-(trifluoromethyl)benzamide;
N-((4-(aminomethyl)phenyl)sulfonyl)-4-(1-(3-(cyclopentyloxy)phenyl)ethoxy)-2,3-difluorobenzamide;
N-((4-(aminomethyl)phenyl)sulfonyl)-4-(1-(3-((1-methyl-1H-pyrazol-4-yl)oxy)phenyl)ethoxy)-3-(trifluoromethyl)benzamide;
N-((4-(aminomethyl)phenyl)sulfonyl)-4-(1-(3-((2-methylpentyl)oxy)phenyl)ethoxy)-3-(trifluoromethyl)benzamide;
N-((4-(aminomethyl)phenyl)sulfonyl)-2,3-dichloro-4-((3-phenoxybenzyl)oxy)benzamide;
N-((4-(aminomethyl)phenyl)sulfonyl)-2,3-dichloro-4-(2-(3-phenoxyphenyl)propoxy)benzamide;
4-(1-(3-(cyclopropylmethoxy)phenyl)ethoxy)-N-((1-(2-(dimethylamino)ethyl)-1H-pyrazol-4-yl)sulfonyl)-2,3-difluorobenzamide;
N-((4-(aminomethyl)phenyl)sulfonyl)-2,3-difluoro-4-((3-phenoxybenzyl)oxy)benzamide; and
N-((4-(2-(1H-imidazol-1-yl)ethoxy)phenyl)sulfonyl)-4-(1-(5-ethoxypyridin-2-yl)ethoxy)-2,3-difluorobenzamide;
N-[4-(2-aminoethyl)phenyl]sulfonyl-2,3-difluoro-4-[(3-phenoxyphenyl)methoxy]benzamide;
N-tert-butylsulfonyl-4-[1-(5-ethoxy-2-pyridyl)ethoxy]benzamide;
N-tert-butylsulfonyl-4-[1-(5-ethoxy-2-pyridyl)cyclobutoxy]-2,3-difluoro-benzamide;
N-tert-butylsulfonyl-4-[3-(5-ethoxy-2-pyridyl)oxetan-3-yl]oxy-2,3-difluoro-benzamide;
N-tert-butylsulfonyl-6-[rac-(1R)-1-(3-phenoxyphenyl)ethoxy]pyridazine-3-carboxamide;
N-tert-butylsulfonyl-2-[rac-(1R)-1-(3-phenoxyphenyl)ethoxy]-4-(trifluoromethyl)pyrimidine-5-carboxamide;
N-tert-butylsulfonyl-5-chloro-6-[rac-(1R)-1-(3-phenoxyphenyl)ethoxy]pyridine-3-carboxamide;
4-[1-(benzofuran-5-yl)ethoxy]-3-(trifluoromethyl)benzoic acid;
N-tert-butylsulfonyl-4-[1-(5-phenoxypyrimidin-2-yl)ethoxy]-3-(trifluoromethyl)benzamide;
N-tert-butylsulfonyl-4-[1-[3-methyl-4-(2-pyridyloxy)phenyl]ethoxy]-3-(trifluoromethyl)benzamide;
N-tert-butylsulfonyl-4-[2-hydroxy-1-[5-(2-pyridyloxy)-2-pyridyl]ethoxy]-3-(trifluoromethyl)benzamide;
N-tert-butylsulfonyl-4-[1-methyl-1-(5-pyrimidin-2-yloxy-2-pyridyl)ethoxy]-3-(trifluoromethyl)benzamide;
N-tert-butylsulfonyl-4-[1-(5-ethoxy-2-pyridyl)-3-hydroxy-propoxy]-3-(trifluoromethyl)benzamide;
N-tert-butylsulfonyl-4-[3-(dimethylamino)-1-(5-ethoxy-2-pyridyl)propoxy]-3-(trifluoromethyl)benzamide;
N-tert-butylsulfonyl-4-[1-(5-ethoxy-2-pyridyl)-3-methoxy-propoxy]-3-(trifluoromethyl)benzamide;
N-tert-butylsulfonyl-4-[1-(5-ethoxy-2-pyridyl)-2-methoxy-ethoxy]-3-(trifluoromethyl)benzamide;
N-tert-butylsulfonyl-5-[1-[3-(cyclopropylmethoxy)phenyl]ethoxy]pyridine-2-carboxamide;
N-tert-butylsulfonyl-6-[1-[3-(cyclopropylmethoxy)phenyl]ethoxy]pyridazine-3-carboxamide;
N-tert-butylsulfonyl-4-[1-(5-ethoxy-2-pyridyl)cyclopropoxy]-2,3-difluoro-benzamide;
N-tert-butylsulfonyl-8-[(1R)-1-[3-(cyclopropylmethoxy)phenyl]ethoxy]imidazo[1,5-a]pyridine-5-carboxamide; and
4-[(1R)-1-[5-(cyclopropylmethoxy)-2-pyridyl]propoxy]-N-[4-[2-(dimethylamino)ethoxy]phenyl]sulfonyl-2,3-difluoro-N-methyl-benzamide.

### 4.2.1. Methods of Synthesis

Compounds of the invention can be prepared by methods known in the art, by the general and specific methods disclosed herein, and by adaptation or modification of these methods using approaches well known in the art. Scheme 1 represents one general synthetic approach that may be used to prepare compounds encompassed by this invention.

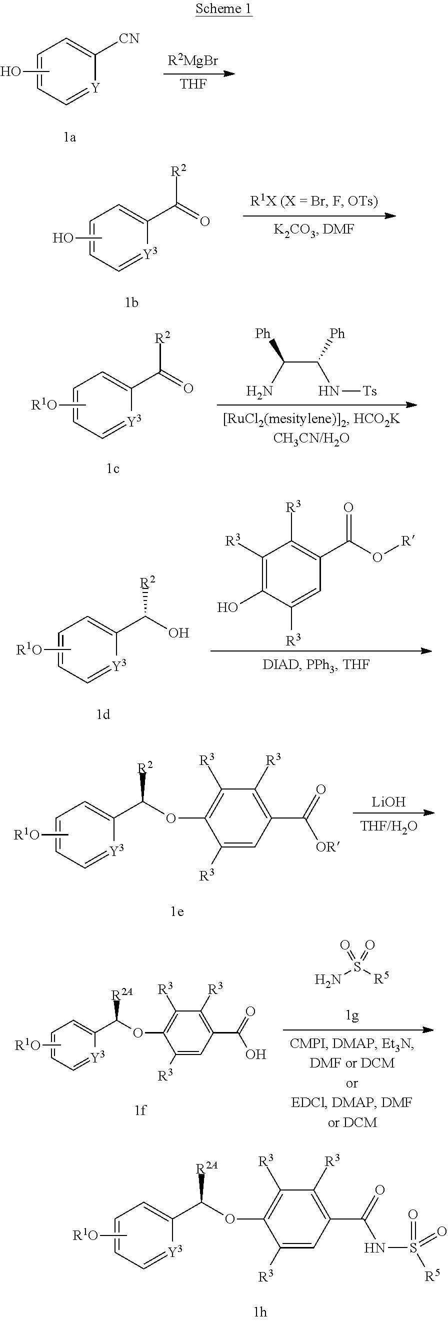

wherein R′ is lower alkyl (e.g., methyl) and the various other substituents (e.g., Y³, R¹) are defined herein. In this approach, a nitrile is converted to an ethyl ketone 1b using the corresponding Grignard reagent.

The desired aryl moiety (e.g., phenyl, pyridyl) is coupled to R¹ using a base mediated SN₂Ar reaction. The ketone is reduced under conditions sufficient to afford the desired enantiomer of the corresponding alcohol Id using a chiral catalyst, which in turn undergoes Mitsunobu coupling with an optionally substituted methyl 4-hydroxybenzoate to afford esterified core 1e, which in turn is converted to the carboxylic acid if following saponification with a suitable agent, such as LiOH, NaOH, or trimethylsilyl iodide (TMSI). The acid is then coupled with the desired sulfonamide 1g to afford compound 1h using, for example, a base (e.g., 4-dimethylaminopyridine (DMAP), Et₃N, ⁱPr₂NEt) and suitable coupling reagents such as 1-ethyl-3-(3-dimethylaminopropyl) carbodiimide hydrochloride (EDCl), benzotriazol-1-yloxytripyrrolidinophosphonium hexafluorophosphate (PyBOP), 1-[bis(dimethylamino)methylene]-1H-1,2,3-triazolo[4,5-b]pyridinium 3-oxide hexafluorophosphate (HATU), and carbonyldiimidazole (CDI). (Various coupling methods in addition to those described herein are known in the art. See, e.g., Chen, L., and Luo, G., “Facile synthesis of acyl sulfonamides from carboxylic acids using the Mukaiyama reagent”, Tetrahedron Let., 2019; 60:268-271.) The resulting product 1h may undergo additional reactions (e.g., deprotection) as needed.

Racemic forms of compounds prepared according to Scheme 1 may be prepared by adapting this approach to begin with Scheme 2:

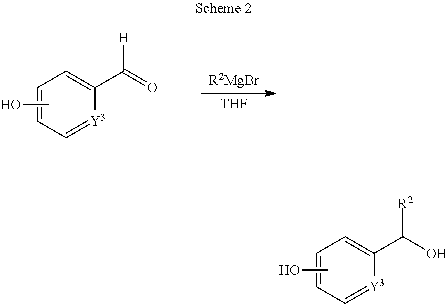

If desired, enantiomers of the final racemic product can be isolated by methods known in the art, such as chiral chromatography.

Other compounds of the invention may be prepared using the general approach shown below in Scheme 3:

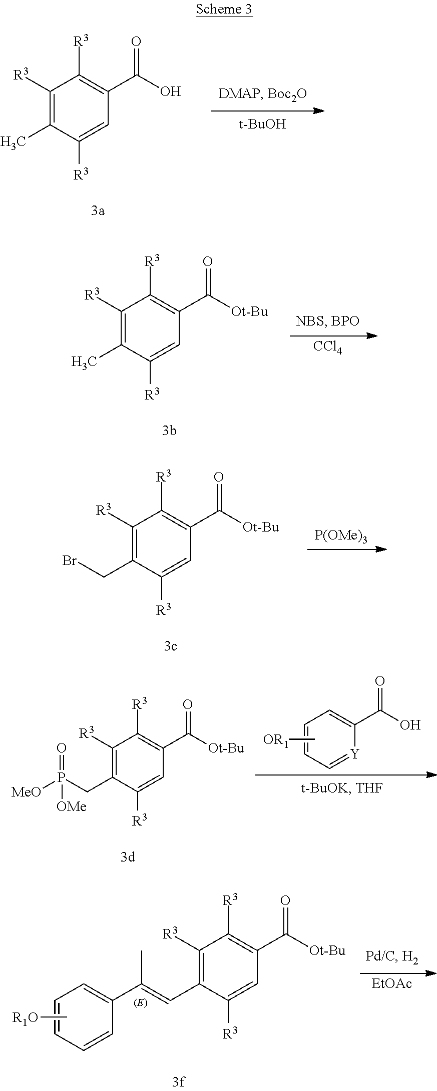

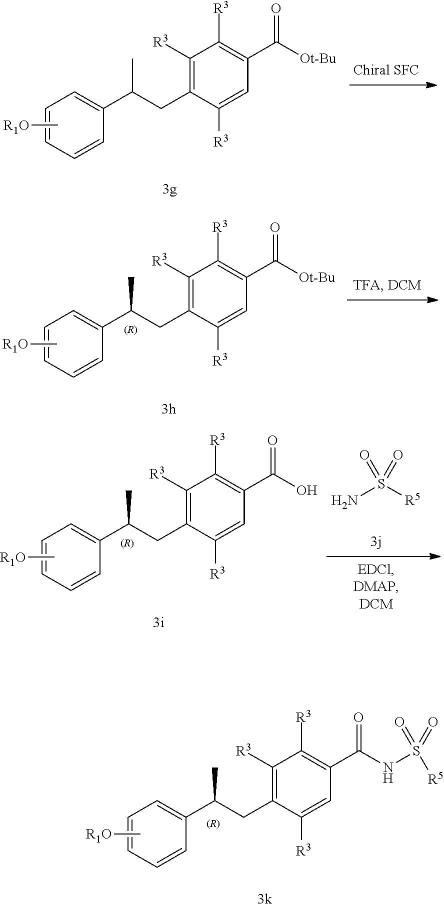

wherein the various substituents (e.g., Y³, R³) are defined herein. In this approach, the acid moiety of 3a is protected under suitable conditions to provide intermediate 3b, which is then contacted with N-bromosuccinimide (NBS) and benzoyl peroxide (BPO) under conditions sufficient to afford the mono-brominated compound 3c. That compound then undergoes a Michaelis-Arbuzov reaction to afford intermediate 3d, which is then reacted with the desired substituted benzoic acid under conditions sufficient to afford alkene 3f. Reduction of 3f affords 3g which, if desired, is resolved to the R enantiomer 3h using chiral chromatograph (e.g., supercritical fluid (SFC) chromatography). (This step may be avoided if a racemic mixture of products is desired.) The carboxylic acid of that compound is then deprotected (e.g., using trifluoroacetic acid (TFA) in dichloromethane (DCM)) to afford intermediate 3i, which is then contacted with the desired sulfone 3j under conditions sufficient (e.g., EDCI, DMAP, DCM) to afford 3k.

### 4.3. METHODS OF USE

This invention encompasses methods of inhibiting ACSL5, which comprise contacting ACSL5 with a compound disclosed herein. The invention further encompasses a method of treating, managing, or preventing a disease or disorder associated with ACSL5 (e.g., its overexpression), which comprises administering to a subject in need thereof a therapeutically or prophylactically effective amount of a compound disclosed herein. Examples of such diseases and disorders include metabolic diseases and disorders and cancer.

This invention encompasses methods of treating, managing, or preventing a metabolic disease or disorder. Examples of metabolic diseases and disorders that may be treated, managed, or prevented using compounds of the invention include metabolic dysfunction-associated steatohepatitis (MASH), metabolic syndrome, non-alcoholic fatty liver disease (NAFLD), obesity, and type 2 diabetes. This invention further encompasses a method of improving insulin sensitivity in a patient in need thereof, which comprises administering to the patient a compound of the invention.

Examples of cancers that may be treated, managed, or prevented using compounds of the invention include cancers wherein ACSL5 is overexpressed as compared to healthy, normal cells.

Examples of such cancers include acute myeloid leukemia (AML), colorectal cancer, and breast cancer.

### 4.4. PHARMACEUTICAL COMPOSITIONS

Compounds of the invention may be incorporated into formulations suitable for administration to a patient. While the route of administration (e.g., oral, parenteral, intravenous, intramuscular, topical, subcutaneous) will depend on the particular indication being treated, a preferred route is oral.

Thus, the present compounds may be systemically administered (e.g., orally) in combination with a pharmaceutically acceptable vehicle such as an inert diluent or an assimilable edible carrier. They may be enclosed in hard or soft shell gelatin capsules or may be compressed into tablets. For oral therapeutic administration, an active compound may be combined with one or more excipients in the form of ingestible tablets, buccal tablets, capsules, caplets, troches, elixirs, suspensions, syrups, and wafers.

Compounds may also be administered intravenously or intraperitoneally by infusion or injection. Solutions of an active compound or its salts may be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

Pharmaceutical dosage forms suitable for injection or infusion may include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, compounds are typically administered as compositions containing a dermatologically acceptable carrier, which may be a solid or a liquid. Examples of solid carriers include finely divided solids such as talc, clay, microcrystalline cellulose, silica, and alumina. Useful liquid carriers include water, alcohols or glycols or water-alcohol/glycol blends, in which an active compound can be dissolved or dispersed at effective levels, optionally with the aid of non-toxic surfactants. Adjuvants such as fragrances and additional antimicrobial agents may be added to optimize the properties for a given use. The resultant liquid compositions can be applied from absorbent pads, used to impregnate bandages and other dressings, or sprayed onto the affected area using pump-type or aerosol sprayers.

### 4.5. EXAMPLES

Certain embodiments of the present invention are described in the examples provided below. Some common abbreviations used in the examples include the following:
CMBP Cyanomethylene)tributylphosphorane
CMPI 2-Chloro-1-methylpyridinium iodide
DAST Diethylaminosulfur trifluoride
DCM Dichloromethane
DIAD Diisopropyl azodicarboxylate
DIPEA Di-isopropylethylamine
DMAP 4-(Dimethylamino)pyridine
DMF N,N-dimethylformamide
DMSO Dimethylsulfoxide
EDCI 1-Ethyl-3-(3-dimethylaminopropyl)carbodiimide
FCC Flash column chromatography
h Hour(s)
HATU (1-[Bis(dimethylamino)methylene]-1H-1,2,3-triazolo[4,5-b]pyridinium 3-oxid hexafluorophosphate)
HPLC High performance liquid chromatography
ip Intraperitoneal administration
LCMS Liquid chromatography-mass spectrometry
min Minute(s)
NCS N-Chlorosuccinimide
NMI 1-Methylimidazole
NMP N-Methyl-2-pyrrolidone
PE Petroleum ether
po Oral administration
qd Once a day
Rt Retention time
RT Room temperature
sc Subcutaneous administration
SFC Supercritical Fluid Chromatography
TCFH Chloro-N,N,N′,N′-tetramethylfoOHamidinium hexafluorophosphate
TFA Trifluoroacetic acid
THF Tetrahydrofuran

Some compounds were characterized using the LCMS conditions provided in Table 1:

TABLE 1

| | | | | Flow rate | |
|---|---|---|---|---|---|
| Method | Column | Mobile Phase (A/B) | Gradient (% A) | (mL/min) | Temp. (° C.) |
| A | Acquity UPLC HSS C18 | MeCN/H₂O | 5-95% (5.6 min); | 0.4 | 40 |
| | 1.8 μm, 100 × 2.1 mm | (+0.1% HCOOH) | 95% (0.8 min) | | |
| B | Acquity UPLC BEH C18 | MeCN/H₂O | 5-95% (5.6 min); | 0.4 | 40 |
| | 1.7 μm, 100 × 2.1 mm | (+10 mM (NH₄)HCO₃) | 95% (0.8 min) | | |
| C | Acquity UPLC BEH C18 | MeCN/H₂O | 5-95% (5.6 min); | 0.4 | 40 |
| | 1.7 μm, 100 × 2.1 mm | (+0.1% HCOOH) | 95% (0.8 min) | | |
| D | Waters CORTECS C18 | MeCN/H₂O | 5-95% (1.4 min); | 1.8 | 50 |
| | 2.7 μm, 4.6 × 30 mm | (+0.05% HCOOH) | 95% (0.6 min) | | |
| E | Waters Xbridge C18 | MeCN/H₂O | 5-95% (1.8 min); | 1.0 | 50 |
| | 3.5 μm, 2.1 × 50 mm | (+0.1% NH₄OH) | 95% (0.7 min) | | |
| F | HALO C18 | MeCN/H₂O | 5-95% (1.4 min); | 1.8 | 50 |
| | 2.7 μm, 3.0 × 30 mm | (+0.05% TFA) | 95% (0.6 min) | | |
| G | Acquity UPLC BEH C18 | MeCN/H₂O | 5-95% (1.25 min); | 0.8 | 40 |
| | 1.7 μm, 100 × 2.1 mm | (+0.1% HCOOH) | 95% (0.75 min) | | |
| H | Acquity UPLC BEH C18 | MeCN/H₂O | 8-97% (1.9 min); | 0.8 | 40 |
| | 1.7 μm, 50 × 2.1 mm | (+0.03% NH₄OH) | 97% (0.1 min) | | |
| I | Kinetex C18 | MeCN/H₂O | 5-95% (1.8 min); | 1.8 | 50 |
| | 2.6 μm, 4.6 mm × 50 mm | (+0.05% TFA) | 95% (0.7 min) | | |
| J | Waters Xbridge C18 | MeCN/H₂O | 5-95% (1.8 min); | 1.8 | 50 |
| | 2.5 μm, 2.5 × 30 mm | (+0.1% NH₄OH) | 95% (0.7 min) | | |
| K | HALO C18 | MeCN/H₂O | 5-95% (1.4 min); | 1.8 | 50 |
| | 2.7 μm, 3.0 × 30 mm | (+0.05% HCOOH) | 95% (0.6 min) | | |

Preparative HPLC methods used to purify compounds are set forth below in Table 2 (mobile phase gradients are provided for each specific example). All methods had a 20.0 mL/min flowrate and were conducted at RT.

TABLE 2

| Method | Column | Mobile Phase (A/B) |
|---|---|---|
| A | Phenomenex Gemini C18, 5 μm, 150 × 21.2 mm | MeCN/H₂O (+0.1% HCOOH) |
| B | Phenomenex Gemini C18, 5 μm, 150 × 21.2 mm | MeCN/H₂O (+0.05% HCOOH) |
| C | Phenomenex Gemini C18, 5 μm, 150 × 21.2 mm | MeCN/H₂O (+0.1% NH₄OH) |
| D | Phenomenex Gemini C18, 5 μm, 150 × 21.2 mm | MeCN/H₂O (+0.05% NH₄OH) |
| E | Waters Xbridge OBD C18, 5 μm, 150 × 19.0 mm | MeCN/H₂O (+0.05% NH₄OH) |
| F | Waters Xbridge OBD C18, 5 μm, 150 × 19.0 mm | MeCN/H₂O (+0.1% HCOOH) |
| G | YMC C18, 5 μm, 150 × 20.0 mm | MeCN/H₂O (+0.1% TFA) |
| H | Waters Sunfire C18, 10 μm, 150 × 19.0 mm | MeCN/H₂O (+10 mM (NH₄)HCO₃) |
| I | Waters Xbridge Phenyl C18, 10 μm, 150 × 19.0 mm | MeCN/H₂O (+10 mM (NH₄)HCO₃) |
| J | Waters Sunfire C18, 10 μm, 150 × 19.0 mm | MeCN/H₂O (+0.1% HCOOH) |
| K | Luna Phenyl-Hexyl, 1 μm, 150 × 21.2 mm | MeOH/H₂O (+0.1% HCOOH) |

Preparative chiral SFC methods used to purify compounds are set forth below in Table 3:

TABLE 3

| | | | Flow rate | |
|---|---|---|---|---|
| Method | Column | Mobile Phase | (ml/min) | Temp. (° C.) |
| A | Daicel CHIRALPAK AD-H | 70/30 | 38 | 40 |
| | 5 μm, 250 × 20 mm | CO₂/MeOH [+0.1% NH₃] | | |
| B | Daicel CHIRALPAK AD-10 | 70/30 | 80 | 40 |
| | 10 μm, 250 × 30 mm | CO₂/MeOH [+0.1% HCOOH] | | |
| C | Daicel CHIRALPAK AD-H | 80/20 | 90 | 40 |
| | 5 μm, 250 × 20 mm | CO₂/MeOH [+0.1% HCOOH] | | |
| D | Daicel CHIRALPAK OJ-10 | 80/20 | 68 | 40 |
| | 5 μm, 250 × 20 mm | CO₂/MeOH [+0.1% HCOOH] | | |
| E | Daicel CHIRALPAK AD-10 | 80/20 | 88 | 40 |
| | 5 μm, 250 × 20 mm | CO₂/MeOH [+0.1% HCOOH] | | |
| F | Daicel CHIRALPAK AD-10 SFC | 60/40 | 75 | 40 |
| | 10 μm, 250 × 30 mm | CO₂/MeOH [+0.1% HCOOH] | | |
| G | Daicel CHIRALPAK IG SFC | 80/20 | 48 | 40 |
| | 5 μm, 250 × 21 mm | CO₂/MeOH [+0.1% HCOOH] | | |
| H | Daicel CHIRALPAK AD-H | 80/20 | 90 | 40 |
| | 5 μm, 250 × 20 mm | CO₂/MeOH [+0.1% HCOOH] | | |
| I | Daicel CHIRALPAK AD-H | 70/30 | 48 | 40 |
| | 5 μm, 250 × 20 mm | CO₂/MeOH [+0.1% HCOOH] | | |
| J | Daicel CHIRALPAK OD-H | 75/25 | 48 | 40 |
| | 5 μm, 250 × 20 mm | CO₂/MeOH [+0.1% HCOOH] | | |
| K | Daicel CHIRALPAK IG SFC | 60/40 | 48 | 40 |
| | 5 μm, 250 × 20 mm | CO₂/MeOH [+0.1% HCOOH] | | |

### 4.5.1. Example 1. Preparation of 4-(1-(5-ethoxypyridin-2-yl)ethoxy)-2,3-difluorobenzoic acid (Acid A)

### Step 1: 1-(5-ethoxypyridin-2-yl)ethan-1-ol

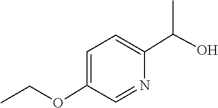

To a solution of 5-ethoxypyridine-2-carbaldehyde (4.02 g, 26.6 mmol) in THF (130 mL) under argon cooled in an ice bath was added methylmagnesium bromide solution in diethyl ether (3M, 13 mL, 39.9 mmol) dropwise and the resulting mixture was stirred for 4 h, allowed to warm to RT and stirred for 2 h. The reaction mixture was cooled in an ice bath, quenched with saturated aqueous NH₄Cl (45 mL), allowed to warm to RT and extracted with EtOAc (x 3). The organic layers were separated, combined, washed with brine and evaporated in vacuo to give the title compound (4.41 g, 99% yield) as a brown oil.

¹H NMR (300 MHz, CDCl₃, ppm) δ 8.21 (t, J=1.7 Hz, 1H), 7.21-7.19 (m, 2H), 4.85 (dd, J=3.0, 6.2 Hz, 1H), 4.08 (q, J=7.0 Hz, 2H), 3.95 (d, J=3.4 Hz, 1H), 1.50-1.41 (m, 6H).

### Step 2: methyl 4-(1-(5-ethoxypyridin-2-yl)ethoxy)-2,3-difluorobenzoate

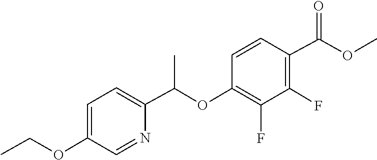 [SMILES: CCOc1ccc(C(C)Oc2ccc(C(=O)OC)c(F)c2F)nc1]

1-(5-Ethoxypyridin-2-yl)ethan-1-ol (2.50 g, 15.0 mmol), methyl 2,3-difluoro-4-hydroxy-benzoate (2.81 g, 15.0 mmol) and PPh₃ (4.71 g, 17.9 mmol) were added together in THF (100 mL) and the resulting mixture was stirred at RT under N₂ for 15 min. The mixture was cooled in an ice bath and DIAD (3.5 mL, 17.9 mmol) was added. The resulting mixture was allowed to warm to RT and stirred for 4 h. The reaction mixture was concentrated in vacuo. The residue was purified by silica gel flash chromatography (gradient 0-50% EtOAc/cyclohexane) to give the title compound (2.68 g, 53% yield) as a yellow oil. LCMS (Method H) MS (ES+) [M+H]⁺=338, Rt=1.55 min. ¹H NMR (300 MHz, CDCl₃, ppm) δ 8.23 (s, 1H), 7.57-7.50 (m, 1H), 7.35-7.30 (m, 1H), 7.20-7.14 (m, 1H), 6.68 (t, J=7.7 Hz, 1H), 5.49 (q, J=6.5 Hz, 1H), 4.15-4.03 (m, 2H), 3.88 (s, 3H), 1.73 (d, J=6.1 Hz, 3H), 1.43 (t, J=7.1 Hz, 3H).

### Step 3: 4-(1-(5-ethoxypyridin-2-yl)ethoxy)-2,3-difluorobenzoic acid

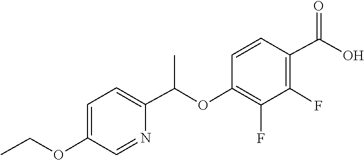 [SMILES: CCOc1ccc(C(C)Oc2ccc(C(=O)O)c(F)c2F)nc1]

To a solution of methyl 4-(1-(5-ethoxypyridin-2-yl)ethoxy)-2,3-difluorobenzoate (2.68 g, 7.94 mmol) in MeOH (17.8 mL), H₂O (17.8 mL) and THF (8.9 mL) was added LiOH H₂O (1.00 g, 23.8 mmol) and the resulting mixture was stirred at RT for 2 h. The solvent was removed in vacuo and the residue was acidified with 1M aqueous HCl and extracted with EtOAc (x 3). The organic layers were separated, combined, washed with brine, dried over Na₂SO₄ and concentrated in vacuo to give the title compound (2.61 g, quant.) as a yellow solid. LCMS (Method G) MS (ES+) [M+H]⁺=324, Rt=1.57 min. ¹H NMR (300 MHz, DMSO-d₆, ppm) δ 13.14 (s, 1H), 8.26-8.24 (m, 1H), 7.59-7.51 (m, 1H), 7.41-7.38 (m, 2H), 7.02-6.95 (m, 1H), 5.66 (q, J=6.4 Hz, 1H), 4.09 (dd, J=6.6, 13.9 Hz, 2H), 1.64 (d, J=6.4 Hz, 3H), 1.32 (t, J=6.9 Hz, 3H).

### 4.5.2. Example 2. Preparation of 4-(1-(3-(cyclopropylmethoxy)phenyl)ethoxy)-2,3-difluorobenzoic acid (Acid B)

### Step 1: methyl 4-(1-(3-(cyclopropylmethoxy)phenyl)ethoxy)-2,3-difluorobenzoate

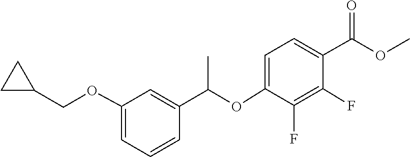 [SMILES: COC(=O)c1ccc(OC(C)c2cccc(OCC3CC3)c2)c(F)c1F]

To a solution of PPh₃ (939 mg, 3.58 mmol) in THF (10 mL) at 0° C. was added DIAD (724 mg, 3.58 mmol) and the resulting mixture was stirred for 20 min. Methyl 2,3-difluoro-4-hydroxybenzoate (337 mg, 1.79 mmol), followed by a solution of 1-(3-(cyclopropylmethoxy)phenyl)ethan-1-ol (344 mg, 1.79 mmol) in THF (3 mL) were added dropwise. The reaction mixture was allowed to warm to RT and stirred for 18 h. H₂O (30 mL) was added, and the mixture was extracted with EtOAc (15 mL×3). The organic layers were separated, combined, washed with brine (5 mL×3), dried over Na₂SO₄ and concentrated in vacuo. The residue was purified by silica gel flash chromatography (gradient 1-10% EtOAc/PE) to give the title compound (498 mg, 76% yield) as a yellow solid.

¹H NMR (400 MHz, CDCl₃, ppm) δ 7.57-7.44 (m, 1H), 7.30-7.17 (m, 1H), 6.92 (dd, J=4.2, 2.1 Hz, 2H), 6.84-6.76 (m, 1H), 6.67-6.54 (m, 1H), 5.37 (q, J=6.4 Hz, 1H), 3.86 (s, 3H), 3.84-3.70 (m, 2H), 1.69 (d, J=6.4 Hz, 3H), 1.38-1.10 (m, 1H), 0.73-0.54 (m, 2H), 0.40-0.29 (m, 2H).

### Step 2: 4-(1-(3-(cyclopropylmethoxy)phenyl)ethoxy)-2,3-difluorobenzoic acid

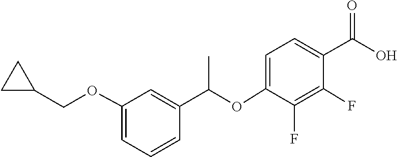 [SMILES: CC(Oc1ccc(C(=O)O)c(F)c1F)c1cccc(OCC2CC2)c1]

A mixture of methyl 4-(1-(3-(cyclopropylmethoxy)phenyl)ethoxy)-2,3-difluorobenzoate (180 mg, 0.50 mmol) and LiOH H₂O (63 mg, 1.50 mmol) in THF/H₂O (3:1, 6 mL) was stirred at 40° C. for 4 h. After cooling to RT, AcOH (4 mL) and H₂O (10 mL) were added. The mixture was extracted with DCM (10 mL×4). The organic layers were separated, combined, dried over Na₂SO₄ and concentrated in vacuo. The residue was purified by prep-HPLC (Method A, gradient: 50-90%) to give the title compound (98 mg, 55% yield) as a white solid. LCMS (Method D) MS (ESI) [M+H]⁺=349, Rt=1.28 min. ¹H NMR (400 MHz, CDCl₃, ppm) δ 7.66-7.55 (m, 1H), 7.30-7.22 (m, 1H), 6.94-6.89 (m, 2H), 6.84-6.77 (m, 1H), 6.67-6.55 (m, 1H), 5.38 (q, J=6.4 Hz, 1H), 3.88-3.68 (m, 2H), 1.71 (d, J=6.4 Hz, 3H), 1.35-1.18 (m, 1H), 0.72-0.57 (m, 2H), 0.44-0.27 (m, 2H).

### 4.5.3. Example 3. Preparation of (R)-4-(1-(5-(cyclopropylmethoxy)pyridin-2-yl)propoxy)-2,3-difluorobenzoic acid (Acid C)

### Step 1: 1-(5-hydroxypyridin-2-yl)propan-1-one

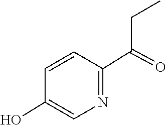

Under N₂, to a solution of 5-hydroxypyridine-2-carbonitrile (50 g, 416.28 mmol) in THF (400 mL) was added ethylmagnesium bromide (624 mL, 1.25 mol, 2M in THF) at 0° C. The reaction mixture was allowed to warm to RT and stirred for 0.5 h, and then heated to 60° C. for 3 h. The reaction mixture was acidified with 3 N HCl aqueous solution to pH 4-5. The mixture was stirred at RT for 30 min. The pH was adjusted to 7 using saturated aqueous NaHCO₃ solution and extracted with EtOAc (400 mL×7). The combined organic layers were dried over Na₂SO₄ and concentrated in vacuo to give the title compound (53 g, 80% yield) as a yellow solid. LCMS (Method D) MS (ESI) [M+H]⁺=152, Rt=0.64 min

### Step 2: 1-(5-(cyclopropylmethoxy)pyridin-2-yl)propan-1-one

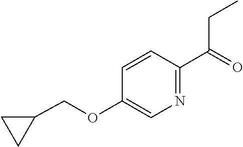

A mixture of 1-(5-hydroxypyridin-2-yl)propan-1-one (53 g, 350 mmol), K₂CO₃ (96.7 g, 700 mmol) and (bromomethyl)cyclopropane (70.8 g, 525 mmol) in DMF (800 mL) was stirred at 90° C. for 1 h. The solvent was removed in vacuo. The residue was purified by silica gel flash chromatography (gradient 0-20% EtOAc/PE) to give the title compound (59 g, 80% yield) as a colorless oil. LCMS (Method D) MS (ESI) [M+H]⁺=206, Rt=1.35 min Step 3: (S)-1-(5-(cyclopropylmethoxy)pyridin-2-yl)propan-1-ol

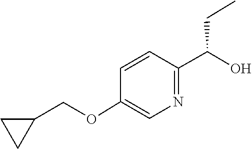

A mixture of [RuCl₂(mesitylene)]₂ (1.87 g, 3.66 mmol) and N-[(1S,2S)-2-amino-1,2-diphenylethyl]-4-methyl-1-sulfonylaniline (2.15 g, 5.85 mmol) in CH₃CN (300 mL) and H₂O (300 mL) was degassed and purged with N₂ (x 3) and then heated at 40° C. for 1 h. The mixture was cooled to 0° C. and treated with a solution of 1-[5-(cyclopropylmethoxy)pyridin-2-yl]propan-1-one (59 g, 287 mmol) in CH₃CN (40 mL), followed by potassium formate (121 g, 1.43 mol). The resulting mixture was stirred at RT for 12 h, diluted with H₂O (500 mL) and extracted with EtOAc (500 mL×3). The organic layers were separated, combined, dried over Na₂SO₄ and concentrated in vacuo. The residue was purified by silica gel flash chromatography (gradient 0-35% EtOAc/PE) to give the title compound (54 g, 86% yield) as a colorless oil. LCMS (Method D) MS (ESI) [M+H]⁺=208, Rt=0.88 min.

### Step 4: methyl (R)-4-(1-(5-(cyclopropylmethoxy)pyridin-2-yl)propoxy)-2,3-difluorobenzoate

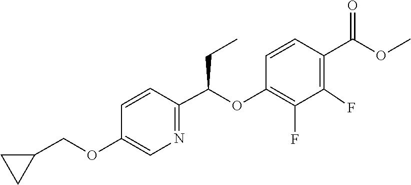 [SMILES: CC[C@@H](Oc1ccc(C(=O)OC)c(F)c1F)c1ccc(OCC2CC2)cn1]

To a solution of PPh₃ (171 g, 649 mmol) in dry THF (2.5 L) at 0° C. was added DIAD (131 g, 647 mmol) dropwise and the resulting mixture was stirred for 20 min. Then, a solution of (1S)-1-(5-(cyclopropylmethoxy)pyridin-2-yl)propan-1-ol (54 g, 259 mmol) and methyl 2,3-difluoro-4-hydroxybenzoate (53.7 g, 285 mmol) in dry THF (500 mL) was added dropwise. The resulting mixture was allowed to warm to RT and stirred for 16 h. The solvent was removed in vacuo and the residue was purified by silica gel flash chromatography (gradient 0-40% EtOAc/PE) to give the title compound (65 g, 66% yield) as a colorless oil. LCMS (Method D) MS (ESI) [M+H]⁺=378, Rt=1.58 min.

### Step 5: (R)-4-(1-(5-(cyclopropylmethoxy)pyridin-2-yl)propoxy)-2,3-difluorobenzoic acid

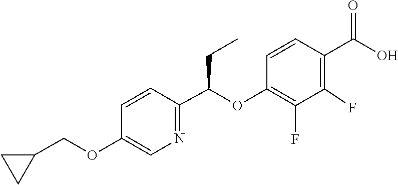 [SMILES: CC[C@@H](Oc1ccc(C(=O)O)c(F)c1F)c1ccc(OCC2CC2)cn1]

A mixture of methyl 4-[(1R)-1-[5-(cyclopropylmethoxy)pyridin-2-yl]propoxy]-2,3-difluorobenzoate (65 g, 173 mmol) and LiOH H₂O (12.4 g, 518 mmol) in THF/H₂O (2:1, 650 mL) was stirred at RT for 16 h. The reaction mixture was acidified with HCOOH to pH=6-7 and extracted with EtOAc (200 mL×3). The organic layers were separated, combined, dried over Na₂SO₄ and concentrated in vacuo. The residue was suspended in CH₃CN (330 mL) and the resulting mixture was heated at 50° C. until the solids had dissolved. Apure sample of (R)-4-(1-(5-(cyclopropylmethoxy)pyridin-2-yl)propoxy)-2,3-difluorobenzoic acid (2 mg) was added and the solution was cooled to RT slowly and left to stand for 18 h. The resulting solid was collected by filtration, washed with CH₃CN (20 mL×2) and dried to give the title compound (49 g, >99% ee, 78% yield) as a white solid. LCMS (Method E) MS (ESI) [M+H]⁺=364, Rt=1.22 min. ¹H NMR (400 MHz, DMSO-d6, ppm) δ 13.13 (s, 1H), 8.26 (s, 1H), 7.57-7.48 (m, 1H), 7.38-7.28 (m, 2H), 6.96-6.87 (m, 1H), 5.41 (t, J=6.5 Hz, 1H), 3.87 (d, J=7.1 Hz, 2H), 2.08-1.90 (m, 2H), 1.27-1.12 (m, 1H), 0.91 (t, J=7.3 Hz, 3H), 0.64-0.42 (m, 2H), 0.41-0.20 (m, 2H).

### 4.5.4. Example 4. Preparation of (R)-2,3-difluoro-4-(1-(5-(2-methoxyethoxy)pyridin-2-yl)propoxy)benzoic acid (Acid D)

### Step 1: 1-(5-(2-methoxyethoxy)pyridin-2-yl)propan-1-one

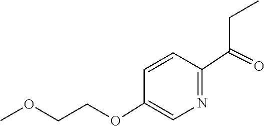

To a solution of 1-(5-hydroxypyridin-2-yl)propan-1-one (9.4 g, 62.2 mmol, Acid C, Step 1) and K₂CO₃ (12.9 g, 93.3 mmol) in DMF (150 mL) was added 1-bromo-2-methoxyethane (10.4 g, 74.6 mmol). The mixture was stirred at 90° C. for 2 h. DMF was removed in vacuo and H₂O (100 mL) was added. The mixture was extracted with DCM (50 mL×6). The combined organic layers were washed with brine (10 mL×3), dried over Na₂SO₄ and concentrated in vacuo. The residue was purified by silica gel flash chromatography (gradient 0-10% EtOAc/PE) to give the title compound (11.3 g, 86% yield) as a pale yellow solid. LCMS (Method D) MS (ESI) [M+H]⁺=210, Rt=0.87 min.

Step 2: (S)-1-(5-(2-methoxethoxy)pyridin-2-yl)propan-1-ol

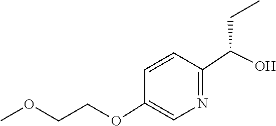

A mixture of [RuCl₂(mesitylene)]₂ (68 mg, 0.13 mmol), N-[(1S,2S)-2-amino-1,2-diphenylethyl]-4-methyl-1-sulfonylaniline (97 mg, 0.27 mmol) in MeCN (10 mL) and H₂O (10 mL) was stirred at 40° C. under N₂ for 1 h. 1-[5-(2-methoxyethoxy)pyridin-2-yl]propan-1-one (3.7 g, 17.7 mmol) and MeCN (30 mL) were added and the mixture was cooled to 5° C. Potassium formate (7.44 g, 88.4 mmol) in H₂O (5 mL) was added and the reaction mixture was slowly warmed to RT over 16 h. The reaction mixture was concentrated in vacuo and the residue was extracted with DCM (20 mL×6). The organic layers were separated, combined, dried over Na₂SO₄ and concentrated in vacuo. The residue was purified by silica gel flash chromatography (gradient 0-7% DCM/MeOH) to give the title compound (3.68 g, 92% yield) as a brown liquid. LCMS (Method E) MS (ESI) [M+H]⁺=212, Rt=1.11 min.

### Step 3: methyl (R)-2,3-difluoro-4-(1-(5-(2-methoxyethoxy)pyridin-2-yl)propoxy)benzoate

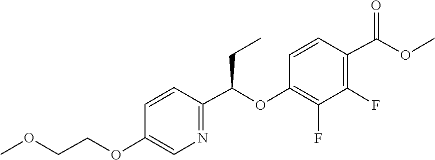

Under an argon atmosphere, (1S)-1-[5-(2-methoxyethoxy)pyridin-2-yl]propan-1-ol (3.24 g, 15.3 mmol), PPh₃ (1.41 g, 53.7 mmol), and methyl 2,3-difluoro-4-hydroxybenzoate (3.12 g, 53.7 mmol) were dissolved in dry THF (150 mL). The reaction mixture was stirred for 5 min, cooled to 0° C. and DIAD (1.09 g, 53.7 mmol) was added dropwise. The resulting mixture was stirred for 15 min, allowed to warm to RT and stirred for 12 h. The solvent was removed in vacuo and the residue was dissolved in H₂O (50 mL) and extracted with EtOAc (50 mL×4). The organic layers were separated, combined, dried over Na₂SO₄ and concentrated in vacuo. The residue was purified by silica gel flash chromatography (gradient 0-29% EtOAc/PE) to give the title compound (5.2 g, 79% yield) as a brown oil. LCMS (Method D) MS (ESI) [M+H]⁺=382, Rt=1.20 min.

### Step 4: (R)-2,3-difluoro-4-(1-(5-(2-methoxyethoxy)pyridin-2-yl)propoxy)benzoic acid

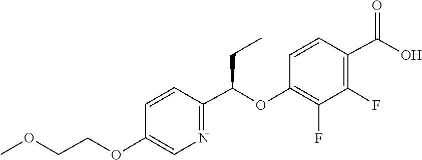 [SMILES: CC[C@@H](Oc1ccc(C(=O)O)c(F)c1F)c1ccc(OCCOC)cn1]

A mixture of methyl (R)-2,3-difluoro-4-(1-(5-(2-methoxyethoxy)pyridin-2-yl)propoxy)benzoate (5.2 g, 13.64 mmol) and LiOH H₂O (1.72 g, 40.91 mmol) in THF (30 mL) and H₂O (30 mL) was stirred for 12 h at 25° C. The reaction mixture was acidified with AcOH to pH=6-7 and extracted with DCM (40 mL×5). The organic layers were separated, combined, dried over Na₂SO₄ and concentrated in vacuo. The residue was purified by silica gel flash chromatography (gradient 0-6% DCM/MeOH). Further purification by chiral SFC (Method A) gave the title compound (1.24 g, >99% ee, 25% yield) as a yellow oil. LCMS (Method D) MS (ESI) [M+H]⁺=368, Rt=1.02 min. ¹H NMR (400 MHz, DMSO-d6, ppm) δ 13.17 (s, 1H), 8.32-8.25 (m, 1H), 7.58-7.49 (m, 1H), 7.44-7.34 (m, 2H), 6.96-6.89 (m, 1H), 5.42 (t, J=6.5 Hz, 1H), 4.22-4.08 (m, 2H), 3.72-3.61 (m, 2H), 3.29 (s, 3H), 2.12-1.91 (m, 2H), 0.91 (t, J=7.4 Hz, 3H).

### 4.5.5. Example 5. Preparation of (R)-2,3-difluoro-4-(1-(5-(pyridin-2-yloxy)pyridin-2-yl)propoxy)benzoic acid (Acid E)

### Step 1: 1-(5-(pyridin-2-yloxy)pyridin-2-yl)propan-1-one

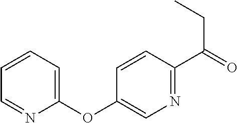 [SMILES: CCC(=O)c1ccc(Oc2ccccn2)cn1]

A mixture of 1-(5-hydroxypyridin-2-yl)propan-1-one (126 g, 834 mmol, Acid C, Step 1), 2-fluoropyridine (162 g, 1.67 mol) and K₂CO₃ (346 g, 2501 mmol) in NMP (1 L) was heated at 140° C. for 18 h. The mixture was poured into H₂O (3 L) and then extracted with EtOAc (800 mL×3). The combined organic layers were died over Na₂SO₄ and concentrated in vacuo. The residue was purified by silica gel flash chromatography (gradient 0-30% EtOAc/PE) to give the title compound (125 g, 65% yield) as an off-white solid. LCMS (Method D) MS (ESI) [M+H]⁺=229, Rt=1.25 mins. ¹H NMR (400 MHz, DMSO-d6, ppm) δ 8.58 (d, J=2.6 Hz, 1H), 8.20-8.18 (m, 1H), 8.05-8.03 (m, 1H), 7.97-7.93 (m 1H), 7.80-7.77 (m, 1H), 7.25-7.21 (m, 2H), 3.18 (q, J=7.3 Hz, 2H), 1.11 (t, J=7.3 Hz, 3H).

### Step 2: (S)-1-(5-(pyridin-2-yloxy)pyridin-2-yl)propan-1-ol

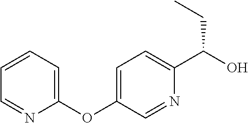 [SMILES: CC[C@H](O)c1ccc(Oc2ccccn2)cn1]

A mixture of [RuCl₂(mesitylene)]₂ (5.62 g, 10.95 mmol) and N-[(1S,2S)-2-amino-1,2-diphenylethyl]-4-methyl-1-sulfonylaniline (8.05 g, 21.9 mmol) in MeCN (300 mL) and H₂O (300 mL) was degassed and purged with N₂ for 3 times and the resulting mixture was stirred at 40° C. for 1 h. A solution of 1-[5-(pyridin-2-yloxy)pyridin-2-yl]propan-1-one (125 g, 548 mmol) in MeCN (1 L) was added at 0° C. HCO₂K (276 g, 3.29 mol) was added, and the resulting mixture was allowed to warm to RT and stirred for 12 h. The mixture was diluted with H₂O (1 L) and extracted with EtOAc (700 mL×3). The combined organic layers were dried over Na₂SO₄ and concentrated in vacuo. The residue was purified by silica gel flash chromatography (gradient 0-50% EtOAc/PE) to give the title compound (123 g, 89% yield) as a yellow oil. LCMS (Method E) MS (ESI) [M+H]⁺=231, Rt=1.26 min. ¹H NMR (400 MHz, DMSO-d6, ppm) δ 8.33 (d, J=2.6 Hz, 1H), 8.14-8.12 (m, 1H), 7.94-7.83 (m, 1H), 7.61-7.58 (m, 1H), 7.53-7.50 (m, 1H), 7.17-7.08 (m, 2H), 5.33 (d, J=4.8 Hz, 1H), 4.54-4.50 (m, 1H), 1.88-1.74 (m, 1H), 1.71-1.58 (m, 1H), 0.88 (t, J=7.4 Hz, 3H).

### Step 3: methyl (R)-2,3-difluoro-4-(1-(5-(pyridin-2-yloxy)pyridin-2-yl)propoxy)benzoate

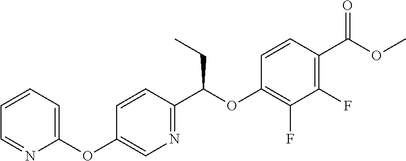 [SMILES: CC[C@@H](Oc1ccc(C(=O)OC)c(F)c1F)c1ccc(Oc2ccccn2)cn1]

Under N₂, to a solution of PPh₃ (420 g, 1.60 mol) in dry THF (3 L) added DIAD (324 g, 1.60 mol) dropwise at 0° C. and the mixture was stirred for 20 min. A solution of (S)-1-(5-(pyridin-2-yloxy)pyridin-2-yl)propan-1-ol (123 g, 534 mmol) and methyl 2,3-difluoro-4-hydroxybenzoate (121 g, 641 mmol) in dry THF (1 L) was added dropwise. The resulting mixture was stirred at RT for 16 h. The solvent was removed in vacuo. The residue was purified by silica gel flash chromatography (gradient 0-70% EtOAc/PE) to give the title compound (230 g, 75% yield) as a brown oil. LCMS (Method E) MS (ESI) [M+H]⁺=401, Rt=1.85 min. ¹H NMR (400 MHz, DMSO-d6, ppm) δ 8.18-8.11 (m, 1H), 7.95-7.85 (m, 1H), 7.69-7.50 (m, 3H), 7.22-7.09 (m, 2H), 7.05-7.00 (m, 1H), 5.55 (t, J=6.4 Hz, 1H), 3.82 (s, 3H), 2.11-2.04 (m, 2H), 0.96 (t, J=7.2 Hz, 3H).

### Step 4: (R)-2,3-difluoro-4-(1-(5-(pyridin-2-yloxy)pyridin-2-yl)propoxy)benzoic acid

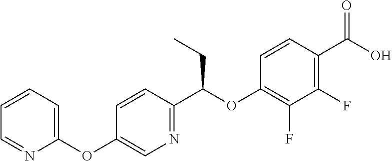 [SMILES: CC[C@@H](Oc1ccc(C(=O)O)c(F)c1F)c1ccc(Oc2ccccn2)cn1]

A mixture of methyl (R)-2,3-difluoro-4-(1-(5-(pyridin-2-yloxy)pyridin-2-yl)propoxy)benzoate (230 g, 574.5 mmol) and LiOH—H₂O (27.52 g, 1149 mmol) in THF/H₂O (2/1, 3 L) was stirred at 40° C. for 2 h. The reaction mixture was acidified with HCOOH to pH=6-7 and extracted with EtOAc (300 mL×3). The combined organic layers were dried over Na₂SO₄ and concentrated in vacuo. The residue was purified by chiral SFC (Method B) to give the title compound (90 g, >99% ee, 41% yield) as an off-white solid. LCMS (Method D) MS (ESI) [M+H]⁺=387, Rt=1.32 mins. ¹H NMR (400 MHz, DMSO-d6, ppm) δ 13.20 (s, 1H), 8.48 (d, J=2.6 Hz, 1H), 8.19-8.12 (m, 1H), 7.95-7.86 (m, 1H), 7.68-7.65 (m, 1H), 7.63-7.56 (m, 1H), 7.54-7.52 (m, 1H), 7.23-7.10 (m, 2H), 7.00 (t, J=7.8 Hz, 1H), 5.54 (t, J=6.4 Hz, 1H), 2.18-1.98 (m, 2H), 0.98 (t, J=7.2 Hz, 3H).

### 4.5.6. Example 6. Preparation of 2,3-difluoro-4-((R)-1-(5-((trans)-3-methoxycyclobutoxy)pyridin-2-yl)propoxy)benzoic acid (Acid F)

### Step 1: (cis)-3-hydroxycyclobutyl 4-methylbenzenesulfonate

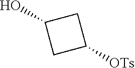 [SMILES: Cc1ccc(S(=O)(=O)O[C@H]2C[C@@H](O)C2)cc1]

A mixture of (cis)-3-(benzyloxy)cyclobutyl 4-methylbenzenesulfonate (2 g, 6.0 mmol) and Pd/C (200 mg) in THF (20 mL) was stirred under H₂ at RT for 2 h. The mixture was filtered through celite and concentrated in vacuo to give the title compound (1.6 g, 98% yield) as a colorless oil. LCMS (Method F) MS (ESI) [M+H]⁺=243, Rt=0.88 mins.

### Step 2: (cis)-3-methoxycyclobutyl 4-methylbenzenesulfonate

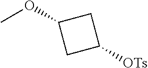 [SMILES: CO[C@H]1C[C@@H](OS(=O)(=O)c2ccc(C)cc2)C1]

A mixture of (cis)-3-hydroxycyclobutyl 4-methylbenzenesulfonate (1.00 g, 4.13 mmol), 1,8-bis(dimethylamino)naphthalene (4.39 g, 20.5 mmol) and trimethyloxonium tetrafluoroborate (3.03 g, 20.5 mmol) in DCM (40 mL) was stirred at RT for 18 h. The reaction mixture was filtered, and the filtrate was concentrated in vacuo. The residue was purified by silica gel flash chromatography (gradient 10-20% EtOAc/PE) to give the title compound (1.1 g, 88% yield) as a colorless oil. ¹H NMR (400 MHz, CDCl₃, ppm) δ 7.83-7.73 (m, 2H), 7.38-7.30 (m, 2H), 4.48 (p, J=7.3 Hz, 1H), 3.57-3.39 (m, 1H), 3.18 (s, 3H), 2.79-2.55 (m, 2H), 2.45 (s, 3H), 2.17-2.00 (m, 2H).

### Step 3: 1-(5-((trans)-3-methoxycyclobutoxy)pyridin-2-yl)propan-1-one

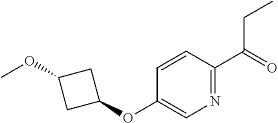 [SMILES: CCC(=O)c1ccc(O[C@H]2C[C@H](OC)C2)cn1]

A mixture of (cis)-3-methoxycyclobutyl 4-methylbenzenesulfonate (550 mg, 2.15 mmol), 1-(5-hydroxypyridin-2-yl)propan-1-one (324 mg, 2.15 mmol, Acid C, Step 1) and K₂CO₃ (593 mg, 4.29 mmol) in DMF (10 mL) was heated at 100° C. for 12 h. The reaction mixture was concentrated in vacuo and the residue was purified by silica gel flash chromatography (gradient 20-30% EtOAc/PE) to give the title compound (330 mg, 56% yield) as a colorless oil. ¹H NMR (400 MHz, CDCl₃, ppm) δ 8.22 (d, J=2.8 Hz, 1H), 8.03 (d, J=8.7 Hz, 1H), 7.14 (dd, J=8.7, 2.9 Hz, 1H), 4.99-4.89 (m, 1H), 4.31-3.95 (m, 1H), 3.29 (s, 3H), 3.24-3.13 (m, 2H), 2.66-2.38 (m, 4H), 1.22-1.19 (m, 3H).

### Step 4: (S)-1-(5-((trans)-3-methoxycyclobutoxy)pyridin-2-yl)propan-1-ol

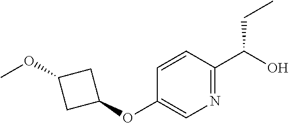 [SMILES: CC[C@H](O)c1ccc(O[C@H]2C[C@H](OC)C2)cn1]

Under N₂, a mixture of N-[(1S,2S)-2-amino-1,2-diphenylethyl]-4-methyl-1-sulfonylaniline (20.6 mg, 0.0561 mmol) and [RuCl₂(mesitylene)]₂ (16.4 mg, 0.028 mmol) in MeCN/H₂O (1/1, 3 mL) was stirred at 40° C. for 1 h. The mixture was cooled to 0° C. and a solution of 1-(5-((trans)-3-methoxycyclobutoxy)pyridin-2-yl)propan-1-one (330 mg, 1.40 mmol) in MeCN (1 mL) and HCOOK (590 mg, 7.01 mol) in H₂O (1 ml) were added dropwise. The reaction mixture was slowly allowed to warm to RT and stirred for an additional 12 h. The mixture was quenched with H₂O (20 mL) and extracted with EtOAc (20 mL×3). The combined organic layers were dried over Na₂SO₄ and concentrated in vacuo. The residue was purified by silica gel flash chromatography (gradient 30-50% EtOAc/PE) to give the title compound (160 mg, 41% yield) as a colorless oil. LCMS (Method D) MS (ESI) [M+H]⁺=238, Rt=0.75 min. ¹H NMR (400 MHz, DMSO-d₆, ppm) δ 8.07 (d, J 2.8 Hz, 1H), 7.37-7.35 (m, 1H), 7.25-7.22 (m, 1H), 5.16-5.15 (m, 1H), 4.90-4.85 (m, 1H), 4.45-4.43 (m, 1H), 4.09-4.05 (m, 1H), 3.17 (s, 3H), 2.42-2.39 (m, 2H), 2.35-2.22 (m, 2H), 1.79-1.67 (m, 1H), 1.65-1.51 (m, 1H), 0.82 (t, J 7.4 Hz, 3H).

### Step 5: methyl 2,3-difluoro-4-((R)-1-(5-((trans)-3-methoxycyclobutoxy)pyridin-2-yl)propoxy)benzoate

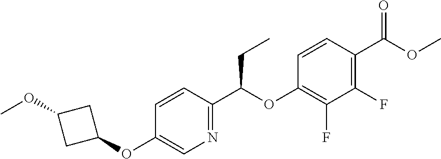

To a solution of (S)-1-(5-((trans)-3-methoxycyclobutoxy)pyridin-2-yl)propan-1-ol (130 mg, 0.55 mmol) and methyl 2,3-difluoro-4-hydroxybenzoate (113 mg, 0.60 mmol) in toluene (5 mL) was added CMBP (264 mg, 1.09 mmol). The reaction mixture was sealed and stirred at 120° C. for 16 h. The solvent was removed in vacuo. The residue was purified by silica gel flash chromatography (gradient 0-20% EtOAc/PE) to give the title compound (100 mg, 40% yield) as a yellow solid. LCMS (Method I) MS (ESI) [M+H]⁺=408, Rt=1.76 min. ¹H NMR (400 MHz, DMSO-d₆, ppm) δ 8.15 (d, J 2.8 Hz, 1H), 7.54-7.49 (m, 1H), 7.34-7.24 (m, 1H), 7.05 (dd, J 8.6, 2.8 Hz, 1H), 6.73-6.54 (m, 1H), 5.25-5.22 (m, 1H), 4.90-4.75 (m, 1H), 4.16-4.13 (m, 1H), 3.87 (s, 3H), 3.27 (s, 3H), 2.53-2.31 (m, 4H), 2.16-2.04 (m, 2H), 1.03 (t, J 7.4 Hz, 3H).

### Step 6: 2,3-difluoro-4-((R)-1-(5-((trans)-3-methoxycyclobutoxy)pyridin-2-yl)propoxy)benzoic acid

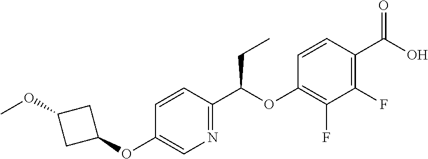 [SMILES: CC[C@@H](Oc1ccc(C(=O)O)c(F)c1F)c1ccc(O[C@H]2C[C@H](OC)C2)cn1]

A mixture of methyl 2,3-difluoro-4-((R)-1-(5-((trans)-3-methoxycyclobutoxy)pyridin-2-yl)propoxy)benzoate (1.5 g, 3.68 mmol) and LiOH H₂O (265 mg, 11.0 mmol) in THF/H₂O (3/1, 20 mL) was stirred for 16 h at RT. The reaction mixture was diluted with water (20 mL) and acidified to pH 5-6 with HCOOH. The mixture was extracted with EtOAc (20 mL×5). The combined organic layers were dried over Na₂SO₄ and concentrated in vacuo. The residue was purified by chiral SFC (Method C) to give the title compound (0.75 g, >99% ee, 95% purity, 52% yield) as an off-white solid. LCMS (Method K) MS (ESI) [M+H]⁺=394, Rt=1.36 min. ¹H NMR (400 MHz, DMSO-d₆, ppm) δ 13.18 (s, 1H), 8.19-8.18 (m, 1H), 7.54 (t, J 8.5 Hz, 1H), 7.38-7.36 (m, 1H), 7.28-7.25 (m, 1H), 6.93 (t, J 8.1 Hz, 1H), 5.42 (t, J 6.4 Hz, 1H), 4.93-4.79 (m, 1H), 4.12-3.99 (m, 1H), 3.15 (s, 3H), 2.43-2.38 (m, 2H), 2.33-2.21 (m, 2H), 2.09-1.86 (m, 2H), 0.91 (t, J 7.3 Hz, 3H).

### 4.5.7. Example 7. Preparation of (R)-4-(1-(5-(pyridin-2-yloxy)pyridin-2-yl)propoxy)-3-(trifluoromethyl)benzoic acid (Acid G)

### Step 1: methyl (R)-4-(1-(5-(pyridin-2-yloxy)pyridin-2-yl)propoxy)-3-(trifluoromethyl)benzoate

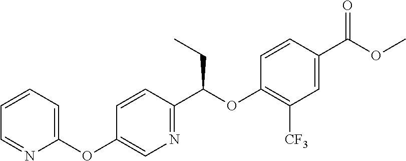 [SMILES: CC[C@@H](Oc1ccc(C(=O)OC)cc1C(F)(F)F)c1ccc(Oc2ccccn2)cn1]

To a solution of PPh₃ (13.6 g, 52 mmol) in dry THF (20 mL) under a N₂ atmosphere was added DIAD (10.5 g, 52 mmol). The reaction mixture was stirred at RT for 20 minutes. A solution of (1S)-1-[5-(pyridin-2-yloxy)pyridin-2-yl]propan-1-ol (4.0 g, 17 mmol, Acid E, Step 2) and methyl 4-hydroxy-3-(trifluoromethyl)benzoate (4.6 g, 21 mmol) in dry THF (20 mL) was slowly added dropwise. The reaction mixture was stirred at RT for 2 h. The solvent was removed in vacuo. The residue was purified by silica gel flash chromatography (eluent 3:1 PE/EtOAc) to give the title compound (11.0 g, 88% yield) as a white solid. LCMS (Method D) MS (ESI) [M+H]⁺=433, Rt=1.62 minutes.

### Step 2: (R)-4-(1-(5-(pyridin-2-yloxy)pyridin-2-yl)propoxy)-3-(trifluoromethyl)benzoic acid

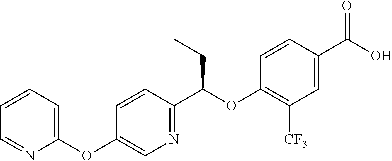 [SMILES: CC[C@@H](Oc1ccc(C(=O)O)cc1C(F)(F)F)c1ccc(Oc2ccccn2)cn1]

A mixture of methyl (R)-4-(1-(5-(pyridin-2-yloxy)pyridin-2-yl)propoxy)-3-(trifluoromethyl)benzoate (11.0 g, 25.4 mmol), LiOH—H₂O (1.21 g, 50 mmol) in THF (30 mL) and H₂O (15 mL) was stirred at RT for 16 h. The reaction mixture was acidified with HOAc to pH 5˜6 and extracted with EtOAc (20 mL×3). The combined organic layers were dried over Na₂SO₄, concentrated in vacuo and lyophilized. (R)-4-(1-(5-(pyridin-2-yloxy)pyridin-2-yl)propoxy)-3-(trifluoromethyl)benzoic acid (3.0 g) was purified by chiral SFC (Method D) to give the title compound (2.0 g, >99% ee, 19% yield) as a white solid. LCMS (Method D) MS (ESI) [M+H]⁺=419, Rt=1.42 min. ¹H NMR (400 MHz, DMSO-d6, ppm) δ 13.09 (s, 1H), 8.48 (d, J=2.5 Hz, 1H), 8.17-8.11 (m, 2H), 8.11-8.05 (m, 1H), 7.93-7.86 (m, 1H), 7.66 (dd, J=8.6, 2.7 Hz, 1H), 7.39 (d, J=8.6 Hz, 1H), 7.21-7.10 (m, 3H), 5.62 (t, J=6.1 Hz, 1H), 2.22-1.86 (m, 2H), 0.97 (t, J=7.3 Hz, 3H).

### 4.5.8. Example 8. Preparation of (R)-4-(1-(5-(pyridin-2-yloxy)pyridin-2-yl)ethoxy)-3-(trifluoromethyl)benzoic acid (Acid H)

### Step 1: 1-(5-(pyridin-2-yloxy)pyridin-2-yl)ethan-1-one

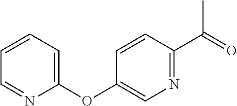 [SMILES: CC(=O)c1ccc(Oc2ccccn2)cn1]

A mixture of 1-(5-hydroxypyridin-2-yl)ethanone (5.1 g, 37 mmol), 2-fluoropyridine (7.22 g, 74 mmol) and K₂CO₃ (15.4 g, 110 mmol) in NMP (50 mL) was stirred at 140° C. for 16 h. The solution was diluted with H₂O (100 mL), extracted with EtOAc (100 mL×2). The combined organic layers were washed with brine (30 mL×3), dried over anhydrous Na₂SO₄ and concentrated in vacuo. The residue was purified by silica gel flash chromatography (eluent 6:1 PE/EtOAc) to give the title compound (5.0 g, 63% yield) as a yellow solid. LCMS (Method D) MS (ESI) [M+H]⁺=215, Rt=1.11 min.

### Step 2: (S)-1-(5-(pyridin-2-yloxy)pyridin-2-yl)ethan-1-ol

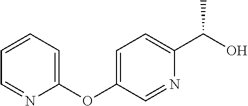 [SMILES: C[C@H](O)c1ccc(Oc2ccccn2)cn1]

A mixture of [RuCl₂(mesitylene)]₂ (0.24 g, 0.40 mmol) and N-[(1S,2S)-2-amino-1,2-diphenylethyl]-4-methyl-1-sulfonylaniline (0.34 g, 0.90 mmol) in CH₃CN (20 mL) and H₂O (20 mL) was degassed and purged with N₂ (x 3) and stirred at 40° C. for 1 h. The reaction mixture was cooled to 0° C. and a solution of 1-(5-(pyridin-2-yloxy)pyridin-2-yl)ethan-1-one (5.0 g, 23 mmol) in CH₃CN (20 mL), followed by a solution of HCOOK (9.80 g, 120 mmol) in H₂O (10 mL) were added. The resulting mixture was stirred at RT for 15 h. The solution was extracted with EtOAc (150 mL×2), and the combined organic layers were washed with brine (50 mL), dried over Na₂SO₄ and concentrated in vacuo. The residue was purified by silica gel flash chromatography (eluent 1:1 PE/EtOAc) to give the title compound (5.0 g, 99% yield) as a yellow oil. LCMS (Method D) MS (ESI) [M+H]⁺=217, Rt=0.83 min.

### Step 3: methyl (R)-4-(1-(5-(pyridin-2-yloxy)pyridin-2-yl)ethoxy)-3-(trifluoromethyl)benzoate

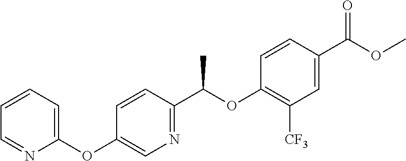 [SMILES: COC(=O)c1ccc(O[C@H](C)c2ccc(Oc3ccccn3)cn2)c(C(F)(F)F)c1]

To a solution of (S)-1-(5-(pyridin-2-yloxy)pyridin-2-yl)ethan-1-ol (3.0 g, 14 mmol), methyl 4-hydroxy-3-(trifluoromethyl)benzoate (3.67 g, 17 mmol) and PPh₃ (10.9 g, 42 mmol) in THF (60 mL) was added DIAD (8.43 g, 42 mmol) at 0° C. under N₂. The reaction mixture was stirred for 1.5 hours at RT. The solution was diluted with H₂O (100 mL) and extracted with EtOAc (100 mL×2). The combined organic layers were washed with brine (50 mL), dried over Na₂SO₄ and concentrated in vacuo. The residue was purified by silica gel flash chromatography (eluent 8:1 PE/EtOAc) to give the title compound (5.0 g, 27% yield) as a yellow oil. LCMS (Method D) MS (ESI) [M+H]⁺=419, Rt=1.58 min.

### Step 4: (R)-4-(1-(5-(pyridin-2-yloxy)pyridin-2-yl)ethoxy)-3-(trifluoromethyl)benzoic acid

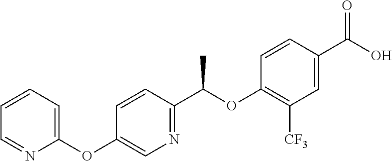 [SMILES: C[C@@H](Oc1ccc(C(=O)O)cc1C(F)(F)F)c1ccc(Oc2ccccn2)cn1]

To a solution of methyl 4-[(1R)-1-[5-(cyclopropylmethoxy)pyridin-2-yl]propoxy]-2,3-difluorobenzoate (5.0 g, 12 mmol) THF (40 mL) and H₂O (20 mL) at RT was added LiOH—H₂O (1.15 g, 48 mmol). The resulting solution was stirred at RT for 3 h. The pH was adjusted to around 5 by addition of AcOH. The solution was extracted with DCM (150 mL×2). The combined organic layers were washed with brine, dried over Na₂SO₄ and concentrated under reduced pressure. The residue was purified by silica gel flash chromatography (eluent 60:1 DCM/MeOH). Further purification was carried out by chiral SFC (Method E) to give the title compound (2.1 g, >99% ee, 43% yield) as a white solid. LCMS (Method D) MS (ESI) [M+H]⁺=405, Rt=1.37 min. ¹H NMR (400 MHz, DMSO-d6, ppm) δ 13.12 (s, 1H), 8.47 (d, J=2.7 Hz, 1H), 8.14-8.10 (m, 3H), 7.94-7.86 (m, 1H), 7.69 (dd, J=8.6, 2.7 Hz, 1H), 7.48 (d, J=8.6 Hz, 1H), 7.31 (d, J=9.2 Hz, 1H), 7.21-7.11 (m, 2H), 5.84 (q, J=6.3 Hz, 1H), 1.67 (d, J=6.4 Hz, 3H).

### 4.5.9. Example 9. Preparation of (R)-2,3-difluoro-4-(1-(5-(pyridin-2-yloxy)pyridin-2-yl)ethoxy)benzoic acid (Acid I)

### Step 1: methyl (R)-2,3-difluoro-4-(1-(5-(pyridin-2-yloxy)pyridin-2-yl)ethoxy)benzoate

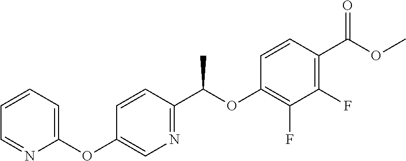 [SMILES: COC(=O)c1ccc(O[C@H](C)c2ccc(Oc3ccccn3)cn2)c(F)c1F]

PPh₃ (7.24 g, 28 mmol) and DIAD (5.58 g, 28 mmol) were dissolved in THF (20 mL) under a N₂ atmosphere and stirred for 30 min at 0° C. A solution of (1S)-1-[5-(pyridin-2-yloxy)pyridin-2-yl]ethanol (2.0 g, 9.2 mmol, Acid H, Step 2) and methyl 2,3-difluoro-4-hydroxybenzoate (2.1 g, 11 mmol) in THF (10 mL) was added dropwise. The resulting mixture was stirred at RT for 2 h. The solution was diluted with H₂O (50 mL) and extracted with EtOAc (100 mL×2). The combined organic layers were washed with brine (50 mL), dried over Na₂SO₄ and concentrated in vacuo. The residue was purified by silica gel flash chromatography (eluent 8:1 PE/EtOAc) to give the title compound (3.0 g, 64% yield) as a yellow oil. LCMS (Method D) MS (ESI) [M+H]⁺=387, Rt=1.43 min.

### Step 2: (R)-2,3-difluoro-4-(1-(5-(pyridin-2-yloxy)pyridin-2-yl)ethoxy)benzoic acid

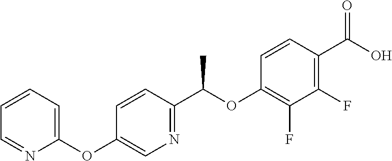 [SMILES: C[C@@H](Oc1ccc(C(=O)O)c(F)c1F)c1ccc(Oc2ccccn2)cn1]

To a solution of methyl (R)-2,3-difluoro-4-(1-(5-(pyridin-2-yloxy)pyridin-2-yl)ethoxy)benzoate (8.0 g, 21 mmol) in THF (40 mL) and H₂O (20 mL) at RT was added LiOH—H₂O (1.98 g, 83 mol). The resulting solution was stirred for 2 h. The pH was adjusted to around 5 by addition of AcOH. The solution was extracted with DCM (80 mL×2). The combined organic layers were washed with brine (50 mL), dried over Na₂SO₄, filtered and concentrated in vacuo. The residue was purified by silica gel flash chromatography (eluent 60:1 DCM/MeOH). Further purification was carried out by chiral SFC (Method F) to give the title compound (1.8 g, >99% ee, 23% yield) as a white solid.

LCMS (Method D) MS (ESI) [M+H]⁺=373, Rt=1.26 min. ¹H NMR (400 MHz, DMSO-d6, ppm) δ 13.24 (s, 1H), 8.47 (d, J=2.5 Hz, 1H), 8.15 (dd, J=4.9, 1.2 Hz, 1H), 7.95-7.84 (m, 1H), 7.72-7.51 (m, 3H), 7.23-7.11 (m, 2H), 7.05 (t, J=8.2 Hz, 1H), 5.77 (q, J=6.3 Hz, 1H), 1.70 (d, J=6.4 Hz, 3H).

### 4.5.10. Example 10. Preparation of (R)-4-(1-(5-(cyclopropylmethoxy)pyridin-2-yl)propoxy)-3-(trifluoromethyl)benzoic acid (Acid J)

### Step 1: methyl (R)-4-(1-(5-(cyclopropylmethoxy)pyridin-2-yl)propoxy)-3 (trifluoromethyl)benzoate

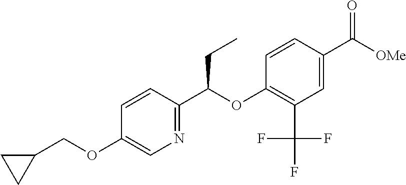 [SMILES: CC[C@@H](Oc1ccc(C(=O)OC)cc1C(F)(F)F)c1ccc(OCC2CC2)cn1]

Under N₂, a mixture of PPh₃ (11.4 g, 44 mol) and DIAD (8.80 g, 44 mmol) in 100 mL of anhydrous THF was stirred at 0° C. for 30 minutes. Then, a solution of (S)-1-(5-(cyclopropylmethoxy)pyridin-2-yl)propan-1-ol (3.00 g, 15 mmol, Acid C, Step 3) and methyl 4-hydroxy-3-(trifluoromethyl)benzoate (3.51 g, 16 mmol) in THF (50 mL) was added dropwise. The mixture was stirred at RT for 3 hours. The solution was diluted with EtOAc (200 mL) and water (100 mL). The layers were separated and the organic layer was washed with brine (50 mL), dried over anhydrous Na₂SO₄, filtered, and the solvent was removed under reduced pressure. The residue was purified by silica gel flash chromatography (eluent: EtOAc/PE=1/8) to give the title compound (4.5 g, 68% yield) as a yellow oil.

LCMS (Method K) MS (ESI) [M+H]⁺=410.1, Rt=1.71 min. ¹H NMR (400 MHz, DMSO-d6, ppm) δ 8.29 (d, J=2.8 Hz, 1H), 8.12-8.09 (m, 1H), 8.80-8.03 (m, 1H), 7.38 (dd, J=8.7, 2.9 Hz, 1H), 7.25 (d, J=8.7 Hz, 1H), 7.15 (d, J=8.9 Hz, 1H), 5.52 (t, J=6.2 Hz, 1H), 3.87 (d, J=7.1 Hz, 2H), 3.83 (s, 3H), 2.08-1.91 (m, 2H), 1.22-1.14 (m, 1H), 0.94 (t, J=7.3 Hz, 3H), 0.61-0.52 (m, 2H), 0.37-0.26 (m, 2H).

### Step 2: (R)-4-(1-(5-(cyclopropylmethoxy)pyridin-2-yl)propoxy)-3-(trifluoromethyl)benzoic acid

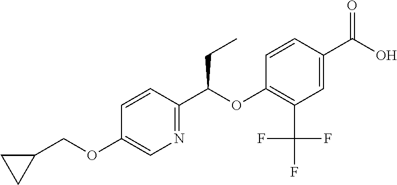 [SMILES: CC[C@@H](Oc1ccc(C(=O)O)cc1C(F)(F)F)c1ccc(OCC2CC2)cn1]

To a solution of methyl (R)-4-(1-(5-(cyclopropylmethoxy)pyridin-2-yl)propoxy)-3-(trifluoromethyl)benzoate (4.50 g, 11 mmol) in THF (30 mL) and H₂O (10 mL) at RT was added LiOH H₂O (1.05 g, 44 mmol). The resulting solution was stirred at RT for 2 h. The solution was diluted H₂O (100 mL) and acidified with HCOOH until pH 5-6. The solution was extracted with EtOAc (100 mL×2). The combined organic layers were washed with saturated NaCl (50 mL), dried over anhydrous Na₂SO₄, filtered and the solvent was removed under reduced pressure. The residue was purified by chiral SFC (Method G) to give the title compound (1.8 g, >99% ee, 42% yield) as a white solid.

LCMS (Method K) MS (ESI) [M+H]⁺=396.1, Rt=1.52 min. ¹H NMR (400 MHz, DMSO-d₆, ppm) δ 13.09 (s, 1H), 8.28 (d, J=2.7 Hz, 1H), 8.10 (d, J=1.9 Hz, 1H), 8.03 (dd, J=8.8, 2.0 Hz, 1H), 7.38 (dd, J=8.7, 2.9 Hz, 1H), 7.24 (d, J=8.7 Hz, 1H), 7.11 (d, J=8.9 Hz, 1H), 5.51 (t, J=6.1 Hz, 1H), 3.87 (d, J=7.1 Hz, 2H), 2.07-1.88 (m, 2H), 1.28-1.14 (m, 1H), 0.93 (t, J=7.3 Hz, 3H), 0.64-0.49 (m, 2H), 0.37-0.23 (m, 2H).

### 4.5.11. Example 11. Preparation of 4-((R)-1-(5-(trans-3-methoxycyclobutoxy)pyridin-2-yl)propoxy)-3-(trifluoromethyl)benzoic acid (Acid K)

### Step 1: methyl 4-((R)-1-(5-(trans-3-methoxycyclobutoxy)pyridin-2-yl)propoxy)-3-(trifluoromethyl)benzoate

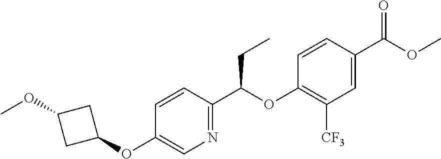

Under N₂, to a solution of (S)-1-(5-((trans)-3-methoxycyclobutoxy)pyridin-2-yl)propan-1-ol (2.80 g, 11.8 mmol, Acid F, Step 4) and methyl 4-hydroxy-3-(trifluoromethyl)benzoate (2.72 g, 12.4 mmol) in toluene (40 mL) was added CMBP (5.69 g, 23.6 mmol). The reaction mixture was sealed and stirred at 120° C. for 16 h. The solvent was removed under reduced pressure. The residue was purified by silica gel flash chromatography (eluent: EtOAc in PE, 0-20%) to afford the title compound (2.3 g, 90% purity, 40% yield) as a yellow solid.

LCMS (Method K) MS (ESI) [M+H]⁺=440.1, Rt=1.67 min. ¹H NMR (400 MHz, CDCl₃, ppm) δ 8.25 (d, J 1.9 Hz, 1H), 8.15 (d, J 2.7 Hz, 1H), 7.99 (dd, J 8.8, 2.0 Hz, 1H), 7.23 (d, J 8.7 Hz, 1H), 7.03 (dd, J 8.7, 2.8 Hz, 1H), 6.87 (d, J 8.8 Hz, 1H), 5.41-5.31 (m, 1H), 4.84-4.80 (m, 1H), 4.15-4.09 (m, 1H), 3.87 (s, 3H), 3.26 (s, 3H), 2.54-2.25 (m, 4H), 2.09-2.00 (m, 2H), 1.04 (t, J 7.4 Hz, 3H).

### Step 2: 4—((R)-1-(5-(trans-3-methoxycyclobutoxy)pyridin-2-yl)propoxy)-3-(trifluoromethyl)benzoic acid

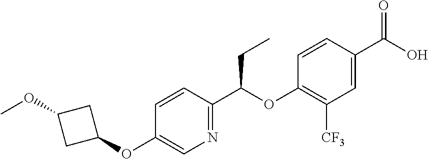 [SMILES: CC[C@@H](Oc1ccc(C(=O)O)cc1C(F)(F)F)c1ccc(O[C@H]2C[C@H](OC)C2)cn1]

A mixture of methyl 4-((R)-1-(5-(trans-3-methoxycyclobutoxy)pyridin-2-yl)propoxy)-3-(trifluoromethyl)benzoate (2.30 g, 5.23 mmol) and LiOH H₂O (377 mg, 15.7 mmol) in THF/H₂O (3/1, 20 mL) was stirred for 16 h at RT. The reaction mixture was diluted with water (20 mL), acidified with AcOH until pH 4-5. The mixture was extracted with EtOAc (20 mL×5). The combined organic layers were dried over Na₂SO₄ and concentrated in vacuo. The residue was purified by chiral SFC (Method H) to give the title compound (1.15 g, >99% ee, 90% purity, 49% yield,) as a yellow solid.

LCMS (Method K) MS (ESI) [M+H]⁺=426.1, Rt=1.48 min. ¹H NMR (400 MHz, DMSO-d6, ppm) δ 13.07 (s, 1H), 8.20-8.19 (m, 1H), 8.09 (s, 1H), 8.05-8.03 (m, 1H), 7.30-7.23 (m, 2H), 7.11 (d, J 8.8 Hz, 1H), 5.51 (t, J 6.1 Hz, 1H), 4.93-4.78 (m, 1H), 4.14-3.96 (m, 1H), 3.15 (s, 3H), 2.47-2.35 (m, 2H), 2.33-2.21 (m, 2H), 2.05-1.89 (m, 2H), 0.93 (t, J 7.3 Hz, 3H).

### 4.5.12. Example 12. Preparation of (R)-4-(1-(4-(cyclopropylmethoxy)pyridin-2-yl)propoxy)-2,3-difluorobenzoic acid (Acid L)

### Step 1: 1-(4-hydroxypyridin-2-yl)propan-1-one

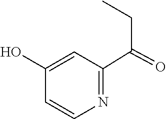

Under N₂, to a solution of 4-hydroxypicolinonitrile (5.00 g, 41.6 mmol) in THF (50 mL) was added EtMgBr (4.7 mL, 83 mmol, 2M in THF) at 0° C. The reaction mixture was stirred for 30 min at 0° C. and then heated to 70° C. for 1 h. The reaction mixture was allowed to cool to RT and was poured into ice-water (150 mL) and then acidified with 3 N HCl until pH 4-5. The mixture was stirred at RT for 10 mins and extracted with EtOAc (200 mL×8). The combined organic layers were died over Na₂SO₄ and concentrated in vacuo. The residue was purified by silica gel flash chromatography (eluent: MeOH/DCM: 0-10%) to give the title compound (5.55 g, 79% yield) as a light yellow solid.

LCMS (Method K) MS (ESI) [M+H]⁺=151.9, Rt=0.48 min. ¹H NMR (400 MHz, methanol-d4, ppm) δ 7.89 (d, J=6.8 Hz, 1H), 7.13-7.07 (m, 1H), 6.65-6.52 (m, 1H), 3.04 (q, J=7.2 Hz, 2H), 1.19 (t, J=7.2 Hz, 3H).

### Step 2: 1-(4-(cyclopropylmethoxy)pyridin-2-yl)propan-1-one

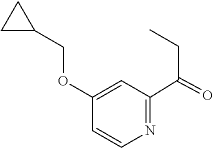

A mixture of 1-(4-hydroxypyridin-2-yl)propan-1-one (4.0 g, 27 mmol), (bromomethyl)cyclopropane (5.4 g, 40 mmol) and K₂CO₃ (11 g, 80 mol) in DMF (80 mL) was stirred at 90° C. for 16 h. The resulting mixture was diluted with water (80 mL) and extracted with EtOAc (60 mL×3). The combined organic phases were washed with brine (15 mL), dried over Na₂SO₄, concentrated under vacuum and the residue was purified by silica gel flash chromatography (eluent: EtOAc/PE, 0-10%) to afford the title compound (5.4 g, 98% yield) as a yellow oil.

LCMS (Method K) MS (ESI) [M+H]⁺=206.2, Rt=1.23 min. ¹H NMR (400 MHz, DMSO-d₆, ppm) δ 8.50 (d, J=5.6 Hz, 1H), 7.41 (d, J=2.6 Hz, 1H), 7.20 (dd, J=5.6, 2.6 Hz, 1H), 3.99 (d, J=7.1 Hz, 2H), 3.15 (q, J=7.3 Hz, 2H), 1.31-1.19 (m, 1H), 1.08 (t, J=7.3 Hz, 3H), 0.66-0.53 (m, 2H), 0.43-0.31 (m, 2H).

### Step 3: (S)-1-(4-(cyclopropylmethoxy)pyridin-2-yl)propan-1-ol

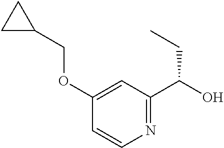

A mixture of [RuCl₂(mesitylene)]₂ (0.28 g, 0.5 mmol) and N-[(1S,2S)-2-amino-1,2-diphenylethyl]-4-methyl-1-sulfonylaniline (0.39 g, 1.0 mol) in MeCN (20 mL) and H₂O (20 mL) was degassed and purged with N₂ for 3 times, and then the mixture was stirred at 40° C. for I h under a N₂ atmosphere. The mixture was cooled to 0° C. and 1-[5-(pyridin-2-yloxy)pyridin-2-yl]ethenone (5.5 g, 27.0 mmol) in MeCN (10 mL) was added. Potassium formate (11.3 g, 130 mmol) in H₂O (30 mL) was added in the mixture and the mixture was stirred at RT for 15 h under a N₂ atmosphere. The solution was diluted with water (100 mL) and extracted with EtOAc (100 mL×2). The combined organic layers were washed with saturated NaCl (50 mL), dried over anhydrous Na₂SO₄, and the solvent was removed under reduced pressure. The residue was purified by silica gel flash chromatography (eluent: PE/EtOAc=2/1) to give the title compound (5.4 g, 84% yield) as a yellow oil.

LCMS (Method K) MS (ESI) [M+H]⁺=208.2, Rt=0.73 min.

### Step 4: (R)-4-(1-(4-(cyclopropylmethoxy)pyridin-2-yl)propoxy)-2,3-difluorobenzoate

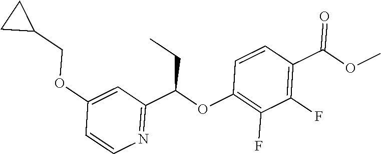 [SMILES: CC[C@@H](Oc1ccc(C(=O)OC)c(F)c1F)c1cc(OCC2CC2)ccn1]

Under N₂, a mixture of PPh₃ (19.8 g, 75 mmol) and DIAD (15.2 g, 75 mmol) in anhydrous THF (100 mL) was stirred at 0° C. for 30 min. A solution of (1S)-1-[4-(cyclopropylmethoxy)pyridin-2-yl]propan-1-ol (5.2 g, 25 mmol) and methyl-2,3-difluoro-4-hydroxybenzoate (6.14 g, 33 mol) in THF (20 mL) was added dropwise and the mixture was stirred at RT for 2 hours. The solution was diluted with water (100 mL) and extracted with EtOAc (100 mL×2). The combined organic layers were washed with saturated NaCl (50 mL), dried over anhydrous Na₂SO₄, filtered and the solvent was removed under reduced pressure. The residue was purified by silica gel flash chromatography (eluent: PE/EtOAc=2/1) to give methyl the title compound (15 g, 100% yield) as a yellow oil.

LCMS (Method K) MS (ESI) [M+H]⁺=378.1, Rt=1.45 min. ¹H NMR (400 MHz, DMSO-d6, ppm) δ 8.37 (d, J=5.7 Hz, 1H), 7.62-7.54 (m, 1H), 6.99-6.92 (m, 2H), 6.89 (dd, J=5.7, 2.5 Hz, 1H), 5.40 (t, J=6.4 Hz, 1H), 3.95-3.86 (m, 2H), 3.82 (s, 3H), 2.04-1.99 (m, 2H), 1.13-1.05 (m, 1H), 0.94 (t, J=9.2, 5.5 Hz, 3H), 0.61-0.51 (m, 2H), 0.37-0.28 (m, 2H).

### Step 5: (R)-4-(1-(4-(cyclopropylmethoxy)pyridin-2-yl)propoxy)-2,3-difluorobenzoic acid

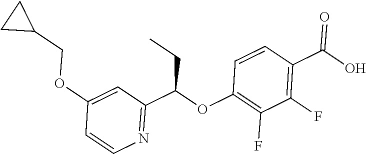 [SMILES: CC[C@@H](Oc1ccc(C(=O)O)c(F)c1F)c1cc(OCC2CC2)ccn1]

To a solution of methyl 4-[(1R)-1-[4-(cyclopropylmethoxy)pyridin-2-yl]propoxy]-2,3-difluorobenzoate (15 g, 40 mmol) in THF (60 mL) and H₂O (30 mL) at RT was added LiOH H₂O (6.66 g, 160 mmol). The resulting solution was stirred at RT for 16 h. The mixture was acidified with AcOH until pH 5-6. The solution was extracted with EtOAc (150 mL×2). The combined organic layers were washed with saturated NaCl (50 mL), dried over anhydrous Na₂SO₄, filtered and the solvent was removed under reduced pressure. The resultant solids were refluxed in MeOH (50 mL) until all fully dissolved. The solution was then cooled to RT slowly and was left to stand at RT for 18 h. The solids were filtered and washed with MeOH (5 mL) to give the title compound (3.3 g, 98% ee, 22% yield) as a white solid.

LCMS (Method K) MS (ESI) [M+H]⁺=364.1, Rt=1.20 min. ¹H NMR (400 MHz, DMSO-d₆, ppm) δ 13.20 (s, 1H), 8.37 (d, J=5.7 Hz, 1H), 7.55 (t, J=7.7 Hz, 1H), 7.03-6.77 (m, 3H), 5.38 (t, J=6.3 Hz, 1H), 4.00-3.78 (m, 2H), 2.15-1.89 (m, 2H), 1.27-1.11 (m, 1H), 0.94 (t, J=7.3 Hz, 3H), 0.63-0.48 (m, 2H), 0.40-0.23 (m, 2H).

### 4.5.13. Example 13. Preparation of (R)-2,3-difluoro-4-(1-(4-(pyridin-2-yloxy)pyridin-2-yl)propoxy)benzoic acid (Acid M)

### Step 1: 1-(4-(pyridin-2-yloxy)pyridin-2-yl)propan-1-one

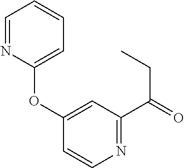 [SMILES: CCC(=O)c1cc(Oc2ccccn2)ccn1]

A mixture of 1-(4-hydroxypyridin-2-yl)propan-1-one (5.55 g, 36.7 mmol, Acid L, Step 1), 2-fluoropyridine (5.34 g. 54.4 mmol) and K₂CO₃ (7.61 g, 54.4 mmol) in DMF (100 mL) was stirred at 140° C. for 36 h. The mixture was allowed to cool to RT and was poured into water (300 mL), and extracted with EtOAc (300 mL×3). The combined organic layers were died over Na₂SO₄ and concentrated in vacuo. The residue was purified by silica gel flash chromatography (eluent: EtOAc/PE: 0-40%) to give the title compound (3.77 g, 41% yield) as a light yellow solid.

LCMS (Method E) MS (ESI) [M+H]⁺=229.2, Rt=1.49 min. ¹H NMR (400 MHz, CDCl₃, ppm) δ 8.63 (d, J=5.5 Hz, 1H), 8.29-8.22 (m, 1H), 7.85-7.75 (m, 2H), 7.28-7.26 (m, 1H), 7.19-7.13 (m, 1H), 7.05 (d, J=8.2 Hz, 1H), 3.24 (q, J=7.3 Hz, 2H), 1.21 (t, J=7.3 Hz, 3H).

### Step 2: (S)-1-(4-(pyridin-2-yloxy)pyridin-2-yl)propan-1-ol

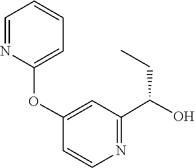 [SMILES: CC[C@H](O)c1cc(Oc2ccccn2)ccn1]

A mixture of [RuCl₂(mesitylene)]₂ (126 mg, 0.24 mmol) and N-((1S,2S)-2-amino-1,2-diphenylethyl)-4-methylbenzenesulfonamide (180 mg, 0.49 mmol) in MeCN (5 mL) and H₂O (5 mL) was degassed and purged with N₂ 3 times, and the mixture was stirred at 40° C. for 1 h under a N₂ atmosphere. The mixture was cooled to 0° C. and 1-(4-(pyridin-2-yloxy)pyridin-2-yl)propan-1-one (2.80 g, 12.3 mmol) in MeCN (10 mL) was added. Potassium formate (5.10 g, 61.3 mmol) in H₂O (10 mL) was added and the mixture was stirred at RT for 16 h under N₂. The mixture was diluted with water (100 mL) and extracted with EtOAc (100 mL×3). The combined organic layers were dried over Na₂SO₄ and concentrated under reduced pressure. The residue was purified by silica gel flash chromatography (eluent: MeOH/DCM: 0-10%) to give the title compound (2.80 g, 89% yield) as a yellow solid.

LCMS (Method K) MS (ESI) [M+H]⁺=230.9, Rt=0.64 min. ¹H NMR (400 MHz, CDCl₃, ppm) δ 8.48 (d, J=5.6 Hz, 1H), 8.33-8.22 (m, 1H), 7.84-7.75 (m, 1H), 7.17-7.12 (m, 1H), 7.07-7.01 (m, 2H), 7.00-6.95 (m, 1H), 4.74-4.60 (m, 1H), 1.95-1.82 (m, 1H), 1.78-1.63 (m, 1H), 0.97 (t, J=7.4 Hz, 3H).

### Step 3: methyl (R)-2,3-difluoro-4-(1-(4-(pyridin-2-yloxy)pyridin-2-yl)propoxy)benzoate

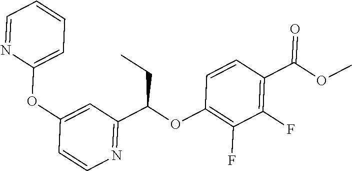 [SMILES: CC[C@@H](Oc1ccc(C(=O)OC)c(F)c1F)c1cc(Oc2ccccn2)ccn1]

Under N₂, to a solution of PPh₃ (9.31 g, 35.5 mmol) in dry THF (160 mL) was added DIAD (7.19 g, 35.5 mol) dropwise at 0° C. and the mixture was stirred at this temperature for 20 min. A solution of (S)-1-(4-(pyridin-2-yloxy)pyridin-2-yl)propan-1-ol (2.73 g, 11.8 mmol) and methyl 2,3-difluoro-4-hydroxybenzoate (2.23 g, 11.8 mmol) in dry THF (40 mL) was added dropwise. The resulting reaction mixture was stirred at RT for 16 h. The solvent was removed under reduced pressure. The residue was purified by silica gel flash chromatography (eluent: EtOAc/PE: 0-50%) to give the title compound (5.3 g, 80% purity, 89% yield) as a yellow oil.

LCMS (Method K) MS (ESI) [M+H]⁺=401.2, Rt=1.50 min. ¹H NMR (400 MHz, CDCl₃, ppm) δ 8.51 (d, J=5.6 Hz, 1H), 8.24-8.16 (m, 1H), 7.82-7.72 (m, 1H), 7.60-7.51 (m, 1H), 7.17-7.11 (m, 2H), 7.05-6.93 (m, 2H), 6.72-6.63 (m, 1H), 5.07-4.83 (m, 1H), 3.89 (s, 3H), 2.22-1.96 (m, 2H), 1.08 (t, J=7.4 Hz, 3H).

### Step 4: (R)-2,3-difluoro-4-(1-(4-(pyridin-2-yloxy)pyridin-2-yl)propoxy)benzoic acid

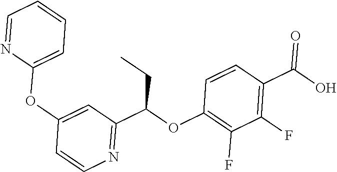 [SMILES: CC[C@@H](Oc1ccc(C(=O)O)c(F)c1F)c1cc(Oc2ccccn2)ccn1]

Methyl (R)-2,3-difluoro-4-(1-(4-(pyridin-2-yloxy)pyridin-2-yl)propoxy)benzoate (5.20 g, 13.0 mmol) and LiOH H₂O (1.25 g, 52.0 mmol) in THF/H₂O/MeOH (75 mL, 2/2/1) was stirred at RT for 3 h. The solvent was removed under reduced pressure and the residue was diluted with water (70 mL). The mixture was acidified with HCOOH to pH 5-6 and was extracted with EtOAc (40 mL×3). The combined organic layers were dried over Na₂SO₄ and concentrated in vacuo. The residue was purified by silica gel flash chromatography (eluent: DCM/MeOH=20/1, with 0.5% FA) to give a light yellow solid. The solids were refluxed in MeOH (16 mL) until fully dissolved. The solution was then cooled to RT slowly and allowed to stand at RT for 18 h. The solids were filtered, washed with MeOH (1 mL) and MeCN (5 mL×2), and dried to give the title compound (2.4 g, 97% ee, 47% yield) as a white solid.

LCMS (Method E) MS (ESI) [M+H]⁺=387.2, Rt=1.07 min. ¹H NMR (400 MHz, DMSO-d6, ppm) δ 13.21 (s, 1H), 8.53 (d, J=5.6 Hz, 1H), 8.25-8.12 (m, 1H), 8.01-7.85 (m, 1H), 7.62-7.50 (m, 1H), 7.33-7.24 (m, 1H), 7.20-7.11 (m, 2H), 7.10-7.01 (m, 1H), 7.00-6.89 (m, 1H), 5.50 (t, J=6.3 Hz, 1H), 2.06-1.98 (m, 2H), 0.95 (t, J=7.4 Hz, 3H).

### 4.5.14. Example 14. Preparation of 4-((R)-1-(5-(trans-3-(difluoromethoxy)cyclobutoxy)-pyridin-2-yl)propoxy)-2,3-difluorobenzoic acid (Acid N)

### Step 1: cis-3-(difluoromethoxy)cyclobutyl 4-methylbenzenesulfonate

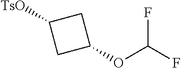 [SMILES: Cc1ccc(S(=O)(=O)O[C@H]2C[C@@H](OC(F)F)C2)cc1]

Under N₂, to a solution of 3-hydroxycyclobutyl 4-methylbenzenesulfonate (6.09 g, 25.1 mmol, Acid F, Step 1) in MeCN (60 mL) was added CuI (957 mg, 5.03 mmol). The mixture was heated to 50° C., and difluoro(fluorosulfonyl)acetic acid (6.71 g, 37.7 mmol) in MeCN (20 mL) was added dropwise at 50° C. over 45 min. The reaction solution was stirred for 1 h at 50° C. The solvent was removed under reduced pressure. The residue was purified by silica gel flash chromatography (eluent: EtOAc/PE: 0-9%) to give the title compound (4.50 g, 92% purity, 27% yield) as a colorless liquid.

¹H NMR (400 MHz, CDCl₃) δ 7.78 (d, J=8.3 Hz, 2H), 7.35 (d, J=8.1 Hz, 2H), 6.11 (t, ²J_{H-F}=73.6 Hz, 1H), 4.48 (p, J=7.2 Hz, 1H), 4.23 (p, J=7.1 Hz, 1H), 2.76-2.66 (m, 2H), 2.46 (s, 3H), 2.39-2.31 (m, 2H).

### Step 2: 1-(5-(trans-3-(difluoromethoxy)cyclobutoxy)pyridin-2-yl)propan-1-one

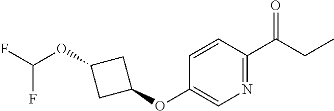 [SMILES: CCC(=O)c1ccc(O[C@H]2C[C@H](OC(F)F)C2)cn1]

A mixture of 1-(5-hydroxypyridin-2-yl)propan-1-one (2.15 g, 14.2 mmol), 3-(difluoromethoxy)cyclobutyl 4-methylbenzenesulfonate (4.37 g, 14.9 mmol) and K₂CO₃ (3.93 g, 28.5 mmol) in DMF (45 mL) was stirred for 6 h at 100° C. The reaction solvent was diluted with brine (50 mL) and extracted with EtOAc (50 mL×4). The combined organic phases were concentrated in vacuum. The residue was purified by silica gel flash chromatography (eluent: EtOAc/PE: 0-9%) to give the title compound (3.05 g, 910% purity, 72% yield) as a pale yellow solid.

LCMS (Method K) MS (ESI) [M+H]⁺=271.9, Rt=1.36 min. ¹H NMR (400 MHz, DMSO-d6, ppm) δ 8.31 (d, J=2.9 Hz, 1H), 7.94 (d, J=8.7 Hz, 1H), 7.41 (dd, J=8.7, 2.9 Hz, 1H), 6.69 (t, J=75.7 Hz, 1H), 5.13-5.05 (m, 1H), 4.90-4.80 (m, 1H), 3.11 (q, J=7.3 Hz, 2H), 2.70-2.62 (m, 2H), 2.57-2.51 (m, 2H), 1.08 (t, J=7.3 Hz, 3H).

### Step 3: (S)-1-(5-(trans-3-(difluoromethoxy)cyclobutoxy)pyridin-2-yl)propan-1-ol

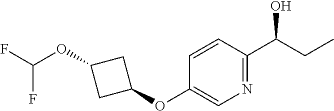 [SMILES: CC[C@H](O)c1ccc(O[C@H]2C[C@H](OC(F)F)C2)cn1]

Under N₂, a mixture of [RuCl₂(mesitylene)]₂ (115 mg, 0.22 mmol), N-[(1S,2S)-2-amino-1,2-diphenylethyl]-4-methyl-1-sulfonylaniline (165 mg, 0.45 mmol) in MeCN (4 mL)/H₂O (4 mL) was stirred for 1 h at 40° C. The reaction was cooled to 5° C. and 1-(5-(trans-3-(difluoromethoxy)cyclobutoxy)pyridin-2-yl)propan-1-one (3.05 g, 11.2 mmol) in MeCN (30 mL) followed by a solution of HCOOK (4.72 g, 56.2 mmol) in H₂O (22 mL) were added to dropwise at 5° C. The reaction solution was warmed to RT and was stirred for 12 h. The reaction solvent was extracted with DCM (50 mL×4). The combined organic phases were concentrated in vacuum. The residue was purified by silica gel flash Chromatography (eluent: MeOH/DCM: 0-6%) to give the title compound (2.70 g, 92% purity, 810% yield) as a yellow oil.

LCMS (Method K) MS (ESI) [M+H]⁺=274.2, Rt=0.95 min. ¹H NMR (400 MHz, CDCl₃) δ 8.14-8.05 (m, 1H), 7.22-7.10 (m, 2H), 6.20 (t, J=74.2 Hz, 1H), 4.97-4.84 (m, 2H), 4.70-4.61 (m, 1H), 2.71-2.52 (m, 4H), 1.93-1.80 (m, 1H), 1.79-1.65 (m, 1H), 0.94 (t, J=7.4 Hz, 3H).

### Step 4: methyl 4-((R)-1-(5-(trans-3-(difluoromethoxy)cyclobutoxy)pyridin-2-yl)propoxy)-2,3-difluorobenzoate

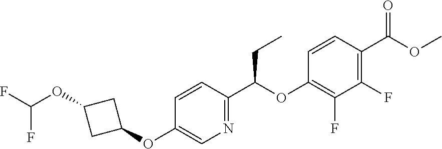 [SMILES: CC[C@@H](Oc1ccc(C(=O)OC)c(F)c1F)c1ccc(O[C@H]2C[C@H](OC(F)F)C2)cn1]

Under N₂, a mixture of (S)-1-(5-(trans-3-(difluoromethoxy)cyclobutoxy)pyridin-2-yl)propan-1-ol (2.69 g, 9.84 mmol), methyl 2,3-difluoro-4-hydroxybenzoate (2.13 g, 11.3 mmol), CMBP (4.75 g, 19.7 mmol) in toluene (30 mL) was stirred for 12 h at 110° C. The solution was concentrated in vacuum. The residue was purified by silica gel flash chromatography (eluent: EtOAc/PE: 0-9%) to give the title compound (2.50 g, 91% purity, 52% yield) as a brown solid.

LCMS (Method K) MS (ESI) [M+H]⁺=444.2, Rt=1.59 min. ¹H NMR (400 MHz, CDCl₃) δ 8.20-8.02 (m, 1H), 7.55-7.46 (m, 1H), 7.32-7.27 (m, J=8.6 Hz, 1H), 7.07-7.02 (m, 1H), 6.68-6.61 (m, 1H), 6.19 (t, J=74.2 Hz, 1H), 5.28-5.23 (m, 1H), 4.94-4.83 (m, 2H), 3.88 (s, 3H), 2.67-2.53 (m, 4H), 2.11-2.03 (m, 2H), 1.03 (t, J=7.4 Hz, 3H).

### Step 5: 4—((R)-1-(5-(trans-3-(difluoromethoxy)cyclobutoxy)pyridin-2-yl)propoxy)-2,3-difluorobenzoic acid

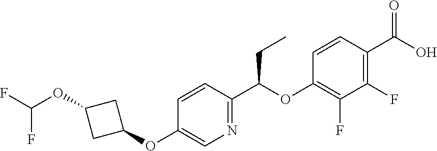 [SMILES: CC[C@@H](Oc1ccc(C(=O)O)c(F)c1F)c1ccc(O[C@H]2C[C@H](OC(F)F)C2)cn1]

To a solution of methyl 4-((R)-1-(5-(trans-3-(difluoromethoxy)cyclobutoxy)pyridin-2-yl)propoxy)-2,3-difluorobenzoate (2.47 g, 5.57 mmol) in THF (21 mL)/H₂O (7 mL) was added LiOH H₂O (668 mg, 27.9 mmol). The mixture was stirred for 7 h at RT. The reaction mixture was acidified with HCOOH to pH=6-7 and was extracted with EtOAc (60 mL×4). The combined organic phases were concentrated in vacuum. The residue was purified by silica gel flash chromatography (eluent: MeOH/DCM: 0-6%) and chiral SFC (Method I) to give the title compound (1.2 g, >99% ee, 92% purity, 46% yield) as a yellow solid.

LCMS (Method K) MS (ESI) [M+H]⁺=430.1, Rt=1.40 min. ¹H NMR (400 MHz, DMSO-d6, ppm) δ 13.17 (s, 1H), 8.23-8.16 (m, 1H), 7.59-7.49 (m, 1H), 7.41-7.33 (m, 1H), 7.31-7.24 (m, 1H), 6.97-6.89 (t, 1H), 6.66 (t, J=75.7 Hz, 1H), 5.42 (t, J=6.5 Hz, 1H), 5.00-4.92 (m, 1H), 4.85-4.77 (m, 1H), 2.64-2.56 (m, 2H), 2.47-2.42 (m, 2H), 2.08-1.93 (m, 2H), 0.91 (t, J=7.3 Hz, 3H). 4.5.15. Example 15. Preparation of (R)-4-(1-(3-(cyclopropylmethoxy)phenyl)propoxy)-2,3-difluorobenzoic acid (Acid 0) Step 1: 1-(3-(cyclopropylmethoxy)phenyl)propan-1-one

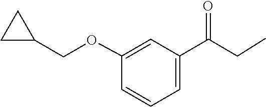

A mixture of 1-(3-hydroxyphenyl)propan-1-one (5.0 g, 33 mmol), (bromomethyl)cyclopropane (9.0 g, 67 mmol) and K₂CO₃ (13.8 g, 100 mmol) in DMF (30 mL) was stirred at 90° C. for 16 h. The resulting mixture was diluted with water (50 mL) and extracted with EtOAc (60 mL×3). The combined organic phases were washed with brine (15 mL), dried over Na₂SO₄, concentrated in vacuo and the residue was purified by silica gel flash chromatography (eluent: PE/EtOAc: 3/1) to give the title compound (6.0 g, 90% purity, 79% yield) as a white solid.

LCMS (Method K) MS (ESI) [M+H]⁺=204.9, Rt=1.43 min. ¹H NMR (400 MHz, DMSO-d6, ppm) δ 7.53 (d, J=7.7 Hz, 1H), 7.43-7.40 (m, 2H), 7.18 (dd, J=7.9, 2.1 Hz, 1H), 3.88 (d, J=7.0 Hz, 2H), 3.03 (q, J=7.2 Hz, 2H), 1.31-1.16 (m, 1H), 1.07 (t, J=7.2 Hz, 3H), 0.72-0.49 (m, 2H), 0.45-0.21 (m, 2H).

### Step 2: (S)-1-(3-(cyclopropylmethoxy)phenyl)propan-1-ol

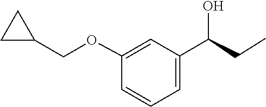

A mixture of [RuCl₂(mesitylene)]₂ (0.25 g, 0.4 mmol), and N-[(1S,2S)-2-amino-1,2-diphenylethyl]-4-methyl-1-sulfonylaniline (0.44 g, 0.9 mmol) in MeCN (5 mL) and H₂O (5 mL) was degassed and purged with N₂ (x 3) and then stirred at 40° C. for 1 h under N₂. A solution of 1-[3-(cyclopropylmethoxy)phenyl]propan-1-one (5 g, 24 mmol) in MeCN (40 mL), followed by HCOOK (10.3 g, 130 mmol) in water (25 mL) was added to the mixture at 0° C. The reaction mixture was slowly warmed to RT over 16 h. The solution was diluted with water (30 mL) and extracted with EtOAc (50 ml×2). The combined organic layers were washed with brine (20 mL), dried over anhydrous Na₂SO₄ and the solvent was removed in vacuo. The residue was purified by silica gel flash chromatography (eluent: PE/EtOAc: 2/1) to give the title compound (5.0 g, 90% purity, 89% yield) as a yellow oil.

¹H NMR (400 MHz, DMSO-d₆, ppm) δ 7.18 (t, J=8.0 Hz, 1H), 6.95-6.79 (m, 2H), 6.79-6.65 (m, 1H), 5.06 (s, 1H), 4.39 (t, J=6.0 Hz, 1H), 3.78 (d, J=6.9 Hz, 2H), 1.60-1.54 (m, 2H), 1.31-1.12 (m, 1H), 0.81 (t, J=7.4 Hz, 3H), 0.61-0.48 (m, 2H), 0.40-0.24 (m, 2H).

### Step 3: methyl (R)-4-(1-(3-(cyclopropylmethoxy)phenyl)propoxy)-2,3-difluorobenzoate

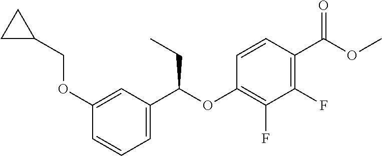 [SMILES: CC[C@@H](Oc1ccc(C(=O)OC)c(F)c1F)c1cccc(OCC2CC2)c1]

Under N₂, a mixture of PPh₃ (19.0 g, 73 mmol) and DIAD (14.7 g, 73 mmol) in anhydrous THF (100 mL) was stirred at 0° C. for 30 min. A solution of (1S)-1-[3-(cyclopropylmethoxy)phenyl]propan-1-ol (5.0 g, 25 mmol) and methyl 2,3-difluoro-4-hydroxybenzoate (5.5 g, 29 mmol) in THF (30 mL) was added dropwise. The resulting mixture was stirred at RT for 16 h. The solution was diluted with water (100 mL) and extracted with EtOAc (100 mL×2). The combined organic layers were washed with brine (50 mL), dried over anhydrous Na₂SO₄ and the solvent was removed in vacuo. The residue was purified by silica gel flash chromatography (eluent: PE/EtOAc: 1/1) to give the title compound (7.0 g, 90% purity, 67% yield) as a yellow oil.

¹H NMR (400 MHz, DMSO-d₆, ppm) δ 7.56 (t, J=7.9 Hz, 1H), 7.25 (t, J=7.9 Hz, 1H), 6.99 (t, J=8.2 Hz, 1H), 6.97-6.93 (m, 2H), 6.87-6.77 (m, 1H), 5.45 (t, J=6.4 Hz, 1H), 3.98-3.59 (m, 5H), 2.10-1.95 (m, 1H), 1.95-1.71 (m, 1H), 1.37-1.09 (m, 1H), 1.07-0.77 (m, 3H), 0.70-0.45 (m, 2H), 0.32-0.29 (m, 2H).

### Step 4: (R)-4-(1-(3-(cyclopropylmethoxy)phenyl)propoxy)-2,3-difluorobenzoic acid)

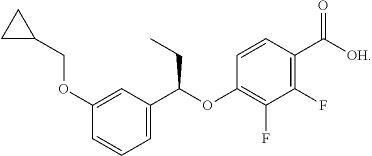 [SMILES: CC[C@@H](Oc1ccc(C(=O)O)c(F)c1F)c1cccc(OCC2CC2)c1]

A mixture of methyl 4-[(1R)-1-[3-(cyclopropylmethoxy)phenyl]propoxy]-2,3-difluorobenzoate (7.0 g, 18 mmol) and LiOH (892 mg, 37 mmol) in THF (50 mL) and H₂O (50 mL) was stirred at RT for 16 h. The pH was adjusted to around 5 by addition of AcOH. The solution was extracted with EtOAc (100 mL×2). The combined organic layers were washed with brine (20 mL), dried over anhydrous Na₂SO₄ and the solvent was removed in vacuo. The residue was purified by chiral SFC (Method J) to give the title compound (3.8 g, 99.5% purity, >99% ee, 59% yield) as a white solid.

LCMS (Method K) MS (ESI) [M+H]⁺=361.1, Rt=1.52 min. ¹H NMR (400 MHz, DMSO-d₆, ppm) δ 13.16 (s, 1H), 7.68-7.37 (m, 1H), 7.25 (t, J=8.1 Hz, 1H), 7.05-6.89 (m, 3H), 6.89-6.66 (m, 1H), 5.43 (t, J=6.5 Hz, 1H), 4.02-3.59 (m, 2H), 2.13-1.92 (m, 1H), 1.92-1.76 (m, 1H), 1.28-1.02 (m, 1H), 0.92 (t, J=7.3 Hz, 3H), 0.71-0.47 (m, 2H), 0.43-0.19 (m, 2H).

### 4.5.16. Example 16. Preparation of 2,3-difluoro-4-((R)-1-(4-(trans-3-methoxycyclobutoxy)pyridin-2-yl)propoxy)benzoic acid (Acid P)

### Step 1: 4-(trans-3-methoxycyclobutoxy)picolinonitrile

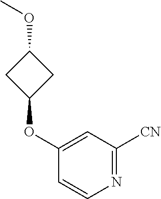

A mixture of 4-hydroxypicolinonitrile (2.71 g, 22.6 mmol), cis-3-methoxycyclobutyl 4-methylbenzenesulfonate (5.8 g, 22.6 mmol) and K₂CO₃ (6.25 g, 45.2 mmol) in DMF (120 mL) was stirred for 5 h at 100° C. The reaction mixture was diluted with brine (500 mL) and extracted with EtOAc (250 mL×3). The combined organic phases were concentrated in vacuo. The residue was purified by silica gel flash chromatography (eluent: EtOAc/PE: 0-30%) to give the title compound (4 g, 90% purity, 78% yield) as a white solid.

LCMS (Method K) MS (ESI) [M+H]⁺=205.2, Rt=1.09 min. ¹H NMR (400 MHz, DMSO-d₆, ppm) δ 8.52 (d, J=5.8 Hz, 1H), 7.59 (d, J=2.5 Hz, 1H), 7.23-7.14 (m, 1H), 5.06-4.95 (m, 1H), 4.12-4.00 (m, 1H), 3.17 (s, 3H), 2.49-2.41 (m, 2H), 2.37-2.56 (m, 2H).

### Step 2: 1-(4-(trans-3-methoxycyclobutoxy)pyridin-2-yl)propan-1-one

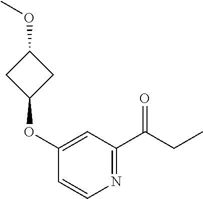 [SMILES: CCC(=O)c1cc(O[C@H]2C[C@H](OC)C2)ccn1]

Under N₂, to a solution of 4-(trans-3-methoxycyclobutoxy)picolinonitrile (3.85 g, 18.8 mmol) in THF (80 mL) was added EtMgBr (18.8 mL, 37.6 mmol, 2M in THF) at 0° C. The reaction mixture was stirred for 0.5 h at 0° C. and then heated to 70° C. for 1 h. The reaction mixture was poured into ice-water (150 mL) and then acidified with 3 N HCl to pH 4-5. The mixture was stirred at RT for 10 min and extracted with EtOAc (200 mL×8). The combined organic layers were dried over Na₂SO₄ and concentrated in vacuo. The residue was purified by silica gel flash chromatography (eluent: EtOAc/PE: 0-40%) to give the title compound (3.4 g, 90% purity, 69% yield) as a light yellow oil.

LCMS (Method K) MS (ESI) [M+H]⁺=235.9, Rt=1.13 min. ¹H NMR (400 MHz, MeOD, ppm) δ 8.44 (d, J=5.6 Hz, 1H), 7.41 (d, J=2.6 Hz, 1H), 6.89-6.80 (m, 1H), 4.97-4.85 (m, 1H), 4.16-4.05 (m, 1H), 3.26 (s, 3H), 3.19 (q, J=7.3 Hz, 2H), 2.53-2.34 (m, 4H), 1.18 (t, J=7.3 Hz, 3H).

### Step 3: (S)-1-(4-(trans-3-methoxycyclobutoxy)pyridin-2-yl)propan-1-ol

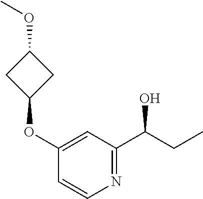 [SMILES: CC[C@H](O)c1cc(O[C@H]2C[C@H](OC)C2)ccn1]

A mixture of [RuCl₂(mesitylene)]₂ (142 mg, 0.27 mmol) and N-((1S,2S)-2-amino-1,2-diphenylethyl)-4-methylbenzenesulfonamide (202 mg, 0.55 mmol) in MeCN (6 mL) and H₂O (6 mL) was degassed and purged with N₂ (x 3) and then stirred at 40° C. for 1 h under N₂. A solution of 1-(4-(trans-3-methoxycyclobutoxy)pyridin-2-yl)propan-1-one (3.25 g, 13.8 mmol) in MeCN (15 mL), followed by HCOOK (5.8 g, 69.0 mmol) in H₂O (15 mL) was added to the mixture at 0° C. The reaction mixture was stirred at RT for 16 h under N₂. The reaction mixture was diluted with water (100 mL) and extracted with EtOAc (100 mL×3). The combined organic layers were dried over Na₂SO₄ and concentrated in vacuo. The residue was purified by silica gel flash chromatography (eluent: MeOH/DCM: 0-10%) to give the title compound (3.0 g, 90% purity, 82% yield) as a yellow solid.

LCMS (Method K) MS (ESI) [M+H]⁺=237.9, Rt=0.60 min. ¹H NMR (400 MHz, CDCl₃, ppm) δ 8.32 (d, J=5.7 Hz, 1H), 6.66-6.55 (m, 2H), 4.92-4.82 (m, 1H), 4.64-4.56 (m, 1H), 4.17-4.09 (m, 1H), 3.27 (s, 3H), 2.53-2.36 (m, 4H), 1.91-1.79 (m, 1H), 1.75-1.61 (m, 1H), 0.94 (t, J=7.4 Hz, 3H).

### Step 4: methyl 2,3-difluoro-4-((R)-1-(4-(trans-3-methoxycyclobutoxy)pyridin-2-yl)propoxy)benzoate

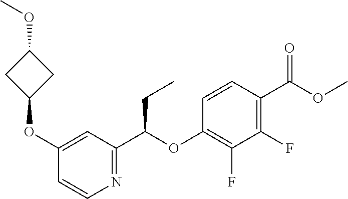

Under N₂, to a solution of PPh₃ (9.60 g, 36.6 mmol) in dry THF (150 mL) was added DIAD (7.41 g, 36.6 mol) dropwise at 0° C. and the mixture was stirred at 0° C. for 20 min. A solution of (S)-1-(4-(trans-3-methoxycyclobutoxy)pyridin-2-yl)propan-1-ol (2.90 g, 12.2 mmol) and methyl 2,3-difluoro-4-hydroxybenzoate (2.29 g, 12.2 mmol) in dry THF (40 mL) was added dropwise. The resulting reaction mixture was stirred at 25° C. for 16 h. The solvent was removed in vacuo. The residue was purified by silica gel flash chromatography (eluent: EtOAc/PE: 0-50%) to give the title compound (4.5 g, 90% purity, 810% yield) as a yellow oil.

LCMS (Method E) MS (ESI) [M+H]⁺=408.4, Rt=1.82 min. ¹H NMR (400 MHz, CDCl₃, ppm) δ 8.36 (d, J=5.7 Hz, 1H), 7.57-7.48 (m, 1H), 6.77 (d, J=2.4 Hz, 1H), 6.67-6.56 (m, 2H), 5.26-5.20 (m, 1H), 4.87-4.77 (m, 1H), 4.13-4.05 (m, 1H), 3.87 (s, 3H), 3.25 (s, 3H), 2.51-2.25 (m, 4H), 2.11-2.01 (m, 2H), 1.05 (t, J=7.4 Hz, 3H).

### Step 5: Preparation of 2,3-difluoro-4-((R)-1-(4-(trans-3-methoxycyclobutoxy)pyridin-2-yl)propoxy)benzoic acid

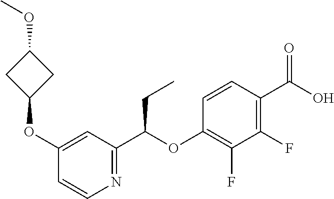 [SMILES: CC[C@@H](Oc1ccc(C(=O)O)c(F)c1F)c1cc(O[C@H]2C[C@H](OC)C2)ccn1]

Methyl 2,3-difluoro-4-((R)-1-(4-(trans-3-methoxycyclobutoxy)pyridin-2-yl)propoxy)benzoate (4.5 g, 11.0 mmol) and LiOH (10.5 g, 44.1 mmol) in THF/H₂O/MeOH (60 mL, 2/2/1) was stirred at RT for 2 h. The solvent was removed in vacuo and the residue was diluted with water (70 mL). The mixture was acidified with HCOOH to pH 5-6 and extracted with EtOAc (40 mL×3). The combined organic layers were dried over Na₂SO₄ and concentrated in vacuo. The residue was purified by silica gel flash chromatography (eluent: DCM/MeOH: 20/1 and 0.5% FA), followed by chiral SFC (Method K) to give the title compound (2.8 g, >99% ee, 95% purity, 61% yield) as a light yellow solid.

LCMS (Method K) MS (ESI) [M+H]⁺=394.1, Rt=1.13 min. ¹H NMR (400 MHz, DMSO-d₆, ppm) δ 13.18 (s, 1H), 8.36 (d, J=5.7 Hz, 1H), 7.59-7.50 (m, 1H), 6.95-6.88 (m, 1H), 6.86-6.81 (m, 1H), 6.80-6.76 (m, 1H), 5.41 (t, J=6.3 Hz, 1H), 4.98-4.85 (m, 1H), 4.09-3.95 (m, 1H), 3.15 (s, 3H), 2.46-2.15 (m, 4H), 2.07-1.93 (m, 2H), 0.94 (t, J=7.4 Hz, 3H).

### 4.5.17. Example 17. Preparation of 4-(2-(4-methylpiperazin-1yl)ethoxy)benzenesulfonamide (Example Procedure A)

### Step 1: 4-(2-chloroethoxy)benzenesulfonamide

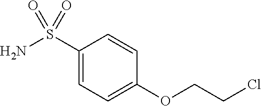 [SMILES: NS(=O)(=O)c1ccc(OCCCl)cc1]

To a solution of 4-hydroxybenzenesulfonamide (3 g, 17.3 mmol) and K₂CO₃ (6 g, 43.3 mmol) in MeCN (60 mL) was added 1-bromo-2-chloroethane (13.2 g, 91.7 mmol) at RT under N₂ atmosphere. The reaction mixture was stirred at 80° C. for 12 h. The reaction was cooled to RT and was concentrated in vacuo to dryness. The residue was diluted with H₂O (50 mL), extracted with EtOAc (50 mL×3). The combined organic layers were washed with H₂O (20 mL) followed by brine (20 mL). The organic phase was dried over anhydrous Na₂SO₄ and concentrated in vacuo. The residue was purified by silica gel flash column (gradient 0-50% EtOAc/PE) to give the title compound (2.5 g, 54% yield) as a white solid. LCMS (Method E) MS (ESI) [M−H]−=234, Rt=0.82 min.

### Step 2: 4-(2-(4-methylpiperazin-1-yl)ethoxy)benzenesulfonamide

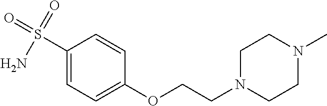 [SMILES: CN1CCN(CCOc2ccc(S(N)(=O)=O)cc2)CC1]

Under N₂, to a solution 4-(2-chloroethoxy)benzenesulfonamide (200 mg, 0.85 mmol) in DMF (2 mL) was added 1-methylpiperazine (170 mg, 1.7 mmol), K₂CO₃ (293 mg, 2.1 mmol), Kl (14 mg, 0.08 mmol) in sequence. The reaction solution was stirred for 12 h at 80° C. The solvent was removed in vacuo. The residue was purified by Prep-TLC (eluent 5:1 CH₂Cl₂/MeOH) to give the title compound (120 mg, 28% yield) as a white solid. LCMS (Method D) MS (ESI) [M+H]⁺=300, Rt=0.94 min. 4.5.18. Example 18. Preparation of 6-[2-(dimethylamino)ethoxy]-2-azaspiro[3,3]heptane-2-sulfonamide (Example Procedure B)

### Step 1: tert-butyl 6-(2-methoxy-2-oxoethoxy)-2-azaspiro[3,3]heptane-2-carboxylate

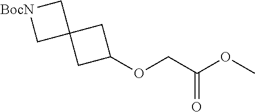 [SMILES: COC(=O)COC1CC2(C1)CN(C(=O)OC(C)(C)C)C2]

Under N₂, to a solution of tert-butyl 6-hydroxy-2-azaspiro[3,3]heptane-2-carboxylate (1.00 g, 4.7 mmol) in DMF (10 mL) was added NaH (60%, 0.17 g, 7.0 mmol) at 0° C. The mixture was then warmed to RT slowly and kept stirring at RT for additional 1 h. Then, methyl 2-bromoacetate (1.44 g, 9.4 mmol) was added. The reaction mixture was stirred at RT for 16 h. The mixture was quenched with ice-water (30 mL) slowly and extracted with EtOAc (50 mL×3). The combined organic layers were dried over Na₂SO₄ and concentrated in vacuo. The residue was purified by silica gel flash chromatography (eluent 1:5 EtOAc/PE) to give the title compound as a colorless oil (750 mg, 53% yield). ¹H NMR (400 MHz, CDCl₃, ppm) δ 4.01-3.93 (m, 3H), 3.88 (d, J=6.6 Hz, 4H), 3.75 (s, 3H), 2.55-2.42 (m, 2H), 2.25-2.12 (m, 2H), 1.42 (s, 9H). (Note: for some examples, Rh-catalyzed O—H insertion with ethyl diazoacetate was used as an alternative method to the alkylation chemistry described above.)

### Step 2: tert-butyl 6-(2-hydroxyethoxy)-2-azaspiro[3,3]heptane-2-carboxylate

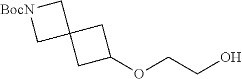 [SMILES: CC(C)(C)OC(=O)N1CC2(CC(OCCO)C2)C1]

To a solution of tert-butyl 6-(2-methoxy-2-oxoethoxy)-2-azaspiro[3,3]heptane-2-carboxylate (450 mg, 1.58 mmol) in THF (4 mL) and MeOH (4 mL) was added NaBH₄(179 mg, 4.73 mmol) at 0° C. and the mixture was then stirred at RT for 2 h. Ice-water (5 mL) was added slowly and the mixture was extracted with EtOAc (20 mL×3). The combined organic layers were dried over Na₂SO₄ and concentrated in vacuo to give the title compound (300 mg, 38% yield) as a colorless oil, which was used directly in next step without further purification. LCMS (Method D) MS (ESI) [M-tBu+H]⁺=202, Rt=1.04 min.

### Step 3: tert-butyl 6-(2-(tosyloxy)ethoxy)-2-azaspiro[3,3]heptane-2-carboxylate

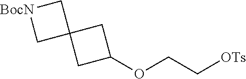 [SMILES: Cc1ccc(S(=O)(=O)OCCOC2CC3(C2)CN(C(=O)OC(C)(C)C)C3)cc1]

To a solution of tert-butyl 6-(2-hydroxyethoxy)-2-azaspiro[3,3]heptane-2-carboxylate (350 mg, 1.36 mmol) in THF (8 mL) were added KOH (381 mg, 6.80 mmol) and TsCl (454 mg, 2.38 mmol) at 0° C. The reaction was then warmed to RT and stirred for 1 h. The mixture was quenched with ice-water (5 mL) and extracted with EtOAc (30 mL×3). The combined organic layers were dried over Na₂SO₄ and concentrated in vacuo. The residue was purified by silica gel flash chromatography (eluent 1:5 EtOAc/PE) to give the title compound (400 mg, 64% yield) as a colorless oil. LCMS (Method D) MS (ESI) [M-tBu+H]⁺=356, Rt=1.46 min.

### Step 4: 2-{2-azaspiro[3,3]heptan-6-yloxy}ethyl 4-methylbenzenesulfonate

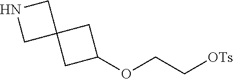 [SMILES: Cc1ccc(S(=O)(=O)OCCOC2CC3(CNC3)C2)cc1]

tert-Butyl 6-(2-{[(4-methylbenzene)sulfonyl]oxy}ethoxy)-2-azaspiro[3,3]heptane-2-carboxylate (800 mg, 1.94 mmol) in DCM/TFA (10/1, 10 mL) was stirred under RT of 2 h. The solvent was removed in vacuo to give the title compound (600 mg, 89% yield) as a colorless oil, which was used directly in the next step without further purification. LCMS (Method D) MS (ESI) [M+H]⁺=312, Rt=0.80 min.

### Step 5: benzyl N-[6-(2-{[(4-methylbenzene)sulfonyl]oxy}ethoxy)-2-azaspiro[3,3]heptane-2-sulfonyl]carbamate

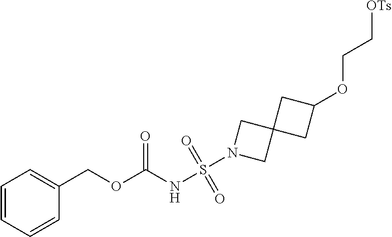 [SMILES: Cc1ccc(S(=O)(=O)OCCOC2CC3(C2)CN(S(=O)(=O)NC(=O)OCc2ccccc2)C3)cc1]

Under N₂, to a solution of benzyl alcohol (248 mg, 2.30 mmol) in DCM (6 mL) was added [(chlorosulfonyl)imino]methanone (295 mg, 2.09 mmol) at 0° C. The reaction mixture was stirred at 0° C. for 30 min and then 2-{2-azaspiro[3,3]heptan-6-yloxy}ethyl 4-methylbenzenesulfonate (650 mg, 2.09 mmol) and Et₃N (465 mg, 4.59 mmol) were added. The reaction mixture was stirred at RT for 1 h. The mixture was quenched with MeOH (10 mL) and concentrated in vacuo. The residue was purified by silica gel flash chromatography (eluent 10:1 DCM/MeOH) to give the title compound (700 mg, 58% yield) as a colorless oil. LCMS (Method D) MS (ESI) [M+H]⁺=525, Rt=1.39 min.

### Step 6: benzyl N-{6-[2-(dimethylamino)ethoxy]-2-azaspiro[3,3]heptane-2-sulfonyl}carbamate

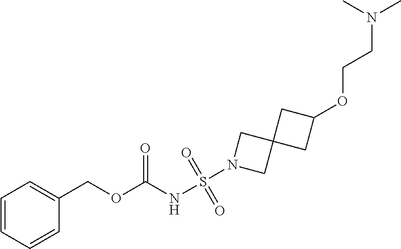 [SMILES: CN(C)CCOC1CC2(C1)CN(S(=O)(=O)NC(=O)OCc1ccccc1)C2]

A mixture of benzyl N-[6-(2-{[(4-methylbenzene)sulfonyl]oxy}ethoxy)-2-azaspiro[3,3]heptane-2-sulfonyl]carbamate (200 mg, 0.38 mmol) in dimethylamine (2M in THF, 2 mL) was stirred at 60° C. for 4 h. The solvent was removed in vacuo. The residue was purified by silica gel flash chromatography (eluent 10:1 DCM/MeOH) to afford the title compound (50 mg, 26% yield) as a white solid. LCMS (Method D) MS (ES+) [M+H]⁺=398, Rt=0.80 min.

### Step 7: 6-[2-(dimethylamino)ethoxy]-2-azaspiro[3,3]heptane-2-sulfonamide

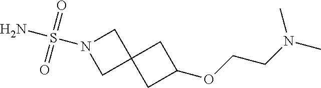 [SMILES: CN(C)CCOC1CC2(C1)CN(S(N)(=O)=O)C2]

Under H₂, a mixture of benzyl N-{6-[2-(dimethylamino)ethoxy]-2-azaspiro[3,3]heptane-2-sulfonyl}carbamate (50 mg, 0.13 mmol) and Pd/C (10 mg) in THF (1 mL) was stirred at RT for 2 h. The mixture was filtered through celite and concentrated in vacuo to give the title compound (20 mg, 54% yield) as a colorless oil. LCMS (Method I) MS (ESI) [M+H]⁺=264, Rt=0.97 min. 4.5.19. Example 19. Preparation of 4-((1-(2-methoxyethyl)piperidin-4-yl)oxy)benzenesulfonamide (Example Procedure C)

### Step 1: 4-hydroxy-N,N-bis(2-methoxy-5-methylphenyl)benzenesulfonamide

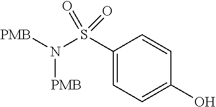

Under N₂, to a mixture of bis[(4-methoxyphenyl)methyl]amine (72 g, 0.28 mol) and 4-hydroxybenzenesulfonyl chloride (49 g, 0.25 mol) in CHCl₃ (450 mL) was added pyridine (150 mL) dropwise at 0° C. The reaction mixture was then stirred at RT for 12 h. The solvent was removed in vacuo. The residue was purified by silica gel flash chromatography (gradient 0-5% DCM/MeOH) to give the title compound (30 g, 29% yield) as a white solid. LCMS (Method D) MS (ESI) [M+Na]⁺=436, Rt=1.32 min.

### Step 2: tert-butyl 4-(4-(N,N-bis(4-methoxybenzyl)sulfamoyl)phenoxy)piperidine-1-carboxylate

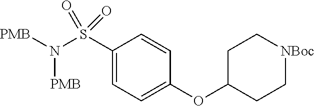

Under N₂, to a solution of 4-hydroxy-N,N-bis(2-methoxy-5-methylphenyl)benzenesulfonamide (10 g, 0.024 mol), tert-butyl 4-hydroxypiperidine-1-carboxylate (6.33 g, 0.031 mol) and PPh₃ (17.77 g, 0.068 mol) in toluene (10 mL) was added DIAD (14.68 g, 0.073 mol) at 0° C. The reaction solution was stirred for 12 h at 80° C. The solvent was removed under vacuum. The residue was purified by silica gel flash chromatography (gradient 0-7% MeOH/DCM) to give the title compound (12 g, 76% yield) as a white solid. LCMS (Method D) MS (ESI) [M+Na]⁺=619, Rt=1.70 min.

(Note: for some examples, boc-protection was not used and Mitsonobu chemistry with tertiary amino alcohols was conducted directly.)

### Step 3: N,N-bis(4-methoxybenzyl)-4-(piperidin-4-yloxy)benzenesulfonamide

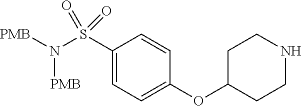

A mixture of tert-butyl 4-(4-(N,N-bis(4-methoxybenzyl)sulfamoyl)phenoxy)piperidine-1-carboxylate (12 g, 0.020 mol) and LiBr (24.44 g, 0.28 mol) in MeCN (120 mL) was stirred at 95° C. for 16 h. The reaction was filtered, and the filtrate was concentrated in vacuo. The residue was purified by silica gel flash chromatography (eluent 20:1 DCM/MeOH) to give the title compound (8.0 g, 70% yield) as a white solid. LCMS (Method D) MS (ESI) [M+H]⁺=497, Rt=1.05 min.

### Step 4: N,N-bis(4-methoxybenzyl)-4-((1-(2-methoxyethyl)piperidin-4-yl)oxy)benzenesulfonamide

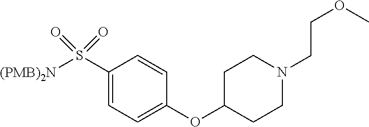

A mixture of N,N-bis(2-methoxy-5-methylphenyl)-4-(piperidin-4-yloxy)benzenesulfonamide (300 mg, 0.60 mmol), 1-bromo-2-methoxyethane (168 mg, 1.20 mmol) and K₂CO₃ (167 mg, 1.20 mmol) in MeCN (8 mL) was stirred at 80° C. for 16 h. The reaction was filtered, and the filtrate was concentrated under vacuum. The residue was purified by silica gel flash chromatography (eluent 10:1 DCM/MeOH) to give the title compound (300 mg, 72% yield) as a yellow oil. LCMS (Method D) MS (ESI) [M+H]⁺=555, Rt=1.09 min. (Note: in some examples, reductive amination chemistry was used instead of amine alkylation.)

### Step 5: 4-((1-(2-methoxyethyl)piperidin-4-yl)oxy)benzenesulfonamide

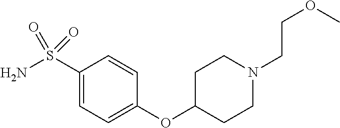 [SMILES: COCCN1CCC(Oc2ccc(S(N)(=O)=O)cc2)CC1]

A mixture of N,N-bis(4-methoxybenzyl)-4-((1-(2-methoxyethyl)piperidin-4-yl)oxy)benzenesulfonamide (300 mg, 0.54 mmol) in DCM (4 mL) and TFA (2 mL) was stirred at RT for 16 h. The reaction mixture was concentrated in vacuo and freeze-dried to give the title compound (200 mg, 94% yield) as a yellow oil. LCMS (Method D) MS (ESI) [M+H]⁺=315, Rt=0.45 min.

### 4.5.20. Example 20. Preparation of 6-((1-methylpiperidin-4-yl)oxy)pyridazine-3-sulfonamide (Example Procedure D)

### Step 1: 3-bromo-6-((1-methylpiperidin-4-yl)oxy)pyridazine

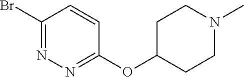

A mixture of 1-methylpiperidin-4-ol (536 mg, 4.65 mmol) and NaH (60%, 414 mg, 10.3 mmol) in THF (15 mL) was stirred at 20° C. for 1 h. Then, 3-bromo-6-chloropyridazine (1.00 g, 5.17 mmol) was added. The resulting solution was stirred for 15 h at 20° C. The reaction mixture was quenched with MeOH (1.5 mL) and then evaporated. The residue was purified by flash chromatography (eluent 40:1 DCM/MeOH) to give the title compound (1.00 g, 71% yield) as a white solid. LCMS (Method D) MS (ESI) [M+H]⁺=272, Rt=0.30 min.

### Step 2: 3-(benzylthio)-6-((1-methylpiperidin-4-yl)oxy)pyridazine

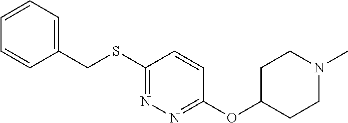 [SMILES: CN1CCC(Oc2ccc(SCc3ccccc3)nn2)CC1]

To a solution of 3-bromo-6-((1-methylpiperidin-4-yl)oxy)pyridazine (500 mg, 2.19 mmol), phenylmethanethiol (409 mg, 3.29 mmol) and DIPEA (568 mg, 4.39 mmol) in 1,4-dioxane (5 mL) was added Pd₂(dba)₃ (201 mg, 0.220 mmol) and Xantphos (191 mg, 0.329 mmol). The resulting solution was stirred at 110° C. for 4 h under N₂. The solution was removed under reduced pressure. The residue was purified by flash chromatography (eluent 40:1 DCM/MeOH) to give the title compound (600 mg, 87% yield) as a yellow solid. LCMS (Method D) MS (ESI) [M+H]⁺=316, Rt=0.88 min

### Step 3: 6-((1-methylpiperidin-4-yl)oxy)pyridazine-3-sulfonamide

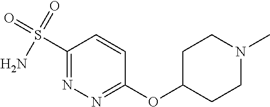 [SMILES: CN1CCC(Oc2ccc(S(N)(=O)=O)nn2)CC1]

To a solution of 3-(benzylthio)-6-((1-methylpiperidin-4-yl)oxy)pyridazine (600 mg, 1.90 mmol) in AcOH (12 mL) and H₂O (3 mL) was added NCS (1.02 g, 7.61 mmol) slowly at 0° C. The reaction mixture was stirred at 0° C. for 1 h. Aforesaid reaction solution was added dropwise a solution of NH3-H₂O [28% w/w, (20 mL)]. The resulting solution was stirred at 0° C. for 30 min The reaction was concentrated in vacuo to dryness. The residue was purified by flash chromatography (eluent 20:1 DCM/MeOH) to give the title compound (180 mg, 35% yield) as a yellow solid. LCMS (Method D) MS (ESI) [M+H]⁺=273, Rt=0.20 min.

### 4.5.21. Example 21. Preparation of 6-[(1-isopropylpiperidin-4-yl)oxy]pyridine-3-sulfonamide (Example Procedure E)

### Step 1: 6-[(1-isopropylpiperidin-4-yl)oxy]pyridine-3-sulfonamide

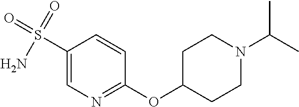 [SMILES: CC(C)N1CCC(Oc2ccc(S(N)(=O)=O)cn2)CC1]

To a solution of 6-chloropyridine-3-sulfonamide (150 mg, 0.78 mmol) in THF (8 mL) was added 1-isopropylpiperidin-4-ol (167 mg, 1.17 mmol) and NaH (74.8 mg, 3.11 mmol). The reaction mixture was irradiated in a microwave reactor at 100° C. for 2.5 h. The mixture was quenched with NH₄Cl aq. (0.1 mL), and the solvent was concentrated in vacuo. The residue was purified by silica gel flash chromatography (gradient 0-50% MeOH/DCM) to give the title compound (100 mg, 38% yield) as a yellow solid. LCMS (Method E) MS (ESI) [M+H]⁺=300, Rt=0.20 min. (Note: in some examples, SNAr chemistry was performed with an amine nucleophile instead of an alcohol, using triethylamine as base instead of NaH.)

### 4.5.22. Example 22. Preparation of 5-((1-methylpiperidin-4-yl)amino)pyridine-2-sulfonamide (Example Procedure F)

### Step 1: 5-bromo-N,N-bis(4-methoxybenzyl)pyridine-2-sulfonamide

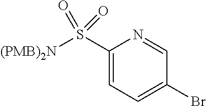

To a solution of bis(4-methoxybenzyl)amine (2.99 g, 0.012 mol) and triethylamine (1.76 g, 0.017 mol) in DCM (20 mL) was added 5-bromopyridine-2-sulfonyl chloride (1.5 g, 0.0058 mol). The resulting solution was stirred at 20° C. for 2 h. The reaction mixture was washed with saturated aq. NH₄Cl (50 mL), dried over anhydrous Na₂SO₄, filtered and the solvent was removed in vacuo. The residue was purified by silica gel flash chromatography (eluent 100:1 DCM/MeOH) to give the title compound (2.1 g, 76% yield) as a white solid. LCMS (Method D) MS (ESI) [M+H]⁺=477, Rt=1.56 min.

### Step 2: N,N-bis(4-methoxybenzyl)-5-((1-methylpiperidin-4-yl)amino)pyridine-2-sulfonamide

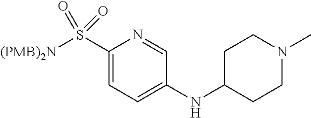

To a solution of 5-bromo-N,N-bis(4-methoxybenzyl)pyridine-2-sulfonamide (500 mg, 1.05 mmol) and 1-methylpiperidin-4-amine (179.40 mg, 1.57 mmol) in Tol (5 mL) was added Pd₂(dba)₃ (192 mg, 0.21 mmol), Xphos (200 mg, 0.42 mmol) and Cs₂CO₃ (1.02 g, 3.14 mmol). The resulting solution was stirred at 100° C. for 6 h under N₂. The solvent was removed in vacuo. The residue was purified by silica gel flash chromatography (eluent 30:1 DCM/MeOH) to give the title compound (180 mg, 34% yield) as a white solid. LCMS (Method D) MS (ESI) [M+H]⁺=511, Rt=1.00 min. (Note: in some examples, alcohols were used as coupling partners instead of amines; BINAP and NaOtBu used.)

### Step 3: 5-((1-methylpiperidin-4-yl)amino)pyridine-2-sulfonamide

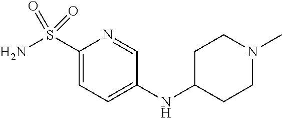 [SMILES: CN1CCC(Nc2ccc(S(N)(=O)=O)nc2)CC1]

To a solution of N,N-bis(4-methoxybenzyl)-5-((1-methylpiperidin-4-yl)amino)pyridine-2-sulfonamide (160 mg, 0.31 mmol) in DCM (2 mL) was added TFA (2 mL) at 20° C. The resulting solution was stirred at 50° C. for 2 h. The solvent was removed in vacuo to give the title compound (200 mg, 100% yield) as a yellow solid. LCMS (Method D) MS (ESI) [M+H]⁺=271, Rt=0.27 min.

### 4.5.23. Example 23. Preparation of 4-{[1-(2-methoxyethyl)piperidin-4-yl]oxy}piperidine-1-sulfonamide (Example Procedure G)

### Step 1: tert-butyl 4-{[1-(2-methoxyethyl)piperidin-4-yl]oxy}piperidine-1-carboxylate

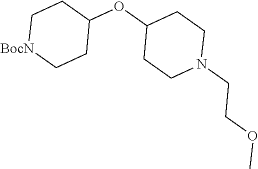 [SMILES: COCCN1CCC(OC2CCN(C(=O)OC(C)(C)C)CC2)CC1]

A mixture of tert-butyl 4-(piperidin-4-yloxy)piperidine-1-carboxylate (500 mg, 1.76 mmol), K₂CO₃ (729 mg, 5.27 mmol), Kl (29 mg, 0.18 mmol) and 1-bromo-2-methoxyethane (367 mg, 2.64 mmol) in MeCN (5 mL) was stirred at 80° C. for 5 h. The reaction mixture was concentrated in vacuo. The residue was purified by silica gel flash chromatography (eluent 1:1 EtOAc/PE) to give the title compound (500 mg, 75% yield) as a yellow solid. LCMS (Method E) MS (ESI) [M+H]⁺=343, Rt=0.74 min. (Note: in some examples, the first step requires O-alkylation of 1-Boc-4-hydroxypiperidine or reductive amination of 1-Boc-piperidine-4-carboxaldehyde.)

### Step 2: 1-(2-methoxyethyl)-4-(piperidin-4-yloxy)piperidine

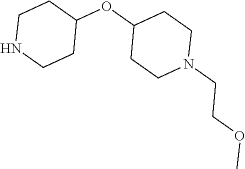 [SMILES: COCCN1CCC(OC2CCNCC2)CC1]

tert-butyl 4-{[1-(2-methoxyethyl)piperidin-4-yl]oxy}piperidine-1-carboxylate (500 mg, 1.46 mmol) in DCM/TFA (10/1, 5 mL) was stirred at RT for 4 h. The reaction mixture was concentrated in vacuo and lyophilized to afford the title compound (300 mg, 76% yield) as a yellow solid. LCMS (Method D) MS (ESI) [M+H]⁺=243, Rt=0.19 min.

### Step 3: tert-butyl N-(4-{[1-(2-methoxyethyl)piperidin-4-yl]oxy}piperidine-1-sulfonyl)carbamate

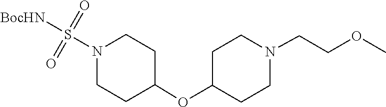 [SMILES: COCCN1CCC(OC2CCN(S(=O)(=O)NC(=O)OC(C)(C)C)CC2)CC1]

To a solution of 1-(2-methoxyethyl)-4-(piperidin-4-yloxy)piperidine (200 mg, 0.83 mmol) and triethylamine (167 mg, 1.65 mmol) in dry DCM (2 mL) was added [(tert-butoxy)carbonyl][4-(dimethyliminio)pyridine-1-sulfonyl]azanide (249 mg, 0.83 mmol) at 0° C. and the reaction was then stirred at RT for 2 h. The final mixture was concentrated in vacuo. The residue was purified by silica gel flash chromatography (eluent 100:1 DCM/MeOH) to give the title compound (200 mg, 52% yield) as a yellow oil. LCMS (Method D) MS (ESI) [M+H]⁺=422, Rt=0.80 min.

### Step 4: 4-{[1-(2-methoxyethyl)piperidin-4-yl]oxy}piperidine-1-sulfonamide

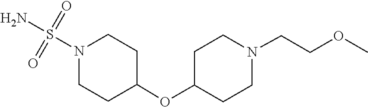 [SMILES: COCCN1CCC(OC2CCN(S(N)(=O)=O)CC2)CC1]

Tert-Butyl N-{6-[(1-methylpiperidin-4-yl)oxy]-2-azaspiro[3,3]heptane-2-sulfonyl}carbamate (200 mg, 0.51 mmol) in DCM/TFA (10/1, 3 mL) was stirred under RT for 4 h. The final mixture was concentrated in vacuo and lyophilized to afford the title compound (120 mg, 65% yield) as a yellow oil.

LCMS (Method D) MS (ESI) [M+H]⁺=322, Rt=0.31 min.

### 4.5.24. Example 24. Preparation of benzyl N-(4-{[1-(cyclopropylmethyl)piperidin-4-yl]oxy}piperidine-1-sulfonyl)carbamate (Example Procedure H)

### Step 1: tert-butyl 4-((1-(N-((benzyloxy)carbonyl)sulfamoyl)piperidin-4-yl)oxy)piperidine-1-carboxylate

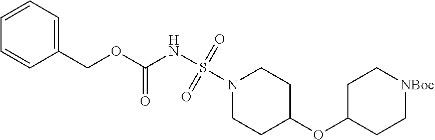 [SMILES: CC(C)(C)OC(=O)N1CCC(OC2CCN(S(=O)(=O)NC(=O)OCc3ccccc3)CC2)CC1]

Under N₂, to a solution of [(chlorosulfonyl)imino]methanone (29.9 g, 0.21 mol) in DCM (300 mL) was added benzyl alcohol (25.1 g, 0.23 mol) at 0° C. The mixture was stirred at 0° C. for 0.5 h. tert-Butyl 4-(piperidin-4-yloxy)piperidine-1-carboxylate (60.0 g, 0.21 mol) and triethylamine (47.0 g, 0.46 mol) were added slowly. The reaction mixture was then warmed to RT and stirred for 16 h. The mixture was quenched with MeOH (30 mL). The solvent was removed in vacuo. The residue was purified by silica gel flash chromatography (gradient 0-10% MeOH/DCM) to give the title compound (66 g, 57% yield) as a colorless oil. ¹H NMR (400 MHz, CDCl₃, ppm) δ 7.38-7.34 (m, 5H), 5.18 (s, 2H), 3.79-3.66 (m, 2H), 3.63-3.49 (m, 4H), 3.33-3.23 (m, 2H), 3.15-3.07 (m, 2H), 1.86-1.63 (m, 6H), 1.52-1.42 (m, 11H). (Note: in some examples, the following two steps were not required when this reaction was performed N-alkylated piperazines: no boc-protection required.)

### Step 2: benzyl((4-(piperidin-4-yloxy)piperidin-1-yl)sulfonyl)carbamate

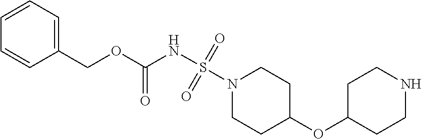 [SMILES: O=C(NS(=O)(=O)N1CCC(OC2CCNCC2)CC1)OCc1ccccc1]

4-{[1-({[(benzyloxy)carbonyl]amino}sulfonyl)piperidin-4-yl]oxy}piperidine-1-carboxylate (66.0 g, 0.10 mol) in DCM/TFA (10/1, 572 mL) was stirred at RT for 4 h. The solvent was concentrated in vacuo to give the title compound (66.0 g, TFA salt) as a colorless oil. LCMS (Method G) MS (ESI) [M+H]⁺=398, Rt=1.03 min.

### Step 3: benzyl N-(4-{[1-(cyclopropylmethyl)piperidin-4-yl]oxy}piperidine-1-sulfonyl)carbamate

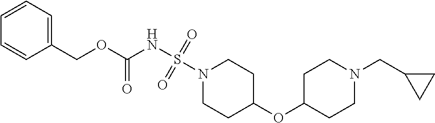 [SMILES: O=C(NS(=O)(=O)N1CCC(OC2CCN(CC3CC3)CC2)CC1)OCc1ccccc1]

To a solution of benzyl N-[4-(piperidin-4-yloxy)piperidine-1-sulfonyl]carbamate (120 mg, 0.30 mmol) in 2,2,2-trifluoroethanol (3 mL) was added cyclopropanecarbaldehyde (169 mg, 2.41 mmol) and NaBH₄ (46 mg, 1.21 mmol). The reaction mixture was stirred at 78° C. for 3 h. The mixture was quenched with H₂O (20 mL) and extracted with EtOAc (20 mL×3). The combined organic layers were dried over Na₂SO₄ and concentrated in vacuo. The residue was purified by silica gel flash chromatography (gradient 0-70% EtOAc/PE) to give the title compound (120 mg, 70% yield) as a yellow oil. LCMS (Method E) MS (ESI) [M+H]⁺=452, Rt=1.03 min. (Note: in some examples, NaBH₃CN is used as the reductant.)

### Step 4: 4-{[1-(cyclopropylmethyl)piperidin-4-yl]oxy}piperidine-1-sulfonamide

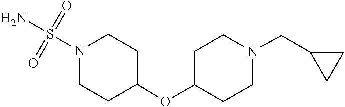 [SMILES: NS(=O)(=O)N1CCC(OC2CCN(CC3CC3)CC2)CC1]

Under H₂, a mixture of benzyl N-(4-{[1-(cyclopropylmethyl)piperidin-4-yl]oxy}piperidine-1-sulfonyl)carbamate (200 mg, 0.44 mmol) and Pd/C (20 mg) in THF (2 mL) was stirred at RT for 12 h. The final mixture was filtered. The filtrate was concentrated in vacuo to give the title compound (120 mg, 68% yield) as a white solid. LCMS (Method E) MS (ESI) [M+H]⁺=318, Rt=0.47 min.

### 4.5.25. Example 25. Preparation of 1-(dimethylamino)-2-methylpropane-2-sulfonamide (Example Procedure I)

### Step 1: methyl 2-(N,N-bis(4-methoxybenzyl)sulfamoyl)acetate

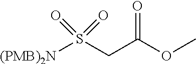

To a solution of bis(4-methoxybenzyl)amine (3.57 g, 13.9 mmol) in CHCl₃:pyridine (v:v=2:1, 45 mL) was added methyl 2-(chlorosulfonyl)acetate (2.0 g, 11.58 mmol) dropwise at 0° C. under an atmosphere of N₂. The resulting solution was slowly warmed to room temperature and stirred for 16 h. The final mixture was quenched with saturated aqueous NH₄Cl solution (50 mL) and extracted with EtOAc (50 mL×3). The combined organic phases were washed with H₂O (20 mL) and brine (20 mL), dried over Na₂SO₄, concentrated in vacuo. The residue was purified by silica gel flash chromatography (gradient 0-30% DCM/PE) to provide the title compound (1.50 g, 33% yield) as a pale yellow solid.

LCMS (Method I) MS (ESI) [M+H+H₂O]⁺=411, Rt=1.70 min.

### Step 2: methyl 2-(N,N-bis(4-methoxybenzyl)sulfamoyl)-2-methylpropanoate

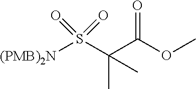

To a solution of methyl 2-(N,N-bis(4-methoxybenzyl)sulfamoyl)acetate (1.50 g, 3.81 mmol) in dry DMF (20 mL) was added NaH (0.38 g, 9.52 mmol) portionwise at 0° C. under an atmosphere of argon. After addition, the solution was stirred at 0° C. for 30 min. Then, Mel (0.59 mL, 9.52 mmol) was added dropwise. The resulting solution was slowly warmed to RT and stirred for 2 h. The reaction mixture was quenched with saturated aqueous NH₄Cl solution (50 mL) and extracted with EtOAc (35 mL×4). The combined organic phases were washed with H₂O (10 mL) and brine (10 mL), dried over Na₂SO₄, concentrated in vacuo. The residue was purified by silica gel flash chromatography (gradient 0-20% EtOAc/PE) to give the title compound (1.10 g, 68% yield) as a white solid. LCMS (Method D) MS (ESI) [M+Na]⁺=444, Rt=1.49 min.

### Step 3: 2—(N,N-bis(4-methoxybenzyl)sulfamoyl)-2-methylpropanoic acid

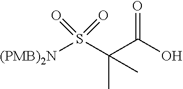

To a solution of methyl 2-(N,N-bis(4-methoxybenzyl)sulfamoyl)-2-methylpropanoate (1.0 g, 2.37 10 mmol) in MeOH/H₂O (v/v=5/1, 10 mL) was added LiOH—H₂O (113.52 mg, 4.74 mmol). The mixture was stirred at room temperature for 16 h. The solvent was removed in vacuo. The aqueous solution was adjusted to pH 2-3 with 2N HCl and extracted with EtOAc (20 mL×2). The combined organic phases were washed with brine (10 mL), dried over Na₂SO₄, concentrated in vacuo to give the title compound (750 mg, crude) as a yellow oil, which was used directly in next step without further purification. LCMS (Method D) MS (ESI) [M+Na]⁺=430, Rt=1.32 min.

### Step 4: 2—(N,N-bis(4-methoxybenzyl)sulfamoyl)-N,N,2-trimethylpropanamide

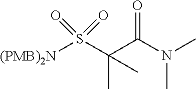

Under N₂, to a solution of 2-(N,N-bis(4-methoxybenzyl)sulfamoyl)-2-methylpropanoic acid (750 mg, 1.85 mmol) in MeCN (8 mL) was added TCFH (780 mg, 2.78 mmol), NMI (0.44 mL, 5.55 mmol) and dimethylamine (167 mg, 3.7 mmol) in sequence. The mixture was stirred for 2 h at RT. The reaction mixture was diluted with aq. NaCl (10 mL) and extracted with EtOAc (8 mL×4). The combined organic phases were concentrated in vacuo. The residue was purified by silica gel flash chromatography (gradient 0-4% MeOH/DCM) to give the title compound (560 mg, 70% yield) as an off white solid. LCMS (Method D) MS (ESI) [M+Na]⁺=435.2, Rt=1.41 min.

### Step 5: 1-(dimethylamino)-N,N-bis(4-methoxybenzyl)-2-methylpropane-2-sulfonamide

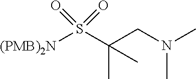

To a solution of 2-(N,N-bis(4-methoxybenzyl)sulfamoyl)-N,N,2-trimethylpropanamide (560 mg, 1.29 mmol) in THF (5 mL) was added dropwise 1M borane-THF complex (6.45 mL, 6.45 mmol) under ice cooling over 3 min. The mixture was stirred at RT for 5 h. The reaction mixture was quenched with 0.5 N aqueous NaOH solution at 0° C., stirred for 30 minutes and extracted with EtOAc (20 mL×4). The organic layers were combined, washed with brine (10 mL), dried over anhydrous Na₂SO₄, and concentrated in vacuo. The residue was purified by silica gel flash chromatography (gradient 0-2% MeOH/DCM) to give the title compound (300 mg, 55% yield) as a white solid. LCMS (Method D) MS (ESI) [M+H]⁺=421, Rt=0.94 min.

### Step 6: 1-(dimethylamino)-2-methylpropane-2-sulfonamide

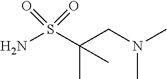

To a solution of 1-(dimethylamino)-N,N-bis(4-methoxybenzyl)-2-methylpropane-2-sulfonamide (300 mg, 0.71 mmol) in DCM (3 mL) was added dropwise TFA (3 mL) and TfOH (1-2 drops). The mixture was stirred RT for 5 min and concentrated in vacuo to give the title compound (200 mg, crude) as a yellow oil, which was used directly in next step without further purification. LCMS (Method D) MS (ESI) [M+H]⁺=181, Rt=0.19 min. 4.5.26. Example 26. Preparation of 4-(2-hydroxy-2-methylpropyl)piperazine-1-sulfonamide and 4-(2-fluoro-2-methylpropyl)piperazine-1-sulfonamide (Example Procedure J)

### Step 1: 2-methyl-1-(piperazin-1-yl)propan-2-ol

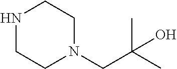

Under N₂, a mixture of piperazine (31.8 g, 369 mmol) and 1-chloro-2-methylpropan-2-ol (10.0 g, 92.0 mmol) in EtOH (200 mL) was stirred at 110° C. for 6 h. The reaction mixture was concentrated in vacuum. The residue was purified by silica gel flash chromatography (eluent: MeOH/DCM: 0-20% with 0.5% NH₄OH) to give the title compound (13.0 g, 90% purity, 22% yield) as a yellow oil. ¹H NMR (400 MHz, CDCl₃, ppm) δ 2.87-2.81 (m, 4H), 2.58-2.53 (m, 4H), 2.25 (s, 2H), 1.11 (s, 6H).

### Step 2: benzyl((4-(2-hydroxy-2-methylpropyl)piperazin-1-yl)sulfonyl)carbamate

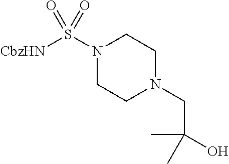 [SMILES: CC(C)(O)CN1CCN(S(=O)(=O)NC(=O)OCc2ccccc2)CC1]

Under N₂, to a solution of 2-methyl-1-(piperazin-1-yl)propan-2-ol (12.6 g, 79.6 mmol) in DCM (200 mL) was added ((benzyloxy)carbonyl)((4-(dimethyliminio)pyridin-1(4H)-yl)sulfonyl)amide (26.7 g, 79.6 mmol) and NEt₃ (20.1 g, 199 mmol) at RT. The mixture was stirred for 1 h at RT. The reaction mixture was concentrated in vacuum. The residue was purified by C18 chromatography (eluent: MeCN/water (+0.1% HCOOH: 0-50%) to give the title compound (15 g, 90% purity, 46% yield) as a white solid. LCMS (Method K) MS (ESI) [M+H]⁺=371.95, Rt=0.74 min. ¹H NMR (400 MHz, DMSO-d₆, ppm) δ 8.14 (s, 1H), 7.42-7.29 (m, 5H), 5.10 (s, 2H), 3.22-3.10 (m, 4H), 2.65-2.54 (m, 4H), 2.25 (s, 2H), 1.08 (s, 6H).

### Step 3a: 4-(2-hydroxy-2-methylpropyl)piperazine-1-sulfonamide

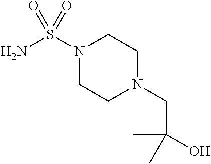

Under H₂, a mixture of benzyl((4-(2-hydroxy-2-methylpropyl)piperazin-1-yl)sulfonyl)carbamate (3.00 g, 8.07 mmol) and Pd/C (0.7 g, 10% wt) in MeOH (30 mL) was stirred for 16 h at RT. The solids were filtered and the filtrate was concentrated in vacuum to give the title compound (1.8 g, 80% purity, 75% yield) as a light yellow solid. LCMS (Method E) MS (ESI) [M+H]⁺=238.2, Rt=0.88 min. ¹H NMR (400 MHz, DMSO-d₆, ppm) δ 6.72 (s, 2H), 4.14 (s, 1H), 2.95-2.90 (m, 4H), 2.61-2.56 (m, 4H), 2.21 (s, 2H), 1.08 (s, 6H).

### Step 3b: ((4-(2-fluoro-2-methylpropyl)piperazin-1-yl)sulfonyl)carbamate

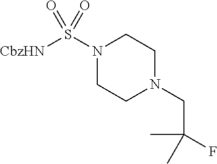 [SMILES: CC(C)(F)CN1CCN(S(=O)(=O)NC(=O)OCc2ccccc2)CC1]

Under N₂, to a solution of benzyl((4-(2-hydroxy-2-methylpropyl)piperazin-1-yl)sulfonyl)carbamate (9.55 g, 25.7 mmol) in DCM (200 mL) was added DAST (4.14 g, 25.7 mmol) at 0° C. The mixture was stirred for 1 hours at RT. The reaction mixture was quenched by water and concentrated in vacuum. The residue was purified by silica gel flash chromatography (eluent: MeOH/DCM: 0-10%) to give the title compound (5.0 g, 90% purity, 47% yield) as a yellow oil. LCMS (Method E) MS (ESI) [M+H]⁺=374.3, Rt=1.02 min. ¹H NMR (400 MHz, CDCl₃, ppm) δ 7.39-7.35 (m, 5H), 5.18 (s, 2H), 3.40-3.35 (m, 4H), 2.64-2.57 (m, 4H), 2.49-2.39 (m, 2H), 1.38 (s, 3H), 1.32 (s, 3H).

### Step 4: 4-(2-fluoro-2-methylpropyl)piperazine-1-sulfonamide

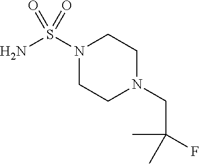

Under H₂, a mixture of benzyl((4-(2-fluoro-2-methylpropyl)piperazin-1-yl)sulfonyl)carbamate (4.6 g, 12.3 mmol) and Pd/C (0.65 g, 10% wt) in MeOH (60 mL) was stirred for 16 h at RT. The reaction was filtered and the filtrate was concentrated in vacuum. The residue was freeze-dried to give the title compound (2.85 g, 80% purity, 77% yield) as a light-yellow solid. LCMS (Method F) MS (ESI) [M+H]+=239.9, Rt=0.20 min. ¹H NMR (400 MHz, DMSO-d₆, ppm) δ 6.74 (s, 2H), 2.98-2.90 (m, 4H), 2.62-2.51 (m, 4H), 2.49-2.42 (m, 2H), 1.33 (s, 3H), 1.28 (s, 3H).

### 4.5.27. Example 27. Preparation of (R)-4-(1-(5-(cyclopropylmethoxy)pyridin-2-yl)propoxy)-N-((6-(2-(dimethylamino)ethoxy)-2-azaspiro[3,3]heptan-2-yl)sulfonyl)-2,3-difluorobenzamide (Coupling example procedure A)

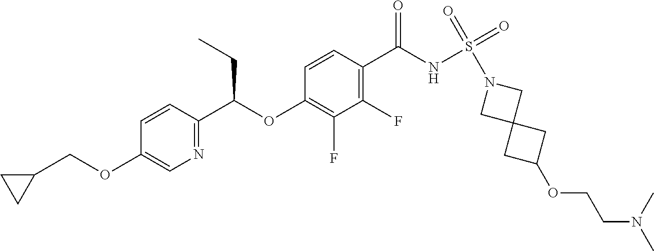 [SMILES: CC[C@@H](Oc1ccc(C(=O)NS(=O)(=O)N2CC3(CC(OCCN(C)C)C3)C2)c(F)c1F)c1ccc(OCC2CC2)cn1]

To a solution of 4-[(1R)-1-[5-(cyclopropylmethoxy)pyridin-2-yl]propoxy]-2,3-difluorobenzoic acid (Acid C, 20 mg, 0.06 mmol) in DCM (1 mL) were added CMPI (29 mg, 0.11 mmol) and DMAP (2 mg, 0.02 mmol) at RT. The mixture was stirred at RT for 20 min and then 6-[2-(dimethylamino)ethoxy]-2-azaspiro[3,3]heptane-2-sulfonamide (example procedure B, 17 mg, 0.07 mmol) and NEt₃ (17 mg, 0.17 mmol) were added. The reaction mixture was stirred at RT for 1 h. The solvent was removed in vacuo. The residue was purified by Prep-HPLC (method A, gradient: 30-80%) to give the title compound (2.1 mg, 95% purity, >99% ee, 6.0% yield) as a white solid. LCMS (Method F) MS (ESI) [M+H]⁺=609.3, Rt=0.97 min. ¹H NMR (400 MHz, DMSO-d₆, ppm) δ 8.24 (s, 1H), 7.43-7.26 (m, 3H), 6.73 (t, J=8.2 Hz, 1H), 5.30 (t, J=6.4 Hz, 1H), 3.86 (d, J=7.1 Hz, 3H), 3.69-3.55 (m, 4H), 3.51-3.42 (m, 2H), 3.00-2.89 (m, 2H), 2.58 (s, 6H), 2.37-2.35 (m, 2H), 2.08-1.87 (m, 4H), 1.22-1.15 (m, 2H), 0.91 (t, J=7.3 Hz, 3H), 0.62-0.52 (m, 2H), 0.35-0.27 (m, 2H).

### 4.5.28. Example 28. Preparation of (R)—N-((1-(dimethylamino)-2-methylpropan-2-yl)sulfonyl)-2,3-difluoro-4-(1-(5-(pyridin-2-yloxy)pyridin-2-yl)propoxy)benzamide

### Coupling Example Procedure B

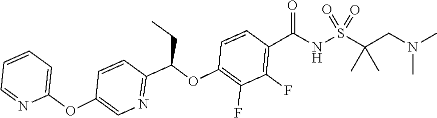 [SMILES: CC[C@@H](Oc1ccc(C(=O)NS(=O)(=O)C(C)(C)CN(C)C)c(F)c1F)c1ccc(Oc2ccccn2)cn1]

To a solution of (R)-2,3-difluoro-4-(1-(5-(pyridin-2-yloxy)pyridin-2-yl)propoxy)benzoic acid (Acid E, 25 mg, 0.064 mmol) in dry DMF (2 mL) was added CMPI (32.7 mg, 0.128 mmol) and DMAP (3.9 mg, 0.032 mmol). After addition, the solution was stirred at 25° C. for 30 min. Then, 1-(dimethylamino)-2-methylpropane-2-sulfonamide (example procedure I, 23.1 mg, 0.128 mmol) and NEt₃ (0.031 mL, 0.224 mmol) were added. The mixture was stirred at room temperature for 5 h. The reaction mixture was diluted with NaCl aqueous (5 mL) and extracted with EtOAc (10 mL×4). The combined organic phases were concentrated in vacuum. The residue was purified by Prep-HPLC (method B, gradient: 30-50%) to give the title compound (4.2 mg, 99.4% purity, >99% ee, 11.9% yield) as a white solid. LCMS (Method D) MS (ESI) [M+H]⁺=549.1, Rt=1.11 min. ¹H NMR (400 MHz, DMSO-d6, ppm) δ 10.12 (s, 1H), 8.45 (d, J 2.6 Hz, 1H), 8.15-8.13 (m, 1H), 7.92-7.87 (m, 1H), 7.66-7.63 (m, 1H), 7.51-7.45 (m, 2H), 7.18-7.12 (m, 2H), 6.89 (t, J 8.4 Hz, 1H), 5.45 (t, J 6.3 Hz, 1H), 3.36 (s, 2H), 2.86 (s, 6H), 2.08-2.00 (m, 2H), 1.34 (s, 6H), 0.96 (t, J 7.3 Hz, 3H).

### 4.5.29. Example 29. Preparation of 4-(1-(3-(cyclopropylmethoxy)phenyl)ethoxy)-N-((1-(2-(dimethylamino)ethyl)-1H-pyrazol-4-yl)sulfonyl)-2,3-difluorobenzamide (Coupling example procedure C)

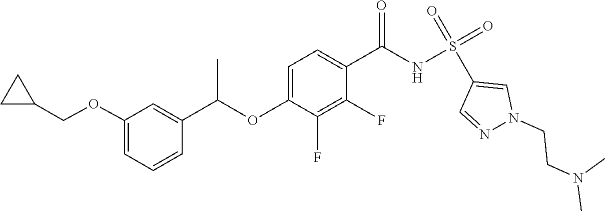 [SMILES: CC(Oc1ccc(C(=O)NS(=O)(=O)c2cnn(CCN(C)C)c2)c(F)c1F)c1cccc(OCC2CC2)c1]

To a solution of 8-[1-(3-propoxyphenyl)ethoxy]imidazo[1,2-a]pyridine-5-carboxylic acid (Acid B, 463 mg, 1.33 mmol) and EDCI (255 mg, 1.33 mmol) in DCM (2 mL) were added 2-methylpropane-2-sulfonamide (example procedure D, 145 mg, 0.66 mmol) and DMAP (162 mg, 1.33 mmol). The reaction mixture was stirred at RT for 18 h. The solvent was removed in vacuo and the residue was purified by Prep-HPLC (method A, gradient: 30-35-45%) to give the title compound (12.1 mg, 95.2% purity, 3.1% yield) as a white solid. LCMS (Method D) MS (ESI) [M+H]⁺=549.2, Rt=1.01 min.

### 4.5.30. Example 30. Preparation of Additional Compounds

The compounds listed below in Table 4 were prepared according to the general approach shown above in Scheme 1. They synthesized by coupling carboxylic acids A-P with sulfonamides or sulfamides (commercially available [CAS; number provided at bottom of table] or synthesized according to example procedures A-J) using coupling conditions A-D. The compounds were purified using prep HPLC methods A-K (or alternative methods such as reverse phase C18 chromatography or prep TLC). NMR and LCMS data (methods A-J) are also provided. (Captions in the table are CA=Carboxylic Acid; CP=Coupling Partner; CC=Coupling Conditions.)

TABLE 4

| | | | | Purification | | |
|---|---|---|---|---|---|---|
| Compound Name | CA | CP | CC | (Gradient) | LCMS | NMR |
| 4-(1-(3-(cyclopropylmethoxy)phenyl)- | B | D | C | Method A | m/z = 549.2 | ¹H NMR (400 MHz, DMSO-d6, ppm) δ 8.14 (d, J = 5.1 Hz, 1 H), 7.69 (s, 1 H), 7.34 (t, J = 8.5 Hz, |
| ethoxy)-N-((1-(2-(dimethylamino)ethyl)-1H- | | | | (30-35-45) | [M + H]+, Rt = | 1 H), 7.23 (t, J = 8.1 Hz, 1 H), 6.93 (d, J = 6.7 Hz, 2 H), 6.81 (t, J = 8.1 Hz, 2 H), 5.57 (q, J = 6.2 |
| pyrazol-4-yl)sulfonyl)-2,3-difluorobenzamide | | | | | 1.01 mins | Hz, 1 H), 4.41 (t, J = 5.9 Hz, 2 H), 3.85-3.70 (m, 2 H), 3.41-3.35 (m, 2 H), 2.61 (s, 6 H), 1.57 (d, J = |
| | | | | | (Method D) | 6.3 Hz, 3 H), 1.28-1.14 (m, 1 H), 0.58-0.50 (m, 2 H), 0.35-0.26 (m, 2 H) |
| (R)-4-(1-(5-(cyclopropylmethoxy)pyridin-2- | C | A | D | Method B | m/z = 632.2 | ¹H NMR (400 MHz, DMSO-d6, ppm) δ 8.24 (s, 1 H), 7.81 (d, J = 8.7 Hz, 2 H), 7.37-7.31 (m, 2 H), |
| yl)propoxy)-2,3-difluoro-N-((4-(2- | | | | (30-35-45) | [M + H]+, Rt = | 7.27 (t, J = 7.8 Hz, 1 H), 7.06 (d, J = 8.7 Hz, 2 H), 6.80 (t, J = 7.9 Hz, 1 H), 5.35 (t, J = 6.4 Hz, 1 H), |
| morpholinoethoxy)phenyl)sulfonyl)benzamide | | | | | 1.24 mins | 4.27 (t, J = 4.8 Hz, 2 H), 3.85 (d, J = 7.0 Hz, 2 H), 3.78-3.59 (m, 4 H), 3.18-3.00 (m, 2 H), 2.98-2.75 |
| | | | | | (Method D) | (m, 4 H), 2.06-1.91 (m, 2 H), 1.23-1.18 (m, 1 H), 0.89 (t, J = 7.3 Hz, 3 H), 0.56 (q, J = 5.4 Hz, 2 H), |
| | | | | | | 0.31 (d, J = 4.6 Hz, 2 H) |
| (R)-4-(1-(5-(cyclopropylmethoxy)pyridin-2- | C | A | C | Method A | m/z = 590.2 | ¹H NMR (400 MHz, DMSO-d6, ppm) δ 8.23 (dd, J = 2.6, 1.0 Hz, 1 H), 7.82-7.67 (m, 2 H), 7.38- |
| yl)propoxy)-N-((4-(2- | | | | (30-35-45) | [M + H]+, Rt = | 7.21 (m, 3 H), 7.03-6.91 (m, 2 H), 6.83-6.65 (m, 1 H), 5.30 (t, J = 6.5 Hz, 1 H), 4.31 (t, J = 5.1 Hz, |
| (dimethylamino)ethoxy)phenyl)sulfonyl)-2,3- | | | | | 1.21 mins | 2 H), 3.85 (d, J = 7.0 Hz, 2 H), 3.52-3.36 (m, 2 H), 2.78 (s, 6 H), 2.11-1.82 (m, 2 H), 1.30-1.08 (m, |
| difluorobenzamide | | | | | (Method D) | 1 H), 0.89 (t, J = 7.3 Hz, 3 H), 0.65-0.47 (m, 2 H), 0.40-0.21 (m, 2 H) |
| (R)-4-(1-(5-(cyclopropylmethoxy)pyridin-2- | C | A | C | Method A | m/z = 645.3 | ¹H NMR (400 MHz, CDCl3, ppm) δ 8.23 (d, J = 2.8 Hz, 1 H), 8.03-7.91 (m, 2 H), 7.57-7.41 (m, 1 |
| yl)propoxy)-2,3-difluoro-N-((4-(2-(4- | | | | (30-80) | [M + H]+, Rt = | H), 7.27-7.20 (m, 1 H), 7.13 (dd, J = 8.7, 2.8 Hz, 1 H), 6.95-6.84 (m, 2 H), 6.61 (t, J = 8.0 Hz, 1 H), |
| methylpiperazin-1- | | | | | 1.21 mins | 5.27-5.13 (m, 1 H), 4.12 (t, J = 5.3 Hz, 2 H), 3.91-3.71 (m, 2 H), 2.90 (t, J = 5.4 Hz, 3 H), 2.85-2.80 |
| yl)ethoxy)phenyl)sulfonyl)benzamide | | | | | (Method D) | (m, 7 H), 2.55 (s, 3 H), 2.23-1.85 (m, 2 H), 1.42-1.12 (m, 1 H), 1.01 (t, J = 7.4 Hz, 3 H), 0.77-0.56 |
| | | | | | | (m, 2 H), 0.37-0.33 (m, 2 H) |
| (R)-N-((4-(2-(1H-imidazol-1- | D | A | D | C18 (5-95 | m/z = 615.4 | ¹H NMR (400 MHz, DMSO-d6, ppm) δ 8.25 (d, J = 2.6 Hz, 1 H), 8.02 (s, 1 H), 7.75-7.71 (m, 2 |
| yl)ethoxy)phenyl)sulfonyl)-2,3-difluoro-4-(1- | | | | MeCN/H₂O + | [M − H]−, Rt = | H), 7.40-7.25 (m, 4 H), 7.06 (s, 1 H), 6.97-6.93 (m, 2 H), 6.74 (t, J = 7.9 Hz, 1 H), 5.32 (t, J = 6.4 |
| (5-(2-methoxyethoxy )pyridin-2- | | | | 0.1% | 3.69 mins | Hz, 1 H), 4.41 (t, J = 4.9 Hz, 2 H), 4.31 (t, J = 5.0 Hz, 2 H), 4.16-4.12 (m, 2 H), 3.66-3.62 (m, 2 |
| yl)propoxy)benzamide | | | | HCOOH) | (Method A) | H), 3.29 (s, 3 H), 2.04-1.90 (m, 2 H), 1.27-1.22 (m, 1 H), 0.92-0.87 (m, 3 H) |
| (R)-N-((4-(2- | D | A | D | C18 (5-95 | m/z = 594.4 | ¹H NMR (400 MHz, DMSO-d6, ppm) δ 8.25 (d, J = 2.6 Hz, 1 H), 7.75-7.69 (m, 2 H), 7.40-7.29 |
| (dimethylamino)ethoxy)phenyl)sulfonyl)-2,3- | | | | MeCN/H₂O + | [M + H]+, Rt = | (m, 3 H), 6.97-6.91 (m, 2 H), 6.72 (t, J = 7.8 Hz, 1 H), 5.30 (t, J = 6.5 Hz, 1 H), 4.20 (t, J = 5.2 Hz, |
| difluoro-4-(1-(5-(2-methoxyethoxy)pyridin-2- | | | | 0.1% | 3.54 mins | 2 H), 4.16-4.12 (m, 2 H), 3.66-3.61 (m, 2 H), 3.28 (s, 3 H), 3.11 (s, 2 H), 2.57 (s, 6 H), 2.05- |
| yl)propoxy)benzamide | | | | HCOOH) | (Method A) | 1.90 (m, 2 H), 0.90 (t, J = 7.3 Hz, 3 H) |
| R)-4-(1-(5-(cyclopropylmethoxy)pyridin-2- | C | A | C | Method C | m/z = 616.2 | ¹H NMR (400 MHz, DMSO-d6, ppm) δ 9.65 (s, 1 H), 8.23 (d, J = 2.0 Hz, 1 H), 7.75 (d, J = 8.8 Hz, |
| yl)propoxy)-2,3-difluoro-N-((4-(2- | | | | (35-40) | [M + H]+, Rt = | 2 H), 7.40-7.22 (m, 3 H), 6.98 (d, J = 8.8 Hz, 2 H), 6.77-6.67 (m, 1 H), 5.30 (t, J = 6.5 Hz, 1 H), |
| (pyrrolidin-1- | | | | | 1.30 mins | 4.41-4.18 (m, 2 H), 3.85 (d, J = 7.1 Hz, 2 H), 3.66-3.45 (m, 2 H), 3.22-2.80 (m, 4 H), 2.09-1.73 (m, |
| yl)ethoxy)phenyl)sulfonyl)benzamide | | | | | (Method E) | 6 H), 1.28-1.13 (m, 1 H), 0.89 (t, J = 7.3 Hz, 3 H), 0.64-0.45 (m, 2 H), 0.43-0.20 (m, 2 H) |
| 4-((R)-1-(5-(cyclopropylmethoxy)pyridin-2- | C | A | C | Method G | m/z = 634.2 | ¹H NMR (400 MHz, DMSO-d6, ppm) δ 8.23 (d, J = 2.2 Hz, 1 H), 7.73 (d, J = 8.7 Hz, 2 H), 7.35- |
| yl)propoxy)-2,3-difluoro-N-((4-(2-((S)-3- | | | | (45-55) | [M + H]+, Rt = | 7.27 (m, 3 H), 6.95 (d, J = 8.3 Hz, 2 H), 6.73 (t, J = 8.3 Hz, 1 H), 5.35-5.15 (m, 2 H), 4.26-4.06 (m, |
| fluoropyrrolidin-1- | | | | | 1.32 mins | 2 H), 3.85 (d, J = 7.1 Hz, 2 H), 3.30 (s, 2 H), 3.15-2.71 (m, 4 H), 2.30-2.06 (m, 2 H), 2.01-1.94 (m, |
| yl)ethoxy)phenyl)sulfonyl)benzamide | | | | | (Method E) | 2 H), 1.21-1.18 (m, 1 H), 0.89 (t, J = 7.4 Hz, 3 H), 0.59-0.53 (m, 2 H), 0.34-0.28 (m, 2 H) |
| (R)-4-(1-(5-(cyclopropylmethoxy)pyridin-2- | C | H | A | Method A | m/z = 623.7 | ¹H NMR (400 MHz, DMSO-d6, ppm) δ 8.24 (s, 1 H), 7.45-7.16 (m, 3 H), 6.81-6.71 (m, 1 H), 5.32 |
| yl)propoxy)-2,3-difluoro-N-((4-((1- | | | | (20-70) | [M + H]+, Rt = | (t, J = 7.4 Hz, 1 H), 3.86 (d, J = 7.1 Hz, 2 H), 3.70-3.58 (m, 1 H), 3.51-3.42 (m, 1 H), 3.41-3.35 (m, |
| methylpiperidin-4-yl)oxy)piperidin-1- | | | | | 1.08 mins | 2 H), 3.23-3.08 (m, 2 H), 3.07-2.93 (m, 2 H), 2.88-2.74 (m, 2 H), 2.72 (s, 3 H), 2.05-1.94 (m, 2 H), |
| yl)sulfonyl)benzamide | | | | | (Method D) | 1.84-1.49 (m, 6 H), 1.47-1.33 (m, 2 H), 1.23-1.14 (m, 1 H), 0.91 (t, J = 7.3 Hz, 3 H), 0.60-0.51 (m, |
| | | | | | | 2 H), 0.36-0.27 (m, 2 H) |
| (R)-4-(1-(5-(cyclopropylmethoxy)pyridin-2- | C | B | A | Method A | m/z = 609.3 | ¹H NMR (400 MHz, DMSO-d6, ppm) δ 8.24 (s, 1 H), 7.43-7.26 (m, 3 H), 6.73 (t, J = 8.2 Hz, 1 H), |
| yl)propoxy)-N-((6-(2- | | | | (30-80) | [M + H]+, Rt = | 5.30 (t, J = 6.4 Hz, 1 H), 3.86 (d, J = 7.1 Hz, 3 H), 3.69-3.55 (m, 4 H), 3.51-3.42 (m, 2 H), 3.00-2.89 |
| (dimethylamino)ethoxy)-2- | | | | | 0.97 mins | (m, 2 H), 2.58 (s, 6 H), 2.37-2.35 (m, 2 H), 2.08-1.87 (m, 4 H), 1.22-1.15 (m, 2 H), 0.91 (t, J = 7.3 |
| azaspiro[3.3]heptan-2-yl)sulfonyl)-2,3- | | | | | (Method F) | Hz, 3 H), 0.62-0.52 (m, 2 H), 0.35-0.27 (m, 2 H) |
| difluorobenzamide | | | | | | |
| (R)-4-(1-(5-(cyclopropylmethoxy)pyridin-2- | C | B | A | Method A | m/z = 635.3 | ¹H NMR (400 MHz, DMSO-d6, ppm) δ 8.24 (s, 1 H), 7.42-7.24 (m, 3 H), 6.73 (t, J = 8.0 Hz, 1 H), |
| yl)propoxy)-2,3-difluoro-N-((6-(2- | | | | (30-80) | [M + H]+, Rt = | 5.30 (t, J = 6.4 Hz, 1 H), 3.87 (d, J = 7.0 Hz, 2 H), 3.83-3.78 (m, 1 H), 3.64 (s, 2 H), 3.58 (s, 2 H), |
| (pyrrolidin-1-yl)ethoxy)-2- | | | | | 1.01 mins | 3.43-3.36 (m, 4 H), 2.80-2.62 (m, 4 H), 2.37-2.28 (m, 2 H), 2.03-1.91 (m, 4 H), 1.77-1.66 (m, 4 H), |
| azaspiro[3.3]heptan-2-yl)sulfonyl)benzamide | | | | | (Method F) | 1.25-1.15 (m, 1 H), 0.91 (t, J = 7.3 Hz, 3 H), 0.63-0.44 (m, 2 H), 0.36-0.27 (m, 2 H) |
| 4-((R)-1-(5-(cyclopropylmethoxy)pyridin-2- | C | C | C | Method D | m/z = 602.2 | ¹H NMR (400 MHz, DMSO-d6, ppm) δ 8.23 (d, J = 1.4 Hz, 1 H), 7.75 (d, J = 8.5 Hz, 2 H), 7.39- |
| yl)propoxy)-2,3-difluoro-N-((4-(((S)-1- | | | | (20-70) | [M + H]+, Rt = | 7.21 (m, 3 H), 6.95 (d, J = 8.5 Hz, 2 H), 6.73 (t, J = 8.3 Hz, 1 H), 5.30 (t, J = 6.2 Hz, 1 H), 5.20-5.12 |
| methylpyrrolidin-3- | | | | | 1.01 mins | (m, 1 H), 3.85 (d, J = 7.0 Hz, 2 H), 3.60-5.39 (m, 3 H), 3.27-3.12 (m, 2 H), 2.83 (s, 3 H), 2.15-2.03 |
| yl)oxy)phenyl)sulfonyl)benzamide | | | | | (Method D) | (m, 1 H), 2.04-1.85 (m, 2 H), 1.26-1.13 (m, 1 H), 0.89 (t, J = 7.2 Hz, 3 H), 0.60-0.51 (m, 2 H), 0.34- |
| | | | | | | 0.26 (m, 2 H) |
| 4-((R)-1-(5-(cyclopropylmethoxy)pyridin-2- | C | C | C | Method D | m/z = 602.1 | ¹H NMR (400 MHz, DMSO-d6, ppm) δ 8.23 (d, J = 1.8 Hz, 1 H), 7.73 (d, J = 8.8 Hz, 2 H), 7.37- |
| yl)propoxy)-2,3-difluoro-N-((4-(((R)-1- | | | | (20-80) | [M + H]+, Rt = | 7.27 (m, 3 H), 6.92 (d, J = 8.8 Hz, 2 H), 6.72 (t, J = 7.8 Hz, 1 H), 5.30 (t, J = 6.5 Hz, 1 H), 5.16-5.07 |
| methylpyrrolidin-3- | | | | | 1.43 mins | (m, 1 H), 3.85 (d, J = 7.1 Hz, 2 H), 3.47-3.35 (m, 4 H), 3.16-3.05 (m, 1 H), 2.76 (s, 3 H), 2.06-1.87 |
| yl)oxy)phenyl)sulfonyl)benzamide | | | | | (Method E) | (m, 3 H), 1.24-1.16 (m, 1 H), 0.89 (t, J = 7.4 Hz, 3 H), 0.59-0.51 (m, 2 H), 0.33-0.28 (m, 2 H) |
| (R)-4-(1-(5-(cyclopropylmethoxy)pyridin-2- | C | C | C | Method D | m/z = 630.2 | ¹H NMR (400 MHz, DMSO-d6, ppm) δ 9.07 (br, 1 H), 8.23 (d, J = 2.1 Hz, 1 H), 7.73 (d, J = 8.6 |
| yl)propoxy)-N-((4-((1-ethylpiperidin-4- | | | | (40-85) | [M + H]+, Rt = | Hz, 2 H), 7.36-7.26 (m, 3 H), 6.99 (d, J = 8.4 Hz, 2 H), 6.73 (t, J = 8.2 Hz, 1 H), 5.30 (t, J = 6.5 Hz, |
| yl)oxy)phenyl)sulfonyl)-2,3- | | | | | 1.13 mins | 1 H), 4.85-4.55 (m, 1 H), 3.85 (d, J = 7.1 Hz, 2 H), 3.20-2.91 (m, 5 H), 2.17-1.72 (m, 6 H), 1.27- |
| difluorobenzamide | | | | | (Method D) | 1.10 (m, 5 H), 0.89 (t, J = 7.4 Hz, 3 H), 0.58-0.52 (m, 2 H), 0.33-0.26 (m, 2 H) |
| (R)-4-(1-(5-(cyclopropylmethoxy)pyridin-2- | C | C | C | Method D | m/z = 588.1 | ¹H NMR (400 MHz, DMSO-d6, ppm) δ 8.24 (d, J = 1.4 Hz, 1 H), 7.83 (d, J = 8.6 Hz, 2 H), 7.36- |
| yl)propoxy)-2,3-difluoro-N-((4-((1- | | | | (45-95) | [M + H]+, Rt = | 7.25 (m, 3 H), 6.97 (d, J = 8.5 Hz, 2 H), 6.80 (t, J = 7.9 Hz, 1 H), 5.35 (t, J = 6.4 Hz, 1 H), 5.20-5.06 |
| methylazetidin-3- | | | | | 1.05 mins | (m, 1 H), 4.69-4.47 (m, 2 H), 4.18 (d, J = 8.4 Hz, 2 H), 3.86 (d, J = 7.1 Hz, 2 H), 2.90 (s, 3 H), 2.05- |
| yl)oxy)phenyl)sulfonyl)benzamide | | | | | (Method D) | 1.91 (m, 2 H), 1.33-1.12 (m, 2 H), 0.89 (t, J = 7.3 Hz, 3 H), 0.60-0.52 (m, 2 H), 0.35-0.26 (m, 2 H) |
| (R)-4-(1-(5-(cyclopropylmethoxy)pyridin-2- | C | D | A | Method D | m/z = 618.2 | ¹H NMR (400 MHz, DMSO-d6, ppm) δ 9.32 (s, 1 H), 8.24 (d, J = 2.4 Hz, 1 H), 8.01 (d, J = 9.1 Hz, |
| yl)propoxy)-2,3-difluoro-N-((6-((1- | | | | (45-95) | [M + H]+, Rt = | 1 H), 7.43-7.28 (m, 3 H), 7.23 (d, J = 9.1 Hz, 1 H), 6.76 (t, J = 7.9 Hz, 1 H), 5.54-5.36 (m, 1 H), |
| methylpiperidin-4-yl)oxy)pyridazin-3- | | | | | 1.06 mins | 5.32 (t, J = 6.5 Hz, 1 H), 3.86 (d, J = 7.1 Hz, 2 H), 3.30-3.08 (m, 4 H), 2.81 (s, 3 H), 2.05-1.91 (m, 6 |
| yl)sulfonyl)benzamide | | | | | (Method D) | H), 1.30-1.12 (m, 1 H), 0.90 (t, J = 7.3 Hz, 3 H), 0.61-0.51 (m, 2 H), 0.33-0.29 (m, 2 H) |
| (R)-4-(1-(5-(cyclopropylmethoxy)pyridin-2- | C | E | D | Method A | m/z = 617.3 | ¹H NMR (400 MHz, DMSO-d6, ppm) δ 9.30 (br, 1 H), 8.49 (d, J = 2.1 Hz, 1 H), 8.23 (d, J = 1.8 |
| yl)propoxy)-2,3-difluoro-N-((6-((1- | | | | (35-75) | [M + H]+, Rt = | Hz, 1 H), 8.04 (dd, J = 8.6, 2.3 Hz, 1 H), 7.39-7.23 (m, 3 H), 6.80 (d, J = 8.7 Hz, 1 H), 6.76-6.69 |
| methylpiperidin-4-yl)oxy)pyridin-3- | | | | | 1.08 mins | (m, 1 H), 5.35-5.16 (m, 2 H), 3.85 (d, J = 7.1 Hz, 2 H), 3.28-3.06 (m, 3 H), 2.79 (s, 3 H), 2.25-1.67 |
| yl)sulfonyl)benzamide | | | | | (Method D) | (m, 7 H), 1.25-1.14 (m, 1 H), 0.89 (t, J = 7.4 Hz, 3 H), 0.61-0.51 (m, 2 H), 0.37-0.25 (m, 2 H) |
| (R)-4-(1-(5-(cyclopropylmethoxy)pyridin-2- | C | C | C | Method A | m/z = 619.3 | ¹H NMR (400 MHz, CDCl₃, ppm) δ 8.23 (d, J = 2.8 Hz, 1 H), 8.05 (d, J = 8.8 Hz, 2 H), 7.52 (t, J = |
| yl)propoxy)-2,3-difluoro-N-((4-((1-(methyl- | | | | (35-80) | [M + H]+, Rt = | 7.9 Hz, 1 H), 7.25-7.19 (m, 1 H), 7.16-7.10 (m, 1 H), 6.96 (d, J = 8.6 Hz, 2 H), 6.67 (t, J = 7.8 Hz, 1 |
| d3)piperidin-4- | | | | | 1.03 mins | H), 5.31-5.08 (m, 1 H), 4.73-4.60 (m, 1 H), 3.81 (d, J = 6.9 Hz, 2 H), 3.30-3.13 (m, 2 H), 3.06 (t, J = |
| yl)oxy)phenyl)sulfonyl)benzamide | | | | | (Method F) | 10.6 Hz, 2 H), 2.56-2.37 (m, 2 H), 2.18-1.93 (m, 4 H), 1.33-1.20 (m, 1 H), 1.01 (t, J = 7.4 Hz, 3 H), |
| | | | | | | 0.73-0.59 (m, 2 H), 0.40-0.29 (m, 2 H) |
| (R)-4-(1-(5-(cyclopropylmethoxy)pyridin-2- | C | C | D | Method A | m/z = 656.3 | ¹H NMR (400 MHz, DMSO-d6, ppm): δ 9.25 (br, 1 H), 8.24 (s, 1 H), 7.76 (d, J = 8.6 Hz, 2 H), |
| yl)propoxy)-N-((4-((1- | | | | (35-85) | [M + H]+, Rt = | 7.45-7.24 (m, 3 H), 7.02 (d, J = 8.6 Hz, 2 H), 6.75 (t, J = 8.1 Hz, 1 H), 5.32 (t, J = 6.4 Hz, 1 H), |
| (cyclopropylmethyl)piperidin-4- | | | | | 1.06 mins | 4.92-4.51 (m, 1 H), 3.86 (d, J = 7.0 Hz, 2 H), 3.72-3.40 (m, 2 H), 3.24-3.09 (m, 2 H), 3.06-2.91 (m, |
| yl)oxy)phenyl)sulfonyl)-2,3- | | | | | (Method F) | 2 H), 2.33-1.71 (m, 6 H), 1.27-1.14 (m, 1 H), 1.13-0.99 (m, 1 H), 0.90 (t, J = 7.3 Hz, 3 H), 0.70- |
| difluorobenzamide | | | | | | 0.61 (m, 2 H), 0.60-0.53 (m, 2 H), 0.40-0.34 (m, 2 H), 0.35-0.27 (m, 2 H) |
| (R)-4-(1-(5-(cyclopropylmethoxy)pyridin-2- | C | C | A | Method D | m/z = 644.2 | ¹H NMR (400 MHz, DMSO-d6, ppm) δ 9.01 (s, 1 H), 8.26-8.20 (m, 1 H), 7.72 (d, J = 7.6 Hz, 2 H), |
| yl)propoxy)-2,3-difluoro-N-((4-((1- | | | | (30-65) | [M + H]+, Rt = | 7.36-7.28 (m, 3 H), 7.03-6.92 (m, 2 H), 6.72 (t, J = 7.9 Hz, 1 H), 5.29 (t, J = 6.5 Hz, 1 H), 4.87-4.52 |
| isopropylpiperidin-4- | | | | | 1.37 mins | (m, 1 H), 3.85 (d, J = 7.1 Hz, 2 H), 3.56-3.35 (m, 2 H), 3.20-3.04 (m, 2 H), 2.26-1.74 (m, 7 H), |
| yl)oxy)phenyl)sulfonyl)benzamide | | | | | (Method E) | 1.30-1.18 (m, 7 H), 0.89 (t, J = 7.3 Hz, 3 H), 0.58-0.50 (m, 2 H), 0.35-0.27 (m, 2 H) |
| (R)-4-(1-(5-(cyclopropylmethoxy)pyridin-2- | C | E | B | Method A | m/z = 616.2 | ¹H NMR (400 MHz, DMSO-d6, ppm) δ 9.18 (br, 1 H), 8.33 (s, 1 H), 8.23 (s, 1 H), 7.71 (d, J = 8.8 |
| yl)propoxy)-2,3-difluoro-N-((6-((1- | | | | (25-70) | [M + H]+, Rt = | Hz, 1 H), 7.36-7.24 (m, 3 H), 7.07-6.94 (m, 1 H), 6.72 (t, J = 8.0 Hz, 1 H), 6.41 (d, J = 8.6 Hz, 1 H), |
| methylpiperidin-4-yl)amino)pyridin-3- | | | | | 1.07 mins | 5.30 (s, 1 H), 4.05-3.92 (m, 1 H), 3.86 (d, J = 7.1 Hz, 2 H), 3.48-3.34 (m, 1 H), 3.16-2.97 (m, 2 H), |
| yl)sulfonyl)benzamide | | | | | (Method D) | 2.74 (s, 3 H), 2.14-1.88 (m, 4 H), 1.72-1.45 (m, 2 H), 1.26-1.13 (m, 1 H), 0.89 (t, J = 7.3 Hz, 4 H), |
| | | | | | | 0.60-0.49 (m, 2 H), 0.34-0.25 (m, 2 H) |
| 4-((R)-1-(5-(cyclopropylmethoxy)pyridin-2- | C | C | B | Method D | m/z = 644.5 | ¹H NMR (400 MHz, DMSO-d6, ppm) δ 9.34 (br, 1 H), 8.23 (d, J = 1.9 Hz, 1 H), 7.71 (d, J = 8.5 |
| yl)propoxy)-2,3-difluoro-N-((4-((1,2,2- | | | | (25-65) | [M + H]+, Rt = | Hz, 2 H), 7.36-7.25 (m, 3 H), 6.95 (d, J = 8.7 Hz, 2 H), 6.75-6.68 (m, 1 H), 5.29 (t, J = 6.4 Hz, 1 H), |
| trimethylpiperidin-4- | | | | | 1.44 mins | 4.86-4.63 (m, 1 H), 3.85 (d, J = 7.1 Hz, 2 H), 3.29-3.19 (m, 2 H), 2.80-2.59 (m, 3 H), 2.45-2.39 (m, |
| yl)oxy)phenyl)sulfonyl)benzamide | | | | | (Method E) | 1 H), 2.28-2.17 (m, 1 H), 2.06-1.81 (m, 3 H), 1.75-1.59 (m, 1 H), 1.34 (s, 6 H), 1.23-1.15 (m, 1 H), |
| | | | | | | 0.89 (t, J = 7.4 Hz, 3 H), 0.59-0.50 (m, 2 H), 0.35-0.25 (m, 2 H) |
| (R)-4-(1-(5-(cyclopropylmethoxy)pyridin-2- | C | B | A | Method D | m/z = 623.3 | ¹H NMR (400 MHz, DMSO-d6, ppm) δ 9.54 (br, 1 H), 8.24 (s, 1 H), 7.39-7.26 (m, 3 H), 6.72 (t, J = |
| yl)propoxy)-2,3-difluoro-N-((4-(2- | | | | (45-95) | [M + H]+, Rt = 1 | 8.1 Hz, 1 H), 5.29 (t, J = 6.4 Hz, 1 H), 3.86 (d, J = 7.0 Hz, 2 H), 3.65 (t, J = 4.9 Hz, 2 H), 3.45-3.34 |
| (pyrrolidin-1-yl)ethoxy)piperidin-1- | | | | | mins (Method | (m, 2 H), 3.30-3.01 (m, 7 H), 2.78 (t, J = 9.6 Hz, 2 H), 2.05-1.78 (m, 8 H), 1.54-1.40 (m, 2 H), 1.20- |
| yl)sulfonyl)benzamide | | | | | F) | 1.17 (m, 1 H), 0.90 (t, J = 7.3 Hz, 3 H), 0.56-0.53 (m, 2 H), 0.32-0.29 (m, 2 H) |
| (R)-4-(1-(5-(cyclopropylmethoxy)pyridin-2- | C | D | B | Method D | m/z= 618.5 | ¹H NMR (400 MHz, DMSO-d6, ppm) δ 9.32 (s, 1 H), 8.85 (s, 2 H), 8.23 (s, 1 H), 7.41-7.28 (m, 3 |
| yl)propoxy)-2,3-difluoro-N-((2-((1- | | | | (20-60) | [M + H]+, Rt = | H), 6.73 (t, J = 8.0 Hz, 1 H), 5.31 (t, J = 6.5 Hz, 1 H), 5.26-5.12 (m, 1 H), 3.85 (d, J = 7.1 Hz, 2 H), |
| methylpiperidin-4-yl)oxy)pyrimidin-5- | | | | | 1.32 mins | 3.26-2.98 (m, 4 H), 2.82-2.67 (m, 2 H), 2.19-1.84 (m, 6 H), 1.22-1.15 (m, 2 H), 0.89 (t, J = 7.3 Hz, |
| yl)sulfonyl)benzamide | | | | | (Method E) | 3 H), 0.59-0.51 (m, 2 H), 0.33-0.28 (m, 2 H) |
| 4-((R)-1-(5-(cyclopropylmethoxy)pyridin-2- | C | C | B | Method A | m/z = 628.2 | ¹H NMR (400 MHz, DMSO-d6, ppm) δ 8.23 (s, 1 H), 7.71 (d, J = 8.6 Hz, 2 H), 7.37-7.23 (m, 3 H), |
| yl)propoxy)-2,3-difluoro-N-((4-(((1R,4R,5R)- | | | | (40-75) | [M + H]+, Rt = | 6.91 (d, J = 8.7 Hz, 2 H), 6.71 (t, J = 8.0 Hz, 1 H), 5.29 (t, J = 4.2 Hz 1 H), 4.92-4.81 (m, 1 H), |
| 2-methyl-2-azabicyclo[2.2.1]heptan-5- | | | | | 1.15 mins | 3.89-3.79 (m, 3 H), 3.60-3.58 (m 1 H), 3.01-2.94 (m, 1 H), 2.93-2.84 (m, 1 H), 2.72 (s, 3 H), 2.36 - |
| yl)oxy)phenyl)sulfonyl)benzamide | | | | | (Method D) | 2.39 (m, 1 H), 2.00-1.92 (m, 3 H), 1.82-1.72 (m, 1 H), 1.63-1.52 (m, 1 H), 1.26-1.13 (m, 1 H), 0.89 |
| | | | | | | (t, J = 7.3 Hz, 3 H), 0.58-0.53 (m, 2 H), 0.33-0.29 (m, 2 H) |
| 4-((R)-1-(5-(cyclopropylmethoxy)pyridin-2- | C | B | B | Method C | m/z = 635.2 | ¹H NMR (400 MHz, DMSO-d6, ppm) δ 8.26-8.22 (m, 1 H), 7.38-7.33 (m, 2 H), 7.32-7.23 (m, 1 H), |
| yl)propoxy)-2,3-difluoro-N-((5-(2- | | | | (40-60) | [M + H]+, Rt = | 6.75 (t, J = 8.0 Hz, 1 H), 5.31 (t, J = 5.3 Hz, 1 H), 3.94 (s, 1 H), 3.86 (d, J = 7.1 Hz, 2 H), 3.65-3.55 |
| (pyrrolidin-1-yl)ethoxy)-2- | | | | | 1.13 mins | (m, 3 H), 3.32-3.22 (m, 7 H), 2.73-2.64 (m, 1 H), 2.57 (s, 1 H), 2.15-2.10 (m, 1 H), 1.98-1.94 (m, 2 |
| azabicyclo[2.2.1]heptan-2- | | | | | (Method D) | H), 1.91-1.80 (m, 4 H), 1.76-1.65 (m, 1 H), 1.43 (d, J = 9.1 Hz, 1 H), 1.32 (d, J = 12.9 Hz, 1 H), |
| yl)sulfonyl)benzamide | | | | | | 1.26-1.16 (m, 1 H), 0.91 (t, J = 7.4 Hz, 3 H), 0.58-0.54(m, 2 H), 0.33-0.29 (m, 2 H) |
| (R)-4-(1-(5-(cyclopropylmethoxy)pyridin-2- | C | C | B | Prep TLC (9:1 | m/z = 666.5 | ¹H NMR (400 MHz, DMSO-d6, ppm) δ 12.14 (s, 1 H), 8.23 (s, 1 H), 7.83 (d, J = 8.9 Hz, 2 H), |
| yl)propoxy)-N-((4-((1-(2,2- | | | | DCM:MeOH + | [M + H]+, Rt = | 7.38-7.32 (m, 2 H), 7.28-7.22 (m, 1 H), 7.13 (d, J = 8.9 Hz, 2 H), 6.87 (t, J = 7.9 Hz, 1 H), 6.31-6.00 |
| difluoroethyl)piperidin-4- | | | | 0.5% | 1.45 mins | (m, 1 H), 5.39 (t, J = 6.5 Hz, 1 H), 4.61-4.49 (m, 1 H), 3.86 (d, J = 7.1 Hz, 2 H), 3.02-2.72 (m, 4 H), |
| yl)oxy)phenyl)sulfonyl)-2,3- | | | | NH₄OH) | (Method E) | 2.53-2.50 (m, 2 H), 2.03-1.93 (m, 4 H), 1.71-1.62 (m, 2 H), 1.21-1.16 (m, 1 H), 0.89 (t, J = 7.4 Hz, |
| difluorobenzamide | | | | | | 3 H), 0.59-0.54 (m, 2 H), 0.33-0.28 (m, 2 H) |
| (R)-4-(1-(5-(cyclopropylmethoxy)pyridin-2- | C | D | A | Method B | m/z= 618.3 | ¹H NMR (400 MHz, DMSO-d6, ppm) δ 9.61 (s, 1 H), 8.24 (s, 1 H), 8.04 (d, J = 9.1 Hz, 1 H), 7.42- |
| yl)propoxy)-2,3-difluoro-N-((6-(2- | | | | (45-95) | [M + H]+, Rt = | 7.26 (m, 4 H), 6.76 (t, J = 8.1 Hz, 1 H), 5.32 (t, J = 6.4 Hz, 1 H), 4.81-4.72 (m, 2 H), 3.86 (d, J = 7.1 |
| (pyrrolidin-1-yl)ethoxy)pyridazin-3- | | | | | 1.08 mins | Hz, 2 H), 3.73-3.42 (m, 4 H), 3.25-2.91 (m, 2 H), 2.03-1.91 (m, 6 H), 1.23-1.15 (m, 1 H), 0.90 (t, |
| yl)sulfonyl)benzamide | | | | | (Method D) | J = 7.3 Hz, 3 H), 0.56-0.53 (m, 2 H), 0.34-0.28 (m, 2 H) |
| (R)-4-(1-(5-(cyclopropylmethoxy)pyridin-2- | C | E | C | Method D | m/z= 616.3 | ¹H NMR (400 MHz, DMSO-d6, ppm) δ 9.48 (s, 1 H), 8.38 (s, 1 H), 8.23 (s, 1 H), 7.76 (d, J = 8.7 |
| yl)propoxy)-2,3-difluoro-N-((6-((2- | | | | (25-70) | [M + H]+, Rt = | Hz, 1 H), 7.40-7.21 (m, 3 H), 7.15 (s, 1 H), 6.72 (t, J = 7.8 Hz, 1 H), 6.48 (d, J = 8.7 Hz, 1 H), 5.30 |
| (pyrrolidin-1-yl)ethyl)amino)pyridin-3- | | | | | 1.11 mins | (t, J = 6.4 Hz, 1 H), 3.85 (d, J = 7.0 Hz, 2 H), 3.59 (d, J = 5.7 Hz, 2 H), 3.30-3.13 (m, 6 H), 2.03- |
| yl)sulfonyl)benzamide | | | | | (Method D) | 1.86 (m, 6 H), 1.25-1.12 (m, 1 H), 0.89 (t, J = 7.3 Hz, 3 H), 0.65-0.48 (m, 2 H), 0.38-0.21 (m, 2 H) |
| (R)-4-(1-(5-(cyclopropylmethoxy)pyridin-2- | C | C | A | Method E | m/z = 616 | ¹H NMR (400 MHz, DMSO-d6, ppm) δ 8.23 (dd, J = 2.5, 0.8 Hz, 1 H), 7.76-7.69 (m, 2 H), 7.37- |
| yl)propoxy)-2,3-difluoro-N-((4-((1- | | | | (20-75) | [M + H]+, Rt = | 7.26 (m, 3 H), 6.97 (d, J = 8.9 Hz, 2 H), 6.78-6.67 (m, J = 7.7 Hz, 1 H), 5.30 (t, J = 6.5 Hz, 1 H), |
| methylpiperidin-4- | | | | | 1.3 mins | 4.69-4.60 (m, 1 H), 3.85 (d, J = 7.1 Hz, 2 H), 3.26-3.02 (m, 4 H), 2.74 (s, 3 H), 2.13-1.79 (m, 6 H), |
| yl)oxy)phenyl)sulfonyl)benzamide | | | | | (Method E) | 1.23-1.17 (m, 2 H), 0.90 (t, J = 7.4 Hz, 3 H), 0.59-0.50 (m, 2 H), 0.35-0.26 (m, 2 H) |
| (R)-4-(1-(5-(cyclopropylmethoxy)pyridin-2- | C | F | A | Method B | m/z = 617.4 | ¹H NMR (400 MHz, DMSO-d6, ppm) δ 9.29 (s, 1 H), 8.27-8.14 (m, 2 H), 7.85 (d, J = 8.8 Hz, 1 H), |
| yl)propoxy)-2,3-difluoro-N-((5-((1- | | | | (45-95) | [M + H]+, Rt = | 7.51 (dd, J = 8.7, 2.4 Hz, 1 H), 7.36-7.30 (m, 3 H), 6.74 (t, J = 8.1 Hz, 1 H), 5.31 (t, J = 6.5 Hz, 1 |
| methylpiperidin-4-yl)oxy)pyridin-2- | | | | | 1.02 mins | H), 4.87-4.61 (m, 1 H), 3.86 (d, J = 7.0 Hz, 2 H), 3.26-3.02 (m, 4 H), 2.79 (s, 3 H), 2.21-1.65 (m, 6 |
| yl)sulfonyl)benzamide | | | | | (Method D) | H), 1.23-1.17 (m, 1 H), 0.90 (t, J = 7.3 Hz, 3 H), 0.57-0.53 (m, 2 H), 0.32-0.29 (m, 2 H) |
| (R)-4-(1-(5-(cyclopropylmethoxy)pyridin-2- | C | F | A | Method B | m/z = 616.4 | ¹H NMR (400 MHz, DMSO-d6, ppm) δ 8.24 (s, 1 H), 7.89 (s, 1 H), 7.68 (d, J = 8.2 Hz, 1 H), 7.36- |
| yl)propoxy)-2,3-difluoro-N-((5-((1- | | | | (45-95) | [M + H]+, Rt = | 7.27 (m, 3 H), 6.99 (d, J = 8.3 Hz, 1 H), 6.78 (t, J = 7.9 Hz, 1 H), 6.45 (br, 1 H), 5.33 (t, J = 6.3 Hz, |
| methylpiperidin-4-yl)amino)pyridin-2- | | | | | 1.01 mins | 1 H), 3.86 (d, J = 7.1 Hz, 2 H), 3.65-3.34 (m, 3 H), 3.18-2.91 (m, 2 H), 2.76 (s, 3 H), 2.07-1.91 (m, |
| yl)sulfonyl)benzamide | | | | | (Method D) | 5 H), 1.72-1.42 (m, 2 H), 1.25-1.16 (m, 1 H), 0.90 (t, J = 7.3 Hz, 3 H), 0.58-0.53 (m, 2 H), 0.32- |
| | | | | | | 0.29 (m, 2 H) |
| (R)-4-(1-(5-(cyclopropylmethoxy)pyridin-2- | C | E | B | Method A | m/z = 644.3 | ¹H NMR (400 MHz, DMSO-d6, ppm) δ 8.33 (d, J = 2.4 Hz, 1 H), 8.26 (s, 1 H), 7.72 (dd, J = 8.7, |
| yl)propoxy)-2,3-difluoro-N-((6-((1- | | | | (10-90) | [M + H]+, Rt = | 2.4 Hz, 1 H), 7.37-7.22 (m, 3 H), 7.04 (s, 1 H), 6.71 (t, J = 8.2 Hz, 1 H), 6.41 (d, J = 8.8 Hz, 1 H), |
| isopropylpiperidin-4-yl)amino)pyridin-3- | | | | | 1.14 mins | 5.29 (t, J = 6.5 Hz, 1 H), 4.13-3.95 (m, 1 H), 3.85 (d, J = 7.0 Hz, 2 H), 3.48-3.35 (m, 1 H), 3.29-3.19 |
| yl)sulfonyl)benzamide | | | | | (Method D) | (m, 2 H), 3.18-3.02 (m, 2 H), 2.20-2.07 (m, 2 H), 2.06-1.87 (m, 2 H), 1.72-1.52 (m, 2 H), 1.42-1.04 |
| | | | | | | (m, 7 H), 0.89 (t, J = 7.3 Hz, 3 H), 0.61-0.47 (m, 2 H), 0.45-0.26 (m, 2 H) |
| (R)-4-(1-(5-(cyclopropylmethoxy)pyridin-2- | C | E | B | Method A | m/z = 617.2 | ¹H NMR (400 MHz, DMSO-d6, ppm) δ 9.58 (s, 1 H), 8.54 (d, J = 2.1 Hz, 1 H), 8.23 (d, J = 2.4 Hz, |
| yl)propoxy)-2,3-difluoro-N-((6-(2- | | | | (30-70) | [M + H]+, Rt = | 1 H), 8.09 (dd, J = 8.6, 2.4 Hz, 1 H), 7.38 - 7.22 (m, 3 H), 6.89 (d, J = 8.6 Hz, 1 H), 6.74 (t, J = 7.8 |
| (pyrrolidin-1-yl)ethoxy)pyridin-3- | | | | | 1.1 mins | Hz, 1 H), 5.31 (t, J = 6.4 Hz, 1 H), 4.61 - 4.59 (m, 2 H), 3.85 (d, J = 7.1 Hz, 2 H), 3.67-3.48 (m, 4 |
| yl)sulfonyl)benzamide | | | | | (Method D) | H), 3.17-3.01 (m, 2 H), 2.05 - 1.90 (m, 4 H), 1.90-1.71 (m, 2 H), 1.23 - 1.17 (m, 1 H), 0.89 (t, J = |
| | | | | | | 7.3 Hz, 3 H), 0.60 - 0.50 (m, 2 H), 0.35 - 0.25 (m, 2 H) |
| (R)-4-(1-(5-(cyclopropylmethoxy)pyridin-2- | C | E | D | Method A | m/z = 646 | ¹H NMR (400 MHz, DMSO-d6, ppm) δ 8.94 (br, 1 H), 8.49 (s, 1 H), 8.23 (s, 1 H), 8.05-8.02 (m, 1 |
| yl)propoxy)-2,3-difluoro-N-((6-((1- | | | | (30-60) | [M + H]+, Rt = | H), 7.34-7.29 (m, 3 H), 6.84-6.70 (m, 2 H), 5.35-5.28 (m, 2 H), 3.85 (d, J = 7.1 Hz, 2 H), 3.51-3.42 |
| isopropylpiperidin-4-yl)oxy)pyridin-3- | | | | | 1.5 mins | (m, 2 H), 3.21-3.08 (m, 2 H), 2.36-1.74 (m, 7 H), 1.26-1.22 (m, 6 H), 1.21-1.16 (m, 1 H), 0.89 (t, |
| yl)sulfonyl)benzamide | | | | | (Method E) | J = 7.3 Hz, 3 H), 0.60-0.51 (m, 2 H), 0.32-0.29 (m, 2 H) |
| 4-((R)-1-(5-(cyclopropylmethoxy)pyridin-2- | C | C | A | Method B | m/z = 644.5 | ¹H NMR (400 MHz, DMSO-d6, ppm) δ 8.23 (s, 1 H), 7.73 (d, J = 8.8 Hz, 2 H), 7.35-7.26 (m, 3 H), |
| yl)propoxy)-2,3-difluoro-N-((4-(2-((R)-1- | | | | (45-95) | [M + H]+, Rt = | 6.92 (d, J = 8.8 Hz, 2 H), 6.72 (t, J = 7.9 Hz, 1 H), 5.29 (t, J = 6.5 Hz, 1 H), 4.16-4.03 (m, 2 H), 3.85 |
| methylpiperidin-2- | | | | | 1.12 mins | (d, J = 7.1 Hz, 2 H), 3.14-2.93 (m, 1 H), 2.79 (s, 3 H), 2.30-2.16 (m, 1 H), 1.99-1.90 (m, 5 H), 1.69- |
| yl)ethoxy)phenyl)sulfonyl)benzamide | | | | | (Method D) | 1.44 (m, 6 H), 1.25-1.13 (m, 1 H), 0.89 (t, J = 7.3 Hz, 3 H), 0.59-0.52 (m, 2 H), 0.34-0.27 (m, 2 H) |
| (R)-4-(1-(5-(cyclopropylmethoxy)pyridin-2- | C | C | C | Method D | m/z = 630.3 | ¹H NMR (400 MHz, DMSO-d6, ppm) δ 8.26-8.18 (m, 1 H), 7.77 (d, J = 8.4 Hz, 2 H), 7.46-7.16 |
| yl)propoxy)-2,3-difluoro-N-((4-(2-(piperidin- | | | | (55-75) | [M + H]+, Rt = | (m, 3 H), 7.00 (d, J = 8.5 Hz, 2 H), 6.74 (t, J = 8.1 Hz, 1 H), 5.31 (t, J = 6.5 Hz, 1 H), 4.42-4.26 (m, |
| 1-yl)ethoxy)phenyl)sulfonyl)benzamide | | | | | 1.04 mins | 2 H), 3.85 (d, J = 7.1 Hz, 2 H), 3.62-3.39 (m, 2 H), 3.11-2.85 (s, 2 H), 2.56-2.53 (m, 2 H), 2.12-1.89 |
| | | | | | (Method F) | (m, 2 H), 1.86-1.55 (m, 4 H), 1.58-1.28 (m, 2 H), 1.28-1.12 (m, 1 H), 0.89 (t, J = 7.4 Hz, 3 H), 0.59- |
| | | | | | | 0.53 (m, 2 H), 0.34-0.27 (m, 2 H) |
| (R)-2,3-difluoro-N-((4-((1-methylpiperidin-4- | E | H | B | Method A | m/z = 646.3 | ¹H NMR (400 MHz, MeOH-d4, ppm) δ 8.38 (d, J = 2.5 Hz, 1 H), 8.11 (d, J = 3.2 Hz, 1 H), 7.92- |
| yl)oxy)piperidin-1-yl)sulfonyl)-4-(1-(5- | | | | (45-85) | [M + H]+, Rt = | 7.79 (m, 1 H), 7.62-7.59 (m, 1 H), 7.53 (d, J = 8.6 Hz, 1 H), 7.34 (t, J = 8.2 Hz, 1 H), 7.16-7.13 (m, |
| (pyridin-2-yloxy)pyridin-2- | | | | | 1.02 mins | 1 H), 7.07 (d, J = 8.3 Hz, 1 H), 6.72 (t, J = 7.6 Hz, 1 H), 5.34 (t, J = 6.8 Hz, 1 H), 3.86-3.73 (m, 1 |
| yl)propoxy)benzamide | | | | | (Method D) | H), 3.60-3.58 (m, 1 H), 3.56-3.43 (m, 2 H), 3.32-3.16 (m, 2 H), 3.18-2.98 (m, 4 H), 2.80 (s, 3 H), |
| | | | | | | 2.18-2.01 (m, 2 H), 2.00-1.75 (m, 6 H), 1.71-1.57 (m, 2 H), 1.06 (t, J = 7.4 Hz, 3 H) |
| (R)-4-(1-(5-(cyclopropylmethoxy)pyridin-2- | C | D | B | Method A | m/z = 630.6 | ¹H NMR (400 MHz, DMSO-d6, ppm) δ 9.02 (br, 1 H), 8.83 (s, 1 H), 8.24 (s, 1 H), 8.12-8.09 (m, 1 |
| yl)propoxy)-N-((6-((4-ethylpiperazin-1- | | | | (25-70) | [M + H]+, Rt = | H), 7.45 (d, J = 7.9 Hz, 1 H), 7.33-7.29 (m, 3 H), 6.73 (t, J = 8.2 Hz, 1 H), 5.30 (t, J = 6.4 Hz, 1 H), |
| yl)methyl)pyridin-3-yl)sulfonyl)-2,3- | | | | | 1.27 mins | 3.85 (d, J = 7.0 Hz, 2 H), 3.73 (s, 2 H), 3.51-3.36 (m, 2 H), 3.16-2.91 (m, 6 H), 2.44-2.41 (m, 2 H), |
| difluorobenzamide | | | | | (Method E) | 2.03-1.91(m, 2 H), 1.19-1.16 (m, 4 H), 0.89 (t, J = 7.3 Hz, 3 H), 0.60-0.45 (m, 2 H), 0.37-0.23 |
| | | | | | | (m, 2 H) |
| (R)-4-(1-(5-(cyclopropylmethoxy)pyridin-2- | C | C | B | Method A | m/z = 660.2 | ¹H NMR (400 MHz, MeOH-d4, ppm) δ 8.17 (s, 1 H), 7.91 (d, J = 8.8 Hz, 2 H), 7.38-7.25 (m, 3 H), |
| yl)propoxy)-2,3-difluoro-N-((4-((1-(2- | | | | (35-80) | [M + H]+, Rt = | 7.04 (d, J = 8.9 Hz, 2 H), 6.63 (t, J = 8.1 Hz, 1 H), 5.28-5.14 (m, 1 H), 4.78-4.62 (m, 2 H), 3.87 (d, J = |
| methoxyethyl)piperidin-4- | | | | | 1.15 mins | 7.0 Hz, 2 H), 3.76-3.62 (m, 2 H), 3.41 (s, 3 H), 3.38-3.31 (m, 3 H), 2.26-2.14 (m, 2 H), 2.13-2.01 |
| yl)oxy)phenyl)sulfonyl)benzamide | | | | | (Method D) | (m, 3 H), 2.01-1.92 (m, 1 H), 1.27-1.18 (m, 3 H), 0.99 (t, J = 7.4 Hz, 3 H), 0.64-0.54 (m, 2 H), 0.38- |
| | | | | | | 0.30 (m, 2 H) |
| (R)-4-(1-(5-(cyclopropylmethoxy)pyridin-2- | C | C | A | Method B | m/z = 674.3 | ¹H NMR (400 MHz, DMSO-d6, ppm) δ 8.79 (br, 1 H), 8.24 (s, 1 H), 7.74 (d, J = 8.7 Hz, 2 H), 7.36- |
| yl)propoxy)-2,3-difluoro-N-((4-((1-(2- | | | | (45-95) | [M + H]+, Rt = | 7.27 (m, 3 H), 7.01 (d, J = 8.5 Hz, 2 H), 6.75 (t, J = 7.9 Hz, 1 H), 5.35-5.00 (m, 2 H), 4.66 (br, 1 H), |
| hydroxy-2-methylpropyl)piperidin-4- | | | | | 1.1 mins | 3.86 (d, J = 7.1 Hz, 2 H), 3.19-2.85 (m, 4 H), 2.24-1.84 (m, 7 H), 1.29-1.08 (m, 8 H), 0.89 (t, J = 7.3 |
| yl)oxy)phenyl)sulfonyl)benzamide | | | | | (Method D) | Hz, 3 H), 0.59-0.52 (m, 2 H), 0.34-0.27 (m, 2 H) |
| (R)-4-(1-(5-(cyclopropylmethoxy)pyridin-2- | C | G | C | Method B | m/z= 681.3 | ¹H NMR (400 MHz, DMSO-d6, ppm) δ 8.25 (s, 1 H), 7.39-7.23 (m, 3 H), 6.76 (t, J = 8.0 Hz, 1 H), |
| yl)propoxy)-2,3-difluoro-N-((4-((1-(2- | | | | (30-50) | [M + H]+, Rt = | 5.32 (t, J = 6.4 Hz, 1 H), 3.86 (d, J = 7.1 Hz, 2 H), 3.60-3.45 (m, 2 H), 3.37-3.30 (m, 5 H), 3.22-3.07 |
| hydroxy-2-methylpropyl)piperidin-4- | | | | | 1.14 mins | (m, 2 H), 2.82-2.79 (m, 4 H), 2.06-1.86 (m, 4 H), 1.85-1.60 (m, 4 H), 1.45-1.36 (m, 2 H), 1.26-1.12 |
| yl)oxy)piperidin-1-yl)sulfonyl)benzamide | | | | | (Method D) | (m, 7 H), 0.91 (t, J = 7.3 Hz, 3 H), 0.59-0.49 (m, 2 H), 0.35-0.26 (m, 2 H) |
| (R)-4-(1-(5-(cyclopropylmethoxy)pyridin-2- | C | G | A | Method D | m/z = 667.3 | ¹H NMR (400 MHz, DMSO-d6, ppm) δ 8.24 (s, 1 H), 7.48-7.16 (m, 3 H), 6.75 (t, J = 8.1 Hz, 1 H), |
| yl)propoxy)-2,3-difluoro-N-((4-((1-(2- | | | | (40-85) | [M + H]+, Rt = | 5.31 (t, J = 6.5 Hz, 1 H), 3.86 (d, J = 7.1 Hz, 2 H), 3.66-3.54 (m, 3 H), 3.50-3.40 (m, 1 H), 3.39-3.33 |
| methoxyethyl)piperidin-4-yl)oxy)piperidin-1- | | | | | 1.09 mins | (m, 4 H), 3.29 (s, 3 H), 3.20-2.98 (m, 4 H), 2.78 (t, J = 9.9 Hz, 2 H), 2.09-1.84 (m, 4 H), 1.83-1.73 |
| yl)sulfonyl)benzamide | | | | | (Method D) | (m, 2 H), 1.72-1.51 (m, 2 H), 1.46-1.32 (m, 2 H), 1.27-1.12 (m, 1 H), 0.91 (t, J = 7.3 Hz, 3 H), 0.66- |
| | | | | | | 0.52 (m, 2 H), 0.43-0.26 (m, 2 H) |
| (R)-4-(1-(5-(cyclopropylmethoxy)pyridin-2- | C | H | C | Method D | m/z = 663.3 | ¹H NMR (400 MHz, DMSO-d6, ppm) δ 8.24 (s, 1 H), 7.56-7.16 (m, 3 H), 6.73 (t, J = 7.9 Hz, 1 H), |
| yl)propoxy)-N-((4-((1- | | | | (40-85) | [M + H]+, Rt = | 5.30 (t, J = 6.5 Hz, 1 H), 3.86 (d, J = 7.1 Hz, 2 H), 3.72-3.52 (m, 1 H), 3.50-3.39 (m, 1 H), 3.38-3.33 |
| (cyclopropylmethyl)piperidin-4- | | | | | 1.05 mins | (m, 2 H), 3.18-2.69 (m, 6 H), 2.13-1.67 (m, 8 H), 1.65-1.30 (m, 4 H), 1.27-1.14 (m, 1 H), 1.08-0.95 |
| yl)oxy)piperidin-1-yl)sulfonyl)-2,3- | | | | | (Method F) | (m, 1 H), 0.91 (t, J = 7.3 Hz, 3 H), 0.66-0.45 (m, 4 H), 0.40-0.18 (m, 4 H) |
| difluorobenzamide | | | | | | |
| (R)-4-(1-(5-(cyclopropylmethoxy)pyridin-2- | C | G | B | Method A | m/z = 636.6 | ¹H NMR (400 MHz, DMSO-d6, ppm) δ 8.24 (s, 1 H), 7.40-7.23 (m, 3 H), 6.75 (t, J = 7.9 Hz, 1 H), |
| yl)propoxy)-N-((4-((4-ethylpiperazin-1- | | | | (25-70) | [M + H]+, Rt = | 5.31 (t, J = 6.5 Hz, 1 H), 3.86 (d, J = 7.1 Hz, 2 H), 3.57-3.44 (m, 2 H), 3.10-2.75 (m, 6 H), 2.69-2.50 |
| yl)methyl)piperidin-1-yl)sulfonyl)-2,3- | | | | | 1.48 mins | (m, 6 H), 2.29-2.12 (m, 2 H), 2.07-1.88 (m, 2 H), 1.69 (d, J = 12.1 Hz, 2 H), 1.60-1.43 (m, 1 H), |
| difluorobenzamide | | | | | (Method E) | 1.24-0.99 (m, 6 H), 0.91 (t, J = 7.4 Hz, 3 H), 0.61-0.51 (m, 2 H), 0.35-0.25 (m, 2 H) |
| (R)-2,3-difluoro-4-(1-(5-(2- | D | C | B | C18 (50-60 | m/z = 620.1 | ¹H NMR (400 MHz, DMSO-d6, ppm) δ 9.41 (br, 1 H), 8.26 (s, 1 H), 7.73 (d, J = 8.8 Hz, 2 H), 7.33 |
| methoxyethoxy)pyridin-2-yl)propoxy)-N-((4- | | | | MeCN/H₂O | [M + H]+, Rt = | (m, 3 H), 6.98 (d, J = 8.8 Hz, 2 H), 6.73 (t, J = 8.0 Hz, 1 H), 5.31 (t, J = 6.5 Hz, 1 H), 4.76-4.55 (m, |
| ((1-methylpiperidin-4- | | | | | 1.27 mins | 1 H), 4.16-4.09 (m, 2 H), 3.66-3.61 (m, 2 H), 3.29 (s, 3 H), 3.28-3.00 (m, 4 H), 2.77 (s, 3 H), 2.17- |
| yl)oxy)phenyl)sulfonyl)benzamide | | | | | (Method E) | 1.73 (m, 6 H), 0.90 (t, J = 7.3 Hz, 3 H) |
| (R)-N-((4-(2- | D | G | A | Method D | m/z = 601.2 | ¹H NMR (400 MHz, DMSO-d6, ppm) δ 9.57 (br, 1 H), 8.25 (s, 1 H), 7.41-7.27 (m, 3 H), 6.73 (t, J = |
| (dimethylamino)ethoxy)piperidin-1- | | | | (45-95) | [M + H]+, Rt = | 8.1 Hz, 1 H), 5.31 (t, J = 6.4 Hz, 1 H), 4.19-4.11 (m, 2 H), 3.70-3.61 (m, 4 H), 3.44-3.36 (m, 2 H), |
| yl)sulfonyl)-2,3-difluoro-4-(1-(5-(2- | | | | | 0.94 mins | 3.28-3.23 (m, 4 H), 3.138-3.08 (m, 2 H), 2.83-2.63 (m, 8 H), 2.06-1.81 (m, 4 H), 1.89-1.81 (m, 2 |
| methoxyethoxy)pyridin-2- | | | | | (Method D) | H), 0.91 (t, J = 7.3 Hz, 3 H) |
| yl)propoxy)benzamide | | | | | | |
| (R)-2,3-difluoro-4-(1-(5-(2- | D | H | A | Method D | m/z = 627.2 | ¹H NMR (400 MHz, DMSO-d6, ppm) δ 8.22 (s, 1 H), 7.40 (d, J = 1.7 Hz, 2 H), 7.35-7.25 (m, 1 H), |
| methoxyethoxy)pyridin-2-yl)propoxy)-N-((4- | | | | (45-75) | [M + H]+, Rt = | 6.68-6.60 (m, 1 H), 5.25 (t, J = 6.4 Hz, 1 H), 4.29-4.13 (m, 2 H), 3.83-3.68 (m, 3 H), 3.64-3.55 (m, |
| ((1-methylpiperidin-4-yl)oxy)piperidin-1- | | | | | 0.95 mins | 1 H), 3.55-3.45 (m, 2 H), 3.40 (s, 3 H), 3.28-3.19 (m, 3 H), 3.17-3.01 (m, 4 H), 2.77 (s, 3 H), 2.16- |
| yl)sulfonyl)benzamide | | | | | (Method D) | 1.88 (m, 8 H), 1.69-1.52 (m, 2 H), 1.01 (t, J = 7.4 Hz, 3 H) |
| (R)-N-((4-(2- | E | G | B | Method E | m/z = 620.3 | ¹H NMR (400 MHz, DMSO-d6, ppm) δ 8.44 (d, J = 2.5 Hz, 1 H), 8.16-8.14 (dd, J = 5.0, 1.4 Hz, 1 |
| (dimethylamino)ethoxy)piperidin-1- | | | | (25-65) | [M + H]+, Rt = | H), 7.91-7.87 (m, 1 H), 7.64 (dd, J = 8.5, 2.7 Hz, 1 H), 7.49 (d, J = 8.5 Hz, 1 H), 7.35 (t, J = 8.5 Hz, |
| yl)sulfonyl)-2,3-difluoro-4-(1-(5-(pyridin-2- | | | | | 1.05 mins | 1 H), 7.23-7.06 (m, 2 H), 6.80 (t, J = 7.9 Hz, 1 H), 5.41 (t, J = 6.4 Hz, 1 H), 3.70-3.58 (m, 2 H), |
| yloxy)pyridin-2-yl)propoxy)benzamide | | | | | (Method D) | 3.47-3.34 (m, 3 H), 3.13 (t, J = 7.5 Hz, 2 H), 2.78 (t, J = 9.7 Hz, 2 H), 2.70 (s, 6 H), 2.09-1.93 (m, 2 |
| | | | | | | H), 1.92-1.78 (m, 2 H), 1.49-1.43 (m, 2 H), 0.96 (t, J = 7.3 Hz, 3 H) |
| (R)-4-(1-(5-(cyclopropylmethoxy)pyridin-2- | C | G | A | Method D | m/z = 597.3 | ¹H NMR (400 MHz, DMSO-D6) δ 9.43 (br, 1 H), 8.24 (s, 1 H), 7.39-7.26 (m, 3 H), 6.73 (t, J = 8.0 |
| yl)propoxy)-N-((4-(2- | | | | (45-95) | [M + H]+, Rt = | Hz, 1 H), 5.30 (t, J = 6.4 Hz, 1 H), 3.86 (d, J = 7.0 Hz, 2 H), 3.66 (t, J = 5.1 Hz, 2 H), 3.45-3.37 (m, |
| (dimethylamino)ethoxy)piperidin-1- | | | | | 1.12 mins | 1 H), 3.30-3.25 (m, 2 H), 3.15 (t, J = 4.9 Hz, 2 H), 2.82-2.68 (m, 8 H), 2.08-1.91 (m, 2 H), 1.88- |
| yl)sulfonyl)-2,3-difluorobenzamide | | | | | (Method D) | 1.80 (m, 2 H), 1.51-1.38 (m, 2 H), 1.20-1.15 (m, 1 H), 0.91 (t, J = 7.3 Hz, 3 H), 0.60-0.51 (m, 2 H), |
| | | | | | | 0.34-0.26 (m, 2 H) |
| (R)-2,3-difluoro-N-((4-methylpiperazin-1- | E | CAS1 | B | Method A | m/z = 548.4 | ¹H NMR (400 MHz, DMSO-d6, ppm) δ. 9.69 (br, 1 H), 8.45-8.44 (m, 1 H), 8.16-8.13 (m, 1 H), |
| yl)sulfonyl)-4-(1-(5-(pyridin-2-yloxy)pyridin- | | | | (30-80) | [M + H]+, Rt = | 7.94-7.86 (m, 1 H), 7.65-7.62 (m, 1 H), 7.51-7.48 (m, 1 H), 7.41-7.36 (m, 1 H), 7.22-7.07 (m, 2 H), |
| 2-yl)propoxy)benzamide | | | | | 1.22 mins | 6.82 (t, J = 8.0 Hz, 1 H), 5.43 (t, J = 6.4 Hz, 1 H), 3.25-3.06 (m, 8 H), 2.67 (s, 3 H), 2.10-1.95 (m, 2 |
| | | | | | (Method E) | H), 0.96 (t, J = 7.3 Hz, 3 H) |
| (R)-4-(1-(5-(cyclopropylmethoxy)pyridin-2- | C | CAS2 | B | Method A | m/z = 524.1 | ¹H NMR (400 MHz, methanol-d4, ppm) δ 8.17 (s, 1 H), 7.40-7.34 (m, 3 H), 6.65 (t, J = 7.2 Hz, 1 |
| yl)propoxy)-2,3-difluoro-N-((1- | | | | (25-70) | [M + H]+, Rt = | H), 5.25 (t, J = 5.5 Hz, 1 H), 3.87 (d, J = 7.0 Hz, 2 H), 3.75-3.64 (m, 1 H), 3.56 (d, J = 13.1 Hz, 2 |
| methylpiperidin-4-yl)sulfonyl)benzamide | | | | | 1.02 mins | H), 3.11-2.94 (m, 2 H), 2.84 (s, 3 H), 2.37-2.23 (m, 2 H), 2.12-1.95 (m, 4 H), 1.32-1.19 (m, 1 H), |
| | | | | | (Method D) | 1.01 (t, J = 7.4 Hz, 3 H), 0.65-0.56 (m, 2 H), 0.38-0.29 (m, 2 H) |
| (R)-2,3-difluoro-4-(1-(5-(pyridin-2- | E | H | B | Method A | m/z = 576.2 | ¹H NMR (400 MHz, DMSO-d6, ppm) δ 11.11 (s, 1 H), 8.45 (d, J = 2.7 Hz, 1 H), 8.14 (d, J = 5.1, |
| yloxy)pyridin-2-yl)propoxy)-N-((2,2,4- | | | | (25-65) | [M + H]+, Rt = | 2.0 Hz, 1 H), 7.96-7.82 (m, 1 H), 7.65 (d, J = 8.5, 2.7 Hz, 1 H), 7.51 (d, J = 8.5 Hz, 1 H), 7.31 (t, J = |
| trimethylpiperazin-1-yl)sulfonyl)benzamide | | | | | 0.99 mins | 8.3 Hz, 1 H), 7.24-7.09 (m, 2 H), 6.92 (t, J = 7.8 Hz, 1 H), 5.47 (t, J = 6.3 Hz, 1 H), 3.56 (s, 2 H), |
| | | | | | (Method D) | 2.60-2.50 (m, 2 H), 2.36-2.24 (m, 5 H), 2.09-2.00 (m, 2 H), 1.37 (s, 6 H), 0.96 (t, J = 7.4 Hz, 3 H) |
| (R)-2,3-difluoro-N-((1-methylpiperidin-4- | E | CAS3 | B | Method E | m/z = 547.4 | ¹H NMR (400 MHz, DMSO-d6, ppm) δ 9.16 (br, 1 H), 8.44 (d, J = 2.4 Hz, 1 H), 8.15-8.14 (m, 1 |
| yl)sulfonyl)-4-(1-(5-(pyridin-2-yloxy)pyridin- | | | | (20-95) | [M + H]+, Rt = | H), 7.92-7.87 (m, 1 H), 7.65-7.62 (m, 1 H), 7.49 (d, J = 8.4 Hz, 1 H), 7.39 (t, J = 8.2 Hz, 1 H), 7.18- |
| 2-yl)propoxy)benzamide | | | | | 1.29 mins | 7.12 (m, 2 H), 6.81 (t, J = 8.0 Hz, 1 H), 5.42 (t, J = 6.4 Hz, 1 H), 3.46-3.37 (m, 3 H), 3.00-2.19 (m, |
| | | | | | (Method E) | 2 H), 2.68 (s, 3 H), 2.07-1.99 (m, 4 H), 1.84-1.82 (m, 2 H), 0.96 (t, J = 7.4 Hz, 3 H) |
| (R)-4-(1-(5-(cyclopropylmethoxy)pyridin-2- | C | H | D | Method I | m/z = 553.5 | ¹H NMR (400 MHz, DMSO-d6, ppm) δ 8.25 (s, 1 H), 7.34 (s, 2 H), 7.25 (t, J = 7.5 Hz, 1 H), 6.86 |
| yl)propoxy)-2,3-difluoro-N-((2,2,4- | | | | (40-100) | [M + H]+, Rt = | (t, J = 7.1 Hz, 1 H), 5.36 (t, J = 5.9 Hz, 1 H), 3.86 (d, J = 7.0 Hz, 2 H), 3.57 (s, 3 H), 2.57 (s, 2 H), |
| trimethylpiperazin-1-yl)sulfonyl)benzamide | | | | | 3.86 mins | 2.36 - 2.27 (m, 5 H), 2.07 - 1.91 (m, 2 H), 1.36 (s, 6 H), 1.25 - 1.14 (m, 1 H), 0.90 (t, J = 7.1 Hz, 3 |
| | | | | | (Method A) | H), 0.57 (d, J = 6.4 Hz, 2 H), 0.30 (d, J = 4.3 Hz, 2 H) |
| (R)-4-(1-(5-(cyclopropylmethoxy)pyridin-2- | C | I | B | Method B | m/z = 526.3 | ¹H NMR (400 MHz, DMSO-d6, ppm) δ 10.12 (s, 1 H), 8.25 (s, 1 H), 7.48-7.30 (m, 3 H), 6.82 (t, J = |
| yl)propoxy)-N-((1-(dimethylamino)-2- | | | | (30-50) | [M + H]+, Rt = | 8.0 Hz, 1 H), 5.33 (t, J = 6.4 Hz, 1 H), 3.87 (d, J = 7.1 Hz, 2 H), 3.36 (s, 2 H), 2.86 (s, 6 H), 2.05- |
| methylpropan-2-yl)sulfonyl)-2,3- | | | | | 1.16 mins | 1.92 (m, 2 H), 1.33 (s, 6 H), 1.26-1.15 (m, 1 H), 0.91 (t, J = 7.3 Hz, 3 H), 0.61-0.45 (m, 2 H), 0.39- |
| difluorobenzamide | | | | | (Method D) | 0.29 (m, 2 H) |
| (R)-4-(1-(5-(cyclopropylmethoxy)pyridin-2- | C | CAS4 | D | Method E | m/z = 525.3 | ¹H NMR (400 MHz, DMSO-d6, ppm) δ 9.64 (s, 1 H), 8.24 (dd, J = 0.9, 2.4 Hz, 1 H), 7.38-7.30 |
| yl)propoxy)-2,3-difluoro-N-((4- | | | | (5-60) | [M + H]+, Rt = | (m, 3 H), 6.76 (t, J = 8.0 Hz, 1 H), 5.32 (t, J = 6.4 Hz, 1 H), 3.86 (d, J = 7.1 Hz, 2 H), 3.23-3.04 |
| methylpiperazin-1-yl)sulfonyl)benzamide | | | | | 3.73 mins | (m, 8 H), 2.65 (s, 3 H), 2.06-1.90 (m, 2 H), 1.26-1.15 (m, 1 H), 0.90 (t, J = 7.3 Hz, 3 H), 0.59- |
| | | | | | (Method A) | 0.53 (m, 2 H), 0.34-0.29 (m, 2 H) |
| (R)-N-((1-(dimethylamino)-2-methylpropan- | E | I | B | Method B | m/z = 549.1 | ¹H NMR (400 MHz, DMSO-d6, ppm) δ 10.12 (s, 1 H), 8.45 (d, J = 2.6 Hz, 1 H), 8.15-8.13 (m, 1 |
| 2-yl)sulfonyl)-2,3-difluoro-4-(1-(5-(pyridin- | | | | (30-50) | [M + H]+, Rt = | H), 7.92-7.87 (m, 1 H), 7.66-7.63 (m, 1 H), 7.51-7.45 (m, 2 H), 7.18-7.12 (m, 2 H), 6.89 (t, J = 8.4 |
| 2-yloxy)pyridin-2-yl)propoxy)benzamide | | | | | 1.11 mins | Hz, 1 H), 5.45 (t, J = 6.3 Hz, 1 H), 3.36 (s, 2 H), 2.86 (s, 6 H), 2.08-2.00 (m, 2 H), 1.34 (s, 6 H), |
| | | | | | (Method D) | 0.96 (t, J = 7.3 Hz, 3 H) |
| (R)-N-((4-((1-methylpiperidin-4- | G | H | A | Method A | m/z = 678.5 | ¹H NMR (400 MHz, DMSO-d6, ppm) δ 9.54 (s, 1 H), 8.47 (d, J = 2.7 Hz, 1 H), 8.23-8.09 (m, 2 H), |
| yl)oxy)piperidin-1-yl)sulfonyl)-4-(1-(5- | | | | (20-80) | [M + H]+, Rt = | 8.01 (dd, J = 8.8, 1.8 Hz, 1 H), 7.95-7.82 (m, 1 H), 7.65-7.63 (m, 1 H), 7.37 (d, J = 8.5 Hz, 1 H), |
| (pyridin-2-yloxy)pyridin-2-yl)propoxy)-3- | | | | | 1.1 mins | 7.13-7.12 (m, 2 H), 6.99 (d, J = 8.8 Hz, 1 H), 5.53 (t, J = 6.0 Hz, 1 H), 3.66-3.64 (m, 1 H), 3.48-3.44 |
| (trifluoromethyl)benzamide | | | | | (Method D) | (m, 1 H), 3.38-3.37 (m, 2 H), 3.14-3.05 (m, 4 H), 2.80 (t, J = 10.2 Hz, 2 H), 2.68 (s, 3 H), 2.03-2.00 |
| | | | | | | (m, 2 H), 1.88-1.66 (m, 6 H), 1.43-1.41 (m, 2 H), 0.97 (t, J = 7.3 Hz, 3 H) |
| (R)-N-((4-(2- | G | A | A | Method A | m/z = 645.2 | ¹H NMR (400 MHz, DMSO-d6, ppm) δ 9.52 (s, 1 H), 8.46 (d, J = 2.7 Hz, 1 H), 8.13 (s, 2 H), 7.96- |
| (dimethylamino)ethoxy)phenyl)sulfonyl)-4- | | | | (30-80) | [M + H]+, Rt = | 7.87 (m, 2 H), 7.78 (d, J = 8.7 Hz, 2 H), 7.63 (dd, J = 8.6, 2.6 Hz, 1 H), 7.35 (d, J = 8.6 Hz, 1 H), |
| (1-(5-(pyridin-2-yloxy)pyridin-2-yl)propoxy)- | | | | | 1.09 mins | 7.17-7.11 (m, 2 H), 6.98 (d, J = 8.7 Hz, 3 H), 5.51 (t, J = 6.1 Hz, 1 H), 4.32 (t, J = 4.6 Hz, 2 H), 3.45 |
| 3-(trifluoromethyl)benzamide | | | | | (Method D) | (t, J = 4.6 Hz, 2 H), 2.81 (s, 6 H), 2.02-1.99 (m, 2 H), 0.95 (t, J = 7.3 Hz, 3 H) |
| (R)-N-((4-(2- | E | A | A | Method E | m/z = 613.2 | ¹H NMR (400 MHz, DMSO-d6, ppm) δ 9.60 (s, 1 H), 8.43 (d, J = 2.6 Hz, 1 H), 8.14 (d, J = 5.0 Hz, |
| (dimethylamino)ethoxy)phenyl)sulfonyl)-2,3- | | | | (20-60) | [M + H]+, Rt = | 1 H), 7.89-7.87 (m, 1 H), 7.76 (d, J = 8.7 Hz, 2 H), 7.61 (dd, J = 8.6, 2.8 Hz, 1 H), 7.47 (d, J = 8.6 |
| difluoro-4-(1-(5-(pyridin-2-yloxy)pyridin-2- | | | | | 1.16 mins | Hz, 1 H), 7.35 (t, J = 7.6 Hz, 1 H), 7.10-6.98 (m, 2 H), 6.99 (d, J = 8.7 Hz, 2 H), 6.79 (t, J = 8.3 Hz, |
| yl)propoxy)benzamide | | | | | (Method D) | 1 H), 5.41 (t, J = 6.3 Hz, 1 H), 4.32 (t, J = 4.8 Hz, 2 H), 3.44 (t, J = 4.8 Hz, 2 H), 2.80 (s, 6 H), 2.07- |
| | | | | | | 1.98 (m, 2 H), 0.95 (t, J = 7.3 Hz, 3 H) |
| (R)-N-((4-((1-methylpiperidin-4- | H | H | A | Method D | m/z = 664.2 | ¹H NMR (400 MHz, DMSO-d6, ppm) δ 9.86-8.86 (m, 1 H), 8.46 (d, J = 2.5 Hz, 1 H), 8.21-8.11 (m, |
| yl)oxy)piperidin-1-yl)sulfonyl)-4-(1-(5- | | | | (20-65) | [M + H]+, Rt = | 2 H), 8.05 (d, J = 8.7 Hz, 1 H), 7.93-7.86 (m, 1 H), 7.67 (dd, J = 8.6, 2.7 Hz, 1 H), 7.46 (d, J = 8.6 |
| (pyridin-2-yloxy)pyridin-2-yl)ethoxy)-3- | | | | | 1.16 mins | Hz, 1 H), 7.20-7.08 (m, 3 H), 5.75 (q, J = 6.5 Hz, 1 H), 3.75-3.58 (m, 1 H), 3.53-3.36 (m, 3 H), |
| (trifluoromethyl)benzamide | | | | | (Method F) | 3.21-2.88 (m, 4 H), 2.81 (t, J = 9.8 Hz, 2 H), 2.73-2.65 (m, 3 H), 1.99-1.56 (m, 9 H), 1.49-1.33 (m, |
| | | | | | | 2 H) |
| (R)-N-((4-(2- | H | A | A | Method D | m/z = 631.2 | ¹H NMR (400 MHz, DMSO-d6, ppm) δ 9.61 (s, 1 H), 8.45 (d, J = 2.5 Hz, 1 H), 8.18-8.09 (m, 2 H), |
| (dimethylamino)ethoxy)phenyl)sulfonyl)-4- | | | | (20-70) | [M + H]+, Rt = | 8.02-7.94 (m, 1 H), 7.92-7.85 (m, 1 H), 7.78 (d, J = 8.8 Hz, 2 H), 7.66 (dd, J = 8.6, 2.7 Hz, 1 H), |
| (1-(5-(pyridin-2-yloxy)pyridin-2-yl)ethoxy)- | | | | | 1.14 mins | 7.45 (d, J = 8.6 Hz, 1 H), 7.21-7.06 (m, 3 H), 6.97 (d, J = 8.8 Hz, 2 H), 5.74 (q, J = 6.2 Hz, 1 H), |
| 3-(trifluoromethyl)benzamide | | | | | (Method F) | 4.38-4.25 (m, 2 H), 3.53-3.39 (m, 2 H), 2.80 (s, 6 H), 1.63 (d, J = 6.4 Hz, 3 H) |
| (R)-4-(1-(5-(cyclopropylmethoxy)pyridin-2- | C | H | A | Method D | m/z = 553.2 | ¹H NMR (400 MHz, DMSO-d6, ppm) δ 9.28 (s, 1 H), 8.28-8.20 (m, 1 H), 7.41-7.27 (m, 3 H), 6.75 |
| yl)propoxy)-2,3-difluoro-N-((4- | | | | (20-70) | [M + H]+, Rt = | (t, J = 8.1 Hz, 1 H), 5.32 (t, J = 6.5 Hz, 1 H), 3.86 (d, J = 7.1 Hz, 2 H), 3.34-2.77 (m, 9 H), 2.09- |
| isopropylpiperazin-1-yl)sulfonyl)benzamide | | | | | 1.19 mins | 1.88 (m, 2 H), 1.28-1.13 (m, 7 H), 0.91 (t, J = 7.3 Hz, 3 H), 0.64-0.49 (m, 2 H), 0.38-0.25 (m, 2 H) |
| | | | | | (Method F) | |
| (R)-4-(1-(5-(cyclopropylmethoxy)pyridin-2- | C | H | A | Method B | m/z = 551.4 | ¹H NMR (400 MHz, DMSO-d6, ppm) δ 11.46 (s, 1 H) 8.27-8.22 (m, 1 H), 7.39-7.33 (m, 2 H), 7.33- |
| yl)propoxy)-N-((4-cyclopropylpiperazin-1- | | | | (30-60) | [M + H]+, Rt = | 7.26 (m, 1 H), 6.94-6.85 (m, 1 H), 5.40 (t, J = 6.5 Hz, 1 H), 3.86 (d, J = 7.1 Hz, 2 H), 3.26-3.16 (m, |
| yl)sulfonyl)-2,3-difluorobenzamide | | | | | 1.12 mins | 4 H), 2.89-2.60 (m, 4 H), 2.08-1.77 (m, 3 H), 1.25-1.16 (m, 1 H), 0.91 (t, J = 7.3 Hz, 3 H), 0.60- |
| | | | | | (Method D) | 0.54 (m, 2 H), 0.52-0.36 (m, 4 H), 0.34-0.29 (m, 2 H) |
| 4-((R)-1-(5-(cyclopropylmethoxy)pyridin-2- | C | C | B | Method D | m/z= 616.2 | ¹H NMR (400 MHz, DMSO-d6, ppm) δ 8.23-8.23 (m, 1 H), 7.87-7.71 (m, 2 H), 7.36-7.20 (m, 3 H), |
| yl)propoxy)-2,3-difluoro-N-((4-((1- | | | | (20-70) | [M + H]+, Rt = | 7.08-6.98 (m, 2 H), 6.76-6.72 (m, 1 H), 5.28 (t, J = 6.4 Hz, 1 H), 4.72-4.54 (m, 1 H), 4.26-4.17 (m, |
| methylpiperidin-3- | | | | | 1.21 mins | 1 H), 3.88 (d, J = 6.8 Hz, 2 H), 3.62-3.37 (m, 1 H), 3.24-3.19 (m, 1 H), 2.96-2.91 (m, 1 H), 2.83- |
| yl)oxy)phenyl)sulfonyl)benzamide | | | | | (Method F) | 2.78 (m, 1 H), 2.63-2.57 (m, 2 H), 2.21-1.64 (m, 6 H), 1.23-1.16 (m, 1 H), 0.90 (t, J = 7.4 Hz, 3 H), |
| | | | | | | 0.57-0.54 (m, 2 H), 0.32-0.30 (m, 2 H) |
| (R)-4-(1-(5-(cyclopropylmethoxy)pyridin-2- | C | H | D | Method E | m/z = 539.2 | ¹H NMR (400 MHz, DMSO-d6, ppm) δ 8.26-8.22 (m, 1 H), 7.42-7.22 (m, 3 H), 6.78-6.74 (m, 1 H), |
| yl)propoxy)-2,3-difluoro-N-((4-methyl-1,4- | | | | (20-95) | [M + H]+, Rt = | 5.31 (t, J = 6.4 Hz, 1 H), 3.86 (d, J = 7.0 Hz, 2 H), 3.55-3.49 (m, 2 H), 3.31-3.27 (m, 2 H), 3.22-3.20 |
| diazepan-1-yl)sulfonyl)benzamide | | | | | 1.19 mins | (m, 4 H), 2.71 (s, 3 H), 2.08-1.91 (m, 4 H), 1.24-1.17 (m, 1 H), 0.91 (t, J = 7.2 Hz, 3 H), 0.60-0.52 |
| | | | | | (Method D) | (m, 2 H), 0.36-0.26 (m, 2 H) |
| (R)-4-(1-(5-(cyclopropylmethoxy)pyridin-2- | C | H | B | Method B | m/z = 553.2 | ¹H NMR (400 MHz, MeOD-d, ppm) δ 8.18 (d, J = 2.5 Hz, 1 H), 7.44-7.28 (m, 3 H), 6.66 (t, J = 8.0 |
| yl)propoxy)-2,3-difluoro-N-((3,3,4- | | | | (20-70) | [M + H]+, Rt = | Hz, 1 H), 5.33-5.19 (m, 1 H), 3.87 (d, J = 7.0 Hz, 2 H), 3.48 (s, 2 H), 3.29-3.22 (m, 4 H), 2.74 (s, 3 |
| trimethylpiperazin-1-yl)sulfonyl)benzamide | | | | | 1.05 mins | H), 2.18-1.93 (m, 2 H), 1.37 (s, 6 H), 1.29-1.22 (m, 1 H), 1.01 (t, J = 7.4 Hz, 3 H), 0.69-0.53 (m, 2 |
| | | | | | (Method E) | H), 0.38-0.30 (m, 2 H) |
| 4-((R)-1-(5-(cyclopropylmethoxy)pyridin-2- | C | H | A | Method E | m/z = 553.5 | ¹H NMR (400 MHz, DMSO-d6, ppm) δ 8.98 (s, 1 H), 8.25 (d, J = 2.3 Hz, 1 H), 7.37-7.32 (m, 3 H), |
| yl)propoxy)-2,3-difluoro-N-(((3S,5R)-3,4,5- | | | | (20-60) | [M + H]+, Rt = | 6.75 (t, J = 7.9 Hz, 1 H), 5.31 (t, J = 6.5 Hz, 1 H), 3.86 (d, J = 7.1 Hz, 2 H), 3.54 (brs, 2 H), 3.13- |
| trimethylpiperazin-1-yl)sulfonyl)benzamide | | | | | 1.23 mins | 3.07(m, 2 H), 2.72-2.67 (m, 5 H), 2.06-1.89 (m, 2 H), 1.24-1.16 (m, 7 H), 0.91 (t, J = 7.4 Hz, 3 H), |
| | | | | | (Method D) | 0.59-0.54 (m, 2 H), 0.33-0.29 (m, 2 H) |
| (R)-N-((4-(2- | I | A | A | Method D | m/z = 599.2 | ¹H NMR (400 MHz, DMSO-d6, ppm) δ 9.67 (s, 1 H), 8.43 (d, J = 2.6 Hz, 1 H), 8.14 (dd, J = 4.9, |
| (dimethylamino)ethoxy)phenyl)sulfonyl)-2,3- | | | | (20-60) | [M + H]+, Rt = | 1.3 Hz, 1 H), 7.81-7.86 (m, 1 H), 7.77 (d, J = 8.8 Hz, 2 H), 7.63 (dd, J = 8.5, 2.7 Hz, 1 H), 7.52 (d, |
| difluoro-4-(1-(5-(pyridin-2-yloxy)pyridin-2- | | | | | 1.08 mins | J = 8.5 Hz, 1 H), 7.37 (t, J = 8.5 Hz, 1 H), 7.20-7.10 (m, 2 H), 6.99 (d, J = 8.8 Hz, 2 H), 6.84 (t, J = |
| yl)ethoxy)benzamide | | | | | (Method F) | 8.1 Hz, 1 H), 5.64 (q, J = 6.4 Hz, 1 H), 4.36-4.27 (m, 2 H), 3.46-3.39 (m, 2 H), 2.79 (s, 6 H), 1.65 |
| | | | | | | (d, J = 6.4 Hz, 3 H) |
| (R)-4-(1-(5-(cyclopropylmethoxy)pyridin-2- | C | H | A | Method D | m/z = 569.2 | ¹H NMR (400 MHz, DMSO-d6, ppm) δ 10.22 (br, 1 H), 8.28-8.21 (m, 1 H), 7.40-7.28 (m, 3 H), |
| yl)propoxy)-2,3-difluoro-N-((4-(2- | | | | (20-60) | [M + H]+, Rt = | 6.80 (t, J = 7.9 Hz, 1 H), 5.34 (t, J = 6.5 Hz, 1 H), 3.86 (d, J = 7.1 Hz, 2 H), 3.56 (t, J = 4.8 Hz, 2 H), |
| methoxyethyl)piperazin-1- | | | | | 1.19 mins | 3.30-3.15 (m, 7 H), 3.14-2.79 (m, 6 H), 2.10-1.86 (m, 2 H), 1.29-1.13 (m, 1 H), 0.91 (t, J = 7.3 Hz, |
| yl)sulfonyl)benzamide | | | | | (Method F) | 3 H), 0.63-0.50 (m, 2 H), 0.37-0.26 (m, 2 H) |
| 4-((R)-1-(5-(cyclopropylmethoxy)pyridin-2- | C | H | B | Method B | m/z = 537.2 | ¹H NMR (400 MHz, DMSO-d6, ppm) δ 8.27-8.21 (m, 1 H), 7.38-7.28 (m, 3 H), 6.74 (t, J = 7.9 Hz, |
| yl)propoxy)-2,3-difluoro-N-((6-methyl-3,6- | | | | (30-50) | [M + H]+, Rt = | 1 H), 5.31 (t, J = 6.4 Hz, 1 H), 4.09-3.89 (m, 2 H), 3.86 (d, J = 7.1 Hz, 2 H), 3.75-3.49 (m, 4 H), |
| diazabicyclo[3.1.1]heptan-3- | | | | | 1.03 mins | 2.48-2.36 (m, 4 H), 2.05-1.90 (m, 2 H), 1.87-1.75 (m, 1 H), 1.24-1.16 (m, 1 H), 0.90 (t, J = 7.3 Hz, |
| yl)sulfonyl)benzamide | | | | | (Method D) | 3 H), 0.59-0.50 (m, 2 H), 0.37-0.28 (m, 2 H) |
| 4-((R)-1-(5-(cyclopropylmethoxy)pyridin-2- | C | H | A | Method E | m/z = 539.5 | ¹H NMR (400 MHz, DMSO-d6, ppm) δ 9.66 (s, 1 H), 8.27-8.14 (m, 1 H), 7.37-7.32 (m, 3 H), 6.75 |
| yl)propoxy)-N-(((S)-3,4-dimethylpiperazin-1- | | | | (25-60) | [M + H]+, Rt = | (t, J = 7.9 Hz, 1 H), 5.32 (t, J = 6.5 Hz, 1 H), 3.86 (d, J = 7.1 Hz, 2 H), 3.57-3.40 (m, 2 H), 3.29- |
| yl)sulfonyl)-2,3-difluorobenzamide | | | | | 1.21 mins | 3.20 (m, 1 H), 3.13-2.86 (m, 3 H), 2.79-2.56 (m, 4 H), 2.12-1.80 (m, 2 H), 1.33-1.09 (m, 4 H), 0.91 |
| | | | | | (Method E) | (t, J = 7.3 Hz, 3 H), 0.69-0.45 (m, 2 H), 0.47-0.23 (m, 2 H) |
| 4-((R)-1-(5-(cyclopropylmethoxy)pyridin-2- | C | H | A | Method F | m/z = 539.5 | ¹H NMR (400 MHz, DMSO-d6, ppm) δ 9.54 (s, 1 H), 8.25-8.24 (m, 1 H), 7.41-7.25 (m, 3 H), 6.77 |
| yl)propoxy)-N-(((R)-3,4-dimethylpiperazin-1- | | | | (25-60) | [M + H]+, Rt = | (t, J = 7.9 Hz, 1 H), 5.32 (t, J = 6.5 Hz, 1 H), 3.86 (d, J = 7.1 Hz, 2 H), 3.62-3.44 (m, 2 H), 3.41- |
| yl)sulfonyl)-2,3-difluorobenzamide | | | | | 1.21 mins | 3.34 (m, 1 H), 3.22-2.93 (m, 3 H), 2.85-2.60 (m, 4 H), 2.10-1.88 (m, 2 H), 1.26-1.14 (m, 4 H), 0.91 |
| | | | | | (Method E) | (t, J = 7.4 Hz, 3 H), 0.61-0.53 (m, 2 H), 0.37-0.25 (m, 2 H) |
| (R)-2,3-difluoro-N-((4-((1-(2- | E | G | B | Method F | m/z = 690.7 | ¹H NMR (400 MHz, DMSO-d6, ppm) δ. 8.45 (d, J = 2.4 Hz, 1 H), 8.16-8.13 (m, 1 H), 7.92-7.87 |
| methoxyethyl)piperidin-4-yl)oxy)piperidin-1- | | | | (25-60) | [M + H]+, Rt = | (m, 1 H), 7.65-7.62 (m, 1 H), 7.51-7.48 (m, 1 H), 7.37-7.32 (m, 1 H), 7.21-7.08 (m, 2 H), 6.86-6.81 |
| yl)sulfonyl)-4-(1-(5-(pyridin-2-yloxy)pyridin- | | | | | 1.21 mins | (m, 1 H), 5.43 (t, J = 6.4 Hz, 1 H), 3.64-3.57 (m, 3 H), 3.50-3.42 (m, 2 H), 3.39-3.31 (m, 6 H), 3.14 |
| 2-yl)propoxy)benzamide | | | | | (Method E) | (s, 3 H), 3.03-2.75 (m, 4 H), 2.10-1.97 (m, 2 H), 1.95-1.77 (m, 4 H), 1.74-1.57 (m, 2 H), 1.46-1.37 |
| | | | | | | (m, 2 H), 0.96 (t, J = 7.2 Hz, 3 H) |
| 4-((R)-1-(5-(cyclopropylmethoxy)pyridin-2- | C | H | A | Method A | m/z= 551.1 | ¹H NMR (400 MHz, DMSO-d6, ppm) δ 9.52 (s, 1 H), 8.27-8.22 (m, 1 H), 7.43-7.26 (m, 3 H), 6.76 |
| yl)propoxy)-2,3-difluoro-N-((8-methyl-3,8- | | | | (25-60) | [M + H]+, Rt = | (t, J = 7.9 Hz, 1 H), 5.32 (t, J = 6.5 Hz, 1 H), 3.86 (d, J = 7.1 Hz, 4 H), 3.43-3.34 (m, 2 H), 3.24- |
| diazabicyclo[3.2.1]octan-3- | | | | | 1.33 mins | 3.12 (m, 2 H), 2.49 (s, 3 H), 2.17-1.82 (m, 6 H), 1.32-1.11 (m, 1 H), 0.91 (t, J = 7.3 Hz, 3 H), 0.69- |
| yl)sulfonyl)benzamide | | | | | (Method J) | 0.49 (m, 2 H), 0.36-0.23 (m, 2 H). |
| N-((4-(2- | F | A | A | Method E | m/z = 620.2 | ¹H NMR (400 MHz, DMSO-d6, ppm) δ 9.65 (br, 1 H), 8.15 (d, J = 2.8 Hz, 1 H), 7.76 (d, J = 8.7 |
| (dimethylamino)ethoxy)phenyl)sulfonyl)-2,3- | | | | (20-60) | [M + H]+, Rt = | Hz, 2 H), 7.34-7.21 (m, 3 H), 6.98 (d, J = 8.7 Hz, 2 H), 6.73 (t, J = 8.2 Hz, 1 H), 5.30 (t, J = 6.4 Hz, |
| difluoro-4-((R)-1-(5-((1R,3R)-3- | | | | | 1.25 mins | 1 H), 4.90-4.83 (m, 1 H), 4.35-4.29 (m, 2 H), 4.07-4.00 (m, 1 H), 3.48-3.38 (m, 2 H), 3.15 (s, 3 H), |
| methoxycyclobutoxy)pyridin-2- | | | | | (Method E) | 2.80 (s, 6 H), 2.42-2.37 (m, 2 H), 2.31-2.23 (m, 2 H), 2.03-1.91 (m, 2 H), 0.89 (t, J = 7.3 Hz, 3 H) |
| yl)propoxy)benzamide | | | | | | |
| 2,3-difluoro-4-((R)-1-(5-((1R,3R)-3- | F | H | A | Method A | m/z = 653.3 | ¹H NMR (400 MHz, DMSO-d6, ppm) δ 9.92 (s, 1 H), 8.16 (d, J = 2.8 Hz, 1 H), 7.41-7.16 (m, 3 H), |
| methoxycyclobutoxy)pyridin-2-yl)propoxy)- | | | | (30-60) | [M + H]+, Rt = | 6.78 (t, J = 7.9 Hz, 1 H), 5.33 (t, J = 6.4 Hz, 1 H), 4.93-4.80 (m, 1 H), 4.12-3.98 (m, 1 H), 3.78-3.58 |
| N-((4-((1-methylpiperidin-4-yl)oxy)piperidin- | | | | | 1.13 mins | (m, 1 H), 3.52-3.42 (m, 1 H), 3.43-3.19 (m, 7 H), 3.10-2.92 (m, 2 H), 2.81 (t, J = 10.0 Hz, 2 H), |
| 1-yl)sulfonyl)benzamide | | | | | (Method D) | 2.73 (s, 3 H), 2.44-2.36 (m, 2 H), 2.32-2.23 (m, 2 H), 2.12-1.58 (m, 8 H), 1.48-1.36 (m, 2 H), 0.91 |
| | | | | | | (t, J = 7.3 Hz, 3 H) |
| 4-((R)-1-(5-(cyclopropylmethoxy)pyridin-2- | C | H | A | Method A | m/z = 537.2 | ¹H NMR (400 MHz, DMSO-d6, ppm) δ 8.25-8.24(m, 1 H), 7.38-7.26 (m, 3 H), 6.77-6.72 (m, 1 H), |
| yl)propoxy)-2,3-difluoro-N-(((1S,4S)-5- | | | | (25-70) | [M + H]+, Rt = | 5.31 (t, J = 6.4 Hz, 1 H), 4.27 (s, 1 H), 4.18 (s, 1 H), 3.86 (d, J = 7.0 Hz, 2 H), 3.57-3.37 (m, 3 H), |
| methyl-2,5-diazabicyclo[2.2.1]heptan-2- | | | | | 1.12 mins | 3.15-3.04 (m, 1 H), 2.79 (s, 3 H), 2.21-2.18 (m, 1 H), 2.07-1.87 (m, 3 H), 1.23-1.17 (m, 1 H), 0.91 |
| yl)sulfonyl)benzamide | | | | | (Method K) | (t, J = 7.2 Hz, 3 H), 0.61-0.50 (m, 2 H), 0.33-0.29 (m, 2 H) |
| 4-((R)-1-(5-(cyclopropylmethoxy)pyridin-2- | C | H | A | Method A | m/z = 537.5 | ¹H NMR (400 MHz, DMSO-d6, ppm) δ 9.59 (s, 1 H), 8.24 (d, J = 2.2 Hz, 1 H), 7.38-7.27 (m, 3 H), |
| yl)propoxy)-2,3-difluoro-N-(((1R,4R)-5- | | | | (25-60) | [M + H]+, Rt = | 6.78-6.69 (m, 1 H), 5.30 (t, J = 6.4 Hz, 1 H), 4.24 (s, 1 H), 4.08 (s,1 H), 3.86 (d, J = 7.1 Hz, 2 H), |
| methyl-2,5-diazabicyclo[2.2.1]heptan-2- | | | | | 1.39 mins | 3.45-3.35 (m, 3 H), 3.07-2.94 (m, 1 H), 2.74 (s, 3 H), 2.21-2.13 (m, 1 H), 2.05-1.88 (m, 3 H), 1.24- |
| yl)sulfonyl)benzamide | | | | | (Method E) | 1.18 (m, 1 H), 0.91 (t, J =7.3 Hz, 3 H), 0.60-0.52 (m, 2 H), 0.36-0.27 (m, 2 H) |
| (R)-N-((4-ethylpiperazin-1-yl)sulfonyl)-2,3- | E | H | A | Method A | m/z = 562.1 | ¹H NMR (400 MHz, DMSO-d6, ppm) δ 9.38 (s, 1 H), 8.45 (d, J = 2.6 Hz, 1 H), 8.18-8.09 (m, 1 H), |
| difluoro-4-(1-(5-(pyridin-2-yloxy)pyridin-2- | | | | (20-70) | [M + H]+, Rt = | 7.95-7.86 (m, 1 H), 7.64 (dd, J = 8.5, 2.7 Hz, 1 H), 7.49 (d, J = 8.6 Hz, 1 H), 7.44-7.34 (m, 1 H), |
| yl)propoxy)benzamide | | | | | 0.98 mins | 7.21-7.09 (m, 2 H), 6.82 (t, J = 8.0 Hz, 1 H), 5.43 (t, J = 6.4 Hz, 1 H), 3.33-2.73 (m, 10 H), 2.12- |
| | | | | | (Method K) | 1.95 (m, 2 H), 1.17 (t, J = 7.2 Hz, 3 H), 0.96 (t, J = 7.3 Hz, 3 H) |
| (R)-4-(1-(5-(cyclopropylmethoxy)pyridin-2- | C | H | A | Method A | m/z = 539.1 | ¹H NMR (400 MHz, DMSO-d6, ppm) δ 9.37 (s, 1 H), 8.25 (d, J = 1.6 Hz, 1 H), 7.41-7.30 (m, 3 H), |
| yl)propoxy)-N-((4-ethylpiperazin-1- | | | | (20-70) | [M + H]+, Rt = | 6.76 (t, J = 7.9 Hz, 1 H), 5.32 (t, J = 6.5 Hz, 1 H), 3.86 (d, J = 7.1 Hz, 2 H), 3.34-2.71 (m, 10 H), |
| yl)sulfonyl)-2,3-difluorobenzamide | | | | | 1.02 mins | 2.09-1.85 (m, 2 H), 1.26-1.10 (m, 4 H), 0.91 (t, J = 7.3 Hz, 3 H), 0.61-0.52 (m, 2 H), 0.36-0.27 (m, |
| | | | | | (Method K) | 2 H) |
| (R)-N-((4-methylpiperazin-1-yl)sulfonyl)-4- | G | CAS4 | A | Method A | m/z = 580.1 | ¹H NMR (400 MHz, DMSO-d6, ppm) δ 8.42 (d, J = 2.6 Hz, 1 H), 8.27 (d, J = 1.9 Hz, 1 H), 8.11- |
| (1-(5-(pyridin-2-yloxy)pyridin-2-yl)propoxy)- | | | | (20-90) | [M + H]+, Rt = | 8.09 (m, 1 H), 8.05 (dd, J = 8.7, 1.9 Hz, 1 H), 7.90-7.78 (m, 1 H), 7.59 (dd, J = 8.6, 2.7 Hz, 1 H), |
| 3-(trifluoromethyl)benzamide | | | | | 1.09 mins | 7.44 (d, J = 8.6 Hz, 1 H), 7.15-7.06 (m, 1 H), 7.07 (d, J = 8.3 Hz, 1 H), 6.94 (d, J = 8.8 Hz, 1 H), |
| | | | | | (Method K) | 5.45 (t, J = 6.2 Hz, 1 H), 3.48 (s, 4 H), 3.16 (s, 4 H), 2.77 (s, 3 H), 2.12-2.05 (m, 2 H), 1.07 (t, J = |
| | | | | | | 7.4 Hz, 3 H) |
| (R)-4-(1-(5-(cyclopropylmethoxy)pyridin-2- | J | H | A | Method A | m/z = 655.6 | ¹H NMR (400 MHz, DMSO-d6, ppm) δ 9.63 (s, 1 H), 8.27 (d, J = 2.8 Hz, 1 H), 8.14 (d, J = 1.8 Hz, |
| yl)propoxy)-N-((4-((1-methylpiperidin-4- | | | | (20-60) | [M + H]+, Rt = | 1 H), 8.00-7.91 (m, 1 H), 7.36 (dd, J = 8.7, 2.9 Hz, 1 H), 7.21 (d, J = 8.7 Hz, 1 H), 6.92 (d, J = 8.8 |
| yl)oxy)piperidin-1-yl)sulfonyl)-3- | | | | | 1.09 mins | Hz, 1 H), 5.42 (t, J = 6.1 Hz, 1 H), 3.86 (d, J = 7.1 Hz, 2 H), 3.75-3.56 (m, 1 H), 3.50-3.41 (m, 1 H), |
| (trifluoromethyl)benzamide | | | | | (Method K) | 3.41-3.35 (m, 2 H), 3.25-3.09 (m, 2 H), 3.08-2.86 (m, 2 H), 2.79 (t, J = 10.4 Hz, 2 H), 2.73-2.65 (m, |
| | | | | | | 3 H), 2.08-1.51 (m, 8 H), 1.48-1.33 (m, 2 H), 1.28-1.13 (m, 1 H), 0.92 (t, J = 7.3 Hz, 3 H), 0.64- |
| | | | | | | 0.45 (m, 2 H), 0.38-0.23 (m, 2 H) |
| (R)-4-(1-(5-(cyclopropylmethoxy)pyridin-2- | J | CAS4 | A | Method A | m/z = 557.1 | ¹H NMR (400 MHz, DMSO-d6, ppm) δ 9.62 (s, 1 H), 8.27 (d, J = 2.8 Hz, 1 H), 8.19-8.10 (m, 1 H), |
| yl)propoxy)-N-((4-methylpiperazin-1- | | | | (30-60) | [M + H]+, Rt = | 8.04-7.92 (m, 1 H), 7.37 (dd, J = 8.7, 2.8 Hz, 1 H), 7.22 (d, J = 8.7 Hz, 1 H), 6.94 (d, J = 8.8 Hz, 1 |
| yl)sulfonyl)-3-(trifluoromethyl)benzamide | | | | | 1.09 mins | H), 5.43 (t, J = 6.1 Hz, 1 H), 3.86 (d, J = 7.1 Hz, 2 H), 3.32-2.85 (m, 8 H), 2.69 (s, 3 H), 2.05-1.88 |
| | | | | | (Method K) | (m, 2 H), 1.32-1.12 (m, 1 H), 0.93 (t, J = 7.3 Hz, 3 H), 0.64-0.48 (m, 2 H), 0.37-0.22 (m, 2 H) |
| (R)-N-((4-cyclopropylpiperazin-1- | E | H | A | Method A | m/z = 574.2 | ¹H NMR (400 MHz, DMSO-d6, ppm) δ 11.49 (s, 1 H) 8.48-8.39 (m, 1 H), 8.16-8.11 (m, 1 H), 7.93- |
| yl)sulfonyl)-2,3-difluoro-4-(1-(5-(pyridin-2- | | | | (20-80) | [M + H]+, Rt = | 7.84 (m, 1 H), 7.68-7.60 (m, 1 H), 7.53-7.45 (m, 1 H), 7.38-7.31(m, 1 H), 7.20-7.09 (m, 2 H), 7.00- |
| yloxy)pyridin-2-yl)propoxy )benzamide | | | | | 1.14 mins | 6.90 (m, 1 H), 5.51 (t, J = 6.4 Hz, 1 H), 3.26-3.11 (m, 4 H), 2.85-2.55 (m, 4 H), 2.11-1.98 (m, 2 H), |
| | | | | | (Method K) | 1.92-1.60 (m, 1 H), 0.96(t, J = 7.3 Hz, 3 H), 0.53-0.29 (m, 4 H) |
| (R)-N-((4-cyclohexylpiperazin-1-yl)sulfonyl)- | C | H | B | C18 (5-95 | m/z = 593.5 | ¹H NMR (400 MHz, DMSO-d6, ppm) δ 8.23-8.22 (m, 1 H), 7.34-7.32 (m, 2 H), 7.32-7.28 (m, 1 |
| 4-(1-(5-(cyclopropylmethoxy)pyridin-2- | | | | MeCN/H₂O + | [M + H]+, Rt = | H), 6.81-6.75 (m, 1 H), 5.29 (t, J = 6.4 Hz, 1 H), 3.90 (d, J = 6.8 Hz, 2 H), 3.24 (t, J = 5.0 Hz, 4 H), |
| yl)propoxy)-2,3-difluorobenzamide | | | | 0.1% NH₄OH) | 4.05 mins | 2.82 (t, J = 4.7 Hz, 4 H), 2.66-2.58 (m, 1 H), 2.10-1.92 (m, 2 H), 1.87-1.83 (m, 2 H), 1.78-1.74 |
| | | | | | (Method C) | (m, 2 H), 1.59-1.56 (m, 1 H), 1.31-1.05 (m, 6 H), 0.92 (t, J = 7.4 Hz, 3 H), 0.58-0.53 (m, 2 H), |
| | | | | | | 0.34-0.29 (m, 2 H) |
| (R)-N-((4-cyclohexylpiperazin-1-yl)sulfonyl)- | E | H | B | C18 (5-95 | m/z = 616.2 | ¹H NMR (400 MHz, DMSO-d6, ppm) δ 8.41 (d, J = 2.7 Hz, 1 H), 8.16-8.13 (m, 1 H), 7.88-7.84 |
| 2,3-difluoro-4-(1-(5-(pyridin-2-yloxy)pyridin- | | | | MeCN/H₂O + | [M + H]+, Rt = | (m, 1 H), 7.60 (dd, J = 2.7, 8.5 Hz, 1 H), 7.48 (d, J = 8.5 Hz, 1 H), 7.38-7.32 (m, 1 H), 7.16-7.13 (m, |
| 2-yl)propoxy)benzamide | | | | 0.1% NH₄OH) | 3.9 mins | 1 H), 7.09-7.05 (m, 1 H), 6.89-6.83 (m, 1 H), 5.40 (t, J = 6.4 Hz, 1 H), 3.30 (t, J = 5.1 Hz, 4 H), 2.91 |
| | | | | | (Method C) | (t, J = 5.0 Hz, 4 H), 2.72-2.64 (m, 1 H), 2.14-1.98 (m, 2 H), 1.90-1.86 (m, 2 H), 1.80-1.75 (m, 2 |
| | | | | | | H), 1.60-1.57 (m, 1 H), 1.33-1.04 (m, 5 H), 0.96 (t, J = 7.4 Hz, 3 H) |
| (R)-4-(1-(5-(cyclopropylmethoxy)pyridin-2- | J | A | A | Method D | m/z = 622.2 | ¹H NMR (400 MHz, DMSO-d6, ppm) δ 9.67 (s, 1 H), 8.28-8.21 (m, 1 H), 8.13-8.06 (m, 1 H), 7.90 |
| yl)propoxy)-N-((4-(2- | | | | (25-65) | [M + H]+, Rt = | (dd, J = 8.7, 1.8 Hz, 1 H), 7.77 (d, J = 8.8 Hz, 2 H), 7.38-7.30 (m, 1 H), 7.20 (d, J = 8.7 Hz, 1 H), |
| (dimethylamino)ethoxy)phenyl)sulfonyl)-3- | | | | | 1.15 mins | 6.97 (d, J = 8.8 Hz, 2 H), 6.91 (d, J = 8.9 Hz, 1 H), 5.41 (t, J = 6.1 Hz, 1 H), 4.43-4.18 (m, 2 H), |
| (trifluoromethyl)benzamide | | | | | (Method K) | 3.85 (d, J = 7.1 Hz, 2 H), 3.53-3.41 (m, 2 H), 2.80 (s, 6 H), 2.06-1.83 (m, 2 H), 1.26-1.13 (m, 1 H), |
| | | | | | | 0.91 (t, J = 7.3 Hz, 3 H), 0.63-0.49 (m, 2 H), 0.35-0.26 (m, 2 H) |
| (R)-4-(1-(4-(cyclopropylmethoxy)pyridin-2- | L | H | A | C18 (0-50 | m/z = 623.2 | ¹H NMR (400 MHz, DMSO-d6, ppm) δ 10.05 (s, 1 H), 8.34 (d, J = 5.7 Hz, 1 H), 7.32 (t, J = 7.7 Hz, |
| yl)propoxy)-2,3-difluoro-N-((4-((1- | | | | MeCN/H₂O + | [M + H]+, Rt = | 1 H), 6.94-6.89 (m, 1 H), 6.89-6.83 (m, 1 H), 6.72 (t, J = 8.0 Hz, 1 H), 5.27 (t, J = 6.3 Hz, 1 H), |
| methylpiperidin-4-yl)oxy)piperidin-1- | | | | 0.1% NH₄OH) | 1.02 mins | 3.94-3.81 (m, 2 H), 3.63-3.52 (m, 1 H), 3.48-3.36 (m, 1 H), 3.32-3.21 (m, 2 H), 3.08-2.92 (m, 2 H), |
| yl)sulfonyl)benzamide | | | | | (Method K) | 2.81-2.62 (m, 4 H), 2.54 (s, 3 H), 2.07-1.91 (m, 2 H), 1.90-1.73 (m, 4 H), 1.67-1.51 (m, 2 H), 1.45- |
| | | | | | | 1.30 (m, 2 H), 1.24-1.11 (m, 1 H), 0.93 (t, J = 7.3 Hz, 3 H), 0.62-0.47 (m, 2 H), 0.37-0.25 (m, 2 H) |
| (R)-4-(1-(4-(cyclopropylmethoxy)pyridin-2- | L | A | A | Method D | m/z = 590.2 | ¹H NMR (400 MHz, DMSO-d6, ppm) δ 9.71 (s, 1 H), 8.33 (d, J = 5.7 Hz, 1 H), 7.76 (d, J = 8.7 Hz, |
| yl)propoxy)-N-((4-(2- | | | | (25-65) | [M + H]+, Rt = | 2 H), 7.31 (t, J = 8.2 Hz, 1 H), 6.98 (d, J = 8.7 Hz, 2 H), 6.91-6.88 (m, 1 H), 6.87-6.82 (m, 1 H), |
| (dimethylamino)ethoxy)phenyl)sulfonyl)-2,3- | | | | | 1 mins | 6.72 (t, J = 8.2 Hz, 1 H), 5.27 (t, J = 6.3 Hz, 1 H), 4.32 (t, J = 4.7 Hz, 2 H), 3.95-3.78 (m, 2 H), |
| difluorobenzamide | | | | | (Method K) | 3.50-3.41 (m, 2 H), 2.80 (s, 6 H), 2.07-1.88 (m, 2 H), 1.23-1.10 (m, 1 H), 0.92 (t, J = 7.3 Hz, 3 H), |
| | | | | | | 0.59-0.49 (m, 2 H), 0.36-0.27 (m, 2 H) |
| (R)-4-(1-(4-(cyclopropylmethoxy)pyridin-2- | L | C | A | Method A | m/z=616.3 | ¹H NMR (400 MHz, DMSO-d6, ppm) δ 9.50 (s, 1 H), 8.33 (d, J = 5.7 Hz, 1 H), 7.77-7.67 (d, J = |
| yl)propoxy)-2,3-difluoro-N-((4-((1- | | | | (15-50) | [M + H]+, Rt = | 8.7 Hz, 2 H), 7.37-7.25 (m, 1 H), 7.02-6.93 (m, 2 H), 6.90 (d, J = 2.2 Hz, 1 H), 6.87-6.82 (m, 1 H), |
| methylpiperidin-4- | | | | | 1.03 mins | 6.77-6.67 (m, 1 H), 5.27 (t, J = 6.4 Hz, 1 H), 4.74-4.56 (m, 1 H), 3.96-3.78 (m, 2 H), 3.29-3.02 (m, |
| yl)oxy)phenyl)sulfonyl)benzamide | | | | | (Method K) | 4 H), 2.77 (s, 3 H), 2.15-1.77 (m, 6 H), 1.22-1.11 (m, 1 H), 0.92 (t, J = 7.3 Hz, 3 H), 0.57-0.48 (m, 2 |
| | | | | | | H), 0.36-0.25 (m, 2 H) |
| (R)-2,3-difluoro-N-((4-((1-methylpiperidin-4- | M | H | A | Method A | m/z = 646.3 | ¹H NMR (400 MHz, DMSO-d6, ppm) δ 8.51 (d, J = 5.6 Hz, 1 H), 8.23-8.21 (m, 1 H), 8.03-7.85 |
| yl)oxy )piperidin-1-yl)sulfonyl)-4-(1-(4- | | | | (25-60) | [M + H]+, Rt = | (m, 1 H), 7.34 (t, J = 8.0 Hz, 1 H), 7.28-7.25 (m, 1 H), 7.14 (d, J = 8.2 Hz, 1 H), 7.09-7.08 (m, 1 |
| (pyridin-2-yloxy)pyridin-2- | | | | | 1.01 mins | H), 7.06-7.04 (m, 1 H), 6.75 (t, J = 7.6 Hz, 1 H), 5.38 (t, J = 6.4 Hz, 1 H), 3.62-3.60 (m, 1 H), 3.43- |
| yl)propoxy)benzamide | | | | | (Method K) | 3.41 (m, 3 H), 3.02-3.00 (m, 2 H), 2.72-2.67 (m, 4 H), 2.57 (s, 3 H), 2.02-1.96 (m, 2 H), 1.80-1.78 |
| | | | | | | (m, 4 H), 1.62-1.60 (m, 2 H), 1.39-1.37 (m, 2 H), 0.94 (t, J = 7.3 Hz, 3 H) |
| (R)-2,3-difluoro-N-((4-methylpiperazin-1- | M | CAS4 | A | Method D | m/z = 548.4 | ¹H NMR (400 MHz, DMSO-d6, ppm) δ 9.66 (s, 1 H), 8.52 (d, J = 5.6 Hz, 1 H), 8.26-8.14 (m, 1 H), |
| yl)sulfonyl)-4-(1-(4-(pyridin-2-yloxy)pyridin- | | | | (15-50) | [M + H]+, Rt = | 8.00-7.86 (m, 1 H), 7.42-7.33 (m, 1 H), 7.30-7.25 (m, 1 H), 7.15 (d, J = 8.2 Hz, 1 H), 7.11-7.08 (m, |
| 2-yl)propoxy)benzamide | | | | | 1.16 mins | 1 H), 7.07-7.01 (m, 1 H), 6.85-6.75 (m, 1 H), 5.41 (t, J = 6.3 Hz, 1 H), 3.31-2.87 (m, 8 H), 2.69 (s, 3 |
| | | | | | (Method E) | H), 2.08-1.92 (m, 2 H), 0.95 (t, J = 7.4 Hz, 3 H) |
| (R)-N-((4-cyclopropylpiperazin-1- | M | H | A | Method A | m/z = 574.2 | ¹H NMR (400 MHz, DMSO-d6, ppm) 811.48 (s, 1 H), 8.55-8.46 (m, 1 H), |
| | | | | | | 8.25-8.18 (m, 1 H), 7.98- |
| yl)sulfonyl)-2,3-difluoro-4-(1-(4-(pyridin-2- | | | | (20-70) | [M + H]+, Rt = | 7.90 (m, 1 H), 7.39-7.22 (m, 2 H), 7.19-7.10 (m, 2 H), 7.09-7.01 (m, 1 H), 6.99-6.87 (m, 1 H), |
| yloxy)pyridin-2-yl)propoxy)benzamide | | | | | 1.06 mins | 5.48 (t, J = 6.0 Hz, 1 H), 3.27-3.13 (m, 4 H), 2.81-2.56 (m, 4 H), 2.10-1.97 (m, 2 H), 1.95-1.60 (m, |
| | | | | | (Method K) | 1 H), 0.95 (t, J = 7.1 Hz, 3 H), 0.60-0.24 (m, 4 H) |
| (R)-N-((4-(2- | M | A | A | Method A | m/z= 613.2 | ¹H NMR (400 MHz, DMSO-d6, ppm) δ 9.67 (s, 1 H), 8.55-8.40 (m, 1 H), 8.27-8.11 (m, 1 H), 7.96- |
| (dimethylamino)ethoxy)phenyl)sulfonyl)-2,3- | | | | (25-70) | [M + H]+, Rt = | 7.86 (m, 1 H), 7.81-7.67 (m, 2 H), 7.38-7.30 (m, 1 H), 7.29-7.22 (m, 1 H), 7.16-7.11 (m, 1 H), 7.09- |
| difluoro-4-(1-(4-(pyridin-2-yloxy)pyridin-2- | | | | | 1.01 mins | 7.02 (m, 2 H), 7.01-6.94 (m, 2 H), 6.81-6.71(m, 1 H), 5.38 (t, J = 6.3 Hz, 1 H), 4.40-4.18 (m, 2 H), |
| yl)propoxy)benzamide | | | | | (Method K) | 3.49-3.40 (m, 2 H), 2.81 (s, 6 H), 2.05-1.92 (m, 2 H), 0.93 (t, J = 7.4 Hz, 3 H) |
| (R)-4-(1-(5-(cyclopropylmethoxy)pyridin-2- | C | H | B | C18 (5-95 | m/z = 565.4 | ¹H NMR (400 MHz, DMSO-d6, 80° C., ppm) δ 8.23 (t, J = 1.8 Hz, 1 H), 7.33 (d, J = 1.8 Hz, 2 H), |
| yl)propoxy)-N-((4- | | | | MeCN/H2O + | [M + H]+, Rt = | 7.32-7.28 (m, 1 H), 6.85-6.79 (m, 1 H), 5.31 (t, J = 6.4 Hz, 1 H), 3.90 (d, J = 6.9 Hz, 2 H), 3.32 |
| (cyclopropylmethyl)piperazin-1-yl)sulfonyl)- | | | | 0.1% NH4OH) | 3.90 mins | (t, J = 5.0 Hz, 4 H), 2.88 (t, J = 5.1 Hz, 4 H), 2.60 (d, J = 6.8 Hz, 2 H), 2.12-1.92 (m, 2 H), 1.24- |
| 2,3-difluorobenzamide | | | | | (Method C) | 1.15 (m, 1 H), 0.94-0.89 (m, 4 H), 0.59-0.51 (m, 4 H), 0.34-0.29 (m, 2 H), 0.23-0.18 (m, 2 H) |
| (R)-N-((4-(cyclopropylmethyl)piperazin-1- | E | H | B | C18 (5-95 | m/z = 588.4 | ¹H NMR (400 MHz, DMSO-d6, 80° C., ppm) δ 8.42 (dd, J = 0.6, 2.7 Hz, 1 H), 8.16-8.14 (m, 1 H), |
| yl)sulfonyl)-2,3-difluoro-4-(1-(5-(pyridin-2- | | | | MeCN/H2O + | [M + H]+, Rt = | 7.89-7.84 (m, 1 H), 7.60 (dd, J = 2.7, 8.5 Hz, 1 H), 7.49 (d, J = 8.4 Hz, 1 H), 7.38-7.33 (m, 1 H), |
| yloxy)pyridin-2-yl)propoxy)benzamide | | | | 0.1% NH4OH) | 3.76 mins | 7.17-7.13 (m, 1 H), 7.09-7.06 (m, 1 H), 6.90-6.85 (m, 1 H), 5.41 (t, J = 6.3 Hz, 1 H), 3.32 (t, J = |
| | | | | | (Method C) | 5.1 Hz, 4 H), 2.90 (t, J = 4.9 Hz, 4 H), 2.61 (d, J = 6.9 Hz, 2 H), 2.14-1.98 (m, 2 H), 0.98-0.88 |
| | | | | | | (m, 4 H), 0.57-0.52 (m, 2 H), 0.24-0.19 (m, 2 H) |
| (R)-N-((4-cyclopentylpiperazin-1- | C | H | B | C18 (5-95 | m/z = 579.4 | ¹H NMR (400 MHz, DMSO-d6, 80° C., ppm) δ 8.23 (t, J = 1.7 Hz, 1 H), 7.34-7.28 (m, 3 H), 6.84- |
| yl)sulfonyl)-4-(1-(5- | | | | MeCN/H2O + | [M + H]+, Rt = | 6.78 (m, 1 H), 5.31 (t, J = 6.4 Hz, 1 H), 3.91-3.89 (m, 2 H), 3.28 (t, J = 5.1 Hz, 4 H), 2.84 (t, J = |
| (cyclopropylmethoxy)pyridin-2-yl)propoxy)- | | | | 0.1% NH4OH) | 4 mins | 5.1 Hz, 4 H), 2.10-1.92 (m, 2 H), 1.91-1.81 (m, 2 H), 1.69-1.61 (m, 2 H), 1.57-1.45 (m, 4 H), |
| 2,3-difluorobenzamide | | | | | (Method C) | 1.25-1.15 (m, 1 H), 0.94-0.90 (m, 3 H), 0.59-0.53 (m, 2 H), 0.34-0.29 (m, 2 H) |
| (R)-N-((4-cyclopentylpiperazin-1- | E | H | B | C18 (5-95 | m/z = 602.0 | ¹H NMR (400 MHz, DMSO-d6, 80° C., ppm) δ 8.42 (dd, J = 0.7, 2.7 Hz, 1 H), 8.16-8.14 (m, 1 H), |
| yl)sulfonyl)-2,3-difluoro-4-(1-(5-(pyridin-2- | | | | MeCN/H2O + | [M + H]+, Rt = | 7.89-7.84 (m, 1 H), 7.60 (dd, J = 2.8, 8.5 Hz, 1 H), 7.49 (dd, J = 0.3, 8.5 Hz, 1 H), 7.38-7.32 (m, |
| yloxy)pyridin-2-yl)propoxy)benzamide | | | | 0.1% NH4OH) | 3.82 mins | 1 H), 7.17-7.13 (m, 1 H), 7.09-7.06 (m, 1 H), 6.90-6.84 (m, 1 H), 5.41 (t, J = 6.4 Hz, 1 H), 3.31 |
| | | | | | (Method C) | (t, J = 5.0 Hz, 4 H), 2.87 (t, J = 5.0 Hz, 4 H), 2.11-2.02 (m, 2 H), 1.90-1.83 (m, 2 H), 1.70-1.59 |
| | | | | | | (m, 2 H), 1.55-1.47 (m, 4 H), 0.98-0.94 (m, 4 H) |
| N-((4-(2- | K | A | A | Method D | m/z = 652.5 | ¹H NMR (400 MHz, DMSO-d6, ppm) δ 9.65 (s, 1 H), 8.19-8.14 (m, 1 H), 8.10 (s, 1 H), 7.90 (d, J = |
| (dimethylamino)ethoxy)phenyl)sulfonyl)-4- | | | | (20-60) | [M + H]+, Rt = | 8.7 Hz, 1 H), 7.79-7.72 (m, 2 H), 7.28-7.18 (m, 2 H), 6.99-6.93 (m, 2 H), 6.92 (d, J = 8.9 Hz, 1 H), |
| ((R)-1-(5-((1R,3R)-3- | | | | | 1.44 mins | 5.41 (t, J = 6.0 Hz, 1 H), 4.91-4.81 (m, 1H), 4.30 (t, J = 4.7 Hz, 2 H), 4.07-3.99 (m, 1 H), 3.48-3.37 |
| methoxycyclobutoxy)pyridin-2-yl)propoxy)- | | | | | (Method E) | (m, 2 H), 3.16-3.11 (m, 3 H), 2.79 (s, 6 H), 2.43-2.34 (m, 2 H), 2.32-2.23 (m, 2 H), 2.00-1.90 (m, 2 |
| 3-(trifluoromethyl)benzamide | | | | | | H), 0.91 (t, J = 7.2 Hz, 3 H) |
| (R)-4-(1-(5-(cyclopropylmethoxy)pyridin-2- | J | H | A | Method C | m/z = 571.2 | ¹H NMR (400 MHz, DMSO-d6, ppm) δ 9.25 (s, 1 H), 8.27 (d, J = 2.7 Hz, 1 H), 8.18-8.10 (m, 1 H), |
| yl)propoxy)-N-((4-ethylpiperazin-1- | | | | (35-75) | [M + H]+, Rt = | 8.02-7.94 (m, 1 H), 7.41-7.32 (m, 1 H), 7.22 (d, J = 8.7 Hz, 1 H), 6.94 (d, J = 8.8 Hz, 1 H), 5.43 (t, |
| yl)sulfonyl)-3-(trifluoromethyl)benzamide | | | | | 1.24 mins | J = 6.1 Hz, 1 H), 3.86 (d, J = 7.1 Hz, 2 H), 3.70-3.35 (m, 2 H), 3.25-2.74 (m, 8 H), 2.03-1.87 (m, 2 |
| | | | | | (Method K) | H), 1.27-1.08 (m, 4 H), 0.92 (t, J = 7.3 Hz, 3 H), 0.65-0.47 (m, 2 H), 0.40-0.24 (m, 2 H). |
| 4-((R)-1-(5-((1R,3R)-3- | K | H | A | Method A | m/z = 685.3 | ¹H NMR (400 MHz, DMSO-d6, ppm) δ 8.21-8.13 (m, 2 H), 8.00-7.88 (m, 1 H), 7.29-7.17 (m, 2 H), |
| methoxycyclobutoxy)pyridin-2-yl)propoxy)- | | | | (25-70) | [M + H]+, Rt = | 6.99-6.88 (m, 1 H), 5.44 (t, J = 6.1 Hz, 1 H), 4.91-4.81(m, 1 H), 4.08-4.01 (m, 1 H), 3.76-3.56 (m, 1 |
| N-((4-((1-methylpiperidin-4-yl)oxy)piperidin- | | | | | 1.14 mins | H), 3.51-3.43 (m, 1 H), 3.41-3.34 (m, 2 H), 3.20-3.13 (m, 4 H), 3.10-2.93 (m, 2 H), 2.88-2.78 (m, |
| 1-yl)sulfonyl)-3-(trifluoromethyl)benzamide | | | | | (Method K) | 2 H), 2.72 (s, 3 H), 2.43-2.36 (m, 3 H), 2.32-2.23 (m, 2 H), 2.00-1.78 (m, 6 H), 1.76-1.57 (m, 2 H), |
| | | | | | | 1.47-1.36 (m, 2 H), 0.92 (t, J = 7.3 Hz, 3 H). |
| 4-((R)-1-(5-((1R,3R)-3- | N | CAS4 | A | Method E | m/z = 591.4 | ¹H NMR (400 MHz, DMSO-d6, ppm) δ 9.65 (s, 1 H), 8.21-8.13 (m, 1 H), 7.38-7.24 (m, 3 H), 6.82- |
| (difluoromethoxy)cyclobutoxy)pyridin-2- | | | | (20-60) | [M + H]+, Rt = | 6.70 (m, 1 H), 6.66 (t, 2JH-F =75.7 Hz, 1 H), 5.33 (t, J = 6.5 Hz, 1 H), 5.00-4.90 (m, 1 H), 4.85- |
| yl)propoxy)-2,3-difluoro-N-((4- | | | | | 1.26 mins | 4.76 (m, 1 H), 3.31-2.85 (m, 8 H), 2.68 (s, 3 H), 2.63-2.55 (m, 2 H), 2.47-2.41 (m, 2 H), 2.05-1.89 |
| methylpiperazin-1-yl)sulfonyl)benzamide | | | | | (Method E) | (m, 2 H), 0.91 (t, J = 7.3 Hz, 3 H). |
| (R)-N-((4-ethylpiperazin-1-yl)sulfonyl)-4-(1- | G | H | A | Method A | m/z = 594.2 | ¹H NMR (400 MHz, methanol-d4,, ppm) δ 8.42 (d, J = 2.6 Hz, 1 H), 8.28 (d, J = 1.9 Hz, 1 H), 8.10- |
| (5-(pyridin-2-yloxy)pyridin-2-yl)propoxy)-3- | | | | (30-70) | [M + H]+, Rt = | 8.05 (m, 2 H), 7.95-7.77 (m, 1 H), 7.59 (dd, J = 8.6, 2.7 Hz, 1 H), 7.44 (d, J = 8.6 Hz, 1 H), 7.14 |
| (trifluoromethyl)benzamide | | | | | 1.09 mins | (dd, J = 6.9, 5.3 Hz, 1 H), 7.07 (d, J = 8.3 Hz, 1 H), 6.92 (d, J = 8.8 Hz, 1 H), 5.44 (t, J = 6.2 Hz, 1 |
| | | | | | (Method K) | H), 3.75-3.40 (m, 4 H), 3.25-3.23 (m, 3 H), 3.13-3.07 (m, 3 H), 2.10-2.05 (m, 2 H), 1.30 (t, J = 7.3 |
| | | | | | | Hz, 3 H), 1.07 (t, J = 7.4 Hz, 3 H). |
| 4-((R)-1-(5-((1R,3R)-3- | K | CAS4 | B | Method A | m/z = 587.2 | ¹H NMR (400 MHz, DMSO-d6, ppm) δ 9.69 (s, 1 H), 8.17 (dd, J = 14.9, 2.2 Hz, 2 H), 7.98 (dd, J = |
| methoxycyclobutoxy)pyridin-2-yl)propoxy)- | | | | (20-70) | [M + H]+, Rt = | 8.8, 1.8 Hz, 1 H), 7.28-7.21 (m, 2 H), 6.95 (d, J = 8.8 Hz, 1 H), 5.44 (t, J = 6.1 Hz, 1 H), 4.96-4.70 |
| N-((4-methylpiperazin-1-yl)sulfonyl)-3- | | | | | 1.09 mins | (m, 1 H), 4.11-3.98 (m, 1 H), 3.3.3-3.18 (m, 3 H), 3.15 (s, 3 H), 3.09-2.97 (m, 4 H), 2.69 (s, 3 H), |
| (trifluoromethyl)benzamide | | | | | (Method K) | 2.48-2.35 (m, 3 H), 2.33-2.19 (m, 2 H), 2.04-1.79 (m, 2 H), 0.92 (t, J = 7.3 Hz, 3 H). |
| N-((4-cyclopropylpiperazin-1-yl)sulfonyl)- | F | H | B | Method A | m/z = 581.3 | ¹H NMR (400 MHz, DMSO-d6, ppm) δ 11.48 (s, 1 H), 8.17 (d, J = 2.7 Hz, 1 H), 7.38-7.25 (m, 3 |
| 2,3-difluoro-4-((R)-1-(5-((1R,3R)-3- | | | | (20-70) | [M + H]+, Rt = | H), 6.90 (t, J = 8.0 Hz, 1 H), 5.40 (t, J = 6.5 Hz, 1 H), 4.96-4.79 (m, 1 H), 4.10 -. 98 (m, 1 H), 3.25- |
| methoxycyclobutoxy)pyridin-2- | | | | | 1.1 mins | 3.17 (m, 4 H), 3.15 (s, 3 H), 2.78-2.56 (m, 4 H), 2.43-2.38 (m, 2 H), 2.33-2.22 (m, 2 H), 2.09-1.90 |
| yl)propoxy)benzamide | | | | | (Method K) | (m, 2 H), 1.86-1.71 (m, 1 H), 0.91 (t, J = 7.3 Hz, 3 H), 0.60-0.23 (m, 4 H). |
| (R)-4-(1-(5-(cyclopropylmethoxy)pyridin-2- | C | J | A | Method A | m/z = 583.2 | ¹H NMR (400 MHz, DMSO-d6, ppm) δ 8.25 (s, 1 H), 7.42-7.25 (m, 3 H), 6.84 (t, J = 7.9 Hz, 1 H), |
| yl)propoxy)-2,3-difluoro-N-((4-(2-hydroxy-2- | | | | (30-70) | [M + H]+, Rt = | 5.36 (t, J = 6.4 Hz, 1 H), 4.66 (s, 1 H), 3.86 (d, J = 7.1 Hz, 2 H), 3.30-3.14 (m, 4 H), 3.13-2.52 (m, 6 |
| methylpropyl)piperazin-1- | | | | | 1.09 mins | H), 2.12-1.87 (m, 2 H), 1.28-1.03 (m, 7 H), 0.91 (t, J = 7.3 Hz, 3 H), 0.63-0.51 (m, 2 H), 0.37- 0.27 |
| yl)sulfonyl)benzamide | | | | | (Method K) | (m, 2 H) |
| N-(((S)-3,4-dimethylpiperazin-1-yl)sulfonyl)- | E | H | A | Method D | m/z = 562.2 | ¹H NMR (400 MHz, DMSO-d6, ppm) δ 9.52 (s, 1 H), 8.49-8.41 (m, 1 H), 8.19-8.11 (m, 1 H), 7.94- |
| 2,3-difluoro-4-((R)-1-(5-(pyridin-2- | | | | (20-60) | [M + H]+, Rt = | 7.84 (m, 1 H), 7.69-7.60 (m, 1 H), 7.53-7.05 (m, 1 H), 7.43-7.35 (m, 1 H), 7.21-7.09 (m, 2 H), 6.82 |
| yloxy)pyridin-2-yl)propoxy)benzamide | | | | | 1.01 mins | (t, J = 8.0 Hz, 1H), 5.43 (t, J = 6.3 Hz, 1H), 3.65-3.43 (m, 2 H), 3.24-2.85 (m, 4 H), 2.80-2.59 (m, 4 |
| | | | | | (Method F) | H), 2.12-1.94 (m, 2 H), 1.21 (d, J = 6.2 Hz, 3 H), 0.96 (t, J = 7.3 Hz, 3 H). |
| (R)-4-(1-(3- | O | H | A | FCC (0-25% | m/z= 622.3 | ¹H NMR (400 MHz, DMSO-d6, ppm) δ 9.40 (s, 1 H), 7.32-7.27 (m, 1 H), 7.26-7.21 (m, 1 H), 6.93- |
| (cyclopropylmethoxy)phenyl)propoxy)-2,3- | | | | MeOH/DCM + | [M + H]+, Rt = | 6.90 (m, 2 H), 6.82-6.75 (m, 2 H), 5.32 (t, J = 6.3 Hz, 1 H), 3.82-3.74 (m, 2 H), 3.68-3.58 (m, 1 H), |
| difluoro-N-((4-((1-methylpiperidin-4- | | | | 0.2% NH4OH) | 1.22 mins | 3.45-3.40 (m, 1 H), 3.19-3.06 (m, 2 H), 3.03-2.81 (m, 2 H), 2.77-2.70 (m, 2 H), 2.67 (s, 3 H), 2.03- |
| yl)oxy)piperidin-1-yl)sulfonyl)benzamide | | | | | (Method F) | 1.34 (m, 12 H), 1.21-1.15 (m, 1 H), 0.92 (t, J = 7.3 Hz, 3 H), 0.57-0.52 (m, 2 H), 0.33-0.28 (m, 2 |
| | | | | | | H). |
| 2,3-difluoro-4-((R)-1-(5-((1r,3R)-3- | F | CAS4 | B | Method A | m/z = 555.1 | ¹H NMR (400 MHz, DMSO-d6, ppm) δ 9.63 (s, 1 H), 8.17 (d, J = 2.8 Hz, 1 H), 7.36-3.32 (m, 2 H), |
| methoxycyclobutoxy)pyridin-2-yl)propoxy)- | | | | (20-70) | [M + H]+, Rt = | 7.27-7.24 (m, 1 H), 6.77 (t, J = 7.9 Hz, 1 H), 5.33 (t, J = 6.4 Hz, 1 H), 4.90-4.85 (m, 1 H), 4.12-3.99 |
| N-((4-methylpiperazin-1- | | | | | 1.05 mins | (m, 1 H), 3.32-3.24 (m, 4 H), 3.15 (s, 3 H), 3.07-2.97 (m, 4 H), 2.67 (s, 3 H), 2.43-2.38 (m, 2 H), |
| yl)sulfonyl)benzamide | | | | | (Method F) | 2.33-2.21 (m, 2 H), 2.08-1.86 (m, 2 H), 0.91 (t, J = 7.3 Hz, 3 H). |
| (R)-2,3-difluoro-N-((4-(2-hydroxy-2- | E | J | A | Method A | m/z = 606.5 | ¹H NMR (400 MHz, DMSO-d6, ppm) δ 8.49-8.40 (m, 1 H), 8.19-8.08 (m, 1 H), 7.94-7.84 (m, 1 H), |
| methylpropyl)piperazin-1-yl)sulfonyl)-4-(1- | | | | (30-70) | [M + H]+, Rt = | 7.68-7.60 (m, 1 H), 7.53-7.46 (m, 1 H), 7.42-7.33 (m, 1 H), 7.23-7.06 (m, 2 H), 6.90 (t, J = 7.7 Hz, |
| (5-(pyridin-2-yloxy)pyridin-2- | | | | | 1.29 mins | 1 H), 5.48 (t, J = 6.4 Hz, 1 H), 4.62 (s, 1 H), 3.32-3.17 (m, 5 H), 3.15-2.55 (m, 5 H), 2.18-1.88 (m, 2 |
| yl)propoxy)benzamide | | | | | (Method E) | H), 1.14 (s, 6 H), 0.96 (t, J = 7.3 Hz, 3 H). |
| (R)-2,3-difluoro-N-((4-(2-fluoro-2- | E | J | A | Method A | m/z = 608.5 | ¹H NMR (400 MHz, DMSO-d6, ppm) δ 11.76 (s, 1 H), 8 8.48-8.36 (m, 1 H), 8.15-8.12 (m, 1 H), |
| methylpropyl)piperazin-1-yl)sulfonyl)-4-(1- | | | | (40-90) | [M + H]+, Rt = | 7.94-7.84 (m, 1 H), 7.69-7.60 (m, 1 H), 7.52 (d, J = 8.5 Hz, 1 H), 7.40-7.31 (m, 1 H), 7.22-7.10 (m, |
| (5-(pyridin-2-yloxy)pyridin-2- | | | | | 1.4 mins | 2 H), 6.99 (t, J = 8.0 Hz, 1 H), 5.53 (t, J = 6.4 Hz, 1 H), 3.31-3.10 (m, 5 H), 2.64-2.52 (m, 5 H), |
| yl)propoxy)benzamide | | | | | (Method E) | 2.18-1.95 (m, 2 H), 1.39-1.21 (m, 6 H), 0.96 (t, J = 7.3 Hz, 3 H). |
| (R)-4-(1-(5-(cyclopropylmethoxy)pyridin-2- | C | J | A | Method A | m/z = 585.4 | ¹H NMR (400 MHz, DMSO-d6, ppm) δ 11.74 (s, 1 H), 7.34-7.18 (m, 1 H), 7.44-7.17 (m, 3 H), 6.92 |
| yl)propoxy)-2,3-difluoro-N-((4-(2-fluoro-2- | | | | (50-90) | [M + H]+, Rt = | (t, J = 8.0 Hz, 1 H), 5.41 (t, J = 6.5 Hz, 1 H), 3.87 (d, J = 7.1 Hz, 2 H), 3.30-3.14 (m, 4 H), 2.64- |
| methylpropyl)piperazin-1- | | | | | 1.45 mins | 2.51 (m, 6 H), 2.14-1.91 (m, 2 H), 1.46-1.13 (m, 7 H), 0.91 (t, J = 7.3 Hz, 3 H), 0.64-0.49 (m, 2 H), |
| yl)sulfonyl)benzamide | | | | | (Method E) | 0.39- 0.22 (m, 2 H). |
| (R)-4-(1-(4-(cyclopropylmethoxy)pyridin-2- | L | CAS4 | A | Method A | m/z = 525.2 | ¹H NMR (400 MHz, DMSO-d6, ppm) δ 9.76 (s, 1 H), 8.35 (d, J = 5.7 Hz, 1 H), 7.35 (t, J = 7.7 Hz, |
| yl)propoxy)-2,3-difluoro-N-((4- | | | | (20-80) | [M + H]+, Rt = | 1 H), 6.92-6.91 (m, 1 H), 6.88-6.86 (m, 1 H), 6.76 (t, J = 8.0 Hz, 1 H), 5.29 (t, J = 6.4 Hz, 1 H), |
| methylpiperazin-1-yl)sulfonyl)benzamide | | | | | 0.903 mins | 3.89-3.87 (m, 2 H), 3.26-3.24 (m, 4 H), 3.09-3.07 (m, 4 H), 2.68 (s, 3 H), 2.02-1.96 (m, 2 H), 1.20- |
| | | | | | (Method F) | 1.15 (m, 1 H), 0.93 (t, J = 7.3 Hz, 3 H), 0.72-0.42 (m, 2 H), 0.32-0.30 (m, 2 H). |
| (R)-N-((4-cyclobutylpiperazin-1-yl)sulfonyl)- | C | H | B | FCC (0-10% | m/z = 565.4 | ¹H NMR (400 MHz, DMSO-d6, ppm) δ 9.90 (s, 1 H), 8.26-8.24 (m, 1 H), 7.38-7.30 (m, 3 H), |
| 4-(1-(5-(cyclopropylmethoxy)pyridin-2- | | | | 2M NH₃ in | [M + H]+, Rt = | 6.79 (t, J = 7.9 Hz, 1 H), 5.34 (t, J = 6.4 Hz, 1 H), 3.87 (d, J = 7.0 Hz, 2 H), 3.26 (s, 5 H), 2.88 (s, |
| yl)propoxy)-2,3-difluorobenzamide | | | | MeOH/DCM) | 3.94 mins | 4 H), 2.12-1.92 (m, 6 H), 1.73-1.62 (m, 2 H), 1.26-1.15 (m, 1 H), 0.91 (t, J = 7.4 Hz, 3 H), 0.59- |
| | | | | | (Method C) | 0.54 (m, 2 H), 0.34-0.29 (m, 2 H) |
| (R)-N-((4-cyclobutylpiperazin-1-yl)sulfonyl)- | E | H | B | FCC (0-10% | m/z = 588.4 | ¹H NMR (400 MHz, DMSO-d6, ppm) δ 9.83 (s, 1 H), 8.46-8.44 (m, 1 H), 8.16-8.14 (m, 1 H), |
| 2,3-difluoro-4-(1-(5-(pyridin-2-yloxy)pyridin- | | | | 2M NH₃ in | [M + H]+, Rt = | 7.92-7.87 (m, 1 H), 7.64 (dd, J = 2.8, 8.6 Hz, 1 H), 7.50 (d, J = 8.7 Hz, 1 H), 7.41-7.35 (m, 1 H), |
| 2-yl)propoxy)benzamide | | | | MeOH/DCM) | 3.77 mins | 7.19-7.12 (m, 2 H), 6.84 (t, J = 7.8 Hz, 1 H), 5.44 (t, J = 6.4 Hz, 1 H), 3.22 (s, 5 H), 2.82 (s, 4 H), |
| | | | | | (Method C) | 2.14-1.96 (m, 6 H), 1.74-1.62 (m, 2 H), 0.96 (t, J = 7.4 Hz, 3 H) |
| N-((4-ethylpiperazin-1-yl)sulfonyl)-4-((R)-1- | K | H | A | Method A | m/z = 601.3 | ¹H NMR (400 MHz, methanol-d4, ppm) δ 8.26-8.25 (m, 1 H), 8.13 (d, J = 2.8 Hz, 1 H), 8.03-8.00 |
| (5-((1r,3R)-3-methoxycyclobutoxy)pyridin-2- | | | | (35-95) | [M + H]+, Rt = | (m, 1 H), 7.32-7.29 (m, 1 H), 7.25-7.21 (m, 1 H), 6.86 (d, J = 8.8 Hz, 1 H), 5.35 (t, J = 6.4 Hz, 1 H), |
| yl)propoxy)-3-(trifluoromethyl)benzamide | | | | | 1.13 mins | 4.91-4.88 (m, 1 H), 4.17-4.05 (m, 1 H), 3.64-3.33 (m, 4 H), 3.26-3.21 (m, 5 H), 3.19-3.04 (m, 4 H), |
| | | | | | (Method F) | 2.47-2.37 (m, 4 H), 2.06-1.99 (m, 2 H), 1.29 (t, J = 7.4 Hz, 3 H), 1.02 (t, J = 7.4 Hz, 3 H). |
| 2,3-difluoro-4-((R)-1-(4-((1r,3R)-3- | P | CAS4 | B | Method A | m/z = 555.2 | ¹H NMR (400 MHz, methanol-d4, ppm) δ 8.31 (d, J = 5.8 Hz, 1H), 7.37-7.32 (m, 1H), 6.87-6.86 |
| methoxycyclobutoxy)pyridin-2-yl)propoxy)- | | | | (20-95) | [M + H]+, Rt = | (m, 1H), 6.78-6.76 (m, 1H), 6.70-6.66 (m, 1H), 5.26 (t, J = 6.4 Hz, 1H), 4.94-4.88 (m, 1H), 4.16- |
| N-((4-methylpiperazin-1- | | | | | 0.9 mins | 4.00 (m, 1H), 3.57-3.39 (m, 4H), 3.25 (s, 3H), 3.15-3.04 (m, 4H), 2.73 (s, 3H), 2.53-2.21 (m, 4H), |
| yl)sulfonyl)benzamide | | | | | (Method F) | 2.11-1.99 (m, 2H), 1.04 (t, J = 7.4 Hz, 3H). |
| 2,3-difluoro-4-((R)-1-(4-((1r,3R)-3- | P | H | B | Method A | m/z = 653.2 | ¹H NMR (400 MHz, DMSO-d6, ppm) δ 9.80 (s, 1 H), 8.36-8.34 (m, 1 H), 7.34-7.29 (m, 1 H), 6.87- |
| methoxycyclobutoxy)pyridin-2-yl)propoxy)- | | | | (20-60) | [M + H]+, Rt = | 6.71 (m, 3 H), 5.34 (t, J = 6.4 Hz, 1 H), 4.93-4.86 (m, 1 H), 4.08-3.96 (m, 1 H), 3.74-3.59 (m, 1 H), |
| N-((4-((1-methylpiperidin-4-yl)oxy)piperidin- | | | | | 0.97 mins | 3.55-3.45 (m, 1 H), 3.44-3.36 (m, 2 H), 3.22-2.98 (m, 6 H), 2.93-2.80 (m, 2 H), 2.74 (s, 3 H), 2.46- |
| 1-yl)sulfonyl)benzamide | | | | | (Method F) | 2.14 (m, 5 H), 2.10-1.85 (m, 4 H), 1.84-1.57 (m, 4 H), 1.51-1.33 (m, 2 H), 0.93 (t, J = 7.4Hz, 3 H). |
| (R)-4-(1-(5-(cyclopropylmethoxy)pyridin-2- | C | H | B | Method D | m/z = 595.2 | ¹H NMR (400 MHz, DMSO-d6, ppm) δ 8.30-8.17 (m, 1 H), 7.41-7.24 (m, 3 H), 6.74-6.70 (m, 1 H), |
| yl)propoxy)-2,3-difluoro-N-((4-((1- | | | | (20-95) | [M + H]+, Rt = | 5.30 (t, J = 6.4 Hz, 1 H), 4.37-4.32 (m, 1 H), 4.20-4.05 (m, 2 H), 3.87-3.85 (m, 2 H), 3.73-3.68 (m, |
| methylazetidin-3-yl)oxy)piperidin-1- | | | | | 1.11 mins | 2 H), 3.39-3.35 (m, 2 H), 3.26-3.10 (m, 1 H), 2.76-2.62 (m, 5 H), 2.09-1.90 (m, 2 H), 1.82-1.76 (m, |
| yl)sulfonyl)benzamide | | | | | (Method F) | 2 H), 1.43-1.35 (m, 2 H), 1.25-1.14 (m, 1 H), 0.90 (t, J = 7.4 Hz, 3 H), 0.61-0.51 (m, 2 H), 0.33- |
| | | | | | | 0.29 (m, 2 H). |
| (R)-2,3-difluoro-N-((4-((1-methylazetidin-3- | E | H | B | Method D | m/z= 618.2 | ¹H NMR (400 MHz, DMSO-d6, ppm) δ 8.15-8.11 (m, 1 H), 7.95-7.78 (m, 1 H), 7.64-7.60 (m, 1 H), |
| yl)oxy)piperidin-1-yl)sulfonyl)-4-(1-(5- | | | | (20-60) | [M + H]+, Rt = | 7.48-7.46 (m, 1 H), 7.36-7.31 (m, 1 H), 7.22-7.04 (m, 2 H), 6.79-6.75 (m, 1 H), 5.39 (t, J = 6.4 Hz, |
| (pyridin-2-yloxy)pyridin-2- | | | | | 1.07 mins | 1 H), 4.41-4.26 (m, 1 H), 4.18-4.09 (m, 2 H), 3.74-3.62 (m, 2 H), 3.37-3.34 (m, 3 H), 3.24-3.09 (m, |
| yl)propoxy)benzamide | | | | | (Method F) | 1 H), 2.72-2.62 (m, 5 H), 2.07-1.92 (m, 2 H), 1.79-1.75 (m, 2 H), 1.45-1.32 (m, 2 H), 0.94 (t, J = 7.4 |
| | | | | | | Hz, 3 H). |
| (R)-2,3-difluoro-N-((6-methyl-2,6- | E | H | A | Method A | m/z = 560.1 | ¹H NMR (400 MHz, DMSO-d6, ppm) δ 9.51 (s, 1 H), 8.46-8.44 (m, 1 H), 8.16-8.14 (m, 1 H), 7.96- |
| diazaspiro[3.3]heptan-2-yl)sulfonyl)-4-(1-(5- | | | | (20-60) | [M + H]+, Rt = | 7.83 (m, 1 H), 7.66-7.62 (m, 1 H), 7.51-7.49 (m, 1 H), 7.42-7.38 (m, 1 H), 7.24-7.07 (m, 2 H), 6.84- |
| (pyridin-2-yloxy)pyridin-2- | | | | | 1.02 mins | 6.80 (m, 1 H), 5.42 (t, J = 6.4 Hz, 1 H), 4.15 (s, 4 H), 3.89 (s, 4 H), 2.75 (s, 3 H), 2.08-1.99 (m, 2 |
| yl)propoxy)benzamide | | | | | (Method F) | H), 0.96 (t, J = 7.4 Hz, 3 H). |
| (R)-4-(1-(5-(cyclopropylmethoxy)pyridin-2- | C | H | C | Method A | m/z = 537.2 | ¹H NMR (400 MHz, DMSO-d6, ppm) δ 9.66 (s, 1 H), 8.26-8.24 (m, 1 H), 7.40-7.27 (m, 3 H), 6.77- |
| yl)propoxy)-2,3-difluoro-N-((6-methyl-2,6- | | | | (30-60) | [M + H]+, Rt = | 6.73 (m, 1 H), 5.31 (t, J = 6.4 Hz, 1 H), 4.10 (s, 4 H), 3.90-3.80 (m, 6 H), 2.72 (s, 3 H), 2.10-1.88 |
| diazaspiro[3.3]heptan-2- | | | | | 1.05 mins | (m, 2 H), 1.24-1.17 (m, 1 H), 0.91 (t, J = 7.4 Hz, 3 H), 0.62-0.50 (m, 2 H), 0.39-0.22 (m, 2 H). |
| yl)sulfonyl)benzamide | | | | | (Method F) | |
| 2,3-difluoro-N-((8-methyl-3,8- | E | H | C | Method D | m/z = 574.4 | ¹H NMR (400 MHz, DMSO-d6, ppm) δ 9.50 (s, 1 H), 8.45 (d, J = 2.7 Hz, 1 H), 8.16-8.10 (m, 1 H), |
| diazabicyclo[3.2.1]octan-3-yl)sulfonyl)-4- | | | | (20-60) | [M + H]+, Rt = | 7.94-7.85 (m, 1 H), 7.64 (dd, J = 8.5, 2.7 Hz, 1 H), 7.49 (d, J = 8.6 Hz, 1 H), 7.43-7.35 (m, 1 H), |
| ((R)-1-(5-(pyridin-2-yloxy)pyridin-2- | | | | | 1.31 mins | 7.19-7.15 (m, 1 H), 7.15-7.11 (m, 1 H), 6.83-6.77 (m, 1 H), 5.42 (t, J = 6.3 Hz, 1 H), 3.91-3.81 (m, |
| yl)propoxy)benzamide | | | | | (Method E) | 2 H), 3.40-3.33 (m, 2 H), 3.23-3.11 (m, 2 H), 2.64 (s, 3 H), 2.09-1.97 (m, 4 H), 1.92-1.84 (m, 2 H), |
| | | | | | | 0.96 (t, J = 7.3 Hz, 3 H). |
| 2,3-difluoro-N-((4-(((S)-1-methylpiperidin-3- | E | C | B | Method D | m/z = 639.5 | ¹H NMR (400 MHz, DMSO-d6, ppm) δ 9.27 (s, 1 H), 8.43 (d, J = 2.5 Hz, 1 H), 8.18-8.10 (m, 1 H), |
| yl)oxy)phenyl)sulfonyl)-4-((R)-1-(5-(pyridin- | | | | (20-60) | [M + H]+, Rt = | 7.92-7.85 (m, 1 H), 7.77 (d, J = 8.8 Hz, 2 H), 7.62 (dd, J = 8.5, 2.7 Hz, 1 H), 7.48 (d, J = 8.5 Hz, 1 |
| 2-yloxy)pyridin-2-yl)propoxy)benzamide | | | | | 1.25 mins | H), 7.40-7.30 (m, 1 H), 7.19-7.14 (m, 1 H), 7.12 (d, J = 8.3 Hz, 1 H), 7.06-6.95 (m, 2 H), 6.86-6.76 |
| | | | | | (Method E) | (m, 1 H), 5.42 (t, J = 6.4 Hz, 1 H), 5.11-4.3 (m, 1 H), 3.55-3.32 (m, 2 H), 3.23-2.86 (m, 2 H), 2.74 |
| | | | | | | (s, 3 H), 2.08-1.66 (m, 6 H), 0.95 (t, J = 7.3 Hz, 3 H). |
| N-((4-ethylpiperazin-1-yl)sulfonyl)-2,3- | F | H | A | Method A | m/z = 569.1 | ¹H NMR (400 MHz, DMSO-d6, ppm) δ 9.33 (s, 1 H), 8.17 (d, J = 2.5 Hz, 1 H), 7.37-7.33 (m, 2 H), |
| difluoro-4-((R)-1-(5-((1r,3R)-3- | | | | (20-70) | [M + H]+, Rt = | 7.27-7.24 (m, 1 H), 6.77 (t, J = 8.0 Hz, 1 H), 5.33 (t, J = 6.4 Hz, 1 H), 4.94 -4.77 (m, 1 H), 4.11- |
| methoxycyclobutoxy)pyridin-2- | | | | | 1.01 mins | 3.97 (m, 1 H), 3.39-3.30 (m, 4 H), 3.15 (s, 3 H), 3.09-2.96 (m, 4 H), 2.43-2.38 (m, 3 H), 2.30-2.25 |
| yl)propoxy)benzamide | | | | | (Method F) | (m, 3 H), 2.03-1.93 (m, 2 H), 1.17 (t, J = 7.2 Hz, 3 H), 0.91 (t, J = 7.3 Hz, 3 H). |
| (R)-4-(1-(5-(cyclopropylmethoxy)pyridin-2- | C | H | A | Method D | m/z = 549.2 | ¹H NMR (400 MHz, DMSO-d6, ppm) δ 10.69 (s, 1 H), 8.24 (d, J = 1.3 Hz, 1 H), 7.46-7.13 (m, 3 |
| yl)propoxy)-2,3-difluoro-N-((5-methyl- | | | | (15-40) | [M + H]+, Rt = | H), 6.74 (t, J = 8.2 Hz, 1 H), 5.31 (t, J = 6.4 Hz, 1 H), 4.09-3.90 (m, 8 H), 3.86 (dd, J = 7.0, 1.3 Hz, |
| 3,4,5,6-tetrahydropyrrolo[3,4-c]pyrrol-2(1H)- | | | | | 1.06 mins | 2 H), 2.86 (s, 3 H), 2.08-1.88 (m, 2 H), 1.25-1.12 (m, 1 H), 0.96-0.80 (m, 3 H), 0.59-0.50 (m, 2 H), |
| yl)sulfonyl)benzamide | | | | | (Method F) | 0.34-0.26 (m, 2 H). |
| 2,3-difluoro-N-((4-(((R)-1-methylpiperidin-3- | E | C | B | Method A | m/z = 639.2 | ¹H NMR (400 MHz, DMSO-d6, ppm) δ 9.70 (s, 1 H), 8.46-8.31 (m, 1 H), 8.16-8.08 (m, 1 H), 7.92- |
| yl)oxy)phenyl)sulfonyl)-4-((R)-1-(5-(pyridin- | | | | (30-70) | [M + H]+, Rt = | 7.81 (m, 1 H), 7.80-7.69 (m, 2 H), 7.65-7.55 (m, 1 H), 7.51-7.42 (m, 1 H), 7.40-7.31 (m, 1 H), 7.19- |
| 2-yloxy)pyridin-2-yl)propoxy)benzamide | | | | | 1.11 mins | 7.07 (m, 2 H), 7.05-6.95 (m, 2 H), 6.85-6.74 (m, 1 H), 5.42 (t, J = 6.4 Hz, 1 H), 4.95-4.57 (m, 1 H), |
| | | | | | (Method F) | 3.39-3.25 (m, 2 H), 3.15-2.94 (m, 2 H), 2.74 (s, 3 H), 2.09-1.96 (m, 2 H), 1.92-1.62 (m, 4 H), 0.95 |
| | | | | | | (t, J = 7.3 Hz, 3 H). |
| (R)-2,3-difluoro-N-((5-methyl-3,4,5,6- | E | H | A | Method D | m/z = 572.2 | ¹H NMR (400 MHz, DMSO-d6, ppm) δ 10.28 (s, 1 H), 8.48-8.36 (m, 1 H), 8.17-8.13 (m, 1 H), |
| tetrahydropyrrolo[3,4-c]pyrrol-2(1H)- | | | | (15-40) | [M + H]+, Rt = | 7.93-7.85 (m, 1 H), 7.68-7.59 (m, 1 H), 7.54-7.46 (m, 1 H), 7.43-7.34 (m, 1 H), 7.20-7.09 (m, 2 H), |
| yl)sulfonyl)-4-(1-(5-(pyridin-2-yloxy)pyridin- | | | | | 1 mins | 6.86-6.77 (m, 1 H), 5.43 (t, J = 6.4 Hz, 1 H), 4.35-4.05 (m, 2 H), 3.88-3.64 (m, 4 H), 3.05-2.80 (m, |
| 2-yl)propoxy )benzamide | | | | | (Method F) | 1 H), 2.57 (s, 3 H), 2.13-1.97 (m, 2 H), 1.95-1.81 (m, 1 H), 0.96 (t, J = 7.3 Hz, 3 H) |
| (R)-2,3-difluoro-N-((4-(3- | E | H | B | Method J | m/z = 594.5 | ¹H NMR (400 MHz, DMSO-d6, ppm) δ 8.45 (d, J = 2.4 Hz, 1 H), 8.15-8.12 (m, 1 H), 7.93-7.86 |
| fluoropropyl)piperazin-1-yl)sulfonyl)-4-(1-(5- | | | | (20-80) | [M + H]+, Rt = | (m, 1 H), 7.64 (dd, J = 2.6, 8.4 Hz, 1 H), 7.50 (d, J = 8.5 Hz, 1 H), 7.37 (t, J = 7.8 Hz, 1 H), 7.20- |
| (pyridin-2-yloxy)pyridin-2- | | | | | 3.86 mins | 7.11 (m, 2 H), 6.88 (t, J = 7.9 Hz, 1 H), 5.46 (t, J = 6.1 Hz, 1 H), 4.55 (t, J = 5.6 Hz, 1 H), 4.44 (t, |
| yl)propoxy)benzamide | | | | | (Method C) | J = 5.7 Hz, 1 H), 3.23 (s, 4 H), 2.86 (s, 6 H), 2.10-1.89 (m, 4 H), 0.96 (t, J = 7.3 Hz, 3 H) |
| (R)-4-(1-(5-(cyclopropylmethoxy)pyridin-2- | C | H | B | Method J | m/z = 571.6 | ¹H NMR (400 MHz, DMSO-d6, ppm) δ 8.25-8.24 (m, 1 H), 7.38-7.29 (m, 3 H), 6.80 (t, J = 8.0 |
| yl)propoxy)-2,3-difluoro-N-((4-(3- | | | | (20-80) | [M + H]+, Rt = | Hz, 1 H), 5.35 (t, J = 6.5 Hz, 1 H), 4.55 (t, J = 5.8 Hz, 1 H), 4.43 (t, J = 5.8 Hz, 1 H), 3.86 (d, J = |
| fluoropropyl)piperazin-1- | | | | | 4.08 mins | 7.1 Hz, 2 H), 3.30-3.14 (m, 6 H), 2.84 (s, 4 H), 2.08-1.86 (m, 4 H), 1.26-1.16 (m, 1 H), 0.91 (t, |
| yl)sulfonyl)benzamide | | | | | (Method C) | J = 7.3 Hz, 3 H), 0.59-0.53 (m, 2 H), 0.34-0.29 (m, 2 H) |
| 4-((R)-1-(5-(cyclopropylmethoxy)pyridin-2- | C | C | B | Method A | m/z = 616.2 | ¹H NMR (400 MHz, DMSO-d6, ppm) δ 9.29 (s, 1 H), 8.25-8.21 (m, 1 H), 7.77-7.74 (m, 2 H), 7.40- |
| yl)propoxy)-2,3-difluoro-N-((4-(((S)-1- | | | | (30-60) | [M + H]+, Rt = | 7.25 (m, 3 H), 7.03-7.00 (m, 2 H), 6.76-6.72 (m, 1 H), 5.31 (t, J = 6.4 Hz, 1 H), 4.97-4.65 (m, 1 H), |
| methylpiperidin-3- | | | | | 1.39 mins | 3.85 (d, J = 7.2 Hz, 2 H), 3.31-2.96 (m, 4 H), 2.74 (s, 3 H), 2.13-1.61 (m, 6 H), 1.23-1.16 (m, 1 H), |
| yl)oxy)phenyl)sulfonyl)benzamide | | | | | (Method E) | 0.89 (t, J = 7.4 Hz, 3 H), 0.67-0.46 (m, 2 H), 0.33-0.28 (m, 2 H) |
| 4-((R)-1-(5-(cyclopropylmethoxy)pyridin-2- | C | C | B | Method A | m/z = 616.2 | ¹H NMR (400 MHz, DMSO-d6, ppm) δ 9.49 (s, 1 H), 8.28-8.16 (m, 1 H), 7.80-7.65 (m, 2 H), 7.37- |
| yl)propoxy)-2,3-difluoro-N-((4-(((R)-1- | | | | (30-70) | [M + H]+, Rt = | 7.26 (m, 3 H), 7.05-6.96 (m, 2 H), 6.78-6.68 (m, 1 H), 5.31 (t, J = 6.5 Hz, 1 H), 4.99-4.57 (m, 1 H), |
| methylpiperidin-3- | | | | | 1.16 mins | 3.85 (d, J = 7.1 Hz, 2 H), 3.34-2.09 (m, 4 H), 2.75 (s, 3 H), 2.06-1.66 (m, 6 H), 1.25-1.13 (m, 1 H), |
| yl)oxy)phenyl)sulfonyl)benzamide | | | | | (Method F) | 0.89 (t, J = 7.3 Hz, 3 H), 0.62-0.48 (m, 2 H), 0.36-0.23 (m, 2 H) |
| 2,3-difluoro-N-((6-methyl-3,6- | E | H | C | Method A | m/z = 560.2 | ¹H NMR (400 MHz, DMSO-d6, ppm) δ 10.28 (s, 1 H), 8.48-8.36 (m, 1 H), 8.17-8.13 (m, 1 H), |
| diazabicyclo[3.1.1]heptan-3-yl)sulfonyl)-4- | | | | (20-70) | [M + H]+, Rt = | 7.93-7.85 (m, 1 H), 7.68-7.59 (m, 1 H), 7.54-7.46 (m, 1 H), 7.43-7.34 (m, 1 H), 7.20-7.09 (m, 2 H), (m, 2 H), |
| ((R)-1-(5-(pyridin-2-yloxy)pyridin-2- | | | | | 1 mins | 6.86-6.77 (m, 1 H), 5.43 (t, J = 6.4 Hz, 1 H), 4.35-4.05 (m, 2 H), 3.88-3.64 (m, 4 H), 3.05-2.80 (m, |
| yl)propoxy)benzamide | | | | | (Method F) | 1 H), 2.57 (s, 3 H), 2.13-1.97 (m, 2 H), 1.95-1.81 (m, 1 H), 0.96 (t, J = 7.3 Hz, 3 H) |

### 4.5.31. Example 31. Preparation of 4-(1-(5-ethoxypyridin-2-yl)ethoxy)-2,3-difluoro-N-((4-methylpiperazin-1-yl)sulfonyl)benzamide

### Step 1: 4-(1-(5-ethoxypyridin-2-yl)ethoxy)-2,3-difluorobenzamide

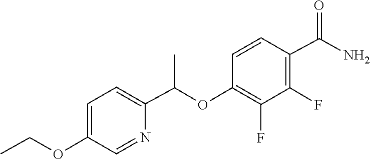 [SMILES: CCOc1ccc(C(C)Oc2ccc(C(N)=O)c(F)c2F)nc1]

4-[1-(5-Ethoxy-2-pyridyl)ethoxy]-2,3-difluoro-benzoic acid (Acid A, 2.61 g, 8.07 mmol), HATU (4.60 g, 12.1 mmol) and DIPEA (5.6 mL, 32.3 mmol) were added together in DMF (50 mL) and the resulting mixture was stirred at RT under N₂ for 20 min. NH4C₁ (864 mg, 16.1 mmol) was added and the mixture stirred for a further 18 h. The reaction mixture was diluted with sat. sodium bicarbonate solution and extracted with EtOAc (x 2). The organic layers were separated, combined, washed with H₂O (x 2) and brine, dried over Na₂SO₄ and concentrated in vacuo. The residue was purified by silica gel flash chromatography (gradient 0-100% EtOAc/cyclohexane) to give the title compound (1.58 g, 60%) as a beige solid. LCMS (Method G) MS (ES+) [M+H]⁺=323, Rt=1.44 min. ¹H NMR (300 MHz, DMSO-d6, ppm) δ 8.24 (s, 1H), 7.59 (d, J=9.3 Hz, 2H), 7.41-7.31 (m, 3H), 6.95 (t, J=7.8 Hz, 1H), 5.63 (q, J=6.0 Hz, 1H), 4.09 (q, J=6.8 Hz, 2H), 1.63 (d, J=6.1 Hz, 3H), 1.32 (t, J=6.9 Hz, 3H).

### Step 2: 4-(1-(5-ethoxypyridin-2-yl)ethoxy)-2,3-difluoro-N-((4-methylpiperazin-1-yl)sulfonyl)benzamide

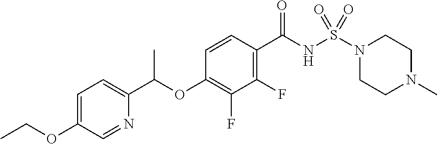 [SMILES: CCOc1ccc(C(C)Oc2ccc(C(=O)NS(=O)(=O)N3CCN(C)CC3)c(F)c2F)nc1]

To a solution of 4-(1-(5-ethoxypyridin-2-yl)ethoxy)-2,3-difluorobenzamide (50.0 mg, 0.155 mmol) and NaH (60%, 12.0 mg, 0.310 mmol) in anhydrous THF (1.5 mL) under N₂ at 0° C. was added 4-methylpiperazine-1-sulfonyl chloride (46.0 mg, 0.233 mmol). The reaction was heated to 40° C. and was stirred for 64 h. The reaction was cooled to RT, diluted with H₂O, and extracted with EtOAc (x 3). The combined organic layers were dried over Na₂SO₄ and concentrated in vacuo. The residue was purified by prep-HPLC (method H, gradient: 5-60%) to give the title compound (24.6 mg, 32%) as an off-white solid. LCMS (Method A) MS (ES+) [M+H]⁺=485.2, Rt=3.54 min. ¹H NMR (400 MHz, DMSO-d6, ppm) δ 9.64 (s, 1H), 8.25 (dd, J=1.3, 2.3 Hz, 1H), 7.42-7.34 (m, 3H), 6.83 (t, J=7.7 Hz, 1H), 5.58 (q, J=6.4 Hz, 1H), 4.12-4.06 (m, 2H), 3.27-3.16 (m, 4H), 3.06 (s, 3H), 2.69 (s, 3H), 2.31 (s, 1H), 1.62 (d, J=6.5 Hz, 3H), 1.33 (t, J=7.0 Hz, 3H).

### 4.5.32. Example 32. Preparation of 4-(1-(5-ethoxypyridin-2-yl)ethoxy)-2,3-difluoro-N-(piperidin-4-ylsulfonyl)benzamide

### Step 1: benzyl 4-[[4-[1-(5-ethoxy-2-pyridyl)ethoxy]-2,3-difluoro-benzoyl]sulfamoyl]-piperidine-1-carboxylate

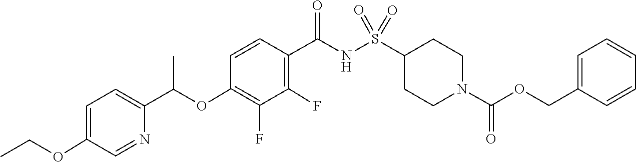 [SMILES: CCOc1ccc(C(C)Oc2ccc(C(=O)NS(=O)(=O)C3CCN(C(=O)OCc4ccccc4)CC3)c(F)c2F)nc1]

To a solution of 4-(1-(5-ethoxypyridin-2-yl)ethoxy)-2,3-difluorobenzamide (120 mg, 0.372 mmol) and NaH (60%, 30 mg, 0.745 mmol) in anhydrous THF (3 mL) under N₂ at 0° C. was added benzyl 4-chlorosulfonylpiperidine-1-carboxylate (148 mg, 0.465 mmol). The reaction was heated to 40° C. and was stirred for 16 h. The reaction was cooled to RT, diluted with H₂O, and extracted with EtOAc (x 3). The combined organic layers were dried over Na₂SO₄ and concentrated in vacuo. The residue was purified by silica gel flash chromatography (gradient 50-100% EtOAc/cyclohexane then 10% DCM/MeOH) to give the title compound (192 mg, 85%) as a yellow oil. LCMS (Method H) MS (ES+) [M+H]⁺=604.4, Rt=1.10 min.

### Step 2: 4-(1-(5-ethoxypyridin-2-yl)ethoxy)-2,3-difluoro-N-(piperidin-4-ylsulfonyl)benzamide

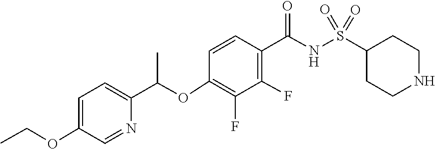 [SMILES: CCOc1ccc(C(C)Oc2ccc(C(=O)NS(=O)(=O)C3CCNCC3)c(F)c2F)nc1]

To a solution of benzyl 4-[[4-[1-(5-ethoxy-2-pyridyl)ethoxy]-2,3-difluoro-benzoyl]sulfamoyl]piperidine-1-carboxylate (130 mg, 0.215 mmol) in EtOH (12 mL) was added Pd/C (10 wt %, 173 mg, 0.163 mmol) and ammonium formate (220 mg, 3.49 mmol). The reaction was heated to 80° C. and was stirred for 2 h. The reaction was cooled to RT and was filtered through a short silica pad. The silica pad was washed with DCM. The filtrate was concentrated in vacuo and the residue was purified by silica gel flash chromatography (gradient 0-20% MeOH/DCM) then prep-HPLC (method H, gradient: 5-60%) to give the title compound (7.7 mg, 7%) as an off-white solid. LCMS (Method B) MS (ES+) [M+H]⁺=470.2, Rt=3.56 min. ¹H NMR (400 MHz, methanol-d4, ppm) δ 8.22-8.21 (m, 1H), 8.03 (s, 1H), 7.99-7.95 (m, 2H), 7.40-7.39 (m, 2H), 7.34 (s, 1H), 7.24-7.19 (m, 1H), 7.16-7.12 (m, 2H), 6.75-6.70 (m, 1H), 5.30-5.26 (m, 1H), 5.16 (s, 2H), 4.18-4.15 (m, 2H), 3.75-3.72 (m, 2H), 3.40 (s, 3H), 2.15-1.94 (m, 2H), 1.00 (t, J=7.4 Hz, 3H).

### 4.5.33. Example 33. Preparation of N-((4-(2-(1H-imidazol-1-yl)ethoxy)phenyl)sulfonyl)-4-(1-(5-ethoxypyridin-2-yl)ethoxy)-2,3-difluorobenzamide

### Step 1: N-[4-(2-chloroethoxy)phenyl]sulfonyl-4-[1-(5-ethoxy-2-pyridyl)ethoxy]-2,3-difluoro-benzamide

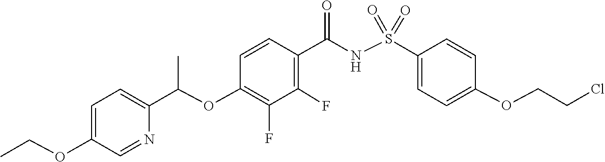 [SMILES: CCOc1ccc(C(C)Oc2ccc(C(=O)NS(=O)(=O)c3ccc(OCCCl)cc3)c(F)c2F)nc1]

To a solution of 4-[1-(5-Ethoxy-2-pyridyl)ethoxy]-2,3-difluoro-benzoic acid (Acid A, 500 mg, 1.55 mmol) in DMF (10 mL) were added 4-(2-chloroethoxy)benzenesulfonamide (547 mg, 2.32 mmol), EDCI (821 μL, 4.64 mmol) and DMAP (567 mg, 4.64 mmol). The reaction was stirred at RT for 18 h. The reaction mixture was concentrated in vacuo and the residue was purified by silica gel flash chromatography (gradient 30-100% EtOAc/cyclohexane) to give the title compound (225 mg, 27%) as a white solid. LCMS (Method G) MS (ES+) [M+H]⁺=541.2, Rt=1.83 min.

### Step 2: N-((4-(2-(1H-imidazol-1-yl)ethoxy)phenyl)sulfonyl)-4-(1-(5-ethoxypyridin-2-yl)ethoxy)-2,3-difluorobenzamide

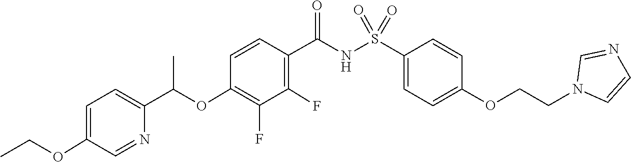 [SMILES: CCOc1ccc(C(C)Oc2ccc(C(=O)NS(=O)(=O)c3ccc(OCCn4ccnc4)cc3)c(F)c2F)nc1]

A solution of N-[4-(2-chloroethoxy)phenyl]sulfonyl-4-[1-(5-ethoxy-2-pyridyl)ethoxy]-2,3-difluoro-benzamide (30 mg, 0.056 mmol), imidazole (4.0 mg, 0.056 mmol) and Cs₂CO₃ (54 mg, 0.166 mmol) in DMF (0.5 mL) was heated to 160° C. under microwave irradiation for 1 h. The reaction was cooled to RT, diluted with H₂O, and extracted with EtOAc (x 3). The combined organic layers were dried over Na₂SO₄ and concentrated in vacuo. The residue was purified by prep-HPLC (method H, gradient: 5-60%) to give the title compound (4.1 mg, 13%) as an off-white solid. LCMS (Method B) MS (ES+) [M+H]⁺=573.2, Rt=3.31 min. ¹H NMR (400 MHz, DMSO-d6) δ 8.28-8.25 (m, 1H), 8.17-8.15 (m, 1H), 7.72-7.69 (m, 2H), 7.43 (s, 1H), 7.30-7.29 (m, 2H), 7.25-7.20 (m, 1H), 7.16 (s, 1H), 6.96-6.92 (m, 2H), 6.79-6.74 (m, 1H), 6.46 (s, 1H), 5.51 (q, J=6.4 Hz, 1H), 4.43-4.39 (m, 2H), 4.29 (t, J=5.1 Hz, 2H), 4.00 (q, J=7.0 Hz, 2H), 1.54 (d, J=6.5 Hz, 3H), 1.25 (t, J=7.0 Hz, 3H).

### 4.5.34. Example 34. Preparation of (R)-4-(1-(5-(cyclopropylmethoxy)pyridin-2-yl)propoxy)-2,3-difluoro-N-((4-(2-(oxetan-3-ylamino)ethoxy)phenyl)sulfonyl)benzamide

### Step 1: (R)—N-((4-(2-chloroethoxy)phenyl)sulfonyl)-4-(1-(5-(cyclopropylmethoxy)pyridin-2-yl)propoxy)-2,3-difluorobenzamide

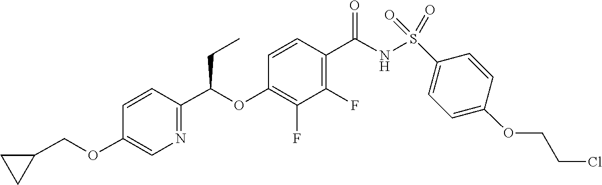 [SMILES: CC[C@@H](Oc1ccc(C(=O)NS(=O)(=O)c2ccc(OCCCl)cc2)c(F)c1F)c1ccc(OCC2CC2)cn1]

A mixture of (R)-4-(1-(5-(cyclopropylmethoxy)pyridin-2-yl)propoxy)-2,3-difluorobenzoic acid (Acid C, 220 mg, 0.61 mmol), DMAP (148 mg, 1.21 mmol), EDCI (255 mg, 1.33 mmol), and 4-(2-chloroethoxy)benzenesulfonamide (200 mg, 0.85 mmol) in DCM (10 mL) was stirred for 12 h at RT. The reaction mixture was quenched with H₂O (10 ml) and extracted with DCM (20 mL). The organic layer was washed with NaCl (aq.) (5 mL), dried over Na₂SO₄ and concentrated in vacuo. The residue was purified by flash chromatography (eluent 1:100 DCM/MeOH) to give the title compound (230 mg, 63% yield) as a white solid. LCMS (Method D) MS (ESI) [M+H]⁺=581.1, Rt=1.49 min.

### Step 2: (R)-4-(1-(5-(cyclopropylmethoxy)pyridin-2-yl)propoxy)-2,3-difluoro-N-((4-(2-(oxetan-3-ylamino)ethoxy)phenyl)sulfonyl)benzamide

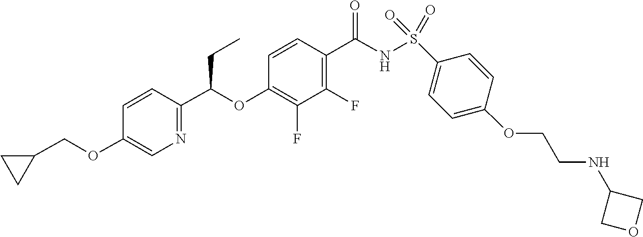 [SMILES: CC[C@@H](Oc1ccc(C(=O)NS(=O)(=O)c2ccc(OCCNC3COC3)cc2)c(F)c1F)c1ccc(OCC2CC2)cn1]

A solution of(R)—N-((4-(2-chloroethoxy)phenyl)sulfonyl)-4-(1-(5-(cyclopropylmethoxy)pyridin-2-yl)propoxy)-2,3-difluorobenzamide (60 mg, 0.10 mmol), Kl (0.9 mg, 0.0051 mmol) in oxetan-3-amine/MeCN (0.3 mL:0.3 mL) was stirred at 80° C. for 4 h. The reaction mixture was concentrated in vacuo and purified by prep-HPLC (method G, gradient: 45-55%) to give the title compound (17.3 mg, 97% purity, >99% ee, 26% yield) as a white solid. LCMS (Method E) MS (ESI) [M+H]⁺=618.2, Rt=1.21 min. ¹H NMR (400 MHz, DMSO-d6, ppm) δ 8.23 (d, J=2.3 Hz, 1H), 7.71 (d, J=8.8 Hz, 2H), 7.36-7.27 (m, 3H), 6.92 (d, J=8.8 Hz, 2H), 6.71 (t, J=8.0 Hz, 1H), 5.30 (t, J=6.4 Hz, 1H), 4.65 (t, J=6.8 Hz, 2H), 4.39 (t, J=6.2 Hz, 2H), 4.10-3.98 (m, 3H), 3.85 (d, J=7.1 Hz, 2H), 2.03-2.88 (m, 2H), 2.02-1.92 (m, 2H), 1.19-1.16 (m, 1H), 0.89 (t, J=7.4 Hz, 3H), 0.58-0.53 (m, 2H), 0.33-0.30 (m, 2H).

### 4.5.35. Example 35. Preparation of (R)—N-((4-(2-(cyclopropylamino)ethoxy)phenyl)-sulfonyl)-4-(1-(5-(cyclopropylmethoxy)pyridin-2-yl)propoxy)-2,3-difluorobenzamide

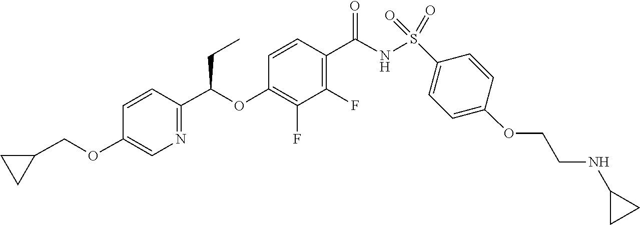 [SMILES: CC[C@@H](Oc1ccc(C(=O)NS(=O)(=O)c2ccc(OCCNC3CC3)cc2)c(F)c1F)c1ccc(OCC2CC2)cn1]

A mixture of (R)—N-((4-(2-chloroethoxy)phenyl)sulfonyl)-4-(1-(5-(cyclopropylmethoxy)pyridin-2-yl)propoxy)-2,3-difluorobenzamide (50 mg, 0.09 mmol) and Kl (1.4 mg, 0.01 mmol) in cyclopropanamine (1 mL) was stirred at 50° C. for 12 h. The reaction mixture was concentrated in vacuo and the residue was purified by prep-HPLC (method C, gradient: 35-40%) to afford the title compound (9.1 mg, 98.5% purity, >99% ee, 13% yield) as a white solid. LCMS (Method E) MS (ESI) [M+H]⁺=602.22, Rt=1.29 min. ¹H NMR (400 MHz, DMSO-d6, ppm) δ 8.73 (s, 1H), 8.23 (s, 1H), 7.76 (d, J=8.2 Hz, 2H), 7.43-7.18 (m, 3H), 7.00 (s, 2H), 6.81-6.66 (m, 1H), 5.30 (t, J=6.9 Hz, 1H), 4.38-4.20 (m, 2H), 3.85 (d, J=6.9 Hz, 2H), 3.56-3.40 (m, 2H), 2.84-2.69 (m, 1H), 2.07-1.87 (m, 2H), 1.28-1.12 (m, 1H), 0.89 (t, J=7.3 Hz, 3H), 0.81-0.71 (m, 4H), 0.61-0.51 (m, 2H), 0.36-0.26 (m, 2H).

### 4.5.36. Example 36. (R)-4-(1-(5-(cyclopropylmethoxy)pyridin-2-yl)propoxy)-2,3-difluoro-N-((4-(2-(methylamino)ethoxy)phenyl)sulfonyl)benzamide

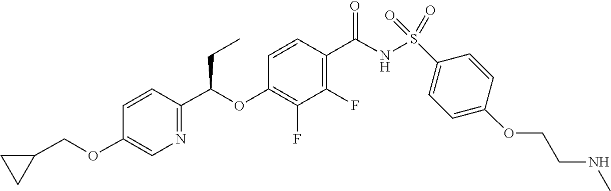 [SMILES: CC[C@@H](Oc1ccc(C(=O)NS(=O)(=O)c2ccc(OCCNC)cc2)c(F)c1F)c1ccc(OCC2CC2)cn1]

A mixture of(R)—N-((4-(2-chloroethoxy)phenyl)sulfonyl)-4-(1-(5-(cyclopropylmethoxy)pyridin-2-yl)propoxy)-2,3-difluorobenzamide (130 mg, 0.18 mmol) and Kl (59 mg, 0.36 mmol) in methylamine (40% in water, 2 mL) was stirred at 50° C. for 3 h. The solution was blown down under a nitrogen stream to a volume of ca. 1 mL. The solution was diluted with MeOH (2 mL) and was loaded onto an SCX-2 cartridge (5 g). The cartridge was washed with MeOH (2CV) and the product was eluted with 2 M ammonical MeOH. The basic fraction was concentrated in vacuo to dryness the residue was purified by prep-HPLC (method K, gradient: 40-100%) to afford the title compound (17.3 mg, >99% ee, 17% yield) as a white solid. LCMS (Method C) MS (ESI) [M+H]⁺=576.6, Rt=4.13 min. ¹H NMR (400 MHz, DMSO-d6, ppm) δ 8.45 (s, 1H), 8.23 (d, J=1.8 Hz, 1H), 7.75-7.72 (m, 2H), 7.34-7.29 (m, 3H), 6.97-6.94 (m, 2H), 6.74-6.68 (m, 1H), 5.29 (t, J=6.5 Hz, 1H), 4.22 (t, J=5.0 Hz, 2H), 3.87-3.84 (m, 2 H), 2.62 (s, 3H), 2.55-2.52 (m, 2H), 2.05-1.90 (m, 2H), 1.25-1.15 (m, 1H), 0.92-0.87 (m, 3H), 0.59-0.53 (m, 2H), 0.33-0.28 (m, 2H).

### 4.5.37. Example 37. Preparation of (R)—N-((4-((1H-imidazol-4-yl)methoxy)phenyl)sulfonyl)-2,3-difluoro-4-(1-(5-(2-methoxyethoxy)pyridin-2-yl)propoxy)benzamide

### Step 1: tert-butyl 4-[(4-sulfamoylphenoxy)methyl]imidazole-1-carboxylate

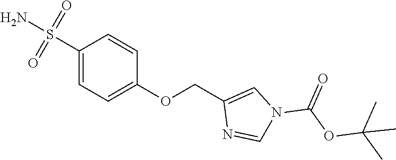 [SMILES: CC(C)(C)OC(=O)n1cnc(COc2ccc(S(N)(=O)=O)cc2)c1]

To a solution of 4-hydroxybenzenesulfonamide (255 mg, 1.47 mmol) in DMF (3.7 mL) was added K₂CO₃ (509 mg, 3.69 mmol) and the resulting mixture was stirred at RT under N₂ for 0.5 h. A solution of tert-butyl 4-(bromomethyl)imidazole-1-carboxylate (385 mg, 1.47 mmol) in DMF (3.70 mL) was added and the mixture was stirred for a further 4 h. The reaction mixture was diluted with EtOAc, filtered and the filtrate was concentrated in vacuo. The residue was triturated with DCM. The solid was removed by filtration and the filtrate was purified by silica gel flash chromatography (gradient 0-100% EtOAc/cyclohexane) to give the title compound (79 mg, 15%) as a white solid. LCMS (Method G) MS (ES+) [M−Boc+H]⁺=254.1, Rt=1.55 min.

### Step 2: tert-butyl 4-[[4-[[2,3-difluoro-4-[(1R)-1-[5-(2-methoxyethoxy)-2-pyridyl]propoxy]benzoyl]sulfamoyl]phenoxy]methyl]imidazole-1-carboxylate

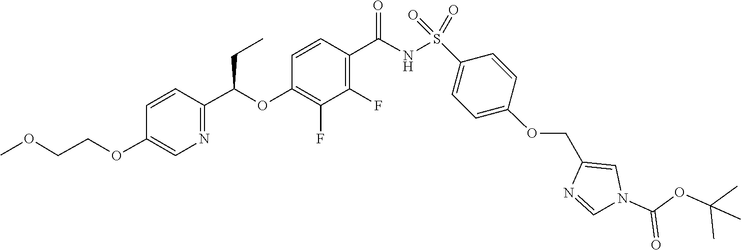 [SMILES: CC[C@@H](Oc1ccc(C(=O)NS(=O)(=O)c2ccc(OCc3cn(C(=O)OC(C)(C)C)cn3)cc2)c(F)c1F)c1ccc(OCCOC)cn1]

2,3-difluoro-4-[(1R)-1-[5-(2-methoxyethoxy)-2-pyridyl]propoxy]benzoic acid (Acid D, 45 mg, 0.12 mmol), tert-butyl 4-[(4-sulfamoylphenoxy)methyl]imidazole-1-carboxylate (65 mg, 0.18 mmol), DMAP (45 mg, 0.368 mmol) and EDCI.HCl (70 mg, 0.37 mmol) were added together in DMF (1.2 mL) and the resulting mixture was stirred at RT for 18 h. The solvent was removed in vacuo and the residue was purified by flash chromatography (gradient 0-100% EtOAc/cyclohexane) to give the title compound (48 mg, 56%) as a white solid. LCMS (Method G) MS (ES+) [M−Boc+H]⁺=603.3, Rt=1.92 min.

### Step 3: (R)—N-((4-((1H-imidazol-4-yl)methoxy)phenyl)sulfonyl)-2,3-difluoro-4-(1-(5-(2-methoxyethoxy)pyridin-2-yl)propoxy)benzamide

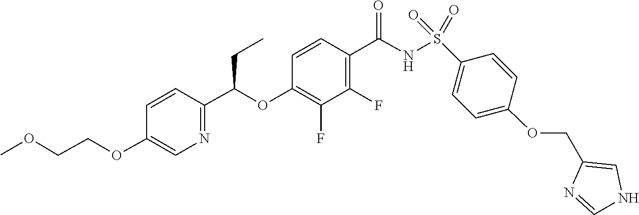 [SMILES: CC[C@@H](Oc1ccc(C(=O)NS(=O)(=O)c2ccc(OCc3c[nH]cn3)cc2)c(F)c1F)c1ccc(OCCOC)cn1]

TFA (0.25 mL) was added to a solution of tert-butyl 4-[[4-[[2,3-difluoro-4-[(1R)-1-[5-(2-methoxyethoxy)-2-pyridyl]propoxy]benzoyl]sulfamoyl]phenoxy]methyl]imidazole-1-carboxylate (48 mg, 0.068 mmol) in DCM (0.25 mL) and the resulting mixture was stirred at RT for 0.5 h. The solvent was removed in vacuo and the residue was purified by reverse phase chromatography (C18, gradient: 10-95% MeCN in H₂O [+0.1% HCOOH]) to give the title compound (30 mg, 72%) as a white amorphous solid. LCMS (Method C) MS (ES+) [M+H]⁺=603.4, Rt=3.04 min. ¹H NMR (400 MHz, methanol-d4, ppm) δ 8.22-8.21 (m, 1H), 8.03 (s, 1H), 7.99-7.95 (m, 2H), 7.40-7.39 (m, 2H), 7.34 (s, 1H), 7.24-7.19 (m, 1H), 7.16-7.12 (m, 2H), 6.75-6.70 (m, 1H), 5.30-5.26 (m, 1H), 5.16 (s, 2H), 4.18-4.15 (m, 2H), 3.75-3.72 (m, 2H), 3.40 (s, 3H), 2.15-1.94 (m, 2H), 1.00 (t, J=7.4 Hz, 3H).

### 4.5.38. Example 38. Preparation of (R)-4-(1-(5-(cyclopropylmethoxy)pyridin-2-yl)propoxy)-2,3-difluoro-N-((4-((1-(2-hydroxyethyl)piperidin-4-yl)oxy)phenyl)sulfonyl)-benzamide

### Step 1: N-({4-[(1-{2-[(tert-butyldiphenylsilyl)oxy]ethyl}piperidin-4-yl)oxy]benzene}sulfonyl)-4-[(1R)-1-[5-(cyclopropylmethoxy)pyridin-2-yl]propoxy]-2,3-difluorobenzamide

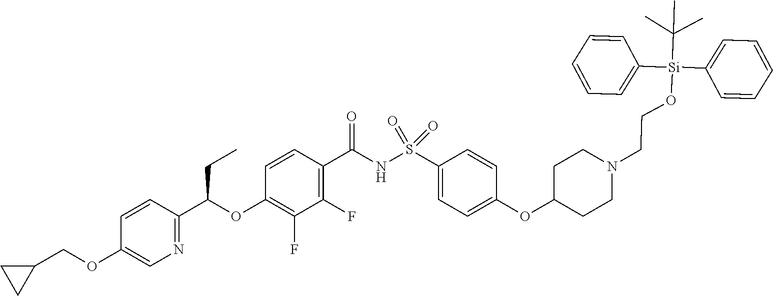 [SMILES: CC[C@@H](Oc1ccc(C(=O)NS(=O)(=O)c2ccc(OC3CCN(CCO[Si](c4ccccc4)(c4ccccc4)C(C)(C)C)CC3)cc2)c(F)c1F)c1ccc(OCC2CC2)cn1]

To a solution of (R)-4-(1-(5-(cyclopropylmethoxy)pyridin-2-yl)propoxy)-2,3-difluorobenzoic acid (Acid C, 49 mg, 0.13 mmol), EDCI (70 mg, 0.37 mmol) and DMAP (41 mg, 0.33 mmol) in DCM (1 mL) was added 4-[(1-{2-[(tert-butyldiphenylsilyl)oxy]ethyl}piperidin-4-yl)oxy]benzenesulfonamide (synthesized analogously to example procedure C, 90 mg, 0.17 mmol) at RT and the reaction was stirred for 3 h. The reaction was concentrated in vacuo and the residue was purified by flash chromatography (gradient 0-50% EtOAc/PE) to give the title compound (60 mg, 37% yield) as a colorless oil. LCMS (Method F) MS (ESI) [M+H]⁺=884.4, Rt=1.37 min.

### Step 2: (R)-4-(1-(5-(cyclopropylmethoxy)pyridin-2-yl)propoxy)-2,3-difluoro-N-((4-((1-(2-hydroxyethyl)piperidin-4-yl)oxy)phenyl)sulfonyl)benzamide

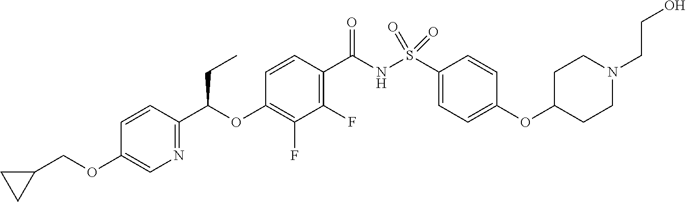 [SMILES: CC[C@@H](Oc1ccc(C(=O)NS(=O)(=O)c2ccc(OC3CCN(CCO)CC3)cc2)c(F)c1F)c1ccc(OCC2CC2)cn1]

N-({4-[(1-{2-[(tert-butyldiphenylsilyl)oxy]ethyl}piperidin-4-yl)oxy]benzene}sulfonyl)-4-[(1R)-1-[5-(cyclopropylmethoxy)pyridin-2-yl]propoxy]-2,3-difluorobenzamide (100 mg, 0.11 mmol) in THF (2 mL) was added TBAF (1 M in THF, 0.44 mL, 0.44 mmol) dropwise. The reaction mixture was stirred at RT for 2 h. The solvent was removed in vacuo and purified by prep-HPLC (method A, gradient: 35-80%) to give the title compound (34.3 mg, 98.6% purity, 46% yield, >99% ee) as a white solid. LCMS (Method F) MS (ESI) [M+H]⁺=884.4, Rt=1.37 min. ¹H NMR (400 MHz, DMSO-d6, ppm) δ 8.23 (s, 1H), 7.74 (d, J=8.7 Hz, 2H), 7.43-7.17 (m, 3H), 6.99 (d, J=8.7 Hz, 2H), 6.73 (t, J=7.9 Hz, 1H), 5.30 (t, J=6.4 Hz, 2H), 4.69 (s, 1H), 3.85 (d, J=7.1 Hz, 2H), 3.77-3.67 (m, 2H), 3.24-2.94 (m, 6H), 2.22-2.07 (m, 2H), 2.06-1.82 (m, 4H), 1.26-1.11 (m, 1H), 0.89 (t, J=7.3 Hz, 3H), 0.64-0.46 (m, 2H), 0.41-0.19 (m, 2H).

### 4.5.39. Example 39. Preparation of (R)-4-(1-(5-(cyclopropylmethoxy)pyridin-2-yl)propoxy)-2,3-difluoro-N-((4-(piperidin-4-yloxy)phenyl)sulfonyl)benzamide

### Step 1: tert-butyl 4-[4-(benzylsulfanyl)phenoxy]piperidine-1-carboxylate

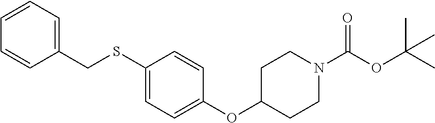 [SMILES: CC(C)(C)OC(=O)N1CCC(Oc2ccc(SCc3ccccc3)cc2)CC1]

Under N₂, to a solution of tert-butyl 4-(4-bromophenoxy)piperidine-1-carboxylate (1.0 g, 2.8 mmol) in 1,4-dioxane (10 mL) was added phenylmethanethiol (0.52 g, 4.2 mmol), Pd₂(dba)₃ (0.26 g, 0.2 mmol), Xantphos (0.32 g, 0.5 mmol) and DIPEA (0.9 g, 7.0 mmol). The mixture was stirred at 110° C. for 16 h. The solvent was removed in vacuo. The residue was purified by silica gel flash chromatography (eluent 40:1 DCM/MeOH) to give the title compound (800 mg, 64% yield) as a yellow oil. LCMS (Method F) MS (ESI) [M+Na]⁺=422.2, Rt=1.53 min.

### Step 2: tert-butyl 4-(4-sulfamoylphenoxy)piperidine-1-carboxylate

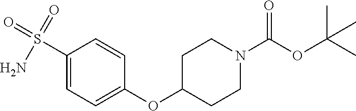 [SMILES: CC(C)(C)OC(=O)N1CCC(Oc2ccc(S(N)(=O)=O)cc2)CC1]

To a solution of tert-butyl 4-[4-(benzylsulfanyl)phenoxy]piperidine-1-carboxylate (270 mg, 0.68 mmol) in AcOH (2 mL) and H₂O (1 mL) was added NCS (360 mg, 2.70 mmol) slowly at 0° C. The reaction mixture was stirred at 0° C. for 1 h. The aforesaid reaction solution was added dropwise to NH₃—H₂O (28% w/w) (20 mL) at 0° C. The resulting solution was stirred at 0° C. for 30 min The reaction mixture was concentrated in vacuo. The residue was purified by silica gel flash chromatography (eluent 20:1 DCM/MeOH) to give the title compound (60 mg, 27% yield) as a yellow solid. LCMS (Method D) MS (ESI) [M-tBu+H]⁺=301.1, Rt=1.14 min.

### Step 3: tert-butyl 4-{4-[({4-[(1R)-1-[5-(cyclopropylmethoxy)pyridin-2-yl]propoxy]-2,3-difluorophenyl}methane)sulfonyl]phenoxy}piperidine-1-carboxylate

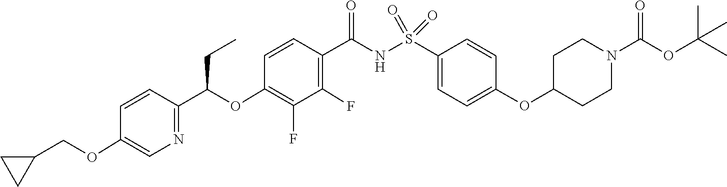 [SMILES: CC[C@@H](Oc1ccc(C(=O)NS(=O)(=O)c2ccc(OC3CCN(C(=O)OC(C)(C)C)CC3)cc2)c(F)c1F)c1ccc(OCC2CC2)cn1]

To a solution of (R)-4-(1-(5-(cyclopropylmethoxy)pyridin-2-yl)propoxy)-2,3-difluorobenzoic acid (Acid C, 150 mg, 0.41 mmol), EDCI (158 mg, 0.83 mmol) and DMAP (101 mg, 0.83 mmol) in DCM (3 mL) was added tert-butyl 4-(4-sulfamoylphenoxy)piperidine-1-carboxylate (177 mg, 0.50 mmol) at RT and the reaction was stirred for 3 h. The reaction was concentrated in vacuo and the residue was purified by silica gel flash chromatography (eluent 30:1 DCM/MeOH) to give the title compound (120 mg, 37% yield) as a white solid. LCMS (Method D) MS (ESI) [M+H]⁺=702.2, Rt=1.51 min.

### Step 4: (R)-4-(1-(5-(cyclopropylmethoxy)pyridin-2-yl)propoxy)-2,3-difluoro-N-((4-(piperidin-4-yloxy)phenyl)sulfonyl)benzamide

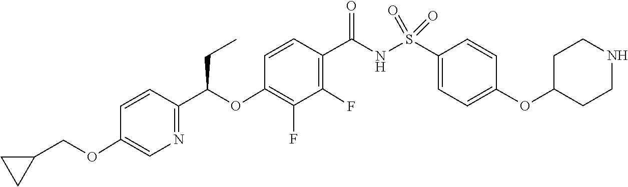 [SMILES: CC[C@@H](Oc1ccc(C(=O)NS(=O)(=O)c2ccc(OC3CCNCC3)cc2)c(F)c1F)c1ccc(OCC2CC2)cn1]

A solution of tert-butyl 4-{4-[({4-[(1R)-1-[5-(cyclopropylmethoxy)pyridin-2-yl]propoxy]-2,3-difluorophenyl}methane)sulfonyl]phenoxy}piperidine-1-carboxylate (70 mg, 0.08 mmol) in DCM/TFA=10:1 (1 mL) was stirred at RT for 2 h. The reaction was concentrated in vacuo and the residue was purified by prep-HPLC (method D, gradient: 45-95%) to give the title compound (23.9 mg, 97.1% purity, 34.7% yield, >99% ee) as a white solid. LCMS (Method D) MS (ESI) [M+H]⁺=602.2, Rt=1.11 min. ¹H NMR (400 MHz, DMSO-d6, ppm) δ 8.31 (br, 1H), 8.23 (s, 1H), 7.71 (d, J=8.7 Hz, 2H), 7.47-7.16 (m, 3H), 6.96 (d, J=8.8 Hz, 2H), 6.71 (t, J=8.0 Hz, 1H), 5.29 (t, J=6.4 Hz, 1H), 4.72-4.56 (m, 1H), 3.85 (d, J=7.1 Hz, 2H), 3.26-3.14 (m, 2H), 3.12-2.96 (m, 2H), 2.13-2.03 (m, 2H), 2.02-1.91 (m, 2H), 1.84-1.73 (m, 2H), 1.29-1.10 (m, 1H), 0.89 (t, J=7.3 Hz, 3H), 0.66-0.44 (m, 2H), 0.36-0.25 (m, 2H).

### 4.5.40. Example 40. Preparation of N-((4-(((1R,4R,5R)-2-azabicyclo[2.2.1]heptan-5-yl)oxy)phenyl)sulfonyl)-4-((R)-1-(5-(cyclopropylmethoxy)pyridin-2-yl)propoxy)-2,3-difluorobenzamide

### Step 1: (9H-fluoren-9-yl)methyl (1R,4R,5R)-5-(4-(N-(4-((R)-1-(5-(cyclopropylmethoxy)-pyridin-2-yl)propoxy)-2,3-difluorobenzoyl)sulfamoyl)phenoxy)-2-azabicyclo[2.2.1]heptane-2-carboxylate

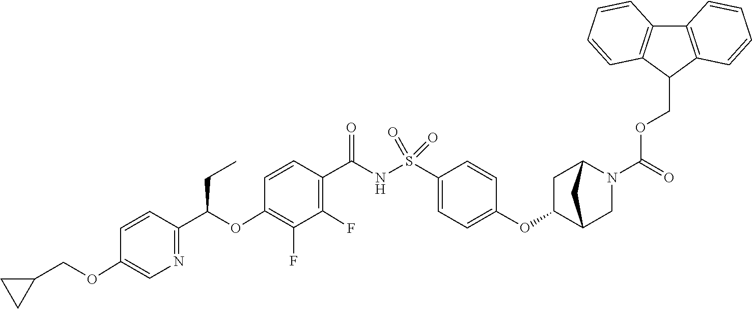 [SMILES: CC[C@@H](Oc1ccc(C(=O)NS(=O)(=O)c2ccc(O[C@@H]3C[C@H]4C[C@@H]3CN4C(=O)OCC3c4ccccc4-c4ccccc43)cc2)c(F)c1F)c1ccc(OCC2CC2)cn1]

To a solution of (R)-4-(1-(5-(cyclopropylmethoxy)pyridin-2-yl)propoxy)-2,3-difluorobenzoic acid (Acid C, 35 mg, 0.10 mmol) and CMPI (38 mg, 0.14 mmol) in DMF (2 mL) was added DMAP (3.5 mg, 0.03 mmol) and the mixture was stirred at RT for 20 min Then, 9H-fluoren-9-ylmethyl (1R,4R,5R)-5-(4-sulfamoylphenoxy)-2-azabicyclo[2.2.1]heptane-2-carboxylate (synthesized using example procedure D, 56 mg, 0.12 mmol) and NEt3 (29 mg, 0.29 mmol) were added. The reaction mixture was stirred at RT for 1 h. The solvent was concentrated in vacuo. The residue was purified by prep-TLC (eluent 20:1 DCM/MeOH) to give the title compound (30 mg, 30% yield) as a yellow oil. LCMS (Method D) MS (ESI) [M+H]⁺=836.3, Rt=1.73 min.

### Step 2: N-((4-(((1R,4R,5R)-2-azabicyclo[2.2.1]heptan-5-yl)oxy)phenyl)sulfonyl)-4-((R)-1-(5-(cyclopropylmethoxy)pyridin-2-yl)propoxy)-2,3-difluorobenzamide

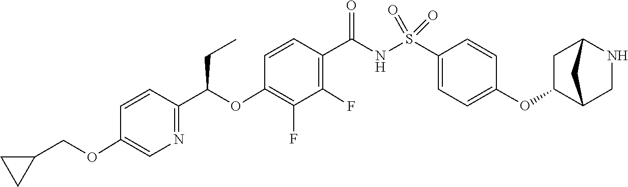 [SMILES: CC[C@@H](Oc1ccc(C(=O)NS(=O)(=O)c2ccc(O[C@@H]3C[C@H]4C[C@@H]3CN4)cc2)c(F)c1F)c1ccc(OCC2CC2)cn1]

A solution of 9H-fluoren-9-ylmethyl (1R,4R,5R)-5-{4-[({4-[(1R)-1-[5-(cyclopropylmethoxy)pyridin-2-yl]propoxy]-2,3-difluorophenyl}methane)sulfonyl]phenoxy}-2-azabicyclo[2.2.1]heptane-2-carboxylate (30 mg, 0.04 mmol) in DCM (1.2 mL) and Et₂NH (0.6 mL) was stirred at RT for 2 h. The reaction was concentrated in vacuo and the residue was purified by prep-HPLC (method D, gradient: 35-65%) to give the title compound (7.4 mg, 98% purity, 33% yield) as a white solid. LCMS (Method E) MS (ESI) [M+H]⁺=614.4, Rt=1.43 min. ¹H NMR (400 MHz, DMSO-d6, ppm) δ 8.23 (s, 1H), 7.71 (d, J=8.8 Hz, 2H), 7.33-7.30 (m, 3H), 6.90 (d, J=8.8 Hz, 2H), 6.71 (t, J=8.0 Hz, 1H), 5.31-5.26 (m, 1H), 4.89-4.86 (m, 1H), 3.97 (s, 1H), 3.85 (d, J=7.1 Hz, 2H), 3.44-3.35 (m, 1H), 2.99-2.94 (m, 2H), 2.34-2.32 (m, 1H), 2.07-1.87 (m, 2H), 1.84-1.77 (m, 1H), 1.73-1.67 (m, 1H), 1.60-1.57 (m, 1H), 1.50-1.48 (m, 1H), 1.23-1.19 (m, 1H), 0.89 (t, J=7.3 Hz, 3H), 0.59-0.50 (m, J=8.0, 1.7 Hz, 2H), 0.33-0.26 (m, 2H).

### 4.5.41. Example 41. Preparation of (R)—N-((4-((1-azabicyclo[2.2.1]heptan-4-yl)oxy)phenyl)sulfonyl)-4-(1-(5-(cyclopropylmethoxy)pyridin-2-yl)propoxy)-2,3-difluorobenzamide

### Step 1: 4-((1-azabicyclo[2.2.1]heptan-4-yl)oxy)-3-nitrobenzenesulfonamide

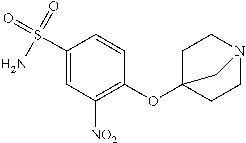 [SMILES: NS(=O)(=O)c1ccc(OC23CCN(CC2)C3)c([N+](=O)[O-])c1]

To a solution of 1-azabicyclo[2.2.1]heptan-4-ol (p-toluenesulfonate salt) (1.0 g, 3.5 mmol) in dry DMF (20 mL) was added NaH (60% in oil, 0.28 g, 7.0 mmol) at 0° C. under an atmosphere of N₂, and the reaction was stirred at this temperature for 30 min. 4-Fluoro-3-nitrobenzenesulfonamide (0.770 g, 3.5 mmol) was added and the resulting solution was slowly warmed to RT and stirred for 2 h. The reaction mixture was quenched with NH₄Cl (aq.) (10 mL) and extracted with EtOAc (10 mL×3). The combined organic phases were washed with brine (5 mL), dried over Na₂SO₄ and then concentrated in vacuo. The residue was purified by silica gel flash chromatography (gradient 0-40% EtOAc/PE) to give the title compound (600 mg, 55% yield) as a white solid. LCMS (Method E) MS (ESI) [M+H]⁺=314.1, Rt=1.05 min.

### Step 2: (R)—N-((4-((1-azabicyclo[2.2.1]heptan-4-yl)oxy)-3-nitrophenyl)sulfonyl)-4-(1-(5-(cyclopropylmethoxy)pyridin-2-yl)propoxy)-2,3-difluorobenzamide

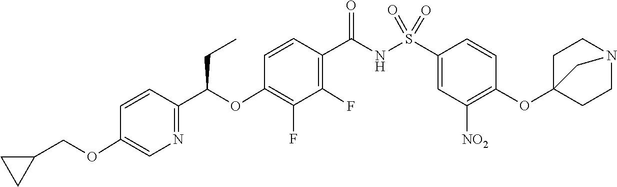 [SMILES: CC[C@@H](Oc1ccc(C(=O)NS(=O)(=O)c2ccc(OC34CCN(CC3)C4)c([N+](=O)[O-])c2)c(F)c1F)c1ccc(OCC2CC2)cn1]

A mixture of (R)-4-(1-(5-(cyclopropylmethoxy)pyridin-2-yl)propoxy)-2,3-difluorobenzoic acid (Acid C, 600 mg, 1.65 mmol), CMPI (614 mg, 2.40 mmol) and DMAP (20 mg, 0.16 mmol) in DCM (10 mL) was stirred at RT for 30 min. 4-((1-Azabicyclo[2.2.1]heptan-4-yl)oxy)-3-nitrobenzenesulfonamide (517 mg, 1.65 mmol) and NEt₃ (0.688 mL, 4.95 mmol) were added. The resulting solution was stirred for 2 h at 25° C. The reaction mixture was quenched with NH4C₁ (aq.) (10 mL) and extracted with EtOAc (10 mL×3). The combined organic phases were washed with brine (10 mL), dried over Na₂SO₄, and concentrated in vacuo. The residue was purified by silica gel flash chromatography (gradient 0-10% MeOH/DCM) to give the tile compound (500 mg, 46% yield) as a yellow solid. LCMS (Method F) MS (ESI) [M+H]⁺=659.3, Rt=1.04 min.

### Step 3: (R)—N-((4-((1-azabicyclo[2.2.1]heptan-4-yl)oxy)-3-aminophenyl)sulfonyl)-4-(1-(5-(cyclopropylmethoxy)pyridin-2-yl)propoxy)-2,3-difluorobenzamide

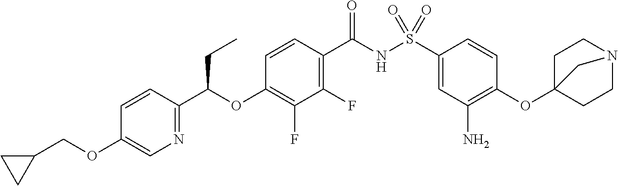 [SMILES: CC[C@@H](Oc1ccc(C(=O)NS(=O)(=O)c2ccc(OC34CCN(CC3)C4)c(N)c2)c(F)c1F)c1ccc(OCC2CC2)cn1]

To a stirred solution of (R)—N-((4-((1-azabicyclo[2.2.1]heptan-4-yl)oxy)-3-nitrophenyl)sulfonyl)-4-(1-(5-(cyclopropylmethoxy)pyridin-2-yl)propoxy)-2,3-difluorobenzamide (500 mg, 0.76 mmol) in MeOH (10 mL) was added Pd/C (50 mg, 10% wt/wt) at RT under N₂ atmosphere. The resulting atmosphere was evacuated under vacuum and purged with H₂ several times. The mixture was stirred for 16 h at RT under H₂. After completion, the mixture was filtered. The filtrate was concentrated in vacuo to give the title compound (300 mg, 63% yield) as a yellow solid. The crude product was used directly in the next step without further purification. LCMS (Method D) MS (ESI) [M+H]⁺=629.2, Rt=1.10 min.

### Step 4: (R)—N-((4-((1-azabicyclo[2.2.1]heptan-4-yl)oxy)phenyl)sulfonyl)-4-(1-(5-(cyclopropylmethoxy)pyridin-2-yl)propoxy)-2,3-difluorobenzamide

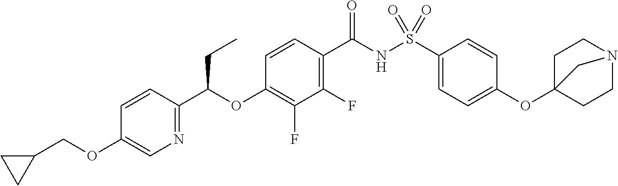 [SMILES: CC[C@@H](Oc1ccc(C(=O)NS(=O)(=O)c2ccc(OC34CCN(CC3)C4)cc2)c(F)c1F)c1ccc(OCC2CC2)cn1]

To a solution of(R)—N-((4-((1-azabicyclo[2.2.1]heptan-4-yl)oxy)-3-aminophenyl)sulfonyl)-4-(1-(5-(cyclopropylmethoxy)pyridin-2-yl)propoxy)-2,3-difluorobenzamide (150 mg, 0.45 mmol) in THF (5 mL) was added t-BuONO (0.267 mL, 2.25 mmol) dropwise at 0° C. under N₂. The mixture was then heated at 60° C. for 2 h. The solvent was removed in vacuo. The residue was purified by Prep-HPLC (method D, gradient: 45-95%) to give the title compound (6.4 mg, 91.6% purity, 2.3% yield) as a yellow solid. LCMS (Method E) MS (ESI) [M+H]⁺=614.5, Rt=1.37 min. ¹H NMR (400 MHz, DMSO-d6, ppm) δ 8.22 (s, 1H), 7.80 (d, J 8.6 Hz, 2H), 7.37-7.24 (m, 3H), 7.06 (d, J 8.7 Hz, 2H), 6.72 (t, J 8.2 Hz, 1H), 5.27 (t, J 6.3 Hz, 1H), 3.88 (d, J 6.9 Hz, 2H), 3.53-3.50 (m, 2H), 3.44-3.27 (m, 4H), 2.21-1.90 (m, 6H), 1.25-1.19 (m, 1H), 0.90 (t, J 7.3 Hz, 3H), 0.61-0.41 (m, 2H), 0.37-0.23 (m, 2H).

### 4.5.42. Example 42. Preparation of (R)-4-(1-(5-(cyclopropylmethoxy)pyridin-2-yl)propoxy)-2,3-difluoro-N-((6-((1-methylpiperidin-4-yl)oxy)-2-azaspiro[3,3]heptan-2-yl)sulfonyl)benzamide

### Step 1: tert-butyl 6-(pyridin-4-yloxy)-2-azaspiro[3,3]heptane-2-carboxylate

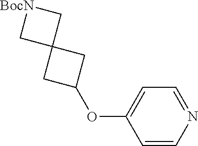 [SMILES: CC(C)(C)OC(=O)N1CC2(CC(Oc3ccncc3)C2)C1]

To a stirred solution of tert-butyl 6-hydroxy-2-azaspiro[3,3]heptane-2-carboxylate (19.0 g, 89.1 mmol) and pyridin-4-ol (8.47 g, 89.1 mmol) in THF (200 mL) was added PPh₃ (30.4 g, 116 mmol) and DIAD (23.42 g, 115.8 mmol). The mixture was stirred at 60° C. for 12 h. The solvent was removed in vacuo. The residue was purified by silica gel flash chromatography (gradient 0-10% MeOH/DCM 0-10%) to give the title compound (17 g, 59% yield) as a yellow solid. LCMS (Method D) MS (ESI) [M+H]⁺=291.4, Rt=0.86 min.

### Step 2: 1-benzyl-4-((2-(tert-butoxycarbonyl)-2-azaspiro[3,3]heptan-6-yl)oxy)pyridin-1-1 μm bromide

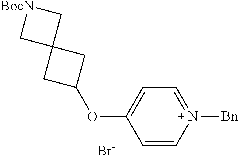 [SMILES: CC(C)(C)OC(=O)N1CC2(CC(Oc3cc[n+](Cc4ccccc4)cc3)C2)C1 ; [Br-]]

A mixture of tert-butyl 6-(pyridin-4-yloxy)-2-azaspiro[3,3]heptane-2-carboxylate (17.0 g, 58.5 mmol) and BnBr (11.0 g, 64.4 mmol) in MeCN (170 mL) was stirred at 80° C. for 16 h. The reaction mixture was cooled to RT. The precipitate was collected by filtration to give the title compound (10 g, 33% yield) as a white solid, which was used directly in the next step without further purification. LCMS (Method D) MS (ESI) [M+H]⁺=461.1, Rt=1.06 min.

### Step 3: tert-butyl 6-((1-benzyl-1,2,3,6-tetrahydropyridin-4-yl)oxy)-2-azaspiro[3,3]heptane-2-carboxylate

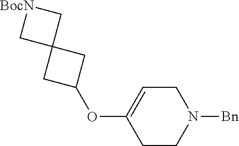 [SMILES: CC(C)(C)OC(=O)N1CC2(CC(OC3=CCN(Cc4ccccc4)CC3)C2)C1]

Under N₂ atmosphere, to a solution of 1-benzyl-4-((2-(tert-butoxycarbonyl)-2-azaspiro[3,3]heptan-6-yl)oxy)pyridin-1-1 μm bromide (10 g, 21.6 mmol) in MeOH (100 mL) was added NaBH₄ (4.9 g, 129.6 mmol) and the reaction mixture was stirred at RT for 12 h. The solvent was removed in vacuo. The residue was purified by silica gel flash chromatography (gradient 0-2% MeOH/DCM) to give the title compound (6 g, 68% yield) as a white solid. ¹H NMR (400 MHz, DMSO-d6, ppm) δ 7.36-7.18 (m, 5H), 4.44 (s, 1H), 4.28-4.20 (m, 1H), 3.87-3.72 (m, 4H), 3.51 (s, 2H), 2.87 (s, 2H), 2.58-2.51 (m, 4H), 2.12-1.95 (m, 4H), 1.35 (s, 9H).

### Step 4: tert-butyl 6-(piperidin-4-yloxy)-2-azaspiro[3,3]heptane-2-carboxylate

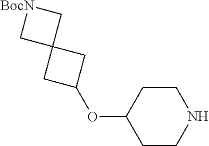 [SMILES: CC(C)(C)OC(=O)N1CC2(CC(OC3CCNCC3)C2)C1]

Under H₂ atmosphere, a mixture of tert-butyl 6-{[1-(1-methylphenyl)-3,6-dihydro-2H-pyridin-4-yl]oxy}-2-azaspiro[3,3]heptane-2-carboxylate (4.5 g, 11.7 mmol), 1,1,2-trichloroethane (1.71 g, 12.8 mmol) and 10% Pd/C (1.24 g, 11.7 mmol) in THF/EtOH (25 mL/17.5 mL) was stirred at RT for 24 h. Then the mixture was filtered through celite. The filtrate was concentrated in vacuo to give the title compound (3.15 g, 82% yield) as a white solid, which was used directly in next step without further purification. LCMS (Method D) MS (ESI) [M+H]⁺=297.4, Rt=1.38 min.

### Step 5: tert-butyl 6-((1-methylpiperidin-4-yl)oxy)-2-azaspiro[3,3]heptane-2-carboxylate

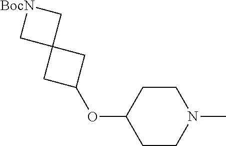 [SMILES: CN1CCC(OC2CC3(C2)CN(C(=O)OC(C)(C)C)C3)CC1]

Under N₂ atmosphere, to a mixture of tert-butyl 6-(piperidin-4-yloxy)-2-azaspiro[3,3]heptane-2-carboxylate (3 g, 10.1 mmol) and paraformaldehyde (1.57 g, 25.3 mmol) in CF₃CH₂OH (30 mL) was added NaBH₄ (1.15 g, 30.3 mmol) and the resulting mixture was stirred at 78° C. for 2 h. The solvent was removed in vacuo. The residue was purified by silica gel flash chromatography (gradient 0-25% MeOH/DCM) to give the title compound (3 g, 86% yield) as a yellow oil. LCMS (Method D) MS (ESI) [M+H]⁺=311.4, Rt=1.60 min.

### Step 6: 6-((1-methylpiperidin-4-yl)oxy)-2-azaspiro[3,3]heptane

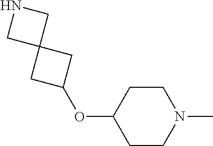

To a solution of tert-butyl 4-(4-{1-[(tert-butoxy)carbonyl]azetidin-3-yl}phenoxy)pyrazole-1-carboxylate (3 g, 9.7 mmol) in dry DCM (30 mL) was added TFA (6 mL) and the reaction was stirred at RT for 1 h. The solvent was removed in vacuo. The residue was purified by silica gel flash chromatography (gradient 0-25% MeOH/DCM with 0.4% NH₃·H₂O) to give the title compound (2 g, 89% yield) as a yellow oil. LCMS (Method D) MS (ESI) [M+H]⁺=211.3, Rt=0.16 min.

### Step 7: (tert-butoxycarbonyl)((4-(dimethyliminio)pyridin-1(4H)-yl)sulfonyl)amide

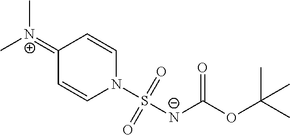 [SMILES: C[N+](C)=c1ccn(S(=O)(=O)[N-]C(=O)OC(C)(C)C)cc1]

To a cooled solution of 2-methylpropan-2-ol (2.73 g, 36.8 mmol) in DCM (80 mL) was added chlorosulfonyl isocyanate (5.21 g, 36.8 mmol) at 0° C. DMAP (8.99 g, 73.6 mmol) was added, and the reaction was stirred at RT for 1 h. The reaction mixture was washed with H₂O (10 mL×3), dried, filtered and concentrated in vacuo to give the title compound (12 g, 97% yield) as a white solid. LCMS (Method E) MS (ESI) [M+H]⁺=302.3, Rt=1.12 min.

### Step 8: tert-butyl((6-((1-methylpiperidin-4-yl)oxy)-2-azaspiro[3,3]heptan-2-yl)sulfonyl)carbamate

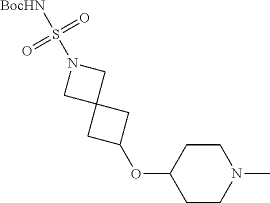 [SMILES: CN1CCC(OC2CC3(C2)CN(S(=O)(=O)NC(=O)OC(C)(C)C)C3)CC1]

To a solution of 6-((1-methylpiperidin-4-yl)oxy)-2-azaspiro[3,3]heptane (1.8 g, 5.4 mmol) and NEt₃ (1.64 g, 16.2 mmol) in dry DCM (20 mL) was added (tert-butoxycarbonyl)((4-(dimethyliminio)pyridin-1(4H)-yl)sulfonyl)amide (3.25 g, 5.4 mmol) at 0° C. and the reaction was stirred at RT for 2 h. The mixture was concentrated in vacuo. The residue was purified by silica gel flash chromatography (gradient 0-20% MeOH/DCM) to give the title compound (0.6 g, 20% yield) as a white solid. LCMS (Method D) MS (ESI) [M+H]⁺=390.5, Rt=0.71 min.

### Step 9: 6-((1-methylpiperidin-4-yl)oxy)-2-azaspiro[3,3]heptane-2-sulfonamide

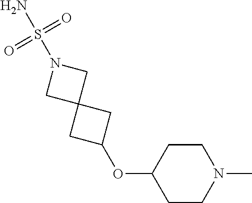 [SMILES: CN1CCC(OC2CC3(C2)CN(S(N)(=O)=O)C3)CC1]

To a solution of tert-butyl((6-((1-methylpiperidin-4-yl)oxy)-2-azaspiro[3,3]heptan-2-yl)sulfonyl)carbamate (400 mg, 1.02 mmol) in dry DCM (4 mL) was added TFA (0.8 mL) and the reaction was stirred at RT for 1 h. The reaction mixture was concentrated in vacuo and lyophilized to give the title compound (400 mg, 94% yield) as a yellow oil. LCMS (Method E) MS (ESI) [M+H]⁺=290.4, Rt=0.3 min.

### Step 10: (R)-4-(1-(5-(cyclopropylmethoxy)pyridin-2-yl)propoxy)-2,3-difluoro-N-((6-((1-methylpiperidin-4-yl)oxy)-2-azaspiro[3,3]heptan-2-yl)sulfonyl)benzamide

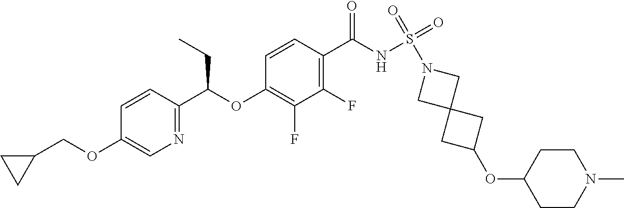 [SMILES: CC[C@@H](Oc1ccc(C(=O)NS(=O)(=O)N2CC3(CC(OC4CCN(C)CC4)C3)C2)c(F)c1F)c1ccc(OCC2CC2)cn1]

A solution of4-[(1R)-1-[5-(cyclopropylmethoxy)pyridin-2-yl]propoxy]-2,3-difluorobenzoic acid (Acid C, 200 mg, 0.55 mmol), CMPI (211 mg, 0.83 mmol) and DMAP (20 mg, 0.17 mmol) in DMF (4 mL) was stirred at RT for 20 min. 6-[(1-methylpiperidin-4-yl)oxy]-2-azaspiro[3,3]heptane-2-sulfonamide (400 mg, 0.97 mmol) and NEt₃ (223 mg, 2.20 mmol) were added. The reaction mixture was stirred at RT for 2 h. The mixture was purified by C18 chromatography (gradient 50-60% MeCN/H₂O) to afford the title compound (67.3 mg, 96.5% purity, 100% ee, 19% yield) as a white solid. LCMS (Method D) MS (ESI) [M+H]⁺=635.7, Rt=1.10 min. ¹H NMR (400 MHz, DMSO-d6, ppm) δ 10.35 (s, 1H), 8.26 (s, 1H), 7.42-7.25 (m, 3H), 6.90 (t, J=7.9 Hz, 1H), 5.39 (t, J=6.4 Hz, 1H), 4.00-3.80 (m, 7H), 3.66-3.52 (m, 1H), 3.20-2.88 (m, 4H), 2.69 (s, 3H), 2.43-2.36 (m, 2H), 2.04-1.95 (m, 4H), 1.92-1.50 (m, 4H), 1.22-1.17 (m, 1H), 0.91 (t, J=7.3 Hz, 3H), 0.65-0.53 (m, 2H), 0.36-0.28 (m, 2H).

### 4.5.43. Example 43. Preparation of (R)-4-(1-(5-(cyclopropylmethoxy)pyridin-2-yl)propoxy)-2,3-difluoro-N-((3-((1-methylpiperidin-4-yl)oxy)azetidin-1-yl)sulfonyl)benzamide

### Step 1: tert-butyl 3-(pyridin-4-yloxy)azetidine-1-carboxylate

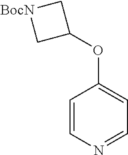 [SMILES: CC(C)(C)OC(=O)N1CC(Oc2ccncc2)C1]

To a solution of 4-chloropyridine hydrochloride (21.7 g, 0.14 mol) in DMSO (250 mL) was added tert-butyl 3-hydroxyazetidine-1-carboxylate (25.0 g, 0.14 mol) and t-BuONa (38.8 g, 0.41 mol). The mixture was stirred at 80° C. for 16 h. The solvent was removed under vacuum. The residue was purified by silica gel column chromatography (eluting with EtOAc/PE, 0% to 50%) to give the title compound (20.0 g, 90% purity, 50% yield) as a yellow oil. LCMS (Method F) MS (ESI) [M+H]⁺=250.95, Rt=0.74 min. ¹H NMR (400 MHz, DMSO-d6, ppm) δ 8.42-8.40 (m, 2H), 6.88-6.86 (m, 2H), 5.20-4.97 (m, 1H), 4.40-4.25 (m, 2H), 3.83-3.79 (m, 2H), 1.39 (s, 9H).

### Step 2: tert-butyl 3-(piperidin-4-yloxy)azetidine-1-carboxylate

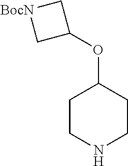 [SMILES: CC(C)(C)OC(=O)N1CC(OC2CCNCC2)C1]

To a solution of tert-butyl 3-(pyridin-4-yloxy)azetidine-1-carboxylate (18.0 g, 71.9 mmol) in AcOH (200 mL) was added PtO2 (3.6 g) in a 500-mL pressure vessel. The reaction mixture was stirred at 40° C. for 48 hours under 3 atmosphere H₂. The mixture was filtered through celite and concentrated under vacuum to give the title compound (14 g, 95% purity, 76% yield) as a yellow oil, which was used directly in next step without further purification. LCMS (Method F) MS (ESI) [M+H]⁺=257.3, Rt=3.53 min. ¹H NMR (400 MHz, DMSO-d6, ppm) δ 4.41-4.33 (m, 1H), 4.10-3.91 (m, 2H), 3.73-3.48 (m, 3H), 3.17-3.03 (m, 2H), 2.94-2.73 (m, 2H), 1.88-1.87 (m, 2H), 1.68-1.52 (m, 2H), 1.37 (s, 9H).

### Step 3: tert-butyl 3-((1-methylpiperidin-4-yl)oxy)azetidine-1-carboxylate

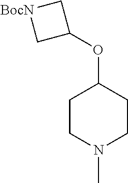 [SMILES: CN1CCC(OC2CN(C(=O)OC(C)(C)C)C2)CC1]

A mixture of tert-butyl 3-(piperidin-4-yloxy)azetidine-1-carboxylate (4.0 g, 15.60 mmol), Paraformaldehyde (1.17 g, 39 mmol) and HCOOH (2.94 mL, 78.0 mmol) in dioxane (50 mL) was stirred at 80° C. for 1 h. The solvent was removed under vacuum. The residue was purified by silica gel chromatography (eluting with MeOH/DCM, 0% to 10%) to give the title compound (3.0 g, 95% purity, 71% yield) as a colorless oil. LCMS (Method F) MS (ESI) [M+H]⁺=271.2, Rt=0.87 min. ¹H NMR (400 MHz, DMSO-d6, ppm) δ 4.41-4.27 (m, 1H), 4.02 (t, J 7.6 Hz, 2H), 3.66-3.64 (m, 2H), 3.54-3.41 (m, 1H), 3.01-3.00 (m, 2H), 2.70-2.69 (m, 2H), 2.51-2.49 (m, 3H), 1.97-1.79 (m, 2H), 1.64-1.62 (m, 2H), 1.37 (s, 9H).

### Step 4: tert-butyl 4-(azetidin-3-yloxy)-1-methylpiperidine

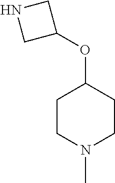

To a solution of tert-butyl 3-((1-methylpiperidin-4-yl)oxy)azetidine-1-carboxylate (3.0 g, 11.1 mmol) in DCM (30 mL) was added TFA (6 mL). The mixture was stirred for 3 hours at RT. The mixture was concentrated under vacuum to afford the title compound (1.5 g, 79% yield) and was used in the next reaction without any further purification. LCMS (Method F) MS (ESI) [M+H]⁺=171.2, Rt=0.21 min.

### Step 5: benzyl((3-((1-methylpiperidin-4-yl)oxy)azetidin-1-yl)sulfonyl)carbamate

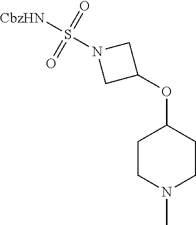 [SMILES: CN1CCC(OC2CN(S(=O)(=O)NC(=O)OCc3ccccc3)C2)CC1]

To a solution of tert-butyl 4-(azetidin-3-yloxy)-1-methylpiperidine (2.9 g, 17.0 mmol) and NEt₃ (7.10 mL, 51.12 mmol) in dry DCM (50 mL) was added ((benzyloxy)carbonyl)((4-(dimethyliminio)pyridin-1(4H)-yl)sulfonyl)amide (6.85 g, 20.5 mmol) at 0° C. and the reaction was stirred at RT for 2 h. The mixture was concentrated in vacuo. The residue was purified by reverse-phas column chromatography (C18 column, eluting with 10% to 90% MeCN/H₂O, containing 0.1% HCOOH) to afford the title compound (1.5 g, 80% purity, 18% yield) as an off-white solid. LCMS (Method F) MS (ESI) [M+H]⁺=384.2, Rt=0.69 min. ¹H NMR (400 MHz, DMSO-d6, ppm) δ 7.42-7.35 (m, 5H), 5.19 (s, 2H), 4.39-4.33 (m, 1H), 4.18-4.11 (m, 2H), 3.90-3.82 (m, 2H), 3.52-3.42 (m, 2H), 3.26-3.213 (m, 1H), 3.07-2.83 (m, 2H), 2.76 (s, 3H), 2.10-1.94 (m, 1H), 1.92-1.75 (m, 2H), 1.59-1.51 (m, 1H).

### Step 6: 3-((1-methylpiperidin-4-yl)oxy)azetidine-1-sulfonamide

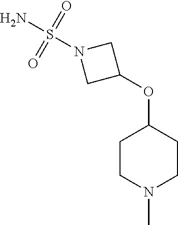 [SMILES: CN1CCC(OC2CN(S(N)(=O)=O)C2)CC1]

To a stirred solution of benzyl((3-((1-methylpiperidin-4-yl)oxy)azetidin-1-yl)sulfonyl)carbamate (1.4 g, 3.65 mmol) in MeOH (20 mL) was added Pd/C (150 mg, 10% wt/wt) at RT under a nitrogen atmosphere. The resulting solution was evacuated under vacuum and purged with H₂ several times. The mixture stirred for 16 hours at RT under H₂ (1 atm). After completion, the mixture was filtered. The filtrate was concentrated under reduced pressure to give the title compound (0.7 g, 80% purity, 62% yield) as an off-white solid. LCMS (Method F) MS (ESI) [M+H]⁺=250.1, Rt=0.21 min. ¹H NMR (400 MHz, MeOD-d, ppm) δ 4.42-4.36 (m, 1H), 3.98-3.93 (m, 2H), 3.70-3.65 (m, 2H), 3.55-3.51 (m, 1H), 3.36-3.30 (m, 2H), 3.22-3.18 (m, 1H), 3.06-3.00 (m, 1H), 2.86 (s, 3H), 2.19-2.16 (m, 1H), 2.05-1.92 (m, 2H), 1.77-1.66 (m, 1H).

### Step 7: (R)-4-(1-(5-(cyclopropylmethoxy)pyridin-2-yl)propoxy)-2,3-difluoro-N-((3-((1-methylpiperidin-4-yl)oxy)azetidin-1-yl)sulfonyl)benzamide

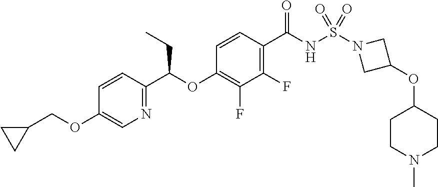 [SMILES: CC[C@@H](Oc1ccc(C(=O)NS(=O)(=O)N2CC(OC3CCN(C)CC3)C2)c(F)c1F)c1ccc(OCC2CC2)cn1]

To a solution of (R)-4-(1-(5-(cyclopropylmethoxy)pyridin-2-yl)propoxy)-2,3-difluorobenzoic acid (Acid C, 300 mg, 0.83 mmol) in DCM (5 mL) was added DMAP (10.1 mg, 0.083 mmol), CMPI (319 mg, 1.25 mmol) at RT and the mixture was stirred for 30 mins. 3-((1-methylpiperidin-4-yl)oxy)azetidine-1-sulfonamide (413.52 mg, 1.18 mmol) was added and the mixture was stirred at RT for 10 mins. Et₃N (252 mg, 2.49 mmol) was added and the mixture was stirred for 1 hour at RT. The mixture was concentrated to dryness and the residue was purified by Prep-HPLC (method A, gradient: 20-80) to give the title compound (240 mg, 99.4% purity, >99% ee, 40% yield) as a yellow solid.

LCMS (Method F) MS (ESI) [M+H]⁺=595.3, Rt=1.06 min. ¹H NMR (400 MHz, DMSO-d₆, ppm) δ 9.39 (s, 1H), 8.26-8.24 (m, 1H), 7.40-7.29 (m, 3H), 6.75 (t, J 8.2 Hz, 1H), 5.32 (t, J 6.4 Hz, 1H), 4.30-4.11 (m, 1H), 3.86 (d, J 7.1 Hz, 2H), 3.80-3.77 (m, 2H), 3.69-3.59 (m, 2H), 3.58-3.48 (m, 1H), 3.18-3.06 (m, 4H), 2.73 (s, 3H), 2.06-1.68 (m, 6H), 1.24-1.18 (m, 1H), 0.91 (t, J 7.3 Hz, 3H), 0.62-0.47 (m, 2H), 0.37-0.20 (m, 2H).

### 4.5.44. Example 44. (R)-2,3-difluoro-N-((3-((1-methylpiperidin-4-yl)oxy)azetidin-1-yl)sulfonyl)-4-(1-(5-(pyridin-2-yloxy)pyridin-2-yl)propoxy)benzamide

### Step 1: (R)-2,3-difluoro-N-((3-((1-methylpiperidin-4-yl)oxy)azetidin-1-yl)sulfonyl)-4-(1-(5-(pyridin-2-yloxy)pyridin-2-yl)propoxy)benzamide

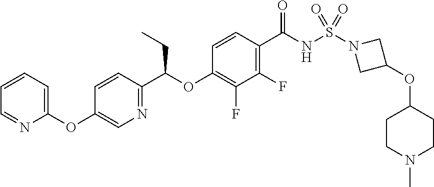 [SMILES: CC[C@@H](Oc1ccc(C(=O)NS(=O)(=O)N2CC(OC3CCN(C)CC3)C2)c(F)c1F)c1ccc(Oc2ccccn2)cn1]

To a solution of (R)-2,3-difluoro-4-(1-(5-(pyridin-2-yloxy)pyridin-2-yl)propoxy)benzoic acid (Acid E, 300 mg, 0.77 mmol) in DCM (5 mL) was added DMAP (9.41 mg, 0.077 mmol), CMPI (296 mg, 1.16 mmol) at RT and the mixture was stirred for 30 mins. 3-((1-methylpiperidin-4-yl)oxy)azetidine-1-sulfonamide (289.22 mg, 1.16 mmol) was added and the mixture was stirred at RT for 10 mins. Et₃N (233.96 mg, 2.31 mmol) was added and the mixture was stirred for 1 hour at RT. The mixture was concentrated to dryness and the residue was purified by Prep-HPLC (method A, gradient: 20-70) to give the title compound (234 mg, 99% purity, >99% ee, 38% yield) as a white solid.

LCMS (Method F) MS (ESI) [M+H]⁺=618.1, Rt=1.02 min. ¹H NMR (400 MHz, DMSO-d₆, ppm) δ 9.33 (s, 1H), 8.45 (d, J 2.5 Hz, 1H), 8.16-8.14 (m, 1H), 7.97-7.78 (m, 1H), 7.65-7.63 (m, 1H), 7.51-7.49 (m, 1H), 7.42-7.38 (m, 1H), 7.25-7.03 (m, 2H), 6.82 (t, J 8.1 Hz, 1H), 5.42 (t, J 6.3 Hz, 1H), 4.30-4.11 (m, 1H), 3.85-3.74 (m, 2H), 3.66-3.55 (m, 3H), 3.23-2.72 (m, 4H), 2.72 (s, 3H), 2.13-1.46 (m, 6H), 0.96 (t, J 7.3 Hz, 3H).

### 4.5.45. Example 45. Biochemical Assays

Various assays were used to determine the ability of compounds to inhibit human and mouse ACSL5, as well as to determine the compounds' cross-reactivity with other isoforms of the enzyme.

### Human Long-chain-fatty-acid--CoA ligase 5 (ACSL5, Uniprot ID Q9ULC5)

Human ACSL5 (33-683) was cloned with a C-terminal 6 His tag and expressed in insect cells (sf9) as per standard insect cell expression protocols. Human ACSL5 was enriched by affinity nickel affinity chromatography followed by Superdex S200 gel filtration column equilibrated in 20 mM HEPES pH 7.8, 500 mM NaCl, 1 mM TCEP, 5% glycerol, 0.1% Triton X¹⁰⁰.

Inhibition of ACSL5 enzyme activity was monitored with a coupled enzyme Amplex Red assay.

The assay is carried out in 384-well microtiterplates. In a total volume of 40 L, compounds are preincubated with 20 nM ACSL5, in a buffer containing 150 mM MOPS-NaOH, 12 mM MgCl2, 0.03% (v/v) Triton X-100, 2.79 U mL-1 Peroxidase and 24 U mL-1 Acyl-CoA Oxidase (pH 7.6). The reaction is started by the addition of substrate and co factors (25 μM oleate, 30 μM CoA, 1 mM ATP and 50 μM Amplex Red) and the fluorescence detected after 10 minutes at 25° C. with a fluorescence reader (BMG-Fluostar; BMG-Technologies) using an excitation wavelength of 535 nm and an emission wavelength of 590 nm.

### Mouse Long-chain-fatty-acid--CoA ligase 5 (ACSL5, Uniprot ID Q8JZRO)

Mouse ACSL5 (33-693) was cloned with a C-terminal 6 His tag and expressed in insect cells (sf9) as per standard insect cell expression protocols. Mouse ACSL5 was enriched by affinity nickel affinity chromatography followed by Superdex S200 gel filtration column equilibrated in 20 mM HEPES pH 7.8, 500 mM NaCl, 1 mM TCEP, 5% glycerol, 0.1% Triton X100.

Inhibition of ACSL5 enzyme activity was monitored with a coupled enzyme Amplex Red assay. The assay is carried out in 384-well microtiterplates. In a total volume of 40 L, compounds are preincubated with 2 nM ACSL5, in a buffer containing 150 mM MOPS-NaOH, 12 mM MgCl2, 0.03% (v/v) Triton X-100, 2.79 U mL-1 Peroxidase and 24 U mL-1 Acyl-CoA Oxidase (pH 7.6). The reaction is started by the addition of substrate and co factors (12 μM oleate, 10 μM CoA, 1 mM ATP and 50 μM Amplex Red) and the fluorescence detected after 10 minutes at 25° C. with a fluorescence reader (BMG-Fluostar; BMG-Technologies) using an excitation wavelength of 535 nm and an emission wavelength of 590 nm.

### Human Long-chain-fatty-acid--CoA ligase 1 (ACSL1, Uniprot ID P33121)

Human ACSL1 (46-698) was cloned with a N-terminal 6 His tag and expressed in insect cells (sf9) as per standard insect cell expression protocols. Human ACSL1 was enriched by affinity nickel affinity chromatography followed by Superdex S200 gel filtration column equilibrated in 20 mM HEPES pH 7.8, 500 mM NaCl, 1 mM TCEP, 5% glycerol, 0.1% Triton X100.

Inhibition of ACSL1 enzyme activity was monitored with a coupled enzyme Amplex Red assay. The assay is carried out in 384-well microtiterplates. In a total volume of 40 L, compounds are preincubated with 50 nM ACSL1, in a buffer containing 150 mM MOPS-NaOH, 12 mM MgCl2, 0.03% (v/v) Triton X-100, 2.79 U mL-1 Peroxidase and 24 U mL-1 Acyl-CoA Oxidase (pH 7.6). The reaction is started by the addition of substrate and co factors (12 μM oleate, 50 μM CoA, 1 mM ATP and 50 μM Amplex Red) and the fluorescence detected after 10 minutes at 25° C. with a fluorescence reader (BMG-Fluostar; BMG-Technologies) using an excitation wavelength of 535 nm and an emission wavelength of 590 nm.

### Human Long-chain-fatty-acid--CoA ligase 6 (ACSL6, Uniprot ID Q9UKU0)

Human ACSL6 (46-6697) was cloned with a C-terminal 6 His tag and expressed in insect cells (sf9) as per standard insect cell expression protocols. Human ACSL6 was prepared as a crude lysate for assay: it was not purified.

Inhibition of ACSL6 enzyme activity was monitored with a coupled enzyme Amplex Red assay. The assay is carried out in 384-well microtiterplates. In a total volume of 40 L, compounds are preincubated with 0.025 mg mL-1 ACSL6 cell lysate, in a buffer containing 150 mM MOPS-NaOH, 12 mM MgCl2, 0.03% (v/v) Triton X-100, 2.79 U mL-1 Peroxidase and 24 U mL-1 Acyl-CoA Oxidase (pH 7.6). The reaction is started by the addition of substrate and co factors (15 μM oleate, 10 μM CoA, 1 mM ATP and 50 μM Amplex Red) and the fluorescence detected after 10 minutes at 25° C. with a fluorescence reader (BMG-Fluostar; BMG-Technologies) using an excitation wavelength of 535 nm and an emission wavelength of 590 nm.

### Cell-based Assays

To assess the cell penetration and cellular activity of the compounds, a cell-based assay (CBA) was used to detect compound potency in living cells. The assay utilized the fluorescent fatty acid analogue B₁₂FA, a boron-dipyrromethene (BODIPY™) labelled C₁₂ fatty acid that can be incorporated into glycerophosphocholine in the cytoplasmic oil droplets of live cells. A concentration-dependent, red-shifted fluorescence can be induced by the formation of BODIPY™ excimers in the oil droplets.

Expi293F cells transfected with full length hACSL5 plasmid were selected using 1 mg/mL G418 culture medium and clones were selected by in vitro whole cell lysate (WCL) assay. The cell passage number used in the Expi293 CBA assay could not be used beyond 10 passages due to a decrease in hACSL5 expression. Therefore, a CBA using FlpIn TRex293 cells for inducible expression of hACSL5 was also established.

FlpIn TRex293 cells transfected with hACSL5 plasmid were selected in culture medium containing 15 g/ml Blasticidin and 150 g/ml Hygromycin. For cell-based assays, stable transfected hACSL5 cells were seeded onto a 96-well plate at 30-50k/well and incubated overnight. For the inducible system, 1 g/ml tetracycline (or doxycycline) was added to culture medium of FlpIn TRex293 hACSL5 cells on the day before the assay to induce expression of hACSL5. The next day, growth media was replaced with culture medium without FBS and glucose (DMEM glucose (-), ThermoFisher) and cells were incubated for an hour before treating cells with serial diluted compounds containing 20 M B₁₂FA (ThermoFisher) for another 3 hours in the above medium. Cells were then fixed in 4% formaldehyde for 10 minutes and washed twice with PBS. The plate was read using the Cytation 5 imaging system with an excitation wavelength of 485 nm and observed emissions at 630 nm and 520 nm. The percentage of inhibition was calculated as:

$$Percent inhibition = 100 * ( A - X ) / ( A - B )$$

wherein A is the ratio of emission intensities at 630 nm/520 nm in the absence of inhibitor (maximum ratio); B is the ratio of the intensities at those wavelengths in the presence of 10 M of the test compound or non-induced cells (minimum ratio); and X is the ratio of the intensities at a given compound concentration. For the inducible system, non-induced cells did not express hACSL5 and the emission ratio was used for a minimum ratio.

### 4.5.46. Example 46. Representative Assay Data

Concentration-response titration points for each compound tested were filled to the Hill equation yielding concentrations of half maximal inhibition (1C₅) and maximal inhibition values. In vitro assay data obtained for some representative compounds of the invention are provided below in Table 5, wherein human ACSL5 and CBA inhibition were measured using the assays described above and: +represents an IC₅₀ >500 nM; ++represents an IC₅₀ of from 100 nM to 500 nM; and +++represents an IC₅₀<100 nM.

TABLE 5

| Compound | hACSL5 IC₅₀ | CBA IC₅₀ |
|---|---|---|
| N-((4-(2-aminoethyl)phenyl)sulfonyl)-2,3-difluoro-4-((3- | ++ | |
| phenoxybenzyl)oxy)benzamide | | |
| N-((2-aminoethyl)sulfonyl)-2,3-dichloro-4-(1-(3- | ++ | |
| phenoxyphenyl)ethoxy)benzamide | | |
| rac-(R)-2,3-dichloro-N-((4-methylpiperazin-1-yl)sulfonyl)-4-(1-(3- | +++ | |
| phenoxyphenyl)ethoxy)benzamide | | |
| rac-(R)-N-((4-(aminomethyl)phenyl)sulfonyl)-2,3-dichloro-4-(1-(3- | +++ | |
| phenoxyphenyl)ethoxy)benzamide | | |
| rac-(R)-N-((2-aminoethyl)sulfonyl)-2,3-dichloro-4-(1-(3- | + | |
| phenoxyphenyl)ethoxy)benzamide | | |
| N-((4-((dimethylamino)methyl)phenyl)sulfonyl)-2,3-difluoro-4-((3- | ++ | |
| phenoxybenzyl)oxy)benzamide | | |
| 2,3-difluoro-N-((4-((methylamino)methyl)phenyl)sulfonyl)-4-((3- | +++ | |
| phenoxybenzyl)oxy)benzamide | | |
| N-((4-(aminomethyl)phenyl)sulfonyl)-2,3-difluoro-4-((4- | + | |
| phenoxybenzyl)oxy)benzamide | | |
| N-((4-(aminomethyl)phenyl)sulfonyl)-4-(1-(3- | ++ | |
| (cyclopentyloxy)phenyl)ethoxy)-2,3-difluorobenzamide | | |
| N-((4-(aminomethyl)phenyl)sulfonyl)-2,3-dichloro-4-((3- | +++ | |
| phenoxybenzyl)oxy)benzamide | | |
| rac-(R)-N-((4-(aminomethyl)phenyl)sulfonyl)-3-methyl-4-(1-(3- | +++ | |
| phenoxyphenyl)ethoxy)benzamide | | |
| N-((4-(aminomethyl)phenyl)sulfonyl)-2,3-difluoro-4-(1-(3- | +++ | |
| phenoxyphenyl)ethoxy)benzamide | | |
| N-((4-(aminomethyl)phenyl)sulfonyl)-4-(1-(3-(thiophen-3- | +++ | |
| yloxy)phenyl)ethoxy)-3-(trifluoromethyl)benzamide | | |
| N-((4-(aminomethyl)phenyl)sulfonyl)-4-(1-(3-(pyridin-3- | +++ | |
| yloxy)phenyl)ethoxy)-3-(trifluoromethyl)benzamide | | |
| N-((4-(aminomethyl)phenyl)sulfonyl)-4-(2-(3-phenoxyphenyl)propoxy)- | ++ | |
| 3-(trifluoromethyl)benzamide | | |
| N-((4-(aminomethyl)phenyl)sulfonyl)-2,3-dichloro-4-(2-(3- | ++ | |
| phenoxyphenyl)propoxy)benzamide | | |
| N-((4-(aminomethyl)phenyl)sulfonyl)-2,3-difluoro-4-(1-(3-(pyridin-3- | +++ | |
| yloxy)phenyl)ethoxy)benzamide | | |
| N-((4-(aminomethyl)phenyl)sulfonyl)-4-(1-(3- | + | |
| (cyclopentyloxy)phenyl)ethoxy)-3-(trifluoromethyl)benzamide | | |
| N-((4-(aminomethyl)phenyl)sulfonyl)-4-(1-(3-(pyrimidin-5- | ++ | |
| yloxy)phenyl)ethoxy)-3-(trifluoromethyl)benzamide | | |
| N-((4-(aminomethyl)phenyl)sulfonyl)-4-(1-(3-(pyridin-2- | +++ | |
| yloxy)phenyl)ethoxy)-3-(trifluoromethyl)benzamide | | |
| N-((4-(aminomethyl)phenyl)sulfonyl)-4-(1-(3-(pyrazin-2- | +++ | |
| yloxy)phenyl)ethoxy)-3-(trifluoromethyl)benzamide | | |
| N-((4-(aminomethyl)phenyl)sulfonyl)-4-(1-(3-((1-methyl-1H-pyrazol-4- | + | |
| yl)oxy)phenyl)ethoxy)-3-(trifluoromethyl)benzamide | | |
| N-((4-(aminomethyl)phenyl)sulfonyl)-4-(1-(3-((2- | + | |
| methylpentyl)oxy)phenyl)ethoxy)-3-(trifluoromethyl)benzamide | | |
| N-((4-(aminomethyl)phenyl)sulfonyl)-4-(1-(3- | ++ | |
| (pentyloxy)phenyl)ethoxy)-3-(trifluoromethyl)benzamide | | |
| N-((4-(aminomethyl)phenyl)sulfonyl)-4-(1-(3- | + | |
| (cyclopentylmethoxy)phenyl)ethoxy)-3-(trifluoromethyl)benzamide | | |
| N-((4-(aminomethyl)phenyl)sulfonyl)-4-(cyclopentylmethoxy)-3- | + | |
| (trifluoromethyl)benzamide | | |
| rac-(R)-N-((2-aminoethyl)sulfonyl)-4-(1-(3-phenoxyphenyl)ethoxy)-3- | ++ | |
| (trifluoromethyl)benzamide | | |
| N-((3-morpholinopropyl)sulfonyl)-4-(1-(5-(pyridazin-3-yloxy)pyridin- | ++ | |
| 2-yl)ethoxy)-3-(trifluoromethyl)benzamide | | |
| 4-(1-(5-ethoxypyridin-2-yl)propoxy)-N-((3-morpholinopropyl)sulfonyl)- | ++ | |
| 3-(trifluoromethyl)benzamide | | |
| 4-(1-(5-ethoxypyridin-2-yl)propoxy)-N-((4-(2- | +++ | |
| (methylamino)ethoxy)phenyl)sulfonyl)-3-(trifluoromethyl)benzamide | | |
| 4-(1-(5-ethoxypyridin-2-yl)ethoxy)-2,3-difluoro-N-((4-methylpiperazin- | + | |
| 1-yl)sulfonyl)benzamide | | |
| 4-(1-(5-ethoxypyridin-2-yl)ethoxy)-2,3-difluoro-N-(piperidin-4- | + | |
| ylsulfonyl)benzamide | | |
| 4-(1-(3-(cyclopropylmethoxy)phenyl)ethoxy)-N-((1-(2- | ++ | + |
| (dimethylamino)ethyl)-1H-pyrazol-4-yl)sulfonyl)-2,3- | | |
| difluorobenzamide | | |
| N-((4-(2-(1H-imidazol-1-yl)ethoxy)phenyl)sulfonyl)-4-(1-(5- | ++ | + |
| ethoxypyridin-2-yl)ethoxy)-2,3-difluorobenzamide | | |
| (R)-4-(1-(5-(cyclopropylmethoxy)pyridin-2-yl)propoxy)-2,3-difluoro-N- | +++ | + |
| ((4-(2-morpholinoethoxy)phenyl)sulfonyl)benzamide | | |
| (R)-4-(1-(5-(cyclopropylmethoxy)pyridin-2-yl)propoxy)-N-((4-(2- | +++ | ++ |
| (dimethylamino)ethoxy)phenyl)sulfonyl)-2,3-difluorobenzamide | | |
| (R)-4-(1-(5-(cyclopropylmethoxy)pyridin-2-yl)propoxy)-2,3-difluoro-N- | +++ | ++ |
| ((4-(2-(4-methylpiperazin-1-yl)ethoxy)phenyl)sulfonyl)benzamide | | |
| (R)-N-((4-(2-(1H-imidazol-1-yl)ethoxy)phenyl)sulfonyl)-2,3-difluoro-4- | +++ | + |
| (1-(5-(2-methoxyethoxy)pyridin-2-yl)propoxy)benzamide | | |
| (R)-N-((4-(2-(dimethylamino)ethoxy)phenyl)sulfonyl)-2,3-difluoro-4- | ++ | + |
| (1-(5-(2-methoxyethoxy)pyridin-2-yl)propoxy)benzamide | | |
| (R)-4-(1-(5-(cyclopropylmethoxy)pyridin-2-yl)propoxy)-2,3-difluoro-N- | +++ | ++ |
| ((4-(2-(pyrrolidin-1-yl)ethoxy)phenyl)sulfonyl)benzamide | | |
| 4-((R)-1-(5-(cyclopropylmethoxy)pyridin-2-yl)propoxy)-2,3-difluoro-N- | +++ | ++ |
| ((4-(2-((S)-3-fluoropyrrolidin-1-yl)ethoxy)phenyl)sulfonyl)benzamide | | |
| (R)-4-(1-(5-(cyclopropylmethoxy)pyridin-2-yl)propoxy)-2,3-difluoro-N- | +++ | ++ |
| ((4-(2-(oxetan-3-ylamino)ethoxy)phenyl)sulfonyl)benzamide | | |
| (R)-N-((4-((1H-imidazol-4-yl)methoxy)phenyl)sulfonyl)-2,3-difluoro-4- | +++ | + |
| (1-(5-(2-methoxyethoxy)pyridin-2-yl)propoxy)benzamide | | |
| (R)-N-((4-(2-(cyclopropylamino)ethoxy)phenyl)sulfonyl)-4-(1-(5- | +++ | +++ |
| (cyclopropylmethoxy)pyridin-2-yl)propoxy)-2,3-difluorobenzamide | | |
| (R)-4-(1-(5-(cyclopropylmethoxy)pyridin-2-yl)propoxy)-2,3-difluoro-N- | +++ | ++ |
| ((4-((1-methylpiperidin-4-yl)oxy)piperidin-1-yl)sulfonyl)benzamide | | |
| (R)-4-(1-(5-(cyclopropylmethoxy)pyridin-2-yl)propoxy)-N-((6-(2- | ++ | + |
| (dimethylamino)ethoxy)-2-azaspiro[3.3]heptan-2-yl)sulfonyl)-2,3- | | |
| difluorobenzamide | | |
| (R)-4-(1-(5-(cyclopropylmethoxy)pyridin-2-yl)propoxy)-2,3-difluoro-N- | +++ | + |
| ((6-(2-(pyrrolidin-1-yl)ethoxy)-2-azaspiro[3.3]heptan-2- | | |
| yl)sulfonyl)benzamide | | |
| 4-((R)-1-(5-(cyclopropylmethoxy)pyridin-2-yl)propoxy)-2,3-difluoro-N- | +++ | ++ |
| ((4-(((S)-1-methylpyrrolidin-3-yl)oxy)phenyl)sulfonyl)benzamide | | |
| 4-((R)-1-(5-(cyclopropylmethoxy)pyridin-2-yl)propoxy)-2,3-difluoro-N- | +++ | ++ |
| ((4-(((R)-1-methylpyrrolidin-3-yl)oxy)phenyl)sulfonyl)benzamide | | |
| (R)-4-(1-(5-(cyclopropylmethoxy)pyridin-2-yl)propoxy)-N-((4-((1- | +++ | +++ |
| ethylpiperidin-4-yl)oxy)phenyl)sulfonyl)-2,3-difluorobenzamide | | |
| (R)-4-(1-(5-(cyclopropylmethoxy)pyridin-2-yl)propoxy)-2,3-difluoro-N- | +++ | ++ |
| ((4-((1-methylazetidin-3-yl)oxy)phenyl)sulfonyl)benzamide | | |
| (R)-4-(1-(5-(cyclopropylmethoxy)pyridin-2-yl)propoxy)-2,3-difluoro-N- | +++ | + |
| ((6-((1-methylpiperidin-4-yl)oxy)pyridazin-3-yl)sulfonyl)benzamide | | |
| (R)-4-(1-(5-(cyclopropylmethoxy)pyridin-2-yl)propoxy)-2,3-difluoro-N- | +++ | ++ |
| ((6-((1-methylpiperidin-4-yl)oxy)pyridin-3-yl)sulfonyl)benzamide | | |
| (R)-4-(1-(5-(cyclopropylmethoxy)pyridin-2-yl)propoxy)-2,3-difluoro-N- | +++ | +++ |
| ((4-((1-(methyl-d3)piperidin-4-yl)oxy)phenyl)sulfonyl)benzamide | | |
| (R)-4-(1-(5-(cyclopropylmethoxy)pyridin-2-yl)propoxy)-N-((4-((1- | +++ | +++ |
| (cyclopropylmethyl)piperidin-4-yl)oxy)phenyl)sulfonyl)-2,3- | | |
| difluorobenzamide | | |
| (R)-4-(1-(5-(cyclopropylmethoxy)pyridin-2-yl)propoxy)-2,3-difluoro-N- | +++ | ++ |
| ((4-((1-(2-hydroxyethyl)piperidin-4-yl)oxy)phenyl)sulfonyl)benzamide | | |
| (R)-4-(1-(5-(cyclopropylmethoxy)pyridin-2-yl)propoxy)-2,3-difluoro-N- | +++ | ++ |
| ((4-((1-isopropylpiperidin-4-yl)oxy)phenyl)sulfonyl)benzamide | | |
| (R)-4-(1-(5-(cyclopropylmethoxy)pyridin-2-yl)propoxy)-2,3-difluoro-N- | +++ | + |
| ((6-((1-methylpiperidin-4-yl)amino)pyridin-3-yl)sulfonyl)benzamide | | |
| 4-((R)-1-(5-(cyclopropylmethoxy)pyridin-2-yl)propoxy)-2,3-difluoro-N- | +++ | ++ |
| ((4-((1,2,2-trimethylpiperidin-4-yl)oxy)phenyl)sulfonyl)benzamide | | |
| (R)-4-(1-(5-(cyclopropylmethoxy)pyridin-2-yl)propoxy)-2,3-difluoro-N- | +++ | + |
| ((4-(piperidin-4-yloxy)phenyl)sulfonyl)benzamide | | |
| (R)-4-(1-(5-(cyclopropylmethoxy)pyridin-2-yl)propoxy)-2,3-difluoro-N- | ++ | |
| ((4-(2-(pyrrolidin-1-yl)ethoxy)piperidin-1-yl)sulfonyl)benzamide | | |
| (R)-4-(1-(5-(cyclopropylmethoxy)pyridin-2-yl)propoxy)-2,3-difluoro-N- | +++ | + |
| ((2-((1-methylpiperidin-4-yl)oxy)pyrimidin-5-yl)sulfonyl)benzamide | | |
| 4-((R)-1-(5-(cyclopropylmethoxy)pyridin-2-yl)propoxy)-2,3-difluoro-N- | +++ | + |
| ((4-(((1R,4R,5R)-2-methyl-2-azabicyclo[2.2.1]heptan-5- | | |
| yl)oxy)phenyl)sulfonyl)benzamide | | |
| 4-((R)-1-(5-(cyclopropylmethoxy)pyridin-2-yl)propoxy)-2,3-difluoro-N- | +++ | ++ |
| ((5-(2-(pyrrolidin-1-yl)ethoxy)-2-azabicyclo[2.2.1]heptan-2- | | |
| yl)sulfonyl)benzamide | | |
| (R)-4-(1-(5-(cyclopropylmethoxy)pyridin-2-yl)propoxy)-N-((4-((1-(2,2- | +++ | ++ |
| difluoroethyl)piperidin-4-yl)oxy)phenyl)sulfonyl)-2,3- | | |
| difluorobenzamide | | |
| N-((4-(((1R,4R,5R)-2-azabicyclo[2.2.1]heptan-5- | +++ | + |
| yl)oxy)phenyl)sulfonyl)-4-((R)-1-(5-(cyclopropylmethoxy)pyridin-2- | | |
| yl)propoxy)-2,3-difluorobenzamide | | |
| (R)-4-(1-(5-(cyclopropylmethoxy)pyridin-2-yl)propoxy)-2,3-difluoro-N- | +++ | + |
| ((6-(2-(pyrrolidin-1-yl)ethoxy)pyridazin-3-yl)sulfonyl)benzamide | | |
| (R)-4-(1-(5-(cyclopropylmethoxy)pyridin-2-yl)propoxy)-2,3-difluoro-N- | +++ | + |
| ((6-((2-(pyrrolidin-1-yl)ethyl)amino)pyridin-3-yl)sulfonyl)benzamide | | |
| (R)-4-(1-(5-(cyclopropylmethoxy)pyridin-2-yl)propoxy)-2,3-difluoro-N- | +++ | +++ |
| ((4-((1-methylpiperidin-4-yl)oxy)phenyl)sulfonyl)benzamide | | |
| (R)-4-(1-(5-(cyclopropylmethoxy)pyridin-2-yl)propoxy)-2,3-difluoro-N- | +++ | + |
| ((5-((1-methylpiperidin-4-yl)oxy)pyridin-2-yl)sulfonyl)benzamide | | |
| (R)-4-(1-(5-(cyclopropylmethoxy)pyridin-2-yl)propoxy)-2,3-difluoro-N- | +++ | + |
| ((5-((1-methylpiperidin-4-yl)amino)pyridin-2-yl)sulfonyl)benzamide | | |
| (R)-4-(1-(5-(cyclopropylmethoxy)pyridin-2-yl)propoxy)-2,3-difluoro-N- | +++ | + |
| ((6-((1-isopropylpiperidin-4-yl)amino)pyridin-3-yl)sulfonyl)benzamide | | |
| (R)-4-(1-(5-(cyclopropylmethoxy)pyridin-2-yl)propoxy)-2,3-difluoro-N- | +++ | ++ |
| ((6-(2-(pyrrolidin-1-yl)ethoxy)pyridin-3-yl)sulfonyl)benzamide | | |
| (R)-4-(1-(5-(cyclopropylmethoxy)pyridin-2-yl)propoxy)-2,3-difluoro-N- | ++ | + |
| ((6-((1-methylpiperidin-4-yl)oxy)-2-azaspiro[3.3]heptan-2- | | |
| yl)sulfonyl)benzamide | | |
| (R)-4-(1-(5-(cyclopropylmethoxy)pyridin-2-yl)propoxy)-2,3-difluoro-N- | +++ | ++ |
| ((6-((1-isopropylpiperidin-4-yl)oxy)pyridin-3-yl)sulfonyl)benzamide | | |
| (R)-N-((4-((1-azabicyclo[2.2.1]heptan-4-yl)oxy)phenyl)sulfonyl)-4-(1- | +++ | ++ |
| (5-(cyclopropylmethoxy)pyridin-2-yl)propoxy)-2,3-difluorobenzamide | | |
| 4-((R)-1-(5-(cyclopropylmethoxy)pyridin-2-yl)propoxy)-2,3-difluoro-N- | +++ | ++ |
| ((4-(2-((R)-1-methylpiperidin-2-yl)ethoxy)phenyl)sulfonyl)benzamide | | |
| (R)-4-(1-(5-(cyclopropylmethoxy)pyridin-2-yl)propoxy)-2,3-difluoro-N- | +++ | +++ |
| ((4-(2-(piperidin-1-yl)ethoxy)phenyl)sulfonyl)benzamide | | |
| (R)-2,3-difluoro-N-((4-((1-methylpiperidin-4-yl)oxy)piperidin-1- | +++ | +++ |
| yl)sulfonyl)-4-(1-(5-(pyridin-2-yloxy)pyridin-2-yl)propoxy)benzamide | | |
| (R)-4-(1-(5-(cyclopropylmethoxy)pyridin-2-yl)propoxy)-N-((6-((4- | +++ | + |
| ethylpiperazin-1-yl)methyl)pyridin-3-yl)sulfonyl)-2,3- | | |
| difluorobenzamide | | |
| (R)-4-(1-(5-(cyclopropylmethoxy)pyridin-2-yl)propoxy)-2,3-difluoro-N- | +++ | +++ |
| ((4-((1-(2-methoxyethyl)piperidin-4-yl)oxy)phenyl)sulfonyl)benzamide | | |
| (R)-4-(1-(5-(cyclopropylmethoxy)pyridin-2-yl)propoxy)-2,3-difluoro-N- | +++ | ++ |
| ((4-((1-(2-hydroxy-2-methylpropyl)piperidin-4- | | |
| yl)oxy)phenyl)sulfonyl)benzamide | | |
| (R)-4-(1-(5-(cyclopropylmethoxy)pyridin-2-yl)propoxy)-2,3-difluoro-N- | +++ | ++ |
| ((4-((1-(2-hydroxy-2-methylpropyl)piperidin-4-yl)oxy)piperidin-1- | | |
| yl)sulfonyl)benzamide | | |
| (R)-4-(1-(5-(cyclopropylmethoxy)pyridin-2-yl)propoxy)-2,3-difluoro-N- | +++ | +++ |
| ((4-((1-(2-methoxyethyl)piperidin-4-yl)oxy)piperidin-1- | | |
| yl)sulfonyl)benzamide | | |
| (R)-4-(1-(5-(cyclopropylmethoxy)pyridin-2-yl)propoxy)-N-((4-((1- | +++ | ++ |
| (cyclopropylmethyl)piperidin-4-yl)oxy)piperidin-1-yl)sulfonyl)-2,3- | | |
| difluorobenzamide | | |
| (R)-4-(1-(5-(cyclopropylmethoxy)pyridin-2-yl)propoxy)-N-((4-((4- | +++ | + |
| ethylpiperazin-1-yl)methyl)piperidin-1-yl)sulfonyl)-2,3- | | |
| difluorobenzamide | | |
| (R)-2,3-difluoro-4-(1-(5-(2-methoxyethoxy)pyridin-2-yl)propoxy)-N- | + | |
| ((4-((1-methylpiperidin-4-yl)oxy)phenyl)sulfonyl)benzamide | | |
| (R)-N-((4-(2-(dimethylamino)ethoxy)piperidin-1-yl)sulfonyl)-2,3- | + | |
| difluoro-4-(1-(5-(2-methoxyethoxy)pyridin-2-yl)propoxy)benzamide | | |
| (R)-2,3-difluoro-4-(1-(5-(2-methoxyethoxy)pyridin-2-yl)propoxy)-N- | + | |
| ((4-((1-methylpiperidin-4-yl)oxy)piperidin-1-yl)sulfonyl)benzamide | | |
| (R)-N-((4-(2-(dimethylamino)ethoxy)piperidin-1-yl)sulfonyl)-2,3- | +++ | ++ |
| difluoro-4-(1-(5-(pyridin-2-yloxy)pyridin-2-yl)propoxy)benzamide | | |
| (R)-4-(1-(5-(cyclopropylmethoxy)pyridin-2-yl)propoxy)-N-((4-(2- | +++ | ++ |
| (dimethylamino)ethoxy)piperidin-1-yl)sulfonyl)-2,3-difluorobenzamide | | |
| (R)-2,3-difluoro-N-((4-methylpiperazin-1-yl)sulfonyl)-4-(1-(5-(pyridin- | +++ | +++ |
| 2-yloxy)pyridin-2-yl)propoxy)benzamide | | |
| (R)-4-(1-(5-(cyclopropylmethoxy)pyridin-2-yl)propoxy)-2,3-difluoro-N- | ++ | + |
| ((1-methylpiperidin-4-yl)sulfonyl)benzamide | | |
| (R)-2,3-difluoro-4-(1-(5-(pyridin-2-yloxy)pyridin-2-yl)propoxy)-N- | +++ | +++ |
| ((2,2,4-trimethylpiperazin-1-yl)sulfonyl)benzamide | | |
| (R)-2,3-difluoro-N-((1-methylpiperidin-4-yl)sulfonyl)-4-(1-(5-(pyridin- | +++ | ++ |
| 2-yloxy)pyridin-2-yl)propoxy)benzamide | | |
| (R)-4-(1-(5-(cyclopropylmethoxy)pyridin-2-yl)propoxy)-2,3-difluoro-N- | +++ | +++ |
| ((2,2,4-trimethylpiperazin-1-yl)sulfonyl)benzamide | | |
| (R)-4-(1-(5-(cyclopropylmethoxy)pyridin-2-yl)propoxy)-N-((1- | ++ | ++ |
| (dimethylamino)-2-methylpropan-2-yl)sulfonyl)-2,3-difluorobenzamide | | |
| (R)-4-(1-(5-(cyclopropylmethoxy)pyridin-2-yl)propoxy)-2,3-difluoro-N- | +++ | +++ |
| ((4-methylpiperazin-1-yl)sulfonyl)benzamide | | |
| (R)-N-((1-(dimethylamino)-2-methylpropan-2-yl)sulfonyl)-2,3-difluoro- | +++ | +++ |
| 4-(1-(5-(pyridin-2-yloxy)pyridin-2-yl)propoxy)benzamide | | |
| (R)-N-((4-((1-methylpiperidin-4-yl)oxy)piperidin-1-yl)sulfonyl)-4-(1- | +++ | +++ |
| (5-(pyridin-2-yloxy)pyridin-2-yl)propoxy)-3- | | |
| (trifluoromethyl)benzamide | | |
| (R)-N-((4-(2-(dimethylamino)ethoxy)phenyl)sulfonyl)-4-(1-(5-(pyridin- | +++ | +++ |
| 2-yloxy)pyridin-2-yl)propoxy)-3-(trifluoromethyl)benzamide | | |
| (R)-N-((4-(2-(dimethylamino)ethoxy)phenyl)sulfonyl)-2,3-difluoro-4- | +++ | ++ |
| (1-(5-(pyridin-2-yloxy)pyridin-2-yl)propoxy)benzamide | | |
| (R)-N-((4-((1-methylpiperidin-4-yl)oxy)piperidin-1-yl)sulfonyl)-4-(1- | +++ | +++ |
| (5-(pyridin-2-yloxy)pyridin-2-yl)ethoxy)-3-(trifluoromethyl)benzamide | | |
| (R)-N-((4-(2-(dimethylamino)ethoxy)phenyl)sulfonyl)-4-(1-(5-(pyridin- | +++ | +++ |
| 2-yloxy)pyridin-2-yl)ethoxy)-3-(trifluoromethyl)benzamide | | |
| (R)-4-(1-(5-(cyclopropylmethoxy)pyridin-2-yl)propoxy)-2,3-difluoro-N- | ++ | ++ |
| ((4-isopropylpiperazin-1-yl)sulfonyl)benzamide | | |
| (R)-4-(1-(5-(cyclopropylmethoxy)pyridin-2-yl)propoxy)-N-((4- | +++ | +++ |
| cyclopropylpiperazin-1-yl)sulfonyl)-2,3-difluorobenzamide | | |
| 4-((R)-1-(5-(cyclopropylmethoxy)pyridin-2-yl)propoxy)-2,3-difluoro-N- | +++ | +++ |
| ((4-((1-methylpiperidin-3-yl)oxy)phenyl)sulfonyl)benzamide | | |
| 2,3-difluoro-N-((4-((1-methylpiperidin-3-yl)oxy)phenyl)sulfonyl)-4- | +++ | +++ |
| ((R)-1-(5-(pyridin-2-yloxy)pyridin-2-yl)propoxy)benzamide | | |
| (R)-4-(1-(5-(cyclopropylmethoxy)pyridin-2-yl)propoxy)-2,3-difluoro-N- | ++ | ++ |
| ((4-methyl-1,4-diazepan-1-yl)sulfonyl)benzamide | | |
| (R)-4-(1-(5-(cyclopropylmethoxy)pyridin-2-yl)propoxy)-2,3-difluoro-N- | +++ | +++ |
| ((3,3,4-trimethylpiperazin-1-yl)sulfonyl)benzamide | | |
| 4-((R)-1-(5-(cyclopropylmethoxy)pyridin-2-yl)propoxy)-2,3-difluoro-N- | +++ | +++ |
| (((3S,5R)-3,4,5-trimethylpiperazin-1-yl)sulfonyl)benzamide | | |
| (R)-N-((4-(2-(dimethylamino)ethoxy)phenyl)sulfonyl)-2,3-difluoro-4- | +++ | ++ |
| (1-(5-(pyridin-2-yloxy)pyridin-2-yl)ethoxy)benzamide | | |
| (R)-4-(1-(5-(cyclopropylmethoxy)pyridin-2-yl)propoxy)-2,3-difluoro-N- | +++ | +++ |
| ((4-(2-methoxyethyl)piperazin-1-yl)sulfonyl)benzamide | | |
| 4-((R)-1-(5-(cyclopropylmethoxy)pyridin-2-yl)propoxy)-2,3-difluoro-N- | +++ | +++ |
| ((6-methyl-3,6-diazabicyclo[3.1.1]heptan-3-yl)sulfonyl)benzamide | | |
| 4-((R)-1-(5-(cyclopropylmethoxy)pyridin-2-yl)propoxy)-N-(((S)-3,4- | +++ | +++ |
| dimethylpiperazin-1-yl)sulfonyl)-2,3-difluorobenzamide | | |
| 4-((R)-1-(5-(cyclopropylmethoxy)pyridin-2-yl)propoxy)-N-(((R)-3,4- | +++ | +++ |
| dimethylpiperazin-1-yl)sulfonyl)-2,3-difluorobenzamide | | |
| (R)-2,3-difluoro-N-((4-((1-(2-methoxyethyl)piperidin-4- | +++ | +++ |
| yl)oxy)piperidin-1-yl)sulfonyl)-4-(1-(5-(pyridin-2-yloxy)pyridin-2- | | |
| yl)propoxy)benzamide | | |
| 4-((R)-1-(5-(cyclopropylmethoxy)pyridin-2-yl)propoxy)-2,3-difluoro-N- | +++ | +++ |
| ((8-methyl-3,8-diazabicyclo[3.2.1]octan-3-yl)sulfonyl)benzamide | | |
| N-((4-(2-(dimethylamino)ethoxy)phenyl)sulfonyl)-2,3-difluoro-4-((R)- | +++ | ++ |
| 1-(5-((1R,3R)-3-methoxycyclobutoxy)pyridin-2-yl)propoxy)benzamide | | |
| 2,3-difluoro-4-((R)-1-(5-((1R,3R)-3-methoxycyclobutoxy)pyridin-2- | +++ | ++ |
| yl)propoxy)-N-((4-((1-methylpiperidin-4-yl)oxy)piperidin-1- | | |
| yl)sulfonyl)benzamide | | |
| 4-((R)-1-(5-(cyclopropylmethoxy)pyridin-2-yl)propoxy)-2,3-difluoro-N- | ++ | ++ |
| (((1S,4S)-5-methyl-2,5-diazabicyclo[2.2.1]heptan-2- | | |
| yl)sulfonyl)benzamide | | |
| 4-((R)-1-(5-(cyclopropylmethoxy)pyridin-2-yl)propoxy)-2,3-difluoro-N- | ++ | ++ |
| (((1R,4R)-5-methyl-2,5-diazabicyclo[2.2.1]heptan-2- | | |
| yl)sulfonyl)benzamide | | |
| (R)-N-((4-ethylpiperazin-1-yl)sulfonyl)-2,3-difluoro-4-(1-(5-(pyridin-2- | +++ | +++ |
| yloxy)pyridin-2-yl)propoxy)benzamide | | |
| (R)-4-(1-(5-(cyclopropylmethoxy)pyridin-2-yl)propoxy)-N-((4- | +++ | +++ |
| ethylpiperazin-1-yl)sulfonyl)-2,3-difluorobenzamide | | |
| (R)-N-((4-methylpiperazin-1-yl)sulfonyl)-4-(1-(5-(pyridin-2- | +++ | +++ |
| yloxy)pyridin-2-yl)propoxy)-3-(trifluoromethyl)benzamide | | |
| (R)-4-(1-(5-(cyclopropylmethoxy)pyridin-2-yl)propoxy)-N-((4-((1- | +++ | +++ |
| methylpiperidin-4-yl)oxy)piperidin-1-yl)sulfonyl)-3- | | |
| (trifluoromethyl)benzamide | | |
| (R)-4-(1-(5-(cyclopropylmethoxy)pyridin-2-yl)propoxy)-N-((4- | +++ | +++ |
| methylpiperazin-1-yl)sulfonyl)-3-(trifluoromethyl)benzamide | | |
| (R)-N-((4-cyclopropylpiperazin-1-yl)sulfonyl)-2,3-difluoro-4-(1-(5- | +++ | +++ |
| (pyridin-2-yloxy)pyridin-2-yl)propoxy)benzamide | | |
| (R)-N-((4-cyclohexylpiperazin-1-yl)sulfonyl)-4-(1-(5- | +++ | + |
| (cyclopropylmethoxy)pyridin-2-yl)propoxy)-2,3-difluorobenzamide | | |
| (R)-N-((4-cyclohexylpiperazin-1-yl)sulfonyl)-2,3-difluoro-4-(1-(5- | +++ | ++ |
| (pyridin-2-yloxy)pyridin-2-yl)propoxy)benzamide | | |
| (R)-4-(1-(5-(cyclopropylmethoxy)pyridin-2-yl)propoxy)-N-((4-(2- | +++ | ++ |
| (dimethylamino)ethoxy)phenyl)sulfonyl)-3-(trifluoromethyl)benzamide | | |
| (R)-4-(1-(4-(cyclopropylmethoxy)pyridin-2-yl)propoxy)-2,3-difluoro-N- | ++ | ++ |
| ((4-((1-methylpiperidin-4-yl)oxy)piperidin-1-yl)sulfonyl)benzamide | | |
| (R)-4-(1-(4-(cyclopropylmethoxy)pyridin-2-yl)propoxy)-N-((4-(2- | ++ | ++ |
| (dimethylamino)ethoxy)phenyl)sulfonyl)-2,3-difluorobenzamide | | |
| (R)-4-(1-(4-(cyclopropylmethoxy)pyridin-2-yl)propoxy)-2,3-difluoro-N- | ++ | ++ |
| ((4-((1-methylpiperidin-4-yl)oxy)phenyl)sulfonyl)benzamide | | |
| (R)-2,3-difluoro-N-((4-((1-methylpiperidin-4-yl)oxy)piperidin-1- | +++ | ++ |
| yl)sulfonyl)-4-(1-(4-(pyridin-2-yloxy)pyridin-2-yl)propoxy)benzamide | | |
| (R)-2,3-difluoro-N-((4-methylpiperazin-1-yl)sulfonyl)-4-(1-(4-(pyridin- | +++ | ++ |
| 2-yloxy)pyridin-2-yl)propoxy)benzamide | | |
| (R)-N-((4-cyclopropylpiperazin-1-yl)sulfonyl)-2,3-difluoro-4-(1-(4- | +++ | ++ |
| (pyridin-2-yloxy)pyridin-2-yl)propoxy)benzamide | | |
| (R)-N-((4-(2-(dimethylamino)ethoxy)phenyl)sulfonyl)-2,3-difluoro-4- | +++ | ++ |
| (1-(4-(pyridin-2-yloxy)pyridin-2-yl)propoxy)benzamide | | |
| (R)-4-(1-(5-(cyclopropylmethoxy)pyridin-2-yl)propoxy)-N-((4- | +++ | ++ |
| (cyclopropylmethyl)piperazin-1-yl)sulfonyl)-2,3-difluorobenzamide | | |
| (R)-N-((4-(cyclopropylmethyl)piperazin-1-yl)sulfonyl)-2,3-difluoro-4- | +++ | +++ |
| (1-(5-(pyridin-2-yloxy)pyridin-2-yl)propoxy)benzamide | | |
| (R)-N-((4-cyclopentylpiperazin-1-yl)sulfonyl)-4-(1-(5- | +++ | ++ |
| (cyclopropylmethoxy)pyridin-2-yl)propoxy)-2,3-difluorobenzamide | | |
| (R)-N-((4-cyclopentylpiperazin-1-yl)sulfonyl)-2,3-difluoro-4-(1-(5- | +++ | ++ |
| (pyridin-2-yloxy)pyridin-2-yl)propoxy)benzamide | | |
| N-((4-(2-(dimethylamino)ethoxy)phenyl)sulfonyl)-4-((R)-1-(5- | ++ | ++ |
| ((1R,3R)-3-methoxycyclobutoxy)pyridin-2-yl)propoxy)-3- | | |
| (trifluoromethyl)benzamide | | |
| (R)-4-(1-(5-(cyclopropylmethoxy)pyridin-2-yl)propoxy)-N-((4- | +++ | +++ |
| ethylpiperazin-1-yl)sulfonyl)-3-(trifluoromethyl)benzamide | | |
| 4-((R)-1-(5-((1R,3R)-3-methoxycyclobutoxy)pyridin-2-yl)propoxy)-N- | +++ | ++ |
| ((4-((1-methylpiperidin-4-yl)oxy)piperidin-1-yl)sulfonyl)-3- | | |
| (trifluoromethyl)benzamide | | |
| 4-((R)-1-(5-((1R,3R)-3-(difluoromethoxy)cyclobutoxy)pyridin-2- | ++ | ++ |
| yl)propoxy)-2,3-difluoro-N-((4-methylpiperazin-1- | | |
| yl)sulfonyl)benzamide | | |
| (R)-N-((4-ethylpiperazin-1-yl)sulfonyl)-4-(1-(5-(pyridin-2- | +++ | +++ |
| yloxy )pyridin-2-yl)propoxy)-3-(trifluoromethyl)benzamide | | |
| 4-((R)-1-(5-((1R,3R)-3-methoxycyclobutoxy)pyridin-2-yl)propoxy)-N- | ++ | ++ |
| ((4-methylpiperazin-1-yl)sulfonyl)-3-(trifluoromethyl)benzamide | | |
| N-((4-cyclopropylpiperazin-1-yl)sulfonyl)-2,3-difluoro-4-((R)-1- | +++ | ++ |
| (5-((1R,3R)-3-methoxycyclobutoxy)pyridin-2- | | |
| yl)propoxy)benzamide | | |
| (R)-4-(1-(5-(cyclopropylmethoxy)pyridin-2-yl)propoxy)-2,3- | +++ | ++ |
| difluoro-N-((4-(2-hydroxy-2-methylpropyl)piperazin-1- | | |
| yl)sulfonyl)benzamide | | |
| N-(((S)-3,4-dimethylpiperazin-1-yl)sulfonyl)-2,3-difluoro-4-((R)-1-(5- | +++ | +++ |
| (pyridin-2-yloxy)pyridin-2-yl)propoxy)benzamide | | |
| (R)-4-(1-(3-(cyclopropylmethoxy)phenyl)propoxy)-2,3-difluoro-N-((4- | ++ | ++ |
| ((1-methylpiperidin-4-yl)oxy)piperidin-1-yl)sulfonyl)benzamide | | |
| 2,3-difluoro-4-((R)-1-(5-((1R,3R)-3-methoxycyclobutoxy)pyridin-2- | ++ | ++ |
| yl)propoxy)-N-((4-methylpiperazin-1-yl)sulfonyl)benzamide | | |
| (R)-2,3-difluoro-N-((4-(2-hydroxy-2-methylpropyl)piperazin-1- | +++ | +++ |
| yl)sulfonyl)-4-(1-(5-(pyridin-2-yloxy)pyridin-2-yl)propoxy)benzamide | | |
| (R)-2,3-difluoro-N-((4-(2-fluoro-2-methylpropyl)piperazin-1- | +++ | +++ |
| yl)sulfonyl)-4-(1-(5-(pyridin-2-yloxy)pyridin-2-yl)propoxy)benzamide | | |
| (R)-4-(1-(5-(cyclopropylmethoxy)pyridin-2-yl)propoxy)-2,3-difluoro-N- | +++ | +++ |
| ((4-(2-fluoro-2-methylpropyl)piperazin-1-yl)sulfonyl)benzamide | | |
| (R)-4-(1-(4-(cyclopropylmethoxy)pyridin-2-yl)propoxy)-2,3-difluoro-N- | ++ | ++ |
| ((4-methylpiperazin-1-yl)sulfonyl)benzamide | | |
| (R)-N-((4-cyclobutylpiperazin-1-yl)sulfonyl)-4-(1-(5- | +++ | +++ |
| (cyclopropylmethoxy)pyridin-2-yl)propoxy)-2,3-difluorobenzamide | | |
| (R)-N-((4-cyclobutylpiperazin-1-yl)sulfonyl)-2,3-difluoro-4-(1-(5- | +++ | +++ |
| (pyridin-2-yloxy)pyridin-2-yl)propoxy)benzamide | | |
| N-((4-ethylpiperazin-1-yl)sulfonyl)-4-((R)-1-(5-((1R,3R)-3- | ++ | |
| methoxycyclobutoxy)pyridin-2-yl)propoxy)-3- | | |
| (trifluoromethyl)benzamide | | |
| 2,3-difluoro-4-((R)-1-(4-((1R,3R)-3-methoxycyclobutoxy)pyridin-2- | + | |
| yl)propoxy)-N-((4-methylpiperazin-1-yl)sulfonyl)benzamide | | |
| 2,3-difluoro-4-((R)-1-(4-((1R,3R)-3-methoxycyclobutoxy)pyridin-2- | + | |
| yl)propoxy)-N-((4-((1-methylpiperidin-4-yl)oxy)piperidin-1- | | |
| yl)sulfonyl)benzamide | | |
| (R)-4-(1-(5-(cyclopropylmethoxy)pyridin-2-yl)propoxy)-2,3-difluoro-N- | ++ | |
| ((4-((1-methylazetidin-3-yl)oxy)piperidin-1-yl)sulfonyl)benzamide | | |
| (R)-2,3-difluoro-N-((4-((1-methylazetidin-3-yl)oxy)piperidin-1- | +++ | |
| yl)sulfonyl)-4-(1-(5-(pyridin-2-yloxy)pyridin-2-yl)propoxy)benzamide | | |
| (R)-2,3-difluoro-N-((6-methyl-2,6-diazaspiro[3.3]heptan-2-yl)sulfonyl)- | ++ | |
| 4-(1-(5-(pyridin-2-yloxy)pyridin-2-yl)propoxy)benzamide | | |
| (R)-4-(1-(5-(cyclopropylmethoxy)pyridin-2-yl)propoxy)-2,3-difluoro-N- | ++ | |
| ((6-methyl-2,6-diazaspiro[3.3]heptan-2-yl)sulfonyl)benzamide | | |
| 2,3-difluoro-N-((8-methyl-3,8-diazabicyclo[3.2.1]octan-3-yl)sulfonyl)- | +++ | |
| 4-((R)-1-(5-(pyridin-2-yloxy)pyridin-2-yl)propoxy)benzamide | | |
| 2,3-difluoro-N-((4-(((S)-1-methylpiperidin-3-yl)oxy)phenyl)sulfonyl)-4- | +++ | |
| ((R)-1-(5-(pyridin-2-yloxy)pyridin-2-yl)propoxy)benzamide | | |
| (R)-4-(1-(5-(cyclopropylmethoxy)pyridin-2-yl)propoxy)-2,3-difluoro-N- | ++ | |
| ((3-((1-methylpiperidin-4-yl)oxy)azetidin-1-yl)sulfonyl)benzamide | | |
| (R)-2,3-difluoro-N-((3-((1-methylpiperidin-4-yl)oxy)azetidin-1- | ++ | |
| yl)sulfonyl)-4-(1-(5-(pyridin-2-yloxy)pyridin-2-yl)propoxy)benzamide | | |
| N-((4-ethylpiperazin-1-yl)sulfonyl)-2,3-difluoro-4-((R)-1-(5-((1R,3R)- | ++ | |
| 3-methoxycyclobutoxy)pyridin-2-yl)propoxy)benzamide | | |
| (R)-4-(1-(5-(cyclopropylmethoxy)pyridin-2-yl)propoxy)-2,3-difluoro-N- | ++ | |
| ((5-methyl-3,4,5,6-tetrahydropyrrolo[3,4-c]pyrrol-2(1H)- | | |
| yl)sulfonyl)benzamide | | |
| 2,3-difluoro-N-((4-(((R)-1-methylpiperidin-3-yl)oxy)phenyl)sulfonyl)- | +++ | |
| 4-((R)-1-(5-(pyridin-2-yloxy)pyridin-2-yl)propoxy)benzamide | | |
| (R)-2,3-difluoro-N-((5-methyl-3,4,5,6-tetrahydropyrrolo[3,4-c]pyrrol- | ++ | |
| 2(1H)-yl)sulfonyl)-4-(1-(5-(pyridin-2-yloxy)pyridin-2- | | |
| yl)propoxy)benzamide | | |
| (R)-2,3-difluoro-N-((4-(3-fluoropropyl)piperazin-1-yl)sulfonyl)-4-(1-(5- | +++ | |
| (pyridin-2-yloxy)pyridin-2-yl)propoxy)benzamide | | |
| (R)-4-(1-(5-(cyclopropylmethoxy)pyridin-2-yl)propoxy)-2,3-difluoro-N- | +++ | |
| ((4-(3-fluoropropyl)piperazin-1-yl)sulfonyl)benzamide | | |
| (R)-4-(1-(5-(cyclopropylmethoxy)pyridin-2-yl)propoxy)-2,3-difluoro-N- | +++ | |
| ((4-(2-(methylamino)ethoxy)phenyl)sulfonyl)benzamide | | |
| 4-((R)-1-(5-(cyclopropylmethoxy)pyridin-2-yl)propoxy)-2,3-difluoro-N- | +++ | |
| ((4-(((S)-1-methylpiperidin-3-yl)oxy)phenyl)sulfonyl)benzamide | | |
| 4-((R)-1-(5-(cyclopropylmethoxy)pyridin-2-yl)propoxy)-2,3-difluoro-N- | ++ | |
| ((4-(((R)-1-methylpiperidin-3-yl)oxy)phenyl)sulfonyl)benzamide | | |
| 2,3-difluoro-N-((6-methyl-3,6-diazabicyclo[3.1.1]heptan-3-yl)sulfonyl)- | +++ | |
| 4-((R)-1-(5-(pyridin-2-yloxy)pyridin-2-yl)propoxy)benzamide | | |

### 4.5.47. Example 47. Efficacy in a Murine Diet-Induced Obesity Model

The effects of four representative compounds of the invention were tested in a mouse model of diet-induced obesity (DIO):
Compound A is (R)—N-(tert-butylsulfonyl)-4-(1-(5-(pyridin-3-yloxy)pyridin-2-yl)ethoxy)-3-(trifluoromethyl)benzamide;
Compound B is (R)-4-(1-(5-(cyclopropylmethoxy)pyridin-2-yl)propoxy)-N-((4-(2-(dimethylamino)ethoxy)phenyl)sulfonyl)-2,3-difluorobenzamide;
Compound C is (R)—N-((4-(2-(dimethylamino)ethoxy)piperidin-1l-yl)sulfonyl)-2,3-difluoro-4-(1-(5-(pyridin-2-yloxy)pyridin-2-yl)propoxy)benzamide; and
Compound D is (R)-2,3-difluoro-N-((4-methylpiperazin-1-yl)sulfonyl)-4-(1-(5-(pyridin-2-yloxy)pyridin-2-yl)propoxy)benzamide.

The mice used in these experiments were C₅₇BL/6J mice fed a high-fat diet (D12492 from Research Diets) ad libitum during the study period. The vehicle and test article formulations were 10% w/v Solutol in water.

### Study Design and Results: Compound A

After a seven-day acclimation period, the DIO model was induced by putting the mice on a high fat diet for ten weeks. After the ten weeks, the DIO mice were equally divided into groups based on body weight, ten mice in each group: the control group (Vehicle) and Compound A at 30 mg/kg.

The animals were pre-dosed for two days prior to formal dosing (once-per-day treatment for every group). From Day 1 to Day 13, the animals in the dosing group were administered orally by gavage at 5:00 p.m. every day as well as weight and food intake were recorded daily. All animals were fasted overnight after dosing on Day 1, Day 4, Day 12 and Day 13, then the blood (retro-orbital bleed) of mice from each group was collected at 7:00 a.m. to separate out the serum on Day 2, Day 5 and Day 13 for measuring total cholesterol levels (TC), triglyceride (TG), aminotransferase alanine (ALT), aspartate aminotransferase (AST) and total bilirubin (TBil) levels. All animals were performed OGTT test (0 min, 30 min, 60 min and 120 min) and measured insulin levels (0 min and 30 min) on Day 14.

### Method

1. Body weight and Food Consumption: Pre-dose every day starting from Day −1 onwards.
2. Day −1 and Day 0: Handle mice and gavage with vehicle.
3. Day 1: Dose˜5:00 PM. Fast overnight.
4. Day 2: Determine total cholesterol levels and triglyceride, ALT, AST, Total Bilirubin levels 7:00 AM. Resume feeding.
5. Day 2-3: Dose˜5:00 PM.
6. Day 4: Dose˜5:00 PM. Fast overnight.
7. Day 5: Determine total cholesterol levels and triglyceride, ALT, AST, Total Bilirubin levels˜7:00 AM. Resume feeding.
8. Day 5˜11: Dose˜5:00 PM.
9. Day 12: Dose˜5:00 PM. Fast overnight.
10. Day 13: Determine total cholesterol levels and triglyceride, ALT, AST, Total Bilirubin levels 7:00 AM. Resume feeding.
11. Day 13: Dose˜5:00 PM. Fast overnight.
12. Day 14: (No drug administration) Oral glucose tolerance test with glucose levels measured at Pre-dose, 30 min, 60 min and 120 min. And insulin levels at Time 0 min, 30 min after the glucose challenge.

### Statistical Analysis

Data was provided as mean±SD. Statistical significance was considered when the P value is less than 0.05. Results were analyzed by SPSS software. Results were analyzed by SPSS software. T-test was used to calculate P value. Statistical significance was considered when the P value is less than 0.05.

### Results

The body weights of the control group mice were decreased from Day −1 to about Day 5. From Day 6 to Day 12, the overall body weight of the mice in the control group stabilized at about 35 g. The mean body weight of the Compound A 30 mg/kg group decreased from Day −1 to Day 12 and was significantly lower than that of the control group from Day 8 onwards (P<0.01 or 0.05 or 0.001). Data from this experiment are presented below in Table 6.

TABLE 6
Effect of Compound A on Body Weight

| | Body Weight (g)/Day | | | | | | |
|---|---|---|---|---|---|---|---|
| Treatment | Day −1 | Day 0 | Day 1 | Day 2 | Day 3 | Day 4 | Day 5 |
| Control | 38.5 ± 1.6 | 38.3 ± 1.6 | 37.9 ± 1.5 | 36.6 ± 1.4 | 36.1 ± 1.3 | 35.5 ± 1.2 | 34.7 ± 1.3 |
| 30 mg/kg | 38.5 ± 1.6 | 38.3 ± 1.4 | 37.9 ± 1.4 | 36.6 ± 1.4 | 35.8 ± 1.5 | 35.3 ± 1.4 | 34.3 ± 1.3 |

| | Body Weight (g)/Day | | | | | | |
|---|---|---|---|---|---|---|---|
| Treatment | Day 6 | Day 7 | Day 8 | Day 9 | Day 10 | Day 11 | Day 12 |
| Control | 35.1 ± 1.3 | 34.9 ± 1.3 | 35.3 ± 1.4 | 35.4 ± 1.5 | 35.8 ± 1.6 | 35.4 ± 1.6 | 35.7 ± 1.7 |
| 30 mg/kg | 34.2 ± 1.2 | 34.0 ± 1.3 | 33.9 ± 1.4* | 33.5 ± 1.4** | 33.4 ± 1.3** | 32.9 ± 1.4** | 32.8 ± 1.3*** |

*P < 0.05,
**P < 0.01,
***P < 0.001 vs. Control;
^{#}P < 0.01,
^{##}P < 0.01 vs. Compound A at 30 mg/kg.

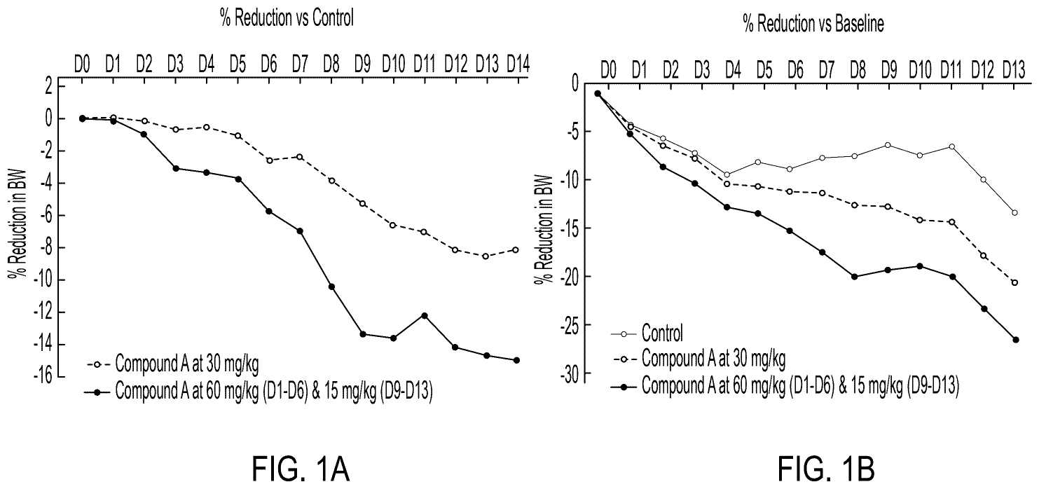

From these data, it is clear that Compound A had a statistically significant effect on the mice's body weights versus control. This is all the more apparent from FIG. 1A, which shows the percent reduction in the treated mice's mean body weight versus those of the control group, and FIG. 1B, which shows the percent reduction in the mean body weight of the treated and control groups versus baseline.

### Study Design and Results: Compounds B-D

In this study, three compounds tested at 30 mg/kg were compared against vehicle control over a month. After their acclimation period, DIO mice were equally divided into groups of ten mice each based on body weight. The animals were pre-dosed for three days prior to formal dosing (once per day treatment every group), as well as weight and food intake were recorded from Day −2 onwards. From Day 1 to Day 27, the animals in the dosing group were administered orally by gavage at 5:00 p.m. every day. All animals were fasted overnight after dosing on Day 28 and Day 29. Then the blood (retro-orbital bleed) of mice from each group was collected at 7:00 a.m. to separate out the serum for measuring total cholesterol levels (TC), triglyceride (TG), aminotransferase alanine (ALT), aspartate aminotransferase (AST) and total bilirubin (TBil) levels on Day 29. On day 30, all animals were performed OGTT test (0 min, 15 min, 30 min, 60 min, 90 min and 120 min) and measured insulin levels (0 min, 15 min, 30 min and 60 min). On day 32, all animals were injected insulin after 4 hours fasting, then performed ITT test (0 min, 15 min, 30 min, 60 min and 90 min). On day 33, Epididymal white adipose tissue (eWAT), jejunum, liver, heart and brain were collected; eWAT, liver and heart weights were recorded. All of the adipose tissue in the abdominal cavity was collected and weighted, and the fatty tissue coefficient (body fat ratio) was calculated and then stored at −80° C. for inspection.

### Method

1. Body weight and Food Consumption: Pre-dose every day from Day −2 onwards.
2. Day −2,—1 and Day 0: Handle mice and gavage with vehicle.
3. Day 1˜27: Dose˜5:00 PM.
4. Day 28: Dose˜5:00 PM. Fast overnight.
5. Day 29: Determine total cholesterol levels and triglyceride, ALT, AST, total bilirubin levels˜7:00 AM. Resume feeding.
6. Day 29: Dose˜5:00 PM. Fast overnight.
7. Day 30: (No drug administration) Oral glucose tolerance test (OGTT) with glucose levels measured at Pre-dose, 15 min, 30 min, 60 min, 90 min and 120 min after the glucose challenge. And insulin levels will be test at time 0 min, 15 min, 30 min and 60 min after the glucose challenge.
8. Day 30˜31: Dose˜5:00 PM.
9. Day 32: (No drug administration) ITT test (two days after the OGTT test): Fast 4 h. After insulin injection, the levels of glucose will be measured at Pre-dose, 15 min, 30 min, 60 min and 90 min.
10. After the ITT test. Collect serum with a terminal bleed from all 50 mice.
11. Collect epidydimal WAT (eWAT) pad, jejunum, liver, heart, and brain.
12. Record eWAT, liver, and heart weights.
13. Before freezing tissue/serum:
   (i) Assess Drug Concentration; serum, liver, eWAT, heart, jujenum, and brain. (25 serum samples; 5 mice/group will progress to biomarker study and remaining 25 serum samples saved for future analysis).
14. Prepare liver, heart and eWAT tissue for histopathology analysis.
   (ii) Liver for all 50 mice; HE staining to assess liver steatosis.
   (iii) Heart or all 50 mice; HE and DAPI staining to assess cardiac morphology. Cut the heart in the traverse plain, in the middle then into cassettes for paraffin embedding.
   (iv) eWAT; one part (5 mice/group) for drug concentration and one part (5 mice/group); HE staining to assess adipocyte morphology
   (v) Brain for all 50 mice; freeze and store for inspection
15. Collect all the adipose tissue in the abdominal cavity, weight, and calculate (include eWAT weight) the fatty tissue coefficient (body fat ratio). Then store at −80 degrees for inspection.

### Statistical Analysis

Data was provided as mean±SD. Statistical significance was considered when the P value is less than 0.05. Results were analyzed by SPSS software. When the data conformed to normal distribution and homogeneity of variance, parametric methods (t-test for two comparisons and one-way analysis of variance (ANOVA) for multiple comparisons) was selected. If the data did not conform to a normal distribution, they were analyzed using nonparametric methods (rank sum test).

### Results

The mean body weight of the control group showed a trend of slow increase from Day −2 to Day 28, while the mean body weights of the treatment groups were consistently lower than that of the control group from Day 2 onwards, and the weight difference gradually increased. The mean body weights of the Compound B and D groups were significantly lower than that of the control group from Day 15 to the end. The mean body weight of the Compound C group was significantly lower than that of the control group from Day 23.

The overall food consumption of mice in the Compound B, C, and D groups was significantly lower than that of the control group.

When the OGTT experiment was performed, the blood glucose of all groups rose sharply from 0 to 15 minutes and followed by a slightly increase from 30 minutes to 120 minutes and reached a peak at 120 minutes. At 0 minutes, the blood glucose of all treatment groups was significantly lower than that of the control group. The blood glucose of each of the treatment groups was lower than that of the control group at 15 minutes, 30 minutes, 60 minutes, and 90 minutes.

When the ITT experiment was performed the blood glucose of all treatment groups showed no significant deference than that of the control group at 0 minutes. The blood glucose of all groups was clearly lower than that of the control group at 15 minutes, 30 minutes, 60 minutes and 90 minutes. The blood glucose of the Compound B group at 30 minutes and 60 minutes, the blood glucose of the Compound C group and the Compound D group at 15 minutes, 30 minutes, 60 minutes and 90 minutes was significantly lower than that of the control group. Thus, each of these compounds provided significant resistance to a decrease in blood glucose caused by injection of insulin in DIO mice.

The eWAT weights of the Compound D group were significantly lower than that of the control group. The eWAT coefficient of the Compound B and D groups was slightly lower than that of the control group, but there was no significant difference in the eWAT coefficient of mice in all groups.

The mean adipose weights of mice in the Compound B, Compound C, and Compound D groups were significantly lower than that of the control group. The adipose coefficients of the treatment groups were also lower than that of the control group.

The liver weights of treatment groups were slightly lower than that of the control group, but there was no significant difference in the liver weights of mice in all groups. There was no significant difference in the heart weights of mice in all groups. Data from this experiment are presented in Table 6.

TABLE 6

| | Body Weight (g)/Day | | | | | | |
|---|---|---|---|---|---|---|---|
| Treatment | Day −2 | Day −1 | Day 0 | Day 1 | Day 2 | Day 3 | Day 4 |
| Control | 44.7 ± 5.6 | 44.4 ± 5.8 | 44.4 ± 5.9 | 44.5 ± 6.1 | 44.3 ± 6.2 | 44.1 ± 6.3 | 44.6 ± 6.4 |
| Compound B | 42.5 ± 4.5* | 41.7 ± 4.6* | 42.1 ± 4.8* | 42.2 ± 4.7* | 42.2 ± 4.5* | 42.3 ± 4.7* | 43 ± 3.9 |
| Compound C | 44.8 ± 5.2 | 44.9 ± 5.3 | 44.9 ± 5.1 | 45 ± 4.9 | 44.8 ± 5 | 44.3 ± 4.9 | 44.3 ± 4.9 |
| Compound D | 44.9 ± 4.1 | 44.8 ± 4.1 | 44.9 ± 4.3 | 44.9 ± 4.3 | 44.3 ± 4 | 43.6 ± 3.9 | 43.7 ± 3.8 |

| | Body Weight (g)/Day | | | | | | |
|---|---|---|---|---|---|---|---|
| Treatment | Day 5 | Day 6 | Day 7 | Day 8 | Day 9 | Day 10 | Day 11 |
| Control | 45 ± 6.3 | 45.3 ± 6.4 | 45.4 ± 6.3 | 45.9 ± 6.5 | 46.4 ± 6.6 | 46.5 ± 6.3 | 46.7 ± 6.2 |
| Compound B | 43.1 ± 3.9 | 43 ± 4.1 | 42.8 ± 4 | 43.1 ± 4.3 | 42.9 ± 4.5 | 43.3 ± 4.5 | 43.1 ± 4.6 |
| Compound C | 44.1 ± 4.9 | 44 ± 4.7 | 43.5 ± 4.9 | 44 ± 5 | 44.2 ± 4.8 | 44.2 ± 4.7 | 44.4 ± 4.8 |
| Compound D | 43.7 ± 3.6 | 43.5 ± 3.6 | 43.4 ± 3.5 | 43.7 ± 3.6 | 43.5 ± 3.6 | 43.3 ± 3.5 | 42.9 ± 3.4 |

| | Body Weight (g)/Day | | | | | | |
|---|---|---|---|---|---|---|---|
| Treatment | Day 12 | Day 13 | Day 14 | Day 15 | Day 16 | Day 17 | Day 18 |
| Control | 46.8 ± 6.2 | 46.5 ± 5.9 | 46.8 ± 5.9 | 47.7 ± 6 | 47.9 ± 6 | 47.8 ± 5.9 | 48 ± 5.9 |
| Compound B | 42.7 ± 4.6 | 42.5 ± 4.6 | 42.3 ± 4.6 | 42.6 ± 4.5* | 42.5 ± 4.5* | 41.7 ± 4.6* | 42.1 ± 4.8* |
| Compound C | 43.9 ± 4.9 | 43.7 ± 4.7 | 43.7 ± 4.7 | 43.9 ± 4.8 | 44.1 ± 4.8 | 43.5 ± 4.7 | 43.6 ± 4.6 |
| Compound D | 42.6 ± 3.4 | 42.3 ± 3.4 | 42.3 ± 3.3 | 42.4 ± 3.2* | 42.3 ± 3.2* | 42.1 ± 3.4* | 42.2 ± 3.4* |

| | Body Weight (g)/Day | | | | | | |
|---|---|---|---|---|---|---|---|
| Treatment | Day 19 | Day 20 | Day 21 | Day 22 | Day 23 | Day 24 | Day 25 |
| Control | 48.3 ± 5.9 | 48.6 ± 5.9 | 49.1 ± 6 | 49.2 ± 6.1 | 49.5 ± 5.9 | 49.8 ± 5.8 | 50.1 ± 5.6 |
| Compound B | 42.2 ± 4.7* | 42.2 ± 4.5* | 42.3 ± 4.7* | 42.4 ± 4.7* | 42.6 ± 4.7* | 42.6 ± 4.4** | 42.5 ± 4.8** |
| Compound C | 43.9 ± 4.9 | 44.1 ± 4.8 | 44.2 ± 5 | 44.1 ± 5.1 | 44.1 ± 5.3* | 43.8 ± 5.3* | 43.9 ± 5.2* |
| Compound D | 42.3 ± 3.5* | 42.3 ± 3.6* | 42.3 ± 3.6** | 42.3 ± 3.7** | 42.3 ± 3.7** | 42.2 ± 3.8** | 42.4 ± 3.4** |

| | Body Weight (g)/Day | | | | | | |
|---|---|---|---|---|---|---|---|
| Treatment | Day 26 | Day 27 | Day 28 | Day 29 | Day 30 | Day 31 | Day 32 |
| Control | 50.3 ± 5.6 | 50.6 ± 5.5 | 50.8 ± 5.5 | 50.1 ± 5.2 | 47.7 ± 5 | 48.1 ± 5.2 | 47.5 ± 5.1 |
| Compound B | 42.8 ± 4.7** | 43 ± 4.8** | 42.9 ± 4.7** | 42.3 ± 4.4** | 40.4 ± 4.2** | 40.9 ± 4.2** | 40.3 ± 4.1** |
| Compound C | 43.9 ± 5.2* | 44.1 ± 5.2* | 43.9 ± 5.3** | 43.2 ± 5.2** | 41.4 ± 5* | 41.6 ± 5.2* | 41 ± 5** |
| Compound D | 42.5 ± 3.5** | 42.5 ± 3.7** | 42.5 ± 3.9*** | 41.8 ± 3.8*** | 39.5 ± 3.6*** | 39.7 ± 3.9*** | 38.9 ± 3.8*** |

*P < 0.05,
**P < 0.01,
***P < 0.001 vs. Control.

The body weight of the control group showed a trend of slow increase from Day −2 to Day 28, while the body weights of the treatment groups were consistently lower than that of the control group from Day 2 onwards, and the weight difference gradually increased. The mean body weights of the Compound B and Compound D groups were significantly lower than that of the control group from Day 15 to the end (P<0.05, P<0.01, P<0.001), while the mean body weight of the Compound C group was significantly lower than that of the control group from Day 23 (P<0.05, P<0.01).

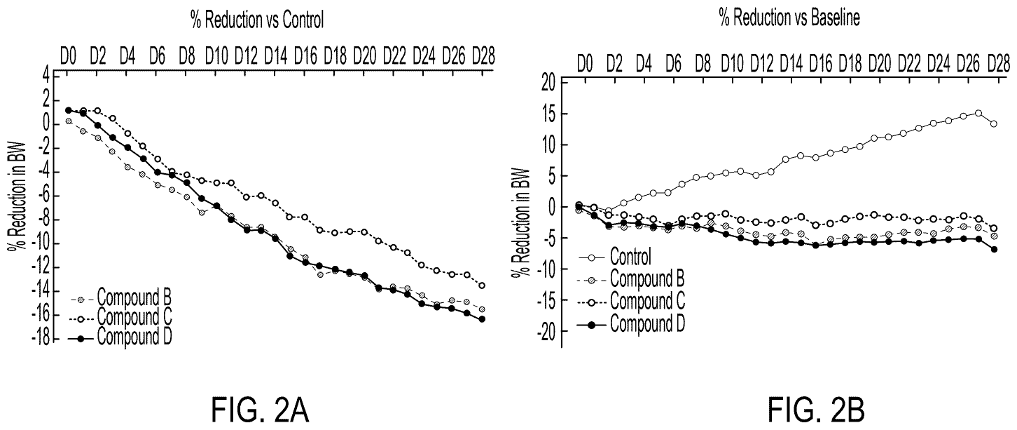

From these data, it is readily apparent that compounds of the invention had a statistically significant effect on the body weight of DIO mice over time. This is all the more apparent from FIG. 2A, which shows the percent reduction in the treated mice's mean body weight versus those of the control group, and FIG. 2B, which shows the percent reduction in the mean body weight of the treated and control groups versus baseline.

All publications (e.g., patents and patent applications) cited above are incorporated herein by reference in their entireties.

1. A compound, which compound is a compound of formula I: 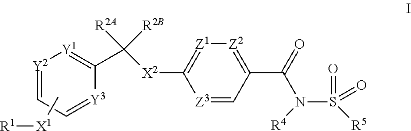
or a pharmaceutically acceptable salt thereof, wherein:
X¹ and X² is independently O or CHR⁶;
each of Y¹, Y², and Y³ is independently N, CR³, or C—X¹—R¹, provided that only one of Y¹, Y², and Y³ may be attached to X¹;
each of Z¹, Z², and Z³ is independently N or CR³;
each R¹ is independently optionally substituted C₁₋₆ hydrocarbyl or 2-9 membered heterocarbyl, which optional substitution is with one or more of halo, R^{1A}, or OR^{1A};
each R^{1A} is independently optionally substituted C₁₋₆ hydrocarbyl, which optional substitution is with one or more of halo;
each of R^{2A} and R^{2B} is independently hydrogen or C₁₋₆ alkyl optionally substituted with one or more halo, or R^{2A} and R^{2B} are taken together to form a C₃₋₅ cycloalkyl or 3-5 membered cycloheteroalkyl;
each R³ is independently hydrogen, halo, C₁₋₆ alkyl optionally substituted with one or more halo;
R⁴ is hydrogen or C₁₋₆ alkyl;
R⁵ is N(R^{5A})₂, NHR^{5A}, or optionally substituted C₁₋₈ hydrocarbyl or 2-9 membered heterocarbyl, which optional substitution is with one or more of halo, hydroxyl, nitrile, RA, OR^{5A}, N(R^{5A})₂, NHR^{5A}, or NH₂;
each R^{5A} is independently optionally substituted C₁₋₈ hydrocarbyl or 2-9 membered heterocarbyl, which optional substitution is with one or more of halo, hydroxyl, nitrile, R^{5B}, OR^{5B}, N(R^{5B})₂, NHR⁵B, or NH₂;
each R^{5B} is independently optionally substituted C₁₋₈ hydrocarbyl or 2-9 membered heterocarbyl, which optional substitution is with one or more of halo, hydroxyl, nitrile, R^{C}, OR^{5C}, N(R^{5C})₂, NHR^{C}, or NH₂;
each R^{5C} is independently optionally substituted C₁₋₈ hydrocarbyl or 2-9 membered heterocarbyl, which optional substitution is with one or more of halo, hydroxyl, nitrile, or NH₂; and
each R⁶ is independently H or C₁₋₆ alkyl.

2. (canceled)

3. (canceled)

4. (canceled)

5. The compound of claim 1, wherein at least one of Y¹, Y², and Y³ is N.

6. (canceled)

7. (canceled)

8. (canceled)

9. (canceled)

10. The compound of claim 1, wherein Z³ is CR³.

11. The compound of claim 1, which is of formula III: 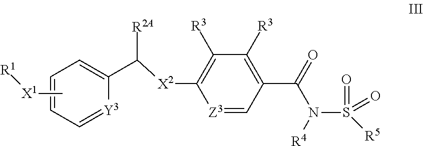
or a pharmaceutically acceptable salt thereof.

12. (canceled)

13. (canceled)

14. The compound of claim 1, which is of formula IV: 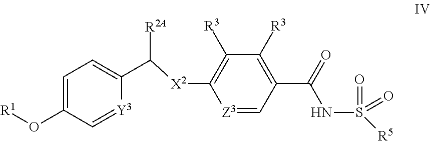
or a pharmaceutically acceptable salt thereof.

15. The compound of claim 14, wherein Z³ is CR³.

16. (canceled)

17. (canceled)

18. The compound of claim 1, which is of formula V(a): 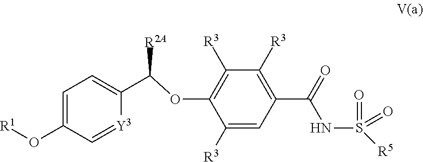
or a pharmaceutically acceptable salt thereof.

19. (canceled)

20. The compound of claim 18, wherein Y³ is N.

21. The compound of claim 20, wherein R⁵is one of:

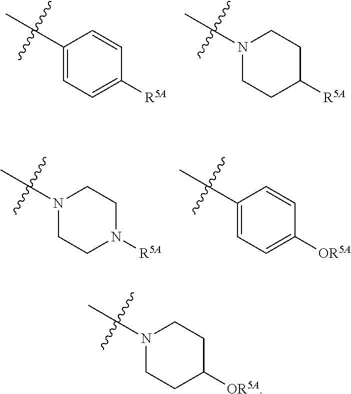

22. (canceled)

23. (canceled)

24. (canceled)

25. (canceled)

26. (canceled)

27. (canceled)

28. The compound of claim 21, wherein R¹ is optionally substituted C₁₋₈ hydrocarbyl.

29. The compound of claim 28, wherein R¹ is cyclopropylmethyl, phenyl, or cyclopropyl.

30. (canceled)

31. (canceled)

32. (canceled)

33. (canceled)

34. The compound of claim 29, wherein R^{2A} is methyl or ethyl.

35. The compound of claim 34, wherein R^{2B} is hydrogen.

36. (canceled)

37. (canceled)

38. (canceled)

39. The compound of claim 35, wherein at least one R³ is fluoro.

40. (canceled)

41. The compound of claim 21, wherein R^{5A} is optionally substituted with OR⁵B.

42. (canceled)

43. The compound of claim 1, which compound is:
N-((4-(2-aminoethyl)phenyl)sulfonyl)-2,3-difluoro-4-((3-phenoxybenzyl)oxy)benzamide;
N-((2-aminoethyl)sulfonyl)-2,3-dichloro-4-(1-(3-phenoxyphenyl)ethoxy)benzamide;
rac-(R)-2,3-dichloro-N-((4-methylpiperazin-1-yl)sulfonyl)-4-(1-(3-phenoxyphenyl)ethoxy)benzamide;
rac-(R)—N-((4-(aminomethyl)phenyl)sulfonyl)-2,3-dichloro-4-(1-(3-phenoxyphenyl)ethoxy)benzamide;
rac-(R)—N-((2-aminoethyl)sulfonyl)-2,3-dichloro-4-(1-(3-phenoxyphenyl)ethoxy)benzamide;
N-((4-((dimethylamino)methyl)phenyl)sulfonyl)-2,3-difluoro-4-((3-phenoxybenzyl)oxy)benzamide;
2,3-difluoro-N-((4-((methylamino)methyl)phenyl)sulfonyl)-4-((3-phenoxybenzyl)oxy)benzamide;
N-((4-(aminomethyl)phenyl)sulfonyl)-2,3-difluoro-4-((4-phenoxybenzyl)oxy)benzamide;
N-((4-(aminomethyl)phenyl)sulfonyl)-4-(1-(3-(cyclopentyloxy)phenyl)ethoxy)-2,3-difluorobenzamide;
N-((4-(aminomethyl)phenyl)sulfonyl)-2,3-dichloro-4-((3-phenoxybenzyl)oxy)benzamide;
rac-(R)—N-((4-(aminomethyl)phenyl)sulfonyl)-3-methyl-4-(1-(3-phenoxyphenyl)ethoxy)benzamide;
N-((4-(aminomethyl)phenyl)sulfonyl)-2,3-difluoro-4-(1-(3-phenoxyphenyl)ethoxy)benzamide;
N-((4-(aminomethyl)phenyl)sulfonyl)-4-(1-(3-(thiophen-3-yloxy)phenyl)ethoxy)-3-(trifluoromethyl)benzamide;
N-((4-(aminomethyl)phenyl)sulfonyl)-4-(1-(3-(pyridin-3-yloxy)phenyl)ethoxy)-3-(trifluoromethyl)benzamide;
N-((4-(aminomethyl)phenyl)sulfonyl)-4-(2-(3-phenoxyphenyl)propoxy)-3-(trifluoromethyl)benzamide;
N-((4-(aminomethyl)phenyl)sulfonyl)-2,3-dichloro-4-(2-(3-phenoxyphenyl)propoxy)benzamide;
N-((4-(aminomethyl)phenyl)sulfonyl)-2,3-difluoro-4-(1-(3-(pyridin-3-yloxy)phenyl)ethoxy)benzamide;
N-((4-(aminomethyl)phenyl)sulfonyl)-4-(1-(3-(cyclopentyloxy)phenyl)ethoxy)-3-(trifluoromethyl)benzamide;
N-((4-(aminomethyl)phenyl)sulfonyl)-4-(1-(3-(pyrimidin-5-yloxy)phenyl)ethoxy)-3-(trifluoromethyl)benzamide;
N-((4-(aminomethyl)phenyl)sulfonyl)-4-(1-(3-(pyridin-2-yloxy)phenyl)ethoxy)-3-(trifluoromethyl)benzamide;
N-((4-(aminomethyl)phenyl)sulfonyl)-4-(1-(3-(pyrazin-2-yloxy)phenyl)ethoxy)-3-(trifluoromethyl)benzamide;
N-((4-(aminomethyl)phenyl)sulfonyl)-4-(1-(3-((1-methyl-1H-pyrazol-4-yl)oxy)phenyl)ethoxy)-3-(trifluoromethyl)benzamide;
N-((4-(aminomethyl)phenyl)sulfonyl)-4-(1-(3-((2-methylpentyl)oxy)phenyl)ethoxy)-3-(trifluoromethyl)benzamide;
N-((4-(aminomethyl)phenyl)sulfonyl)-4-(1-(3-(pentyloxy)phenyl)ethoxy)-3-(trifluoromethyl)benzamide;
N-((4-(aminomethyl)phenyl)sulfonyl)-4-(1-(3-(cyclopentylmethoxy)phenyl)ethoxy)-3-(trifluoromethyl)benzamide;
N-((4-(aminomethyl)phenyl)sulfonyl)-4-(cyclopentylmethoxy)-3-(trifluoromethyl)benzamide;
rac-(R)—N-((2-aminoethyl)sulfonyl)-4-(1-(3-phenoxyphenyl)ethoxy)-3-(trifluoromethyl)benzamide;
N-((3-morpholinopropyl)sulfonyl)-4-(1-(5-(pyridazin-3-yloxy)pyridin-2-yl)ethoxy)-3-(trifluoromethyl)benzamide;
4-(1-(5-ethoxypyridin-2-yl)propoxy)-N-((3-morpholinopropyl)sulfonyl)-3-(trifluoromethyl)benzamide;
4-(1-(5-ethoxypyridin-2-yl)propoxy)-N-((4-(2-(methylamino)ethoxy)phenyl)sulfonyl)-3-(trifluoromethyl)benzamide;
4-(1-(5-ethoxypyridin-2-yl)ethoxy)-2,3-difluoro-N-((4-methylpiperazin-1-yl)sulfonyl)benzamide;
4-(1-(5-ethoxypyridin-2-yl)ethoxy)-2,3-difluoro-N-(piperidin-4-ylsulfonyl)benzamide;
4-(1-(3-(cyclopropylmethoxy)phenyl)ethoxy)-N-((1-(2-(dimethylamino)ethyl)-1H-pyrazol-4-yl)sulfonyl)-2,3-difluorobenzamide;
N-((4-(2-(1H-imidazol-1-yl)ethoxy)phenyl)sulfonyl)-4-(1-(5-ethoxypyridin-2-yl)ethoxy)-2,3-difluorobenzamide;
(R)-4-(1-(5-(cyclopropylmethoxy)pyridin-2-yl)propoxy)-2,3-difluoro-N-((4-(2-morpholinoethoxy)phenyl)sulfonyl)benzamide;
(R)-4-(1-(5-(cyclopropylmethoxy)pyridin-2-yl)propoxy)-N-((4-(2-(dimethylamino)ethoxy)phenyl)sulfonyl)-2,3-difluorobenzamide;
(R)-4-(1-(5-(cyclopropylmethoxy)pyridin-2-yl)propoxy)-2,3-difluoro-N-((4-(2-(4-methylpiperazin-1-yl)ethoxy)phenyl)sulfonyl)benzamide;
(R)—N-((4-(2-(1H-imidazol-1-yl)ethoxy)phenyl)sulfonyl)-2,3-difluoro-4-(1-(5-(2-methoxyethoxy)pyridin-2-yl)propoxy)benzamide;
(R)—N-((4-(2-(dimethylamino)ethoxy)phenyl)sulfonyl)-2,3-difluoro-4-(1-(5-(2-methoxyethoxy)pyridin-2-yl)propoxy)benzamide;
(R)-4-(1-(5-(cyclopropylmethoxy)pyridin-2-yl)propoxy)-2,3-difluoro-N-((4-(2-(pyrrolidin-1-yl)ethoxy)phenyl)sulfonyl)benzamide;
4-((R)-1-(5-(cyclopropylmethoxy)pyridin-2-yl)propoxy)-2,3-difluoro-N-((4-(2-((S)-3-fluoropyrrolidin-1-yl)ethoxy)phenyl)sulfonyl)benzamide;
(R)-4-(1-(5-(cyclopropylmethoxy)pyridin-2-yl)propoxy)-2,3-difluoro-N-((4-(2-(oxetan-3-ylamino)ethoxy)phenyl)sulfonyl)benzamide;
(R)—N-((4-((1H-imidazol-4-yl)methoxy)phenyl)sulfonyl)-2,3-difluoro-4-(1-(5-(2-methoxyethoxy)pyridin-2-yl)propoxy)benzamide;
(R)—N-((4-(2-(cyclopropylamino)ethoxy)phenyl)sulfonyl)-4-(1-(5-(cyclopropylmethoxy)pyridin-2-yl)propoxy)-2,3-difluorobenzamide;
(R)-4-(1-(5-(cyclopropylmethoxy)pyridin-2-yl)propoxy)-2,3-difluoro-N-((4-((1-methylpiperidin-4-yl)oxy)piperidin-1-yl)sulfonyl)benzamide;
(R)-4-(1-(5-(cyclopropylmethoxy)pyridin-2-yl)propoxy)-N-((6-(2-(dimethylamino)ethoxy)-2-azaspiro[3,3]heptan-2-yl)sulfonyl)-2,3-difluorobenzamide;
(R)-4-(1-(5-(cyclopropylmethoxy)pyridin-2-yl)propoxy)-2,3-difluoro-N-((6-(2-(pyrrolidin-1-yl)ethoxy)-2-azaspiro[3,3]heptan-2-yl)sulfonyl)benzamide;
4-((R)-1-(5-(cyclopropylmethoxy)pyridin-2-yl)propoxy)-2,3-difluoro-N-((4-(((S)-1-methylpyrrolidin-3-yl)oxy)phenyl)sulfonyl)benzamide;
4-((R)-1-(5-(cyclopropylmethoxy)pyridin-2-yl)propoxy)-2,3-difluoro-N-((4-(((R)-1-methylpyrrolidin-3-yl)oxy)phenyl)sulfonyl)benzamide;
(R)-4-(1-(5-(cyclopropylmethoxy)pyridin-2-yl)propoxy)-N-((4-((1-ethylpiperidin-4-yl)oxy)phenyl)sulfonyl)-2,3-difluorobenzamide;
(R)-4-(1-(5-(cyclopropylmethoxy)pyridin-2-yl)propoxy)-2,3-difluoro-N-((4-((1-methylazetidin-3-yl)oxy)phenyl)sulfonyl)benzamide;
(R)-4-(1-(5-(cyclopropylmethoxy)pyridin-2-yl)propoxy)-2,3-difluoro-N-((6-((1-methylpiperidin-4-yl)oxy)pyridazin-3-yl)sulfonyl)benzamide;
(R)-4-(1-(5-(cyclopropylmethoxy)pyridin-2-yl)propoxy)-2,3-difluoro-N-((6-((1-methylpiperidin-4-yl)oxy)pyridin-3-yl)sulfonyl)benzamide;
(R)-4-(1-(5-(cyclopropylmethoxy)pyridin-2-yl)propoxy)-2,3-difluoro-N-((4-((1-(methyl-d₃)piperidin-4-yl)oxy)phenyl)sulfonyl)benzamide;
(R)-4-(1-(5-(cyclopropylmethoxy)pyridin-2-yl)propoxy)-N-((4-((1-(cyclopropylmethyl)piperidin-4-yl)oxy)phenyl)sulfonyl)-2,3-difluorobenzamide;
(R)-4-(1-(5-(cyclopropylmethoxy)pyridin-2-yl)propoxy)-2,3-difluoro-N-((4-((1-(2-hydroxyethyl)piperidin-4-yl)oxy)phenyl)sulfonyl)benzamide;
(R)-4-(1-(5-(cyclopropylmethoxy)pyridin-2-yl)propoxy)-2,3-difluoro-N-((4-((1-isopropylpiperidin-4-yl)oxy)phenyl)sulfonyl)benzamide;
(R)-4-(1-(5-(cyclopropylmethoxy)pyridin-2-yl)propoxy)-2,3-difluoro-N-((6-((1-methylpiperidin-4-yl)amino)pyridin-3-yl)sulfonyl)benzamide;
4-((R)-1-(5-(cyclopropylmethoxy)pyridin-2-yl)propoxy)-2,3-difluoro-N-((4-((12,2-trimethylpiperidin-4-yl)oxy)phenyl)sulfonyl)benzamide;
(R)-4-(1-(5-(cyclopropylmethoxy)pyridin-2-yl)propoxy)-2,3-difluoro-N-((4-(piperidin-4-yloxy)phenyl)sulfonyl)benzamide;
(R)-4-(1-(5-(cyclopropylmethoxy)pyridin-2-yl)propoxy)-2,3-difluoro-N-((4-(2-(pyrrolidin-1-yl)ethoxy)piperidin-1-yl)sulfonyl)benzamide;
(R)-4-(1-(5-(cyclopropylmethoxy)pyridin-2-yl)propoxy)-2,3-difluoro-N-((2-((1-methylpiperidin-4-yl)oxy)pyrimidin-5-yl)sulfonyl)benzamide;
4-((R)-1-(5-(cyclopropylmethoxy)pyridin-2-yl)propoxy)-2,3-difluoro-N-((4-(((1R⁴R⁵R)-2-methyl-2-azabicyclo[2.2.1]heptan-5-yl)oxy)phenyl)sulfonyl)benzamide;
4-((R)-1-(5-(cyclopropylmethoxy)pyridin-2-yl)propoxy)-2,3-difluoro-N-((5-(2-(pyrrolidin-1-yl)ethoxy)-2-azabicyclo[2.2.1]heptan-2-yl)sulfonyl)benzamide;
(R)-4-(1-(5-(cyclopropylmethoxy)pyridin-2-yl)propoxy)-N-((4-((1-(2,2-difluoroethyl)piperidin-4-yl)oxy)phenyl)sulfonyl)-2,3-difluorobenzamide;
N-((4-(((1R⁴R⁵R)-2-azabicyclo[2.2.1]heptan-5-yl)oxy)phenyl)sulfonyl)-4-((R)-1-(5-(cyclopropylmethoxy)pyridin-2-yl)propoxy)-2,3-difluorobenzamide;
(R)-4-(1-(5-(cyclopropylmethoxy)pyridin-2-yl)propoxy)-2,3-difluoro-N-((6-(2-(pyrrolidin-1-yl)ethoxy)pyridazin-3-yl)sulfonyl)benzamide;
(R)-4-(1-(5-(cyclopropylmethoxy)pyridin-2-yl)propoxy)-2,3-difluoro-N-((6-((2-(pyrrolidin-1-yl)ethyl)amino)pyridin-3-yl)sulfonyl)benzamide;
(R)-4-(1-(5-(cyclopropylmethoxy)pyridin-2-yl)propoxy)-2,3-difluoro-N-((4-((1-methylpiperidin-4-yl)oxy)phenyl)sulfonyl)benzamide;
(R)-4-(1-(5-(cyclopropylmethoxy)pyridin-2-yl)propoxy)-2,3-difluoro-N-((5-((1-methylpiperidin-4-yl)oxy)pyridin-2-yl)sulfonyl)benzamide;
(R)-4-(1-(5-(cyclopropylmethoxy)pyridin-2-yl)propoxy)-2,3-difluoro-N-((5-((1-methylpiperidin-4-yl)amino)pyridin-2-yl)sulfonyl)benzamide;
(R)-4-(1-(5-(cyclopropylmethoxy)pyridin-2-yl)propoxy)-2,3-difluoro-N-((6-((1-isopropylpiperidin-4-yl)amino)pyridin-3-yl)sulfonyl)benzamide;
(R)-4-(1-(5-(cyclopropylmethoxy)pyridin-2-yl)propoxy)-2,3-difluoro-N-((6-(2-(pyrrolidin-1-yl)ethoxy)pyridin-3-yl)sulfonyl)benzamide;
(R)-4-(1-(5-(cyclopropylmethoxy)pyridin-2-yl)propoxy)-2,3-difluoro-N-((6-((1-methylpiperidin-4-yl)oxy)-2-azaspiro[3,3]heptan-2-yl)sulfonyl)benzamide;
(R)-4-(1-(5-(cyclopropylmethoxy)pyridin-2-yl)propoxy)-2,3-difluoro-N-((6-((1-isopropylpiperidin-4-yl)oxy)pyridin-3-yl)sulfonyl)benzamide;
(R)—N-((4-((1-azabicyclo[2.2.1]heptan-4-yl)oxy)phenyl)sulfonyl)-4-(1-(5-(cyclopropylmethoxy)pyridin-2-yl)propoxy)-2,3-difluorobenzamide;
4-((R)-1-(5-(cyclopropylmethoxy)pyridin-2-yl)propoxy)-2,3-difluoro-N-((4-(2-((R)-1-methylpiperidin-2-yl)ethoxy)phenyl)sulfonyl)benzamide;
(R)-4-(1-(5-(cyclopropylmethoxy)pyridin-2-yl)propoxy)-2,3-difluoro-N-((4-(2-(piperidin-1-yl)ethoxy)phenyl)sulfonyl)benzamide;
(R)-2,3-difluoro-N-((4-((1-methylpiperidin-4-yl)oxy)piperidin-1-yl)sulfonyl)-4-(1-(5-(pyridin-2-yloxy)pyridin-2-yl)propoxy)benzamide;
(R)-4-(1-(5-(cyclopropylmethoxy)pyridin-2-yl)propoxy)-N-((6-((4-ethylpiperazin-1-yl)methyl)pyridin-3-yl)sulfonyl)-2,3-difluorobenzamide;
(R)-4-(1-(5-(cyclopropylmethoxy)pyridin-2-yl)propoxy)-2,3-difluoro-N-((4-((1-(2-methoxyethyl)piperidin-4-yl)oxy)phenyl)sulfonyl)benzamide;
(R)-4-(1-(5-(cyclopropylmethoxy)pyridin-2-yl)propoxy)-2,3-difluoro-N-((4-((1-(2-hydroxy-2-methylpropyl)piperidin-4-yl)oxy)phenyl)sulfonyl)benzamide;
(R)-4-(1-(5-(cyclopropylmethoxy)pyridin-2-yl)propoxy)-2,3-difluoro-N-((4-((1-(2-hydroxy-2-methylpropyl)piperidin-4-yl)oxy)piperidin-1-yl)sulfonyl)benzamide;
(R)-4-(1-(5-(cyclopropylmethoxy)pyridin-2-yl)propoxy)-2,3-difluoro-N-((4-((1-(2-methoxyethyl)piperidin-4-yl)oxy)piperidin-1-yl)sulfonyl)benzamide;
(R)-4-(1-(5-(cyclopropylmethoxy)pyridin-2-yl)propoxy)-N-((4-((1-(cyclopropylmethyl)piperidin-4-yl)oxy)piperidin-1-yl)sulfonyl)-2,3-difluorobenzamide;
(R)-4-(1-(5-(cyclopropylmethoxy)pyridin-2-yl)propoxy)-N-((4-((4-ethylpiperazin-1-yl)methyl)piperidin-1-yl)sulfonyl)-2,3-difluorobenzamide;
(R)-2,3-difluoro-4-(1-(5-(2-methoxyethoxy)pyridin-2-yl)propoxy)-N-((4-((1-methylpiperidin-4-yl)oxy)phenyl)sulfonyl)benzamide;
(R)—N-((4-(2-(dimethylamino)ethoxy)piperidin-1-yl)sulfonyl)-2,3-difluoro-4-(1-(5-(2-methoxyethoxy)pyridin-2-yl)propoxy)benzamide;
(R)-2,3-difluoro-4-(1-(5-(2-methoxyethoxy)pyridin-2-yl)propoxy)-N-((4-((1-methylpiperidin-4-yl)oxy)piperidin-1-yl)sulfonyl)benzamide;
(R)—N-((4-(2-(dimethylamino)ethoxy)piperidin-1-yl)sulfonyl)-2,3-difluoro-4-(1-(5-(pyridin-2-yloxy)pyridin-2-yl)propoxy)benzamide;
(R)-4-(1-(5-(cyclopropylmethoxy)pyridin-2-yl)propoxy)-N-((4-(2-(dimethylamino)ethoxy)piperidin-1-yl)sulfonyl)-2,3-difluorobenzamide;
(R)-2,3-difluoro-N-((4-methylpiperazin-1-yl)sulfonyl)-4-(1-(5-(pyridin-2-yloxy)pyridin-2-yl)propoxy)benzamide;
(R)-4-(1-(5-(cyclopropylmethoxy)pyridin-2-yl)propoxy)-2,3-difluoro-N-((1-methylpiperidin-4-yl)sulfonyl)benzamide;
(R)-2,3-difluoro-4-(1-(5-(pyridin-2-yloxy)pyridin-2-yl)propoxy)-N-((2,2,4-trimethylpiperazin-1-yl)sulfonyl)benzamide;
(R)-2,3-difluoro-N-((1-methylpiperidin-4-yl)sulfonyl)-4-(1-(5-(pyridin-2-yloxy)pyridin-2-yl)propoxy)benzamide;
(R)-4-(1-(5-(cyclopropylmethoxy)pyridin-2-yl)propoxy)-2,3-difluoro-N-((2,2,4-trimethylpiperazin-1-yl)sulfonyl)benzamide;
(R)-4-(1-(5-(cyclopropylmethoxy)pyridin-2-yl)propoxy)-N-((1-(dimethylamino)-2-methylpropan-2-yl)sulfonyl)-2,3-difluorobenzamide;
(R)-4-(1-(5-(cyclopropylmethoxy)pyridin-2-yl)propoxy)-2,3-difluoro-N-((4-methylpiperazin-1-yl)sulfonyl)benzamide;
(R)—N-((1-(dimethylamino)-2-methylpropan-2-yl)sulfonyl)-2,3-difluoro-4-(1-(5-(pyridin-2-yloxy)pyridin-2-yl)propoxy)benzamide;
(R)—N-((4-((1-methylpiperidin-4-yl)oxy)piperidin-1-yl)sulfonyl)-4-(1-(5-(pyridin-2-yloxy)pyridin-2-yl)propoxy)-3-(trifluoromethyl)benzamide;
(R)—N-((4-(2-(dimethylamino)ethoxy)phenyl)sulfonyl)-4-(1-(5-(pyridin-2-yloxy)pyridin-2-yl)propoxy)-3-(trifluoromethyl)benzamide;
(R)—N-((4-(2-(dimethylamino)ethoxy)phenyl)sulfonyl)-2,3-difluoro-4-(1-(5-(pyridin-2-yloxy)pyridin-2-yl)propoxy)benzamide;
(R)—N-((4-((1-methylpiperidin-4-yl)oxy)piperidin-1-yl)sulfonyl)-4-(1-(5-(pyridin-2-yloxy)pyridin-2-yl)ethoxy)-3-(trifluoromethyl)benzamide;
(R)—N-((4-(2-(dimethylamino)ethoxy)phenyl)sulfonyl)-4-(1-(5-(pyridin-2-yloxy)pyridin-2-yl)ethoxy)-3-(trifluoromethyl)benzamide;
(R)-4-(1-(5-(cyclopropylmethoxy)pyridin-2-yl)propoxy)-2,3-difluoro-N-((4-isopropylpiperazin-1-yl)sulfonyl)benzamide;
(R)-4-(1-(5-(cyclopropylmethoxy)pyridin-2-yl)propoxy)-N-((4-cyclopropylpiperazin-1-yl)sulfonyl)-2,3-difluorobenzamide;
4-((R)-1-(5-(cyclopropylmethoxy)pyridin-2-yl)propoxy)-2,3-difluoro-N-((4-((1-methylpiperidin-3-yl)oxy)phenyl)sulfonyl)benzamide;
2,3-difluoro-N-((4-((1-methylpiperidin-3-yl)oxy)phenyl)sulfonyl)-4-((R)-1-(5-(pyridin-2-yloxy)pyridin-2-yl)propoxy)benzamide;
(R)-4-(1-(5-(cyclopropylmethoxy)pyridin-2-yl)propoxy)-2,3-difluoro-N-((4-methyl-1,4-diazepan-1-yl)sulfonyl)benzamide;
(R)-4-(1-(5-(cyclopropylmethoxy)pyridin-2-yl)propoxy)-2,3-difluoro-N-((3,3,4-trimethylpiperazin-1-yl)sulfonyl)benzamide;
4-((R)-1-(5-(cyclopropylmethoxy)pyridin-2-yl)propoxy)-2,3-difluoro-N-(((3S,5R)-3,4,5-trimethylpiperazin-1-yl)sulfonyl)benzamide;
(R)—N-((4-(2-(dimethylamino)ethoxy)phenyl)sulfonyl)-2,3-difluoro-4-(1-(5-(pyridin-2-yloxy)pyridin-2-yl)ethoxy)benzamide;
(R)-4-(1-(5-(cyclopropylmethoxy)pyridin-2-yl)propoxy)-2,3-difluoro-N-((4-(2-methoxyethyl)piperazin-1-yl)sulfonyl)benzamide;
4-((R)-1-(5-(cyclopropylmethoxy)pyridin-2-yl)propoxy)-2,3-difluoro-N-((6-methyl-3,6-diazabicyclo[3,1,1]heptan-3-yl)sulfonyl)benzamide;
4-((R)-1-(5-(cyclopropylmethoxy)pyridin-2-yl)propoxy)-N—(((S)-3,4-dimethylpiperazin-1-yl)sulfonyl)-2,3-difluorobenzamide;
4-((R)-1-(5-(cyclopropylmethoxy)pyridin-2-yl)propoxy)-N—(((R)-3,4-dimethylpiperazin-1-yl)sulfonyl)-2,3-difluorobenzamide;
(R)-2,3-difluoro-N-((4-((1-(2-methoxyethyl)piperidin-4-yl)oxy)piperidin-1-yl)sulfonyl)-4-(1-(5-(pyridin-2-yloxl)pyridin-2-yl)propoxy)benzamide;
4-((R)-1-(5-(cyclopropylmethoxy)pyridin-2-yl)propoxy)-2,3-difluoro-N-((8-methyl-3,8-diazabicyclo[3,2,1]octan-3-yl)sulfonyl)benzamide;
N-((4-(2-(dimethylamino)ethoxy)phenyl)sulfonyl)-2,3-difluoro-4-((R)-1-(5-((1R,3R)-3-methoxycyclobutoxy)pyridin-2-yl)propoxy)benzamide;
2,3-difluoro-4-((R)-1-(5-((1R³R)-3-methoxycyclobutoxy)pyridin-2-yl)propoxy)-N-((4-((1-methylpiperidin-4-yl)oxy)piperidin-1-yl)sulfonyl)benzamide;
4-((R)-1-(5-(cyclopropylmethoxy)pyridin-2-yl)propoxy)-2,3-difluoro-N-(((1S,4S)-5-methyl-2,5-diazabicyclo[2.2.1]heptan-2-yl)sulfonyl)benzamide;
4-((R)-1-(5-(cyclopropylmethoxy)pyridin-2-yl)propoxy)-2,3-difluoro-N-(((1R,4R)-5-methyl-2,5-diazabicyclo[2.2.1]heptan-2-yl)sulfonyl)benzamide;
(R)—N-((4-ethylpiperazin-1-yl)sulfonyl)-2,3-difluoro-4-(1-(5-(pyridin-2-yloxy)pyridin-2-yl)propoxy)benzamide;
(R)-4-(1-(5-(cyclopropylmethoxy)pyridin-2-yl)propoxy)-N-((4-ethylpiperazin-1-yl)sulfonyl)-2,3-difluorobenzamide;
(R)—N-((4-methylpiperazin-1-yl)sulfonyl)-4-(1-(5-(pyridin-2-yloxy)pyridin-2-yl)propoxy)-3-(trifluoromethyl)benzamide;
(R)-4-(1-(5-(cyclopropylmethoxy)pyridin-2-yl)propoxy)-N-((4-((1-methylpiperidin-4-yl)oxy)piperidin-1-yl)sulfonyl)-3-(trifluoromethyl)benzamide;
(R)-4-(1-(5-(cyclopropylmethoxy)pyridin-2-yl)propoxy)-N-((4-methylpiperazin-1-yl)sulfonyl)-3-(trifluoromethyl)benzamide;
(R)—N-((4-cyclopropylpiperazin-1-yl)sulfonyl)-2,3-difluoro-4-(1-(5-(pyridin-2-yloxy)pyridin-2-yl)propoxy)benzamide;
(R)—N-((4-cyclohexylpiperazin-1-yl)sulfonyl)-4-(1-(5-(cyclopropylmethoxy)pyridin-2-yl)propoxy)-2,3-difluorobenzamide;
(R)—N-((4-cyclohexylpiperazin-1-yl)sulfonyl)-2,3-difluoro-4-(1-(5-(pyridin-2-yloxy)pyridin-2-yl)propoxy)benzamide;
(R)-4-(1-(5-(cyclopropylmethoxy)pyridin-2-yl)propoxy)-N-((4-(2-(dimethylamino)ethoxy)phenyl)sulfonyl)-3-(trifluoromethyl)benzamide;
(R)-4-(1-(4-(cyclopropylmethoxy)pyridin-2-yl)propoxy)-2,3-difluoro-N-((4-((1-methylpiperidin-4-yl)oxy)piperidin-1-yl)sulfonyl)benzamide;
(R)-4-(1-(4-(cyclopropylmethoxy)pyridin-2-yl)propoxy)-N-((4-(2-(dimethylamino)ethoxy)phenyl)sulfonyl)-2,3-difluorobenzamide;
(R)-4-(1-(4-(cyclopropylmethoxy)pyridin-2-yl)propoxy)-2,3-difluoro-N-((4-((1-methylpiperidin-4-yl)oxy)phenyl)sulfonyl)benzamide;
(R)-2,3-difluoro-N-((4-((1-methylpiperidin-4-yl)oxy)piperidin-1-yl)sulfonyl)-4-(1-(4-(pyridin-2-yloxy)pyridin-2-yl)propoxy)benzamide;
(R)-2,3-difluoro-N-((4-methylpiperazin-1-yl)sulfonyl)-4-(1-(4-(pyridin-2-yloxy)pyridin-2-yl)propoxy)benzamide;
(R)—N-((4-cyclopropylpiperazin-1-yl)sulfonyl)-2,3-difluoro-4-(1-(4-(pyridin-2-yloxy)pyridin-2-yl)propoxy)benzamide;
(R)—N-((4-(2-(dimethylamino)ethoxy)phenyl)sulfonyl)-2,3-difluoro-4-(1-(4-(pyridin-2-yloxy)pyridin-2-yl)propoxy)benzamide;
(R)-4-(1-(5-(cyclopropylmethoxy)pyridin-2-yl)propoxy)-N-((4-(cyclopropylmethyl)piperazin-1-yl)sulfonyl)-2,3-difluorobenzamide;
(R)—N-((4-(cyclopropylmethyl)piperazin-1-yl)sulfonyl)-2,3-difluoro-4-(1-(5-(pyridin-2-yloxy)pyridin-2-yl)propoxy)benzamide;
(R)—N-((4-cyclopentylpiperazin-1-yl)sulfonyl)-4-(1-(5-(cyclopropylmethoxy)pyridin-2-yl)propoxy)-2,3-difluorobenzamide;
(R)—N-((4-cyclopentylpiperazin-1-yl)sulfonyl)-2,3-difluoro-4-(1-(5-(pyridin-2-yloxy)pyridin-2-yl)propoxy)benzamide;
N-((4-(2-(dimethylamino)ethoxy)phenyl)sulfonyl)-4-((R)-1-(5-((1R3R)-3-methoxycyclobutoxy)pyridin-2-yl)propoxy)-3-(trifluoromethyl)benzamide;
(R)-4-(1-(5-(cyclopropylmethoxy)pyridin-2-yl)propoxy)-N-((4-ethylpiperazin-1-yl)sulfonyl)-3-(trifluoromethyl)benzamide;
4-((R)-1-(5-((1R,3R)-3-methoxyvclobutoxy)pyridin-2-yl)propoxy)-N-((4-((1-methylpiperidin-4-yl)oxy)piperidin-1-yl)sulfonyl)-3-(trifluoromethyl)benzamide;
4-((R)-1-(5-((1R,3R)-3-(difluoromethoxy)cyclobutoxy)pyridin-2-yl)propoxy)-2,3-difluoro-N-((4-methylpiperazin-1-yl)sulfonyl)benzamide;
(R)—N-((4-ethylpiperazin-1-yl)sulfonyl)-4-(1-(5-(pyridin-2-yloxy)pyridin-2-yl)propoxy)-3-(trifluoromethyl)benzamide;
4-((R)-1-(5-((1R,3R)-3-methoxyevclobutoxy)pyridin-2-yl)propoxy)-N-((4-methylpiperazin-1-yl)sulfonyl)-3-(trifluoromethyl)benzamide;
N-((4-cyclopropylpiperazin-1-yl)sulfonyl)-2,3-difluoro-4-((R)-1-(5-((1R,3R)-3-methoxycyclobutoxy)pyridin-2-yl)propoxy)benzamide;
(R)-4-(1-(5-(cyclopropylmethoxy)pyridin-2-yl)propoxy)-2,3-difluoro-N-((4-(2-hydroxy-2-methylpropyl)piperazin-1-yl)sulfonyl)benzamide;
N—(((S)-3,4-dimethylpiperazin-1-yl)sulfonyl)-2,3-difluoro-4-((R)-1-(5-(pyridin-2-yloxy)pyridin-2-yl)propoxy)benzamide;
(R)-4-(1-(3-(cyclopropylmethoxy)phenyl)propoxy)-2,3-difluoro-N-((4-((1-methylpiperidin-4-yl)oxy)piperidin-1-yl)sulfonyl)benzamide;
2,3-difluoro-4-((R)-1-(5-((1R3R)-3-methoxycyclobutoxy)pyridin-2-yl)propoxy)-N-((4-methylpiperazin-1-yl)sulfonyl)benzamide;
(R)-2,3-difluoro-N-((4-(2-hydroxy-2-methylpropyl)piperazin-1-yl)sulfonyl)-4-(1-(5-(pyridin-2-yloxy)pyridin-2-yl)propoxy)benzamide;
(R)-2,3-difluoro-N-((4-(2-fluoro-2-methylpropyl)piperazin-1-yl)sulfonyl)-4-(1-(5-(pyridin-2-yloxy)pyridin-2-yl)propoxy)benzamide;
(R)-4-(1-(5-(cyclopropylmethoxy)pyridin-2-yl)propoxy)-2,3-difluoro-N-((4-(2-fluoro-2-methylpropyl)piperazin-1-yl)sulfonyl)benzamide;
(R)-4-(1-(4-(cyclopropylmethoxy)pyridin-2-yl)propoxy)-2,3-difluoro-N-((4-methylpiperazin-1-yl)sulfonyl)benzamide;
(R)—N-((4-cyclobutylpiperazin-1-yl)sulfonyl)-4-(1-(5-(cyclopropylmethoxy)pyridin-2-yl)propoxy)-2,3-difluorobenzamide;
(R)—N-((4-cyclobutylpiperazin-1-yl)sulfonyl)-2,3-difluoro-4-(1-(5-(pyridin-2-yloxy)pyridin-2-yl)propoxy)benzamide;
N-((4-ethylpiperazin-1-yl)sulfonyl)-4-((R)-1-(5-((1R3R)-3-methoxycyclobutoxy)pyridin-2-yl)propoxy)-3-(trifluoromethyl)benzamide;
2,3-difluoro-4-((R)-1-(4-((1R3R)-3-methoxycyclobutoxy)pyridin-2-yl)propoxy)-N-((4-methylpiperazin-1-yl)sulfonyl)benzamide;
2,3-difluoro-4-((R)-1-(4-((1R3R)-3-methoxycyclobutoxy)pyridin-2-yl)propoxy)-N-((4-((1-methylpiperidin-4-yl)oxy)piperidin-1-yl)sulfonyl)benzamide;
(R)-4-(1-(5-(cyclopropylmethoxy)pyridin-2-yl)propoxy)-2,3-difluoro-N-((4-((1-methylazetidin-3-yl)oxy)piperidin-1-yl)sulfonyl)benzamide;
(R)-2,3-difluoro-N-((4-((1-methylazetidin-3-yl)oxy)piperidin-1-yl)sulfonyl)-4-(1-(5-(pyridin-2-yloxy)pyridin-2-yl)propoxy)benzamide;
(R)-2,3-difluoro-N-((6-methyl-2,6-diazaspiro[3,3]heptan-2-yl)sulfonyl)-4-(1-(5-(pyridin-2-yloxy)pyridin-2-yl)propoxy)benzamide;
(R)-4-(1-(5-(cyclopropylmethoxy)pyridin-2-yl)propoxy)-2,3-difluoro-N-((6-methyl-2,6-diazaspiro[3,3]heptan-2-yl)sulfonyl)benzamide;
23-difluoro-N-((8-methyl-3,8-diazabicyclo[3,2,1]octan-3-yl)sulfonyl)-4-((R)-1-(5-(pyridin-2-yloxy)pyridin-2-yl)propoxy)benzamide;
2,3-difluoro-N-((4-(((S)-1-methylpiperidin-3-yl)oxy)phenyl)sulfonyl)-4-((R)-1-(5-(pyridin-2-yloxy)pyridin-2-yl)propoxy)benzamide;
(R)-4-(1-(5-(cyclopropylmethoxy)pyridin-2-yl)propoxy)-2,3-difluoro-N-((3-((1-methylpiperidin-4-yl)oxy)azetidin-1-yl)sulfonyl)benzamide;
(R)-2,3-difluoro-N-((3-((1-methylpiperidin-4-yl)oxy)azetidin-1-yl)sulfonyl)-4-(1-(5-(pyridin-2-yloxy)pyridin-2-yl)propoxy)benzamide;
N-((4-ethylpiperazin-1-yl)sulfonyl)-2,3-difluoro-4-((R)-1-(5-((1R3R)-3-methoxycyclobutoxy)pyridin-2-yl)propoxy)benzamide;
(R)-4-(1-(5-(cyclopropylmethoxy)pyridin-2-yl)propoxy)-2,3-difluoro-N-((5-methyl-3,4,5,6-tetrahydropyrrolo[3,4-c]pyrrol-2(1H)-yl)sulfonyl)benzamide;
2,3-difluoro-N-((4-(((R)-1-methylpiperidin-3-yl)oxy)phenyl)sulfonyl)-4-((R)-1-(5-(pyridin-2-yloxy)pyridin-2-yl)propoxy)benzamide;
(R)-2,3-difluoro-N-((5-methyl-3,4,5,6-tetrahydropyrrolo[3,4-c]pyrrol-2(1H)-yl)sulfonyl)-4-(1-(5-(pyridin-2-yloxy)pyridin-2-yl)propoxy)benzamide;
(R)-2,3-difluoro-N-((4-(3-fluoropropyl)piperazin-1-yl)sulfonyl)-4-(1-(5-(pyridin-2-yloxy)pyridin-2-yl)propoxy)benzamide;
(R)-4-(1-(5-(cyclopropylmethoxy)pyridin-2-yl)propoxy)-2,3-difluoro-N-((4-(3-fluoropropyl)piperazin-1-yl)sulfonyl)benzamide;
(R)-4-(1-(5-(cyclopropylmethoxy)pyridin-2-yl)propoxy)-2,3-difluoro-N-((4-(2-(methylamino)ethoxy)phenyl)sulfonyl)benzamide;
4-((R)-1-(5-(cyclopropylmethoxy)pyridin-2-yl)propoxy)-2,3-difluoro-N-((4-(((S)-1-methylpiperidin-3-yl)oxy)phenyl)sulfonyl)benzamide;
4-((R)-1-(5-(cyclopropylmethoxy)pyridin-2-yl)propoxy)-2,3-difluoro-N-((4-(((R)-1-methylpiperidin-3-yl)oxy)phenyl)sulfonyl)benzamide; or
2,3-difluoro-N-((6-methyl-3,6-diazabicyclo[3,1,1]heptan-3-yl)sulfonyl)-4-((R)-1-(5-(pyridin-2-yloxy)pyridin-2-yl)propoxy)benzamide;
or a pharmaceutically acceptable salt thereof.

44. A pharmaceutical composition comprising a compound of claim 1 and a pharmaceutically acceptable excipient.

45. A method of preparing a compound of formula 1h: 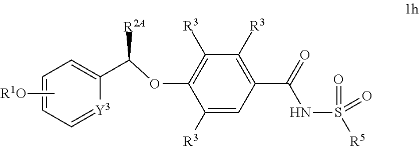
which comprises contacting a compound of formula if: 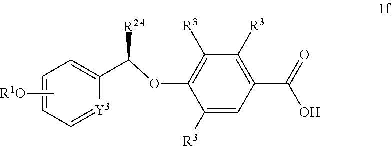
with a compound of formula 1g: 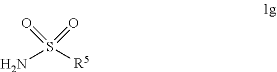
in mixture under conditions sufficient to provide the compound of formula 1h, wherein:
Y³ is N or CR³;
each R¹ is independently optionally substituted C₁₋ₛ hydrocarbyl or 2-9 membered heterocarbyl, which optional substitution is with one or more of halo, RA, or OR^{1A};
each R^{1A} is independently optionally substituted C₁₋₆ hydrocarbyl, which optional substitution is with one or more of halo;
R^{2A} is C₁₋₆ alkyl optionally substituted with one or more halo;
each R³ is independently hydrogen, halo, or C₁₋₆ alkyl optionally substituted with one or more halo;
R⁵ is N(R^{5A})₂, NHR^{5A}, or optionally substituted C₁₋₆ hydrocarbyl or 2-9 membered heterocarbyl, which optional substitution is with one or more of halo, hydroxyl, RA, OR^{5A}, N(RA)₂, NHR^{5A}, or NH₂;
each R^{5A} is independently optionally substituted C₁₋₆ hydrocarbyl or 2-9 membered heterocarbyl, which optional substitution is with one or more halo, hydroxyl, or R^{5B}, OR^{5B}, N(R^{5B})₂, NHR^{5B}, or NH₂;
each R^{5B} is independently optionally substituted C₁₋₈ hydrocarbyl or 2-9 membered heterocarbyl, which optional substitution is with one or more of halo, hydroxyl, or R^{5C}, OR^{5C}, N(R^{5C})₂, NHR^{5C}, or NH₂; and
each R^{5C} is independently optionally substituted C₁₋₈ hydrocarbyl or 2-9 membered heterocarbyl, which optional substitution is with one or more of halo, hydroxyl, or NH₂.

46. (canceled)

47. (canceled)

48. (canceled)

49. (canceled)

50. A method of inhibiting acyl-CoA synthetase 5 (ACSL5), which comprises contacting ACSL5 with a compound of claim 1.

51. A method of treating or managing a metabolic disease or disorder, which comprises administering to a patient in need thereof a therapeutically or prophylactically effective amount of a compound of claim 1.

52. (canceled)

53. (canceled)

54. (canceled)

55. (canceled)
